(19) United States
(12) Patent Application Publication
(10) Pub. No.: US 2024/0368250 A1
(43) Pub. Date: Nov. 7, 2024

(54) POLYPEPTIDES BINDING TO THE NEONATAL FC RECEPTOR

(71) Applicants: Ablynx N.V., Zwijnaarde (BE); Sanofi, Paris (FR)

(72) Inventors: Tom Van Bogaert, Gentbrugge (BE); Judith Verhelst, Gent (BE); Carlo Boutton, Wielsbeke (BE); Wilbert De Witte, Gent (BE)

(73) Assignees: Ablynx N.V., Zwijnaarde (BE); Sanofi, Paris (FR)

(21) Appl. No.: 18/443,751

(22) Filed: Feb. 16, 2024

(30) Foreign Application Priority Data
Feb. 17, 2023 (EP) 23305217.4

(51) Int. Cl. C07K 14/765

(52) U.S. Cl. CPC C07K 14/765; C07K 2317/14; C07K 2317/24; C07K 2317/569; C07K 2317/92; C07K 2319/30

(57) ABSTRACT

The present technology relates to polypeptides binding to the neonatal Fc receptor. More particularly, the present technology provides polypeptides binding to the neonatal Fc receptor and comprising (i) at least one domain comprising a serum albumin protein and/or at least one domain specifically binding to a serum albumin protein and (ii) an Fc domain of an immunoglobulin G (IgG).

### REFERENCE TO AN ELECTRONIC SEQUENCE LISTING

The contents of the electronic sequence listing (A084870230US00-SEQ-JRV.xml; Size: 297,330 bytes; and Date of Creation: Feb. 16, 2024) is herein incorporated by reference in its entirety.

### 1. TECHNOLOGICAL FIELD

The present technology relates to polypeptides binding to the neonatal Fc receptor. More particularly, the present technology provides polypeptides binding to the neonatal Fc receptor and comprising (i) at least one domain comprising a serum albumin protein and/or at least one domain specifically binding to a serum albumin protein and (ii) an Fc domain of an immunoglobulin G (IgG).

The technology further relates to constructs, compounds, molecules or chemical entities that comprise at least one of these polypeptides.

Also, the present technology relates to methods for producing such polypeptides as well as to uses of such polypeptides for diverse applications, including but not limited to the extension of the half-life and/or reduction of clearance in vivo of therapeutic compounds and/or other groups or moieties, and/or the prevention and/or treatment of a disease and/or disorder, such as but not limited to a proliferative disease, an inflammatory disease, an infectious disease or an autoimmune disease.

### 2. TECHNOLOGICAL BACKGROUND

Peptides and proteins are two classes of molecules with attractive possibilities for therapeutic applications. However, the bottleneck for their development to clinically and commercially relevant pharmaceuticals is their short half-life in vivo, which is typically just a few minutes to hours.

The half-life of peptides and proteins in human serum is dictated by several factors, including size, charge, proteolytic sensitivity, nature of their biology, turnover rate of proteins they bind, and others. Those that have a molecular weight smaller than approximately 70 kDa are predominantly eliminated via kidney filtration and generally possess very short serum half-lives. Larger proteins may persist in circulation for several days.

Albumin and IgG, the two most abundant soluble proteins present in blood circulation, are an exception to most proteins in circulation in that they share the remarkable property of having a prolonged serum half-life of about 19 to 21 days in human. A key player in the regulation of plasma half-life of IgG and albumin is a cellular receptor named the neonatal Fc receptor (FcRn). FcRn is a heterodimer consisting of an N-glycosylated transmembrane MHC class I-like heavy chain that is noncovalently associated with soluble b2-microglobulin. Both IgG and albumin are ligands binding to different epitopes of FcRn. Their interaction with FcRn is strictly pH-dependent with strong binding occurring at acidic pH<6.5 and no binding at neutral physiological pH. In general, the cellular model for half-life regulation relies on uptake of IgG or albumin, likely via fluid-phase pinocytosis, followed by binding to FcRn in acidified endosomes, where the receptor predominately resides. The FcRn-IgG or FcRn-albumin complex is then routed away from lysosomal degradation and recycled to the cell surface where exposure to a near neutral blood pH results in release of IgG or albumin into the extracellular environment.

The currently most explored strategies for extending the half-life of peptide- and protein-based therapeutics are based on the above-described FcRn-mediated recycling mechanism by directly or indirectly attaching the therapeutically active compound to either albumin or an Fc domain.

However, up to now, extending the in vivo half-life of therapeutic peptides and small proteins up to or beyond the half-life of albumin or full-length antibodies has not been achieved.

Most protein- or peptide-Fc fusion proteins generated to date have half-life values in humans of only 4 to 21 days. These low values may be due to a lowered affinity to FcRn compared to that of the structure of a conventional antibody. Fc fusion proteins exhibit shorter half-life when compared with the whole IgG (which has a half-life of about 3 weeks). The factors influencing this are complex, and include a generally lower binding affinity to FcRn, lower stability, the clearance pathway of effector molecules and a lack of the Fab domain.

Similarly, even though serum albumin has a half-life in humans of about 19 days, the half-life of for example albiglutide (GLP-1-HSA fusion protein Tanzeum® (US) or Eperzan® (EU) also known as Albugon) is only about 5 days. Thus far, other fusion partners tested in the clinic, such as CTP or ELP have done no better, with the fusion proteins possessing half-life values of 2.5 or 4-5 days, respectively.

For most of the above-described proteins, the best dosing schedule to be expected would be weekly, with some potentially requiring two doses per week. While this is far better than the native peptides or proteins alone, it is still far more frequent dosing than that of most therapeutic antibodies. At present, almost all protein-based drug formulations available in market are administered intravenously or subcutaneously with high dosing at frequent interval, eventually creating dose-fluctuation-related complications and reducing patient compliance vastly.

Accordingly, there is a need for extended serum persistence for peptide and protein-based therapeutics, and/or any other group or moiety, resulting in a more even serum concentration of the drug/moiety, lower dosage without compromising efficacy and lower dosing frequency. This may well translate into less toxicity and side effects, as well as improved compliance.

### 3. SUMMARY OF THE PRESENT TECHNOLOGY

The present inventors have identified polypeptides that bind specifically and/or are otherwise directed to FcRn, which polypeptides comprise (i) at least one domain comprising a serum albumin protein and/or at least one domain specifically binding to a serum albumin protein and (ii) an Fc domain of an immunoglobulin G (IgG).

The FcRn binding polypeptides as provided by the present technology have the advantage of showing a significantly increased in vivo serum half-life and reduced clearance as compared to known half-life extending peptides and proteins, including full length immunoglobulins, as described in the prior art. Accordingly, the polypeptides with an extended in vivo persistence in blood circulation according to the present technology can be used for various applications, including but not limited to prolonging the in vivo half-life of (existing or future) therapeutic compounds and/or reducing its clearance. The benefits of extending the half-life of a therapeutic molecule will be readily apparent to those skilled in the art. Such benefits include lower doses and/or frequencies of administration, which reduce the risk of adverse events in the subject and reduce costs. Accordingly, therapeutics with extended half-life have a substantial added value as regards pharmaceutical significance.

In one aspect, the present technology provides polypeptides, such as FcRn targeting polypeptides, that comprise (i) at least one domain comprising a serum albumin protein and/or at least one domain that specifically binds to a serum albumin protein and (ii) an Fc domain of an immunoglobulin G (IgG) or a fragment thereof.

In particular embodiments, the present technology provides polypeptides, such as FcRn targeting polypeptides that comprise (i) at least one domain comprising a serum albumin protein and (ii) an Fc domain of an immunoglobulin G (IgG) or a fragment thereof.

In particular embodiments, the present technology provides polypeptides, such as FcRn targeting polypeptides that comprise (i) at least one domain that specifically binds to a serum albumin protein and (ii) an Fc domain of an immunoglobulin G (IgG), or a fragment thereof.

In particular embodiments, the present technology provides polypeptides, such as FcRn targeting polypeptides that comprise (i) at least one domain comprising a serum albumin protein and at least one domain that specifically binds to a serum albumin protein and (ii) an Fc domain of an immunoglobulin G (IgG), or a fragment thereof.

In particular embodiments, the at least one domain that comprises a serum albumin protein is a part, a fragment, a derivative or variant of a serum albumin protein.

In particular embodiments, the at least one domain that comprises a serum albumin protein is human serum albumin or a part, a fragment, a derivative or variant of human serum albumin.

In further particular embodiments, the at least one domain specifically binding to a serum albumin protein specifically binds to amino acid residues on the serum albumin protein that are not involved in binding of the serum albumin protein to FcRn.

In further particular embodiments, the at least one domain specifically binding to a serum albumin protein specifically binds to domain II of human serum albumin.

In particular embodiments, the at least one domain that specifically binds to a serum albumin protein is a peptide or protein comprising between 5 and 500 amino acids.

In yet further particular embodiments, the at least one domain specifically binding to a serum albumin protein is chosen from the group consisting of an Affibody® (affibody molecule), a scFv, a Fab, a Designed Ankyrin Repeat Protein (DARPin®), an Albumin Binding Domain (ABD), a Nanofitin® (aka affitin) and an immunoglobulin variable domain sequence (ISVD).

In certain further particular embodiments, the at least one domain specifically binding to a serum albumin protein is at least one ISVD.

In further particular embodiments, the at least one domain specifically binding to a serum albumin protein is at least one ISVD specifically binding to domain II of serum albumin, such as domain II of human serum albumin.

In certain further particular embodiments, the present technology provides FcRn targeting polypeptides, characterized in that the at least one domain specifically binding to a serum albumin protein is at least one ISVD specifically binding to human serum albumin, wherein the ISVD is a (single) domain antibody, a Nanobody® VHH, a VHH, a humanized VHH, or a camelized VH.

In certain further particular embodiments, the at least one domain specifically binding to a serum albumin protein is at least one ISVD specifically binding to human serum albumin, which ISVD essentially consists of 4 framework regions (FR1 to FR4, respectively) and 3 complementarity determining regions (CDR1 to CDR3 respectively), wherein:
a. CDR1 comprises the amino acid sequence according to Kabat numbering of SEQ ID NO: 1 or has 3, 2 or 1 amino acid difference(s) with SEQ ID NO: 1;
b. CDR2 comprises the amino acid sequence according to Kabat numbering of SEQ ID NO: 2 or has 3, 2 or 1 amino acid difference(s) with SEQ ID NO: 2; and
c. CDR3 comprises the amino acid sequence according to Kabat numbering of SEQ ID NO: 3 or has 3, 2 or 1 amino acid difference(s) with SEQ ID NO: 3.

In certain further particular embodiments, the at least one domain specifically binding to a serum albumin protein is at least one ISVD specifically binding to human serum albumin, which ISVD essentially consists of 4 framework regions (FR1 to FR4, respectively) and 3 complementarity determining regions (CDR1 to CDR3 respectively), wherein:
a. CDR1 comprises the amino acid sequence according to AbM numbering of SEQ ID NO: 4 or has 3, 2 or 1 amino acid difference(s) with SEQ ID NO: 4;
b. CDR2 comprises the amino acid sequence according to AbM numbering of SEQ ID NO: 5 or has 3, 2 or 1 amino acid difference(s) with SEQ ID NO: 5; and
c. CDR3 comprises the amino acid sequence according to AbM numbering of SEQ ID NO: 6 or has 3, 2 or 1 amino acid difference(s) with SEQ ID NO: 6.

In certain further particular embodiments, the at least one domain specifically binding to a serum albumin protein is at least one ISVD that specifically binds to human serum albumin and that has:
a. a degree of sequence identity with any one of the sequences with SEQ ID NOs: 7 to 21 or 61-69 (in which the CDR's and any C-terminal extension that may be present are not taken into account for determining the degree of sequence identity) of at least 85%, preferably at least 90%, more preferably at least 95%; and/or
b. no more than 7, preferably no more than 5, such as only 3, 2 or 1 “amino acid differences” (as defined herein, and not taking into account the CDRs and any C-terminal extension that may be present) with the sequence of SEQ ID NOs: 7 to 21 or 61-69.

In further particular embodiments, the at least one domain specifically binding to serum albumin is at least one ISVD specifically binding to human serum albumin and having a sequence that is chosen from the group consisting of SEQ ID NO's: 7 to 21 and 61 to 69.

In yet further particular embodiments, the at least one peptide or protein specifically binding to serum albumin is at least one ISVD specifically binding to serum albumin with a dissociation constant (K_{D}) of between 10⁻⁶ M and 10⁻¹¹ M or less, as determined using Proteon, Kinexa, BLI or SPR.

In particular embodiments, the FcRn binding polypeptides further comprise at least one Fc domain of an IgG chosen from the group consisting of an Fc domain of an immunoglobulin G type 1, (IgG1), an Fc domain of an immunoglobulin G type 2 (IgG2), an Fc domain of an immunoglobulin G type 3 (IgG3) and an Fc domain of an immunoglobulin G type 4 (IgG4), preferably IgG1 or IgG4, even more preferably IgG4.

In further particular embodiments, the at least one Fc domain of an IgG is a native (i.e., wild-type) Fc domain of an IgG or a part or fragment thereof.

In other further embodiments, the at least one Fc domain of an IgG is a variant Fc domain of an IgG or a part or fragment thereof.

In particular embodiments, the at least one Fc domain of an IgG binds FcRn with a K_{D} value of less than about 550, 525, 500, 475, 450, 425, 400, 375, 350, 325, 300, 275, 250, 225, 200, 175, 150, 125, 100 nM at a pH of between about 5.0 and 6.8, preferably at a pH of about 6.0. In further particular embodiments, the at least one Fc domain of an IgG binds FcRn, at a pH of between about 5.0 and 6.8, preferably at a pH of about 6.0, with a K_{D} value of less than about 75 nM, such as less than 50 nM, 25 nM, 20 nM, 17 nM, 15 nM, 10 nM, 5 nM, 2.5 nM, 1 nM (at a pH of between 5.0 and 6.8). In yet further particular embodiments, the at least one Fc domain of an IgG binds FcRn at a pH of between about 5.0 and 6.8, preferably at a pH of about 6.0 with a K_{D} value of between about 250 nM and 1 nM, such as between 100 nM and 1 nM, preferably between 75 nM and 1 nM, such as between 50 nM and 1 nM, most preferably between 25 nM and 1 nm, such as about 20 nM, such as about 17 nM.

In particular embodiments, the polypeptides according to the present technology are such that they have a serum half-life in human (expressed as t_{1/2} beta) that is more than 6 hours, preferably more than 12 hours, more preferably of more than 24 hours, even more preferably more than 72 hours; for example of about one week, two weeks and up to the half-life of serum albumin in man (estimated to be around 19 days), and even up to three weeks, four weeks, one month, two months to three months and more.

In particular embodiments, the polypeptides according to the present technology are such that they have a serum half-life in man that is at least 5%, such as at least 10%, at least 25%, at least 50%, at least 100%, up to 200%, 300%, 400% and 500% or more of the half-life of serum albumin in man.

In particular embodiments, the at least one ISVD contains, compared to any of the sequences of SEQ ID NO's: 7 to 21 or 61-69, one or more mutations that reduce the binding by pre-existing antibodies.

In particular embodiments, the at least one ISVD is a VHH and contains, compared to any of the sequences of SEQ ID NOs: 7 to 21 or 61-69, one or more humanizing substitutions.

In particular embodiments, the present technology provides polypeptides binding to FcRn as described herein, characterized in that the polypeptides further comprise a therapeutic moiety.

In particular embodiments, the present technology provides polypeptides binding to FcRn as described herein, characterized in that the polypeptides further comprise a therapeutic moiety, which comprises an ISVD such as a (single) domain antibody, a Nanobody® VHH, a VHH, a humanized VHH or a camelized VH.

In a further aspect, the present technology provides nucleic acids or nucleic acid sequences encoding polypeptides according to the present technology.

In another aspect, the present technology provides vectors comprising nucleic acids or nucleic acid sequences according to the present technology.

In yet another aspect, the present technology provides host cells or (non-human) host organisms transformed or transfected with the nucleic acids or nucleic acid sequences according to the present technology or with the vectors according to the present technology.

In a further aspect, the present technology provides a method or process for producing the polypeptides according to technology, said method at least comprising the steps of:
a. expressing, in a suitable host cell or (non-human) host organism or in another suitable expression system, a nucleic acid sequence (encoding the polypeptide according to the present technology); optionally followed by:
b. isolating and/or purifying the polypeptides according to the technology.

In yet a further aspect, the present technology provides pharmaceutical compositions comprising the polypeptides according to the present technology, or the polypeptides produced by the processes according to the present technology.

In a further aspect, the present technology provides polypeptides of the technology, or polypeptides produced according to the processes of the technology, for use in treating a subject in need thereof.

In a further aspect, the present technology provides methods for delivering a prophylactic or therapeutic polypeptide to a specific location, tissue or cell type in the body, the methods comprising the steps of administering to a subject, the polypeptides of the present technology, or produced by the processes according to the present technology.

In a further aspect, the present technology provides polypeptides of the present technology, or produced according to the processes of the present technology, for use in delivering a prophylactic or therapeutic polypeptide to a specific location, tissue or cell type in the body.

In yet a further aspect, the present technology provides polypeptides of the present technology, or produced according to the process of the present technology, for use in therapy.

In yet a further aspect, the present technology provides polypeptides of the present technology, or produced according to the process of the present technology, for use in the prevention, treatment or amelioration of a disease selected from the group consisting of a proliferative disease, an inflammatory disease, an infectious disease and an autoimmune disease.

In another aspect, the present technology provides methods for the prevention, treatment or amelioration of a disease selected from the group consisting of a proliferative disease, an inflammatory disease, an infectious disease and an autoimmune disease, comprising at least the step of administering to a subject in need thereof the polypeptide of the present technology, or produced by a method of the present technology.

In a further aspect, the present technology provides kits comprising polypeptides of the present technology, nucleic acids or nucleic acid sequences of the present technology, vectors of the present technology, or host cells of the present technology.

Unless indicated or defined otherwise, all terms used have their usual meaning in the art, which will be clear to the skilled person. Reference is for example made to the standard handbooks mentioned in paragraph a) on page 46 of WO 08/020079.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain, using no more than routine experimentation, many equivalents to the specific embodiments of the technology described herein. Such equivalents are intended to be encompassed by the present technology.

The term “and/or” wherever used herein includes the meaning of “and”, “or” and “all or any other combination of the elements connected by said term”.

The term “about” used in the context of the parameters or parameter ranges of the provided herein shall have the following meanings. Unless indicated otherwise, where the term “about” is applied to a particular value or to a range, the value or range is interpreted as being as accurate as the method used to measure it. If no error margins are specified in the application, the last decimal place of a numerical value indicates its degree of accuracy. Where no other error margins are given, the maximum margin is ascertained by applying the rounding-off convention to the last decimal place, e.g., for a pH value of about pH 2.7, the error margin is 2.65-2.74. However, for the following parameters, the specific margins shall apply: a temperature specified in ° C. with no decimal place shall have an error margin of ±1° C. (e.g., a temperature value of about 50° C. means 50° C.±1° C.); a time indicated in hours shall have an error margin of 0.1 hours irrespective of the decimal places (e.g., a time value of about 1.0 hours means 1.0 hours±0.1 hours; a time value of about 0.5 hours means 0.5 hours±0.1 hours).

In the present application, any parameter indicated with the term “about” is also contemplated as being disclosed without the term “about”. In other words, embodiments referring to a parameter value using the term “about” shall also describe an embodiment directed to the numerical value of said parameter as such. For example, an embodiment specifying a pH of “about pH 2.7” shall also disclose an embodiment specifying a pH of “pH 2.7” as such; an embodiment specifying a pH range of “between about pH 2.7 and about pH 2.1” shall also describe an embodiment specifying a pH range of “between pH 2.7 and pH 2.1”, etc.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

### 4. BRIEF DESCRIPTION OF THE DRAWINGS

FIG. 1. Schematic drawing of the structural format of polypeptides TP003, TP006, TP008, TP009, TP016, TP019 according to specific embodiments of the present technology.

TP006, TP009, TP016 and TP019 are fusion polypeptides according to particular embodiments of the present technology comprising an IgG4 FALA Fc domain (as described herein and created with knob-in hole-technology) linked to (i) a Nanobody® VHH specifically binding to serum albumin (ellipse shape hatched in grey) and/or (ii) to a Nanobody® VHH not binding to serum albumin or any other envisaged target (black ellipse shape). The Fc domain and Nanobody® VHH sequences in these polypeptide constructs were fused via a linker (as described in detail herein) to the N- and/or C-terminus of the Fc chain, i.e., via an IgG1 hinge, e.g., SEQ ID NO.: 38, and/or a GS linker, see, e.g., Table A-2.

P003 and TP008 were made as control fusion polypeptides, comprising the same composition of the corresponding test constructs, i.e., TP009, TP016 and TP006 and TP019, respectively, except that the Nanobody® VHH binding to serum albumin was replaced by a Nanobody® VHH not binding to serum albumin or any other envisaged target (black ellipse shapes). As second control, a full-length monoclonal antibody (TP013) was generated containing the same IgG4 FALA Fc backbone containing knob in hole mutations.

TP016 and TP019 comprise additional amino acid variations (as compared to the native Fc IgG4 domain) in the Fc backbone sequence (i.e., I253A, H310A, H435A). These Fc sequence variants were made to test constructs that showed no binding to FcRn and will be referred to further herein as non-binding Fc-variants.

FIG. 2. Mean (+/−SD, n=2) serum concentration-time profiles with IgG (hIgG) competition of polypeptides TP006, TP009, compared to control Fc-ISVD construct (TP003) or monoclonal antibody (TP013) following intravenous bolus administration at 5-8 mg/kg in female Tg32 mice. To mimic relevant competition with hIgG, Tg32 mice were preloaded intravenously with 4 Privigen® injections of 250 mg/kg, once weekly, with the first administration 2 days prior to initiation of the PK study. Black triangles in the X-axis indicate the timepoints of Privigen injections.

FIG. 3. Schematic drawing of the structural format of polypeptides TP108, TP111, TP117, TP118, TP121 and TP123 according to specific embodiments of the present technology.

FIG. 4. Mean (+/−SD, n=3) plasma concentration-time profiles of polypeptides TP108 and TP111, following intravenous bolus administration at 5 mg/kg in female Tg32 mice.

FIG. 5. Mean (+/−SD, n=3) plasma concentration-time profiles of polypeptides TP117, TP118, TP121 and TP123, following intravenous bolus administration at 5 mg/kg in female Tg32 mice.

FIG. 6. Schematic drawing of the structural format of polypeptides TPP-66143, TPP-66145, TPP-66146, TPP-66147, TPP-66148, TPP-66149, TPP-66144, TPP-66150, TPP-66151, TPP-66152, TPP-66176, TPP-66177, TPP-66153, TPP-66154, TPP-66175 and TPP-66174 according to specific embodiments of the present technology.

FIG. 7. Mean (+/−SD, n=3) plasma concentration-time profiles of ALB23002 ISVD-Fc polypeptides TPP-66144, TPP-66147, with 9 and 35GS linker respectively, compared to control ISVD-Fc polypeptide TPP-66143, following intravenous bolus administration at 5 mg/kg in female Tg32 mice.

FIG. 8. Mean (+/−SD, n=3) plasma concentration-time profiles of Albumin-binding domain-Fc polypeptides TPP-66150, TPP-66151, TPP-66152, compared to ALB23002 ISVD-Fc polypeptide TPP-66147 and control ISVD-Fc polypeptide TPP-66143, following intravenous bolus administration at 5 mg/kg in female Tg32 mice.

FIG. 9. Mean (+/−SD, n=3) plasma concentration-time profiles of different Albumin-binding ISVD-Fc polypeptides TPP-66145, TPP-66146, TPP-66147, TPP66148, TPP-66149 compared to control ISVD-Fc polypeptide TPP-66143, following intravenous bolus administration at 5 mg/kg in female Tg32 mice.

FIG. 10. Mean (+/−SD, n=3) plasma concentration-time profiles of human albumin-Fc polypeptide TPP-66153 and human albumin(QMP)-Fc polypeptide TPP-66154, compared to ALB23002 ISVD-Fc polypeptide TPP-66147 and a control ISVD-Fc polypeptide TPP-66143, following intravenous bolus administration at 5 mg/kg in female Tg32 mice.

FIG. 11. Mean (+/−SD, n=3) plasma concentration-time profiles of ALB23002 ISVD-Fc(YTE) polypeptide TPP-66174 and ALB23002 ISVD-Fc polypeptide TPP-66147 compared to control ISVD-Fc(YTE) polypeptide TPP-66175 and control ISVD-Fc polypeptide TPP-66143, following intravenous bolus administration at 5 mg/kg in female Tg32 mice.

FIG. 12. Mean (+/−SD, n=3) plasma concentration-time profiles of symmetrical ISVD-Fc polypeptides TPP-66176, and symmetrical ALB23002 ISVD-Fc polypeptide TPP-66177 compared to ALB23002 ISVD-Fc polypeptide TPP-66147 and control ISVD-Fc polypeptide TPP-66143, following intravenous bolus administration at 5 mg/kg in female Tg32 mice.

### 5. DETAILED DESCRIPTION

The present inventors have developed novel polypeptides binding to FcRn comprising (i) at least one domain comprising a serum albumin protein and/or at least one domain specifically binding to a serum albumin protein and (ii) an Fc domain of an immunoglobulin G (IgG) or a fragment thereof, preferably a FcRn-binding fragment thereof.

In further particular embodiments, the polypeptides as disclosed herein comprise, in addition to a domain comprising a serum albumin protein and/or a domain specifically binding to a serum albumin protein, as described in detail below, an Fc region of an IgG that may or may not specifically bind to human FcRn (SEQ ID NO: 24) or (polymorphic) variants or isoforms thereof, as also described in detail below.

Isoforms are alternative protein sequences that can be generated from the same gene by a single biological event or by the combination of biological events such as alternative promoter usage, alternative splicing, alternative initiation and ribosomal frameshifting, all as known in the art.

Amino acid residues will be indicated interchangeably herein according to the standard three-letter or one-letter amino acid code, as mentioned in Table B-1 below.

TABLE B-1
Common amino acids

| 1-Letter Code | 3-Letter Code | Amino Acid Name |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Unspecified |
| Y | Tyr | Tyrosine |

When an amino acid residue is indicated as “X” or “Xaa”, it means that the amino acid residue is unspecified, unless the context requires a more limited interpretation. For example, if the description provides an amino acid sequence of a CDR wherein one (or more) of the amino acid residue(s) is (are) indicated with “X”, the description may further specify which amino acid residue(s) is (can be) present at that specific position of the CDR.

Amino acids are those L-amino acids commonly found in naturally occurring proteins and are listed in Table B-1. Those amino acid sequences containing D-amino acids are not intended to be embraced by this definition. Any amino acid sequence that contains post-translationally modified amino acids may be described as the amino acid sequence that is initially translated using the symbols shown in the Table B-1 with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition. The terms “protein”, “peptide”, “protein/peptide”, and “polypeptide” are used interchangeably throughout the disclosure, and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptide from a protein; polypeptides may be made by chemical synthesis or recombinant methods; and that proteins are generally made in vitro or in vivo by recombinant methods as known in the art.

When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first-mentioned nucleotide sequence or amino acid sequence, respectively, but more usually this generally means that the first-mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first-mentioned sequence has actually been generated or obtained (which may for example be by any suitable method described herein). By means of a non-limiting example, when an ISVD is said to comprise a CDR sequence, this may mean that said CDR sequence has been incorporated into the ISVD, but more usually this generally means that the ISVD contains within its sequence a stretch of amino acid residues with the same amino acid sequence as said CDR sequence, irrespective of how said ISVD has been generated or obtained. It should also be noted that when the latter amino acid sequence has a specific biological or structural function, it preferably has essentially the same, a similar or an equivalent biological or structural function in the first-mentioned amino acid sequence (in other words, the first-mentioned amino acid sequence is preferably such that the latter sequence is capable of performing essentially the same, a similar or an equivalent biological or structural function). For example, when an ISVD is said to comprise a CDR sequence or framework sequence, respectively, the CDR sequence and framework are preferably capable, in said ISVD, of functioning as a CDR sequence or framework sequence, respectively. Also, when a nucleotide sequence is said to comprise another nucleotide sequence, the first-mentioned nucleotide sequence is preferably such that, when it is expressed into an expression product (e.g., a polypeptide), the amino acid sequence encoded by the latter nucleotide sequence forms part of said expression product (in other words, that the latter nucleotide sequence is in the same reading frame as the first-mentioned, larger nucleotide sequence).

The term “domain” as used herein generally refers to a globular region of a protein. For example, the term “domain” can refer to a globular region of an antibody, and in particular to a globular region of a heavy chain antibody, or the term “domain” can refer to a polypeptide that essentially consists of a globular region of a polypeptide. In particular embodiments, a domain in the context of the present disclosure essentially consists of a serum albumin protein or a fragment, variant or derivative thereof. In other particular embodiments, a domain in the context of the present disclosure will comprise a globular region of an antibody and will comprise peptide loops (for example 3 or 4 peptide loops) that are stabilized, for example, as a sheet or by disulfide bonds. In particular embodiments, a domain in the context of the present disclosure will essentially consist of a constant region of an antibody, such as an Fc domain of an antibody.

In the context of the present technology, “binding to” a certain target molecule has the usual meaning in the art as understood in the context of proteins and their respective ligands or antibodies and their respective antigens. In certain particular embodiments of the present application, “binding to” refers to the direct and specific interaction between two binding partners or molecules, such as for example a protein and its ligand or an antibody and its antigen. In certain other particular embodiments, “binding to” refers to an indirect interaction between two binding partners or molecules, such as for example when the first binding partner and the second binding partner directly and specifically bind to the same target protein so as to be indirectly linked or indirectly interact with each other via said target protein.

The term “antigenic determinant” refers to the epitope on the antigen recognized by the antigen binding molecule (such as an ISVD or a polypeptide comprising the ISVD) and more in particular by the antigen binding site of said molecule. The terms “antigenic determinant” and “epitope” may also be used interchangeably herein. The antigen binding molecule (such as an antibody, an ISVD, a polypeptide of the present technology, or generally an antigen-binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.

### 5.1 Immunoglobulin Single Variable Domains

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e., a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a VH-VL pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single VH, a single VHH or single VL domain.

As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a VH, VHH, including a camelized VH or humanized VHH. Preferably, it is a VHH, including a camelized VH or humanized VHH. Heavy chain ISVDs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a Nanobody® molecule (as defined herein, and including but not limited to a VHH); other single variable domains, or any suitable fragment of any one thereof.

In particular, the immunoglobulin single variable domain may be a Nanobody® immunoglobulin single variable domain (such as a VHH, including a humanized VHH or camelized VH) or a suitable fragment thereof. [Note: Nanobody® is a registered trademark of Ablynx N.V.]

“VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al., Nature 363: 446-448, 1993). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs, reference is made to the review article by Muyldermans (Reviews in Molecular Biotechnology 74: 277-302, 2001).

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries, e.g., by phage display.

The generation of immunoglobulin sequences, such as Nanobody® VHHs, has been described extensively in various publications, among which WO 94/04678, Hamers-Casterman et al., 1993 and Muyldermans et al., 2001 (Reviews in Molecular Biotechnology 74: 277-302, 2001) can be exemplified. In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens.

The present technology may use immunoglobulin sequences of different origin, comprising mouse, rat, rabbit, donkey, human and camelid immunoglobulin sequences. The technology also includes fully human, humanized or chimeric sequences. For example, the present technology comprises camelid immunoglobulin sequences and humanized camelid immunoglobulin sequences, or camelized domain antibodies, e.g., camelized dAb as described by Ward et al. (see for example WO 94/04678 and Riechmann, Febs Lett., 339:285-290, 1994 and Prot. Eng., 9:531-537, 1996). Moreover, the present technology also uses fused immunoglobulin sequences, e.g., forming a multivalent and/or multispecific construct (for multivalent and multispecific polypeptides containing one or more VHH domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g., toxins, labels, radiochemicals, etc., which are derivable from the immunoglobulin sequences of the present technology.

A “humanized VHH” comprises an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., indicated above). This can be performed in a manner known per se, which will be clear to the skilled person, for example on the basis of the further description herein and the prior art (e.g., WO 2008/020079). Again, it should be noted that such humanized VHHs can be obtained in any suitable manner known per se and thus are not strictly limited to polypeptides that have been obtained using a polypeptide that comprises a naturally occurring VHH domain as a starting material.

A “camelized VH” comprises an amino acid sequence that corresponds to the amino acid sequence of a naturally occurring VH domain, but that has been “camelized”, i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description herein and the prior art (e.g., WO 2008/020079). Such “camelizing” substitutions are preferably inserted at amino acid positions that form and/or are present at the VH-VL interface, and/or at the so-called Camelidae hallmark residues, as defined herein (see for example WO 94/04678 and Davies and Riechmann (1994 and 1996), supra). Preferably, the VH sequence that is used as a starting material or starting point for generating or designing the camelized VH is preferably a VH sequence from a mammal, more preferably the VH sequence of a human being, such as a VH3 sequence. However, it should be noted that such camelized VH can be obtained in any suitable manner known per se and thus are not strictly limited to polypeptides that have been obtained using a polypeptide that comprises a naturally occurring VH domain as a starting material.

A preferred structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

As further described in paragraph q) on pages 58 and 59 of WO 08/020079 (incorporated herein by reference), the amino acid residues of an immunoglobulin single variable domain can be numbered according to the general numbering for VH domains given by Kabat et al. (“Sequence of proteins of immunological interest”, US Public Health Services, NIH Bethesda, MD, Publication No. 91), as applied to VHH domains from Camelids in the article of Riechmann and Muyldermans, 2000 (J. Immunol. Methods 240 (1-2): 185-195; see for example FIG. 2 of this publication). It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

In the present application, unless indicated otherwise, CDR sequences were determined according to the AbM numbering as described in Kontermann and Dübel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods. In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a VL-sequence) and/or from a heavy chain variable domain (e.g., a VH-sequence or VHH sequence). In one particularly preferred aspect, the framework sequences are either framework sequences that have been derived from a VHH-sequence (in which said framework sequences may optionally have been partially or fully humanized) or are conventional VH sequences that have been camelized (as defined herein).

In particular, the framework sequences present in the ISVD sequence used in the present technology may contain one or more of hallmark residues (as defined herein), such that the ISVD sequence is a Nanobody® molecule, such as a VHH, including a humanized VHH or camelized VH. Some preferred, but non-limiting examples of (suitable combinations of) such framework sequences will become clear from the further disclosure herein.

Again, as generally described herein for the immunoglobulin sequences, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived).

However, it should be noted that the present technology is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized VHH sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

As described above, an ISVD may be a Nanobody® VHH or a suitable fragment thereof. For a general description of ISVDs, reference is made to the further description below, as well as to the prior art cited herein. In this respect, it should however be noted that this description and the prior art mainly described ISVDs of the so-called “VH3 class” (i.e., ISVDs with a high degree of sequence homology to human germline sequences of the VH3 class such as DP-47, DP-51 or DP-29). It should however be noted that the present technology in its broadest sense can generally use any type of ISVD, and for example also uses the ISVDs belonging to the so-called “VH4 class” (i.e., ISVDs with a high degree of sequence homology to human germline sequences of the VH4 class such as DP-78), as for example described in WO 2007/118670.

Generally, ISVDs (in particular VHH sequences, including (partially) humanized VHH sequences and camelized VH sequences) can be characterized by the presence of one or more “Hallmark residues” (as described herein) in one or more of the framework sequences (again as further described herein). Thus, generally, a ISVD can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, an ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, an ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table A-0 below.

TABLE A-0
Hallmark Residues in Nanobody ® ISVDs

| Position | Human VH3 | Hallmark Residues |
|---|---|---|
| 11 | L, V; | L, S, V, M, W, F, T, Q, E, A, R, G, K, Y, N, P, I; preferably L |
| | predominantly L | |
| 37 | V, I, F; | F⁽¹⁾, Y, V, L, A, H, S, I, W, C, N, G, D, T, P, preferably F⁽¹⁾ or |
| | usually V | Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or Q⁽³⁾ |
| 45⁽⁸⁾ | L | L⁽²⁾, R⁽³⁾, P, H, F, G, Q, S, E, T, Y, C, I, D, V; preferably L⁽²⁾ or |
| | | R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, N, |
| | | D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; preferably K |
| | | or R; most preferably K |
| 84 | A, T, D; | P⁽⁵⁾, S, H, L, A, V, I, T, F, D, R, Y, N, Q, G, E; preferably P |
| | predominantly A | |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; | Q, L⁽⁷⁾, R, P, E, K, S, T, M, A, H; preferably Q or L⁽⁷⁾ |
| | predominantly L | |

Notes:
In particular, but not exclusively, in combination with KERE or KQRE at positions 43-46.
Usually as GLEW at positions 44-47.
Usually as KERE or KQRE at positions 43-46, e.g., as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
With both GLEW at positions 44-47 and KERE or KQRE at positions 43-46.
Often as KP or EP at positions 83-84 of naturally occurring VHH domains.
In particular, but not exclusively, in combination with GLEW at positions 44-47.
With the proviso that when positions 44-47 are GLEW, position 108 is always Q in (non-humanized) VHH sequences that also contain a W at 103.
The GLEW group also contains GLEW-like sequences at positions 44-47, such as for example GVEW, EPEW, GLER, DQEW, DLEW, GIEW, ELEW, GPEW, EWLP, GPER, GLER and ELEW

### 5.2 Specificity

The terms “specificity”, “binding specifically” or “specific binding” refer to the number of different target molecules, such as antigens, from the same organism to which a particular binding unit, such as an ISVD, can bind with sufficiently high affinity (see below). “Specificity”, “binding specifically” or “specific binding” are used interchangeably herein with “selectivity”, “binding selectively” or “selective binding”. Binding units, such as ISVDs, preferably specifically bind to their designated targets. The specificity/selectivity of a binding unit can be determined based on affinity. The affinity denotes the strength or stability of a molecular interaction. The affinity is commonly given as by the K_{D}, or dissociation constant, which is expressed in units of mol/liter (or M). The affinity can also be expressed as an association constant, K_{A}, which equals 1/K_{D} and is expressed in units of (mol/liter)⁻¹ (or M⁻¹). The affinity is a measure for the binding strength between a moiety and a binding site on the target molecule: the lower the value of the K_{D}, the stronger the binding strength between a target molecule and a targeting moiety. Typically, binding units used in the present technology (such as ISVDs) will bind to their targets with a dissociation constant (K_{D}) of 10⁻⁵ to 10⁻¹² moles/liter or less, 10⁻⁶ to 10⁻² moles/liter or less and preferably 10⁻⁷ to 10⁻¹² moles/liter or less and more preferably 10⁻⁸ to 10⁻¹² moles/liter (i.e., with an association constant (K_{A}) of 10⁵ to 10¹² liter/moles or more, 10⁶ to 10⁻¹² liter/moles or more and preferably 10⁷ to 10¹² liter/moles or more and more preferably 10⁸ to 10¹² liter/moles). Any K_{D} value greater than 10⁻⁴ mol/liter (or any K_{A} value lower than 10⁴ liters/mol) is generally considered to indicate non-specific binding. The K_{D} for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 μM) or less. Accordingly, specific/selective binding may mean that—using the same measurement method, e.g., SPR—a binding unit (or polypeptide comprising the same) binds to FcRn with a K_{D} value of 10⁻⁵ to 10⁻¹² moles/liter or less and binds to related targets with a K_{D} value greater than 10⁻⁴ moles/liter. Thus, the ISVD preferably exhibits at least half the binding affinity, more preferably at least the same binding affinity, to human FcRn as compared to an ISVD consisting of the amino acid of SEQ ID NOs.: 14 or 15, wherein the binding affinity is measured using the same method, such as SPR. Specific binding to a certain target from a certain species does not exclude that the binding unit can also specifically bind to the analogous target from a different species.

In one embodiment, the polypeptide of the present technology binds to HSA with a K_{D} value of about 10⁻⁵ to 10⁻¹² moles/liter or less, such as about 10⁻⁶ to 10⁻¹⁰ moles/liter, such as about 10⁻⁷ to 10⁻¹⁰ moles/liter, or about 10⁻⁷ to 10⁻⁹ moles/liter, such as about 10⁻⁸ to 10⁻⁹ moles/liter e.g., as determined by SPR.

In another embodiment, the polypeptide of the present technology binds to FcRn at pH 6.0 in the absence of HSA with a K_{D} value of about 10⁻⁵ to 10⁻¹² moles/liter or less, such as about 10⁻⁶ to 10⁻¹⁰ moles/liter, such as about 10⁻⁷ to 10⁻¹⁰ moles/liter, or about 10⁻⁷ to 10⁻⁹ moles/liter, or about 10⁻⁶ to 10⁻⁸ moles/liter, or about 10⁻⁶ to 10⁻⁹ moles/liter, e.g., as determined by SPR.

For example, specific binding to human FcRn or to human serum albumin does not exclude that the binding domain (or a polypeptide comprising the same) can also specifically bind to FcRn or serum albumin from cynomolgus monkeys.

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g., of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}]. The affinity of a molecular interaction between two molecules can be measured via different techniques known per se, such as the well-known surface plasmon resonance (SPR) biosensor technique (see for example Ober et al., 2001, Intern. Immunology 13: 30 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnnson et al., (1991, Anal. Biochem. 198: 268-277). Another well-known biosensor technique to determine affinities of biomolecular interactions is bio-layer interferometry (BLI) (see for example Abdiche et al., 2008, Anal. Biochem. 377: 209-217).

The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA). Alternatively, affinities can be measured in Kinetic Exclusion Assay (KinExA) (see for example Drake et al., 2004, Anal. Biochem., 328: 35-43), using the KinExA® platform (Sapidyne Instruments Inc, Boise, USA).

The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen). The GYROLAB® immunoassay system provides a platform for automated bioanalysis and rapid sample turnaround (Fraley et al., 2013, Bioanalysis 5: 1765-74).

In particular embodiments, the polypeptides such as the FcRn targeting polypeptides of the present technology comprise at least one domain that specifically binds to a serum albumin protein with an affinity (K_{A}) of between 10⁶ M⁻¹ and 10¹¹ M⁻¹.

In particular embodiments, the polypeptides such as the FcRn targeting polypeptides comprise at least one domain specifically binding to a serum albumin protein with a dissociation constant (K_{D}) of between 10⁻⁶ M and 10⁻¹¹ M or less. Preferably, the K_{D} is determined by Kinexa, BLI or SPR, for instance as determined by SPR.

In particular embodiments, the polypeptides such as the FcRn targeting polypeptides comprise at least one domain specifically binding to a serum albumin protein with an on-rate constant (kₒₙ) selected from the group consisting of at least about 10²M⁻¹s⁻¹, of at least about 10³M⁻¹s⁻¹, at least about 10⁴M⁻¹s⁻¹, at least about 10⁵M⁻¹s⁻¹, at least about 10⁶M⁻¹s⁻¹, at least about 10⁷M⁻¹s⁻¹, and at least about 10⁸ M⁻¹s⁻¹, preferably as measured by surface plasmon resonance or BLI.

In particular embodiments, the polypeptides such as the FcRn targeting polypeptides comprise at least one domain specifically binding to a serum albumin protein with an off-rate constant (k_{off}) selected from the group consisting of at most about 10⁻¹s⁻¹, at most about 10⁻¹s⁻¹, at most about 10⁻³s⁻¹, of at most about 10⁴s⁻¹, at most about 10⁻⁵s⁻¹, and at most about 10⁻¹s⁻¹, preferably as measured by surface plasmon resonance or BLI.

The polypeptides of the present technology comprising at least one serum albumin binding domain are, in certain embodiments, such that they are cross-reactive between human serum albumin and serum albumin from at least one, preferably from at least two, more preferably from at least three and up to essentially all of the following species of mammal: mouse, dog, rat, rabbit, guinea pig, pig, sheep, cow and cynomolgus monkey.

When an ISVD is said to exhibit “(improved) cross-reactivity for binding to human and non-human primate serum albumin” compared to another ISVD, it means that for said ISVD the ratio of the binding activity (such as expressed in terms of K_{D} or k_{off}) for human serum albumin and for non-human primate serum albumin is lower than that same ratio calculated for the other ISVD in the same assay. Good cross-reactivity for binding to human and non-human primate serum albumin allows for the assessment of toxicity of a serum albumin binding polypeptide according to the present technology in preclinical studies conducted on non-human primates.

For the purposes of comparing two or more immunoglobulin single variable domains or other amino acid sequences such, e.g., the polypeptides of the present technology etc., the percentage of “sequence identity” between a first amino acid sequence and a second amino acid sequence (also referred to herein as “amino acid identity”) may be calculated or determined as described in paragraph f) on pages 49 and 50 of WO 08/020079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein; alternatively, the degree of sequence identity between two amino acid sequences may be calculated using a known computer algorithm for sequence alignment, such as NCBI Blast v2.0, using standard settings. Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two immunoglobulin single variable domains, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein. Examples of conservative substitutions are described herein further below.

Any amino acid substitutions applied to the polypeptides described herein may also be based on the analysis of the frequencies of amino acid variations between homologous proteins of different species developed by Schulz et al., 1978 (Principles of Protein Structure, Springer-Verlag), on the analyses of structure forming potentials developed by Chou and Fasman 1975 (Biochemistry 13: 211) and 1978 (Adv. Enzymol. 47: 45-149), and on the analysis of hydrophobicity patterns in proteins developed by Eisenberg et al., 1984 (Proc. Natl. Acad. Sci. USA 81: 140-144), Kyte & Doolittle 1981 (J Molec. Biol. 157: 105-132), and Goldman et al., 1986 (Ann. Rev. Biophys. Chem. 15: 321-353), all incorporated herein in their entirety by reference. Information on the primary, secondary and tertiary structure of ISVDs is given in the description herein and in the general background art cited above. Also, for this purpose, the crystal structure of a VHH domain from a llama is for example given by Desmyter et al., 1996 (Nature Structural Biology, 3: 803), Spinelli et al., 1996 (Natural Structural Biology 3: 752-757), and Decanniere et al., 1999 (Structure, 7: 361). Further information about some of the amino acid residues that in conventional VH domains form the VH/VL interface and potential camelizing substitutions on these positions can be found in the prior art cited above. Immunoglobulin single variable domains and nucleic acid sequences are said to be “exactly the same” if they have 100% sequence identity (as defined herein) over their entire length.

5.3 First Domain: (i) a Domain Comprising a Serum Albumin Protein and/or a Domain, Such as a Serum Albumin Binding ISVD, that has High Affinity for/Binds Specifically to a Serum Albumin Protein

Human serum albumin (HSA) and IgG, the two most abundant soluble proteins present in blood circulation, are an exception to most proteins in circulation in that they share the remarkable property of having a prolonged serum half-life of about 19 to 21 days in human.

HSA is the most abundant plasma protein in the blood and is a carrier protein involved in many processes that serve to maintain homeostasis in the body, i.e., maintaining the oncotic pressure. Albumins are widely used as drug delivery vehicles due to their high serum concentration, their long half-life, non-toxicity and low immunogenicity and their uptake in benign and tissues, and their ability to bind to a wide variety of drugs (Mishra, V., Heath, RJ., “Structural and biochemical features of human serum albumin essential for eukaryotic cell culture”, Int. J. Mol. Sci. 2021, 22, 8411). HSA has been well characterized as a polypeptide of 585 amino acids, the sequence of which can be found, e.g., in Peters, T., Jr. (1996) “All about Albumin: Biochemistry, Genetics and Medical Applications”, pp 10, Academic Press, Inc., Orlando (ISBN 0-12-552110-3).

This prolonged half-life of serum albumin is primarily due to it being protected from intracellular lysosomal degradation by binding to the neonatal Fc receptor (FcRn). FcRn is a heterodimer consisting of an N-glycosylated transmembrane M HC class I-like heavy chain that is noncovalently associated with soluble b2-microglobulin. Both IgG and albumin are ligands binding to different epitopes of FcRn. In general, the FcRn recycling mechanism is strictly pH-dependent and binding to FcRn is favoured at low pH (e.g., acidic endosomal pH, which is typically below 6.5) following acidification of the endosomal compartment. When albumin/IgG binds to FcRn, it escapes degradation in the lysosome. On return to the cell surface, at extracellular physiological pH (which is typically around pH 7.4), the binding is weakened, resulting in the release of albumin/IgG into the bloodstream (see, e.g., Ward E S, Ober R J., Targeting FcRn to Generate Antibody-Based Therapeutics. Trends Pharmacol Sci., 2018; 39(10):892-904 and Andersen et al., Extending serum half-life of albumin by engineering neonatal Fc receptor (FcRn) binding, JBC, 2014, 289, 19: 13492-13502). Hence, HSA has a characteristic binding to its receptor FcRn, where it binds at pH 6.0 but not at pH 7.4.

A natural variant having lower plasma half-life has been identified (Peach, R. J. and Brennan, S. O. (1991) Biochim Biophys Acta. 1097:49-54) having the substitution D494N. This substitution generated an N-glycosylation site in this variant, which is not present in the wild-type albumin. It is not known whether the glycosylation or the amino acid change is responsible for the change in plasma half-life.

Otagiri et al. (2009), Biol. Pharm, Bull. 32(4), 527-534, discloses that 77 albumin variants are known. Of these, 25 are found in domain III. A natural variant lacking the last 175 amino acids at the carboxy termini has been shown to have reduced half-life (Andersen et al (2010), Clinical Biochemistry 43, 367-372). Iwao et al. (2007) studied the half-life of naturally occurring human albumin variants using a mouse model, and found that K541E and K560E had reduced half-life, E501K and E570K had increased half-life and K573E had almost no effect on half-life (Iwao, et al. (2007) B.B.A. Proteins and Proteomics 1774, 1582-1590).

Galliano et al. (1993) Biochim. Biophys. Acta 1225, 27-32 discloses a natural variant E505K. Minchiotti et al. (1990) discloses a natural variant K536E. Minchiotti et al. (1987) Biochim. Biophys. Acta 916, 411-418 discloses a natural variant K574N. Takahashi et al. (1987) Proc. Natl. Acad. Sci. USA 84, 4413-4417, discloses a natural variant D550G. Carlson et al. (1992). Proc. Nat. Acad. Sci. USA 89, 8225-8229, discloses a natural variant D550A.

In particular, albumin is increasingly being used to improve the pharmacokinetics of short-lived small molecule drugs that are able to bind to albumin and also to bioactive therapeutic peptides and proteins by genetic fusion of such molecules to the N- or C-terminal end of albumin (Nilsen, J., Trabjerg, E., Grevys, A. et al. An intact C-terminal end of albumin is required for its long half-life in humans. Commun Biol, 2020, 3, 181).

For instance, immunoglobulin variable domain sequences (ISVDs) that can bind to serum albumin have been developed and their coupling to therapeutic compounds, moieties, and entities to extend the serum half-life (as defined in these applications) was described for example in WO 2004/041865, WO 2006/122787, WO 2012/175400, WO 2015/173325 and PCT/EP2016/077973. For example, WO 2006/122787 discloses as SEQ ID NO: 62 a humanized serum albumin-binding ISVD called Alb-8 (see SEQ ID NO: 5 herein). WO 2012/175400 discloses as SEQ ID NO: 6 a humanized serum albumin-binding ISVD called Alb-23D. Some other references that disclose ISVDs against serum albumin include WO 2003/035694, WO 2004/003019, EP 2 139 918, WO 2011/006915 and WO 2014/111550. Preferred examples of albumin-binding ISVDs comprise or consist of a polypeptide as defined in SEQ ID NO.: 7-21 or 64-69.

Other albumin binding proteins (ABP) such as albumin-binding DARPins (Designed Ankyrin Repeat Proteins) or Affitins (also known as Nanofitins) have also been described as scaffolds to extend half-life of biologics (see, e.g., Michot N. et al., “Albumin binding Nanofitins, a new scaffold to extend half-life of biologics—a case study with exenatide peptide”, Peptides, 2022, 152:170760 or Steiner D., et al., “Half-life extension using serum albumin-binding DARPin® domains”, Protein Eng Des Sel, 2017, 30(9):583-591). Preferred examples of albumin-binding moieties which are not ISVDs comprise or consist of a polypeptide as defined in SEQ ID NO.: 102-104.

In one embodiment, the polypeptide of the present technology comprises at least one domain comprising a serum albumin protein. In particular embodiments, the serum albumin protein is human serum albumin (“Human serum albumin (1)” as defined in SEQ ID NO: 22, “Human serum albumin (2) (HSA(25-609))” as defined in SEQ ID NO: 23 or HSA(25-609)(E529Q, T551M, K597P) (HSA(QMP)) as defined in SEQ ID NO.: 110) or a polymorphic variant or isoform thereof. Preferably, the serum albumin protein comprised in the polypeptide of the present technology is human serum albumin, and more preferably is or comprises a protein sequence as defined in SEQ ID NO.: 23 or 110.

Accordingly, in particular embodiments, the polypeptides as disclosed herein comprise at least one domain comprising a serum albumin protein and an Fc domain of an IgG, or a fragment thereof. In particular embodiments, the serum albumin protein is human serum albumin (AAA98797 as defined in SEQ ID NO: 22 or P02768-1 as defined in SEQ ID NO: 23, or HSA-QMP as defined in SEQ ID NO.: 110, preferably as defined in SEQ ID NO.: 23 or 110) or a polymorphic variant or isoform thereof.

In particular embodiments, the polypeptides of the present technology comprise at least one serum albumin protein, or a fragment or variant thereof, such as for example but not limited to the albumin proteins, fragments and variants disclosed in WO 2011/124718, WO 2011/051489, WO 2013/075066, WO 2013/135896 and WO 2014/072481.

Polypeptides according to particular embodiments of the present technology comprising at least one serum albumin protein and at least one Fc domain are produced and tested for their beneficial PK properties.

In the context of the present technology, the term “serum albumin protein” means serum albumin, such as human serum albumin or derivatives, variants, or fragments thereof. Preferably the serum albumin protein comprises or consists of a polypeptide as defined in SEQ ID NOs: 22, 23 or 110.

The size of the albumin derivative, variant, or fragment thereof may vary depending on the size of the fragment, number of domains, the size of the non-albumin part of the polypeptide etc. It is preferred however that the albumin derivative, variant, or fragment has a size in the range of 40-80 kDa, preferably in the range of 50-70 kDa, more preferred in the range of 55-65 kDa and most preferred around 60 kDa.

Human serum albumin is the preferred serum albumin protein according to the present technology and is a protein consisting of about 585 amino acid residues and has a molecular weight of about 67 kDa (e.g., SEQ ID NO: 22 or SEQ ID NO: 23, or SEQ ID NO.: 110, preferably SEQ ID NO.: 23 or 110, even more preferably SEQ ID NO.: 23). The skilled person will appreciate that natural alleles may exist having essentially the same properties as human serum albumin but having one or more (several) amino acid changes compared to, e.g., SEQ ID NO: 22, SEQ ID NO: 23 or SEQ ID NO.: 110, and the inventors also contemplate the use of such natural alleles as serum albumin proteins according to the present technology.

According to the present technology the term “(serum) albumin derivative” means a non-natural, engineered molecule comprising or consisting of one or more parts of one or more domains of a serum albumin protein as specified. Serum albumin derivatives may be engineered for increased or decreased FcRn binding. For instance, a serum albumin derivative may be HSA(25-609)(E529Q, T551M, K597P) (SEQ ID NO.: 110, for increased FcRn binding). In a preferred embodiment, the polypeptide of the present technology comprises at least one domain which comprises or consists of a serum albumin derivative, for instance a serum albumin derivative which shows an increased or decreased FcRn binding as compared with the wild-type serum albumin derivative, preferably HSA(25-609)(E529Q, T551M, K597P), as defined in SEQ ID NO.: 110.

The term “(serum) albumin variant” includes an albumin or albumin derivative in which the albumin or albumin derivative is altered by chemical means such as post-translational derivatization or modification of the polypeptide, e.g., PEGylation and/or conjugation of a desirable moiety (such as a therapeutic moiety) to a thiol group, such as provided by an unpaired cysteine. The terms “derivative” and “variant” may or may not be used interchangeably.

The term “(serum) albumin fragment” means a polypeptide having one or more (several) amino acids deleted from the amino and/or carboxyl terminus of a serum albumin protein and/or an internal region of a serum albumin protein that has retained the ability to bind to FcRn. Fragments may comprise or consist of one uninterrupted sequence derived from human serum albumin or it may comprise or consist of two or more sequences derived from human serum albumin.

In particular embodiments, the at least one further moiety that comprises a serum albumin protein is a part, a fragment, a derivative or variant of a serum albumin protein.

In particular embodiments, the at least one domain comprising a serum albumin protein comprises or consist of human serum albumin (HSA) or a part, a fragment, a derivative or variant of human serum albumin.

The sequence of HSA uniprot ID P02768 (SEQ ID NO.: 109) is depicted below:

MKWVTFISLLFLFSSAYSRGVFRRDAHKSEVAHRFKDLGEENFKALVLI
AFAQYLQQCPFEDHVKLVNEVTEFAKTCVADESAENCDKSLHTLFGDKL
CTVATLRETYGEMADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADFVESKDVCKNY
AEAKDVFLGMFLYEYARRHPDYSVVLLLRLAKTYETTLEKCCAAADPHE
CYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

Hence, in one embodiment, the HSA protein comprised in the polypeptide of the present technology comprises or consists of amino acids 25 to 609 of the protein sequence of HSA uniprot ID P02768 (SEQ ID NO.: 23). In another embodiment, the HSA protein comprised in the polypeptide of the present technology comprises or consists of amino acids 25 to 609 of the protein sequence of HSA uniprot ID P02768 with the following mutations: E5290, T551M and K597P (for increased FcRn binding), see SEQ ID NO.: 110.

In other particular embodiments, the polypeptides as disclosed herein comprise at least one domain specifically binding to a serum albumin protein, such as to human serum albumin (AAA98797 as defined in SEQ ID NO: 22, P02768-1 as defined in SEQ ID NO: 23 or HSA(25-609)(E529Q, T551M, K597P) as defined in SEQ ID NO.: 110) or (polymorphic) variants or isoforms thereof.

In yet other particular embodiments, the polypeptides as disclosed herein comprise at least one domain comprising a serum albumin protein and at least one domain specifically binding to a serum albumin protein, such as to human serum albumin (AAA98797 as defined in SEQ ID NO: 22, P02768-1 as defined in SEQ ID NO: 23 or HSA(25-609)(E529Q, T551M, K597P), as defined in SEQ ID NO.: 110)) or (polymorphic) variants or isoforms thereof.

In other embodiments, the polypeptide of the present technology comprises (i) at least one domain specifically binding to a serum albumin protein, and/or a variant thereof, such as to human serum albumin (“Human serum albumin (1)” as defined in SEQ ID NO: 22 or “Human serum albumin (2) (HSA(25-609))” as defined in SEQ ID NO: 23, or HSA(25-609)(E529Q, T551M, K597P), as defined in SEQ ID NO.: 110) or (polymorphic) variants or isoforms thereof.

In further particular embodiments, the (i) at least one domain specifically binding to a serum albumin protein comprised in the polypeptides of the present technology specifically binds to amino acid residues on the serum albumin protein that are not involved in binding of the serum albumin protein to FcRn.

In further particular embodiments, the (i) at least one domain specifically binding to a serum albumin protein comprised in the polypeptides of the present technology specifically binds to domain II of human serum albumin.

In particular embodiments, the (i) at least one domain specifically binding to a serum albumin protein comprised in the polypeptides of the present technology is a peptide or protein comprising between 5 and 500 amino acids.

In particular embodiments of the technology, the at least one domain that specifically binds to a serum albumin protein is such that it binds (at least) to a non-linear epitope that comprises one or more of the amino acid residues within one or more of the following stretches of amino acid residues within the primary sequence of human serum albumin: positions 298-311 (and in particular one or more of Met298, Pro299, Ala300, Asp301, Leu302, Pro303, Ser304, Leu305, Ala306 and Glu311); positions 334 to 341 (and in particular one or more of Tyr334, Arg337, His338, Pro339 and/or Asp340) and/or positions 374-381 (and in particular one or more of Phe374, Asp375, Phe 377, Lys378 and Va1381), with the amino acid residues in human serum albumin being numbered according to the numbering given in Meloun et al., FEBS Letters, 1975, 58, p. 134-137.

In yet further particular embodiments, the (i) at least one domain specifically binding to a serum albumin protein comprised in the polypeptides of the present technology is chosen from the group consisting of an Affibody® (an affibody molecule), a scFv, a Fab, a Designed Ankyrin Repeat Protein (DARPin®), an Albumin Binding Domain (ABD), a Nanofitin® (aka affitin) and an ISVD. Examples of preferred albumin binding domains comprised in the polypeptide of the present technology comprise or consist of a polypeptide as defined in SEQ ID NOs.: 7-21, 61-69 and 102-104, preferably as defined in SEQ ID NOs.: 7-21 and 61-69. In a preferred embodiment, the (i) at least one domain specifically binding to a serum albumin protein comprised in the polypeptides of the present technology is an ISVD, more preferably a VHH, even more preferably comprising or consisting of SEQ ID NO.: 7-21 and 61-69, even more preferably comprising or consisting of: ALB23002 (SEQ ID NO.: 20), Alb23002-A (SEQ ID NO.: 21), HSA006A06 (SEQ ID NO.: 65), ALBX00002 (SEQ ID NO.: 64), ALB11002 (SEQ ID NO.: 13) and T023500029 (SEQ ID NO.: 69), even more preferably comprising or consisting of: HSA006A06 (SEQ ID NO.: 65), ALB11002 (SEQ ID NO.: 13) and ALB23002 (SEQ ID NO.: 20).even more preferably SEQ ID NO.: 20 (Alb23002).

Albumin binding domains (ABD) are described, e.g., in Hopp. J. et al., “The effects of affinity and valency of an albumin-binding domain (ABD) on the half-life of a single-chain diabody-ABD fusion protein”, Protein Eng Des Sel., 2010, 23(11):827-34.

In particular embodiments, the at least one serum albumin binding domain in the polypeptides of the technology is such that it is (at least) cross-reactive between human serum albumin and cynomolgus monkey serum albumin, and preferably also between either human serum albumin and/or cynomolgus monkey serum albumin on the one hand, and at least one, preferably both of rat serum albumin and pig serum albumin on the other hand. For the sake of convenience, in the sequence of serum albumin, the stretches of amino acids that are assumed to be part of the putative epitope of the polypeptides of the present technology have been highlighted. Without being limited to any specific mechanism or hypothesis, it is assumed that the polypeptides of the present technology are (essentially) capable of binding to (one or more amino acid residues within) the corresponding stretches of amino acid residues that are present within the amino acid sequence of those mammalian serum albumin proteins, with which the polypeptides of the present technology are cross-reactive.

Generally, a polypeptide of the present technology comprising at least one serum albumin binding moiety can be considered to be cross-reactive between human serum albumin and serum albumin from one of the above mentioned other species when it can bind to human serum albumin with an affinity less than 500 nM, preferably less than 200 nM, more preferably less than 10 nM; and also to serum albumin from those above-mentioned species with an affinity less than 500 nM, preferably less than 200 nM, more preferably less than 10 nM, again both as determined using SPR.

In certain further particular embodiments, the at least one serum albumin binding domain specifically binds to amino acid residues on human serum albumin that are not involved in binding of human serum albumin to human FcRn.

The (i) at least one domain specifically binding to a serum albumin protein may thus preferably be an albumin binding ISVD as described herein. In certain further particular embodiments, the present technology provides polypeptides as described herein, characterized in that the at least one ISVD specifically binding to serum albumin is a (single) domain antibody, a VHH, a Nanobody® VHH, a humanized VHH, or a camelized VH. Hence, according to particularly preferred embodiments of the present technology, the at least one domain specifically binding to albumin that is comprised in the polypeptides of the present technology is at least one ISVD, specifically binding to (human) serum albumin.

The term “immunoglobulin single variable domain” (ISVD) has been described above in the present description. When the at least one domain specifically binding to a serum albumin protein comprised in the polypeptide of the present technology is an albumin-binding ISVD, it preferably comprises four framework regions (FR1 to FR4 respectively) and three complementarity determining regions (CDR1 to CDR3, respectively).

In certain further particular embodiments, the at least one serum albumin binding domain is an ISVD binding to serum albumin, which essentially consists of 4 framework regions (FR1 to FR4, respectively) and 3 complementarity determining regions (CDR1 to CDR3 respectively), and in which CDR1 is SFGMS (SEQ ID NO: 1), CDR2 is SISGSGSDTLYADSVKG (SEQ ID NO: 2) and CDR3 is GGSLSR (SEQ ID NO: 3), CDR determined according to Kabat definition; and/or in which CDR1 is GFTFRSFGMS (SEQ ID NO: 4), CDR2 is SISGSGSDTL (SEQ ID NO: 5) and CDR3 is GGSLSR (SEQ ID NO: 6), CDR determined according to AbM definition (Kontermann et al. 2010). Preferred CDR and FR regions of the albumin-binding ISVDs comprised in the polypeptide of the present technology are provided in Table A-6. Hence, in one embodiment, the polypeptide of the present technology comprises an albumin-binding (alb-binding) ISVD which comprises CDR and FR regions as described in Table A-6.

Hence, the CDR regions are preferably the following (numbering according to AbM):
a) CDR1 comprises the amino acid sequence of SEQ ID NO: 4 or has 3, 2 or 1 amino acid difference(s) with SEQ ID NO: 4 or CDR1 comprises the amino acid sequence of SEQ ID NO: 77 or has 3, 2 or 1 amino acid difference(s) with SEQ ID NO:
b) CDR2 comprises the amino acid sequence of SEQ ID NO: 5 or has 3, 2 or 1 amino acid difference(s) with SEQ ID NO: 5; and
c) CDR3 comprises the amino acid sequence of SEQ ID NO: 3 or 6 or has 3, 2 or 1 amino acid difference(s) with SEQ ID NO: 3 or 6, and/or
a) CDR1 comprises the amino acid sequence of SEQ ID NO: 80 or has 3, 2 or 1 amino acid difference(s) with SEQ ID NO: 80;
b) CDR2 comprises the amino acid sequence of SEQ ID NO: 81 or has 3, 2 or 1 amino acid difference(s) with SEQ ID NO: 81; and
c) CDR3 comprises the amino acid sequence of SEQ ID NO: 82 or has 3, 2 or 1 amino acid difference(s) with SEQ ID NO: 82, and/or
a) CDR1 comprises the amino acid sequence of SEQ ID NO: 90 or has 3, 2 or 1 amino acid difference(s) with SEQ ID NO: 90;
b) CDR2 comprises the amino acid sequence of SEQ ID NO: 91 or has 3, 2 or 1 amino acid difference(s) with SEQ ID NO: 91; and
c) CDR3 comprises the amino acid sequence of SEQ ID NO: 92 or has 3, 2 or 1 amino acid difference(s) with SEQ ID NO: 92.

The above CDR sequences are determined according to AbM numbering.

In a preferred embodiment, the at least one albumin-binding ISVD (i) comprised in the polypeptide of the present technology comprises a CDR1 comprising the amino acid sequence of SEQ ID NO: 4, a CDR2 comprising the amino acid sequence of SEQ ID NO: 5; and a CDR3 comprising the amino acid sequence of SEQ ID NO: 6, wherein the CDR sequences are determined according to AbM numbering.

In other embodiments, the at least one albumin-binding ISVD (i) comprised in the polypeptide of the present technology comprises a CDR1 comprising the amino acid sequence of SEQ ID NO: 77, a CDR2 comprising the amino acid sequence of SEQ ID NO: 5; and a CDR3 comprising the amino acid sequence of SEQ ID NO: 6, wherein the CDR sequences are determined according to AbM numbering.

In other embodiments, the at least one albumin-binding ISVD (i) comprised in the polypeptide of the present technology comprises a CDR1 comprising the amino acid sequence of SEQ ID NO: 80, a CDR2 comprising the amino acid sequence of SEQ ID NO: 81; and a CDR3 comprising the amino acid sequence of SEQ ID NO: 82, wherein the CDR sequences are determined according to AbM numbering.

In other embodiments, the at least one albumin-binding ISVD (i) comprised in the polypeptide of the present technology comprises a CDR1 comprising the amino acid sequence of SEQ ID NO: 90, a CDR2 comprising the amino acid sequence of SEQ ID NO: 91; and a CDR3 comprising the amino acid sequence of SEQ ID NO: 92, wherein the CDR sequences are determined according to AbM numbering.

If the CRD sequences are determined according to Kabat numbering, the at least one albumin-binding ISVD (i) comprised in the polypeptide of the present technology would preferably comprise the following CDR regions:
a) CDR1 comprises the amino acid sequence of SEQ ID NO: 1 or has 3, 2 or 1 amino acid difference(s) with SEQ ID NO: 1,
b) CDR2 comprises the amino acid sequence of SEQ ID NO: 2 or has 3, 2 or 1 amino acid difference(s) with SEQ ID NO: 2; and
c) CDR3 comprises the amino acid sequence of SEQ ID NO: 3 or has 3, 2 or 1 amino acid difference(s) with SEQ ID NO: 3, and/or
a) CDR1 comprises the amino acid sequence of SEQ ID NO: 78 or has 3, 2 or 1 amino acid difference(s) with SEQ ID NO: 78;
b) CDR2 comprises the amino acid sequence of SEQ ID NO: 79 or has 3, 2 or 1 amino acid difference(s) with SEQ ID NO: 79; and
c) CDR3 comprises the amino acid sequence of SEQ ID NO: 82 or has 3, 2 or 1 amino acid difference(s) with SEQ ID NO: 82,
and/or
a) CDR1 comprises the amino acid sequence of SEQ ID NO: 93 or has 3, 2 or 1 amino acid difference(s) with SEQ ID NO: 93;
b) CDR2 comprises the amino acid sequence of SEQ ID NO: 94 or has 3, 2 or 1 amino acid difference(s) with SEQ ID NO: 94; and
c) CDR3 comprises the amino acid sequence of SEQ ID NO: 92 or has 3, 2 or 1 amino acid difference(s) with SEQ ID NO: 92.

In some embodiments, the at least one albumin-binding ISVD (i) comprised in the polypeptide of the present technology comprises a CDR1 comprising the amino acid sequence of SEQ ID NO: 1, a CDR2 comprising the amino acid sequence of SEQ ID NO: 2; and a CDR3 comprising the amino acid sequence of SEQ ID NO: 3, wherein the CDR sequences are determined according to Kabat numbering.

In other embodiments, the at least one albumin-binding ISVD (i) comprised in the polypeptide of the present technology comprises a CDR1 comprising the amino acid sequence of SEQ ID NO: 78, a CDR2 comprising the amino acid sequence of SEQ ID NO: 79; and a CDR3 comprising the amino acid sequence of SEQ ID NO: 82, wherein the CDR sequences are determined according to Kabat numbering.

In other embodiments, the at least one albumin-binding ISVD (i) comprised in the polypeptide of the present technology comprises a CDR1 comprising the amino acid sequence of SEQ ID NO: 93, a CDR2 comprising the amino acid sequence of SEQ ID NO: 94; and a CDR3 comprising the amino acid sequence of SEQ ID NO: 92, wherein the CDR sequences are determined according to Kabat numbering.

Specific examples of ISVDs specifically binding to HSA are ISVDs that comprises 4 framework regions (FR1 to FR4, respectively) and three complementarity determining regions, wherein the at least one ISVD specifically binding to a serum albumin protein has:
a) a degree of sequence identity with any one of the sequences as defined in SEQ ID NO's: 7 to 21 or 61 to 69 (in which any C-terminal extension that may be present as well as the CDRs are not taken into account for determining the degree of sequence identity) of at least 85%, preferably at least 90%, more preferably at least 95%; and/or
b) no more than 7, preferably no more than 5, such as only 3, 2 or 1 amino acid differences with any one of the sequences as defined in SEQ ID NO's: 7 to 21 or 61 to 69.

In certain further particular embodiments, the polypeptides according to the present technology comprise at least one domain specifically binding to a serum albumin protein, which is at least one ISVD specifically binding to human serum albumin and essentially consists of 4 framework regions (FR1 to FR4, respectively) and 3 complementarity determining regions (CDR1 to CDR3 respectively), wherein:
a. CDR1 comprises the amino acid sequence according to Kabat numbering of SEQ ID NO: 1 or has 3, 2 or 1 amino acid difference(s) with SEQ ID NO: 1;
b. CDR2 comprises the amino acid sequence according to Kabat numbering of SEQ ID NO: 2 or has 3, 2 or 1 amino acid difference(s) with SEQ ID NO: 2; and
c. CDR3 comprises the amino acid sequence according to Kabat numbering of SEQ ID NO: 3 or has 3, 2 or 1 amino acid difference(s) with SEQ ID NO: 3.

In certain further particular embodiments, the polypeptides according to the present technology comprise at least one domain specifically binding to a serum albumin protein, which is at least one ISVD specifically binding to human serum albumin and essentially consists of 4 framework regions (FR1 to FR4, respectively) and 3 complementarity determining regions (CDR1 to CDR3 respectively), wherein:
a. CDR1 comprises the amino acid sequence according to AbM numbering of SEQ ID NO: 4 or has 3, 2 or 1 amino acid difference(s) with SEQ ID NO: 4;
b. CDR2 comprises the amino acid sequence according to AbM numbering of SEQ ID NO: 5 or has 3, 2 or 1 amino acid difference(s) with SEQ ID NO: 5; and
c. CDR3 comprises the amino acid sequence according to AbM numbering of SEQ ID NO: 6 or has 3, 2 or 1 amino acid difference(s) with SEQ ID NO: 6.

In certain further particular embodiments, the at least one ISVD that specifically binds to (human) serum albumin comprised in the polypeptide of the present technology has:
a. a degree of sequence identity with the sequence of SEQ ID NO: 20 (in which the CDR's and any C-terminal extension that may be present are not taken into account for determining the degree of sequence identity) of at least 85%, preferably at least 90%, more preferably at least 95%; and/or
b. no more than 7, preferably no more than 5, such as only 3, 2 or 1 “amino acid differences” (as defined herein, and not taking into account the CDRs and any C-terminal extension that may be present) with the sequence of SEQ ID NO: 20.

Compared to the sequence of SEQ ID NO: 20, the at least one serum albumin binding moiety in the polypeptides of the present technology preferably also contains (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the FcRn binding polypeptide) as described herein.

In certain further particular embodiments, the at least one ISVD that specifically binds to (human) serum albumin comprised in the polypeptide of the present technology has:
a. a degree of sequence identity with the sequence of SEQ ID NO: 21 (in which the CDR's and any C-terminal extension that may be present are not taken into account for determining the degree of sequence identity) of at least 85%, preferably at least 90%, more preferably at least 95%; and/or
b. no more than 7, preferably no more than 5, such as only 3, 2 or 1 “amino acid differences” (as defined herein, and not taking into account the CDRs and any C-terminal extension that may be present) with the sequence of SEQ ID NO: 21.

Compared to the sequence of SEQ ID NO: 21, the at least one serum albumin binding moiety in the polypeptides of the present technology preferably also contains (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the FcRn binding polypeptide) as described herein.

In certain further particular embodiments, the at least one ISVD that specifically binds to (human) serum albumin comprised in the polypeptide of the present technology has:
a. a degree of sequence identity with the sequence of SEQ ID NO: 65 (in which the CDR's and any C-terminal extension that may be present are not taken into account for determining the degree of sequence identity) of at least 85%, preferably at least 90%, more preferably at least 95%; and/or
b. no more than 7, preferably no more than 5, such as only 3, 2 or 1 “amino acid differences” (as defined herein, and not taking into account the CDRs and any C-terminal extension that may be present) with the sequence of SEQ ID NO: 65.

Compared to the sequence of SEQ ID NO: 65, the at least one serum albumin binding moiety in the polypeptides of the present technology preferably also contains (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the FcRn binding polypeptide) as described herein.

In certain further particular embodiments, the at least one ISVD that specifically binds to (human) serum albumin comprised in the polypeptide of the present technology has:
a. a degree of sequence identity with the sequence of SEQ ID NO: 64 (in which the CDR's and any C-terminal extension that may be present are not taken into account for determining the degree of sequence identity) of at least 85%, preferably at least 90%, more preferably at least 95%; and/or
b. no more than 7, preferably no more than 5, such as only 3, 2 or 1 “amino acid differences” (as defined herein, and not taking into account the CDRs and any C-terminal extension that may be present) with the sequence of SEQ ID NO: 64.

Compared to the sequence of SEQ ID NO: 64, the at least one serum albumin binding moiety in the polypeptides of the present technology preferably also contains (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the FcRn binding polypeptide) as described herein.

In certain further particular embodiments, the at least one ISVD that specifically binds to (human) serum albumin comprised in the polypeptide of the present technology has:
a. a degree of sequence identity with the sequence of SEQ ID NO: 13 (in which the CDR's and any C-terminal extension that may be present are not taken into account for determining the degree of sequence identity) of at least 85%, preferably at least 90%, more preferably at least 95%; and/or
b. no more than 7, preferably no more than 5, such as only 3, 2 or 1 “amino acid differences” (as defined herein, and not taking into account the CDRs and any C-terminal extension that may be present) with the sequence of SEQ ID NO: 13.

Compared to the sequence of SEQ ID NO: 13, the at least one serum albumin binding moiety in the polypeptides of the present technology preferably also contains (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the FcRn binding polypeptide) as described herein.

In certain further particular embodiments, the at least one ISVD that specifically binds to (human) serum albumin comprised in the polypeptide of the present technology has:
a. a degree of sequence identity with the sequence of SEQ ID NO: 69 (in which the CDR's and any C-terminal extension that may be present are not taken into account for determining the degree of sequence identity) of at least 85%, preferably at least 90%, more preferably at least 95%; and/or
b. no more than 7, preferably no more than 5, such as only 3, 2 or 1 “amino acid differences” (as defined herein, and not taking into account the CDRs and any C-terminal extension that may be present) with the sequence of SEQ ID NO: 69.

Compared to the sequence of SEQ ID NO: 69, the at least one serum albumin binding moiety in the polypeptides of the present technology preferably also contains (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the FcRn binding polypeptide) as described herein.

For suitable humanizing substitutions (and suitable combinations thereof), reference is for example made to WO 09/138519 (or in the prior art cited in WO 09/138519) and WO 08/020079 (or in the prior art cited in WO 08/020079), as well as Tables A-3 to A-8 from WO 08/020079 (which are lists showing possible humanizing substitutions). Some preferred but non-limiting examples of such humanizing substitutions are Q108L and A14P or a suitable combination thereof. Such humanizing substitutions may also be suitably combined with one or more other mutations as described herein (such as with one or more mutations that reduce binding by pre-existing antibodies).

For suitable mutations that can reduce the binding by pre-existing antibodies (and suitable combinations of such mutations), reference is for example made to WO 2012/175741 and WO 2015/173325 and also to for example WO 2013/024059 and WO 2016/118733.

Amino acid sequence modifications of the polypeptides or ISVDs described herein are contemplated. For example, it may be desirable to improve the binding affinity and/or other biological properties of the polypeptides or ISVDs. Amino acid sequence variants of the polypeptides or ISVDs as described herein are prepared by introducing appropriate nucleotide changes into the nucleic acid encoding the polypeptides or ISVDs, or by peptide synthesis.

Such modifications include, for example, deletions from, and/or insertions into, and/or substitutions of, residues within the polypeptides or ISVDs as described herein. Any combination of deletion, insertion, and substitution is made to arrive at the final construct, provided that the final construct possesses the desired characteristics. The amino acid changes also may alter post-translational processes of the binding molecules, such as changing the number or position of glycosylation sites. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs). The substitutions are preferably conservative substitutions as described herein. Additionally or alternatively, 1, 2, 3, 4, 5, or 6 amino acids may be inserted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted or deleted in each of the FRs.

The international application WO 2006/122787, the content of which is herein incorporated by reference, describes a number of ISVDs against (human) serum albumin. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of biologics according to the present technology.

WO 2012/175400, the content of which is herein incorporated by reference, describes a further improved version of Alb-1, called Alb-23.

In one embodiment, the polypeptide of the present technology comprises a serum albumin binding ISVD selected from Alb-1, Alb-3, Alb-4, Alb-5, Alb-6, Alb-7, Alb-8, Alb-9, Alb-10 (described in WO 2006/122787) and Alb-23. In one embodiment, the serum albumin binding moiety is Alb-8 or Ab-23 or its variants, as shown on pages 7-9 of WO 2012/175400. In one embodiment, the serum albumin binding ISVD is selected from the albumin binders described in WO 2012/175741, WO 2015/173325, WO 2017/080850, WO 2017/085172, WO 2018/104444, WO 2018/134235, and WO 2018/134234, the content of which is herein incorporated by reference. Some preferred serum albumin binders are also shown in Table A-3. Polypeptides comprising at least one of these albumin binding ISVDs were produced and tested for their beneficial PK properties, as described the examples below.

TABLE A-3
Serum albumin binding ISVD sequences (“ID” refers to the SEQ ID NO as used herein)

| Name | ID | Amino acid sequence |
|---|---|---|
| Alb8 | 7 | EVQLVESGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYCTIGGSLSRS |
| | | SQGTLVTVSS |
| Alb23 | 8 | EVQLLESGGGLVQPGGSLRLSCAASGFTFRSFGMSWVRQAPGKGPEWVSSI |
| | | SGSGSDTLYADSVKGRFTISRDNSKNTLYLQMNSLRPEDTAVYYCTIGGSLSRS |
| | | SQGTLVTVSS |
| Alb129 | 9 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTATYYCTIGGSLSRS |
| | | SQGTLVTVSSA |
| Alb132 | 10 | EVQLVESGGGVVQPGGSLRLSCAASGFTFRSFGMSWVRQAPGKGPEWVSSI |
| | | SGSGSDTLYADSVKGRFTISRDNSKNTLYLQMNSLRPEDTATYYCTIGGSLSRS |
| | | SQGTLVTVSSA |
| Alb11 | 11 | EVQLVESGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYCTIGGSLSRS |
| | | SQGTLVTVSS |
| Alb11 (S112K)-A | 12 | EVQLVESGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYCTIGGSLSRS |
| | | SQGTLVKVSSA |
| Alb82 (ALB11002) | 13 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTALYYCTIGGSLSRS |
| | | SQGTLVTVSS |
| Alb82-A (ALB11002-A) | 14 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTALYYCTIGGSLSRS |
| | | SQGTLVTVSSA |
| Alb82-AA | 15 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTALYYCTIGGSLSRS |
| | | SQGTLVTVSSAA |
| Alb82-AAA | 16 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTALYYCTIGGSLSRS |
| | | SQGTLVTVSSAAA |
| Alb82-G | 17 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTALYYCTIGGSLSRS |
| | | SQGTLVTVSSG |
| Alb82-GG | 18 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTALYYCTIGGSLSRS |
| | | SQGTLVTVSSGG |
| Alb82-GGG | 19 | EVQLVESGGGVVQPGNSLRLSCAASGFTFSSFGMSWVRQAPGKGLEWVSSI |
| | | SGSGSDTLYADSVKGRFTISRDNAKTTLYLQMNSLRPEDTALYYCTIGGSLSRS |
| | | SQGTLVTVSSGGG |
| Alb23002 | 20 | EVQLVESGGGVVQPGGSLRLSCAASGFTFRSFGMSWVRQAPGKGPEWVSSI |
| | | SGSGSDTLYADSVKGRFTISRDNSKNTLYLQMNSLRPEDTALYYCTIGGSLSRS |
| | | SQGTLVTVSS |
| Alb223/Alb23002-A | 21 | EVQLVESGGGVVQPGGSLRLSCAASGFTFRSFGMSWVRQAPGKGPEWVSSI |
| | | SGSGSDTLYADSVKGRFTISRDNSKNTLYLQMNSLRPEDTALYYCTIGGSLSRS |
| | | SQGTLVTVSSA |
| Alb23002(E1D) | 61 | DVQLVESGGGVVQPGGSLRLSCAASGFTFRSFGMSWVRQAPGKGPEWVSS |
| | | ISGSGSDTLYADSVKGRFTISRDNSKNTLYLQMNSLRPEDTALYYCTIGGSLSRS |
| | | SQGTLVTVSS |
| T023500029 | 62 | EVQLVESGGGVVQPGDSLRLSCAASGGTFSTYVMGWFRAPGKEREFVSAIS |
| | | QNSIHTYYANSVKGRFTISRDNSKNTVYLQLNSLRPEDTALYYCAASRFTSWY |
| | | TADYEYDYWGQGTLVTVSS |
| AlbX00001 | 63 | EVQLVESGGGVVQPGGSLRLSCAASGLTFSSYAMGWFRQAPGKERERVVSIS |
| | | RGGGYTYYADSVKGRFTISRDNSENTVYLQMNSLRPEDTALYYCAAARYWAT |
| | | GSEYEFDYWGQGTLVTVSS |
| ALBX00002 | 64 | EVQLVESGGGVVQPGGSLRLSCAASGLTFSSYAMGWFRQAPGKERERVVSIS |
| | | RGGGYTYYADSVKGRFTISRDNSKNTVYLQMNSLRPEDTALYYCAAARYWAT |
| | | GSEYEFDYWGQGTLVTVSS |
| HSA006A06 | 65 | EVQLVESGGGLVQPGNSLRLSCAASGFTFRSFGMSWVRQAPGKEPEWVSSI |
| | | SGSGSDTLYADSVKGRFTISRDNAKTTLYLQMNSLKPEDTAVYYCTIGGSLSRS |
| | | SQGTQVTVSS |
| HSA006A06-A | 66 | EVQLVESGGGLVQPGNSLRLSCAASGFTFRSFGMSWVRQAPGKEPEWVSSI |
| | | SGSGSDTLYADSVKGRFTISRDNAKTTLYLQMNSLKPEDTAVYYCTIGGSLSRS |
| | | SQGTQVTVSSA |
| ALB-1 | 67 | AVQLVESGGGLVQPGNSLRLSCAASGFTFRSFGMSWVRQAPGKEPEWVSSI |
| | | SGSGSDTLYADSVKGRFTISRDNAKTTLYLQMNSLKPEDTAVYYCTIGGSLSRS |
| | | SQGTQVTVSS |
| ALBX00002-A | 68 | EVQLVESGGGVVQPGGSLRLSCAASGLTFSSYAMGWFRQAPGKERERVVSIS |
| | | RGGGYTYYADSVKGRFTISRDNSKNTVYLQMNSLRPEDTALYYCAAARYWAT |
| | | GSEYEFDYWGQGTLVTVSSA |
| T0235002C06(L11V, | 69 | EVQLVESGGGVVQPGDSLRLSCAASGGTFSTYVMGWFRQAPGKEREFVSAI |
| T14P, D74S, K83R, | | SQNSIHTYYANSVKGRFTISRDNSKNTVYLQLNSLRPEDTALYYCAASRFTSWY |
| V89L)-A (T023500029-A) | | TADYEYDYWGQGTLVTVSSA |

In some preferred embodiments, the at least one ISVD specifically binding to a serum albumin protein comprised in the polypeptide of the present technology has a sequence that is chosen from the group consisting of SEQ ID NOs: 7 to 21 or 61 to 69 (Table A-3).

In particular embodiments of the present technology, the polypeptides of the present technology comprise at least one serum albumin binding ISVD having the full amino acid sequence of ALB23002 (SEQ ID NO: 20, see Table A-3).

In particular embodiments of the present technology, the polypeptides of the present technology comprise at least one serum albumin binding ISVD having the full amino acid sequence of Alb223 (SEQ ID NO: 21, see Table A-3).

In some embodiments, the amino acid sequence of the at least one ISVD specifically binding to a serum albumin protein comprised in the polypeptides of the present technology has a sequence identity of more than 90%, such as more than 95%, or more than 99% with Alb-23002 (SEQ ID NO: 20), with Alb223 (SEQ ID NO.: 21), with Alb23002(E1D) (SEQ ID NO: 61), with ALBX00001 (SEQ ID NO: 63), with ALBX00002 (SEQ ID NO: 64), with Alb82 (SEQ ID NO: 13), with T023500029 (SEQ ID NO: 62), with HSA006A06 (SEQ ID NO: 65), or with HSA006A06-A (SEQ ID NO.: 66).

In some embodiments, the amino acid sequence of the at least one ISVD specifically binding to a serum albumin protein comprised in the polypeptides of the present technology has a sequence identity of more than 90%, such as more than 95%, or more than 99% with ALB23002 (SEQ ID NO.: 20), Alb23002-A (SEQ ID NO.: 21), HSA006A06 (SEQ ID NO.: 65), ALBX00002 (SEQ ID NO.: 64), ALB11002 (SEQ ID NO.: 13) and T023500029 (SEQ ID NO.: 69).

Also in a preferred embodiment, the amino acid sequence of an ISVD binding to human serum albumin may have a sequence identity of more than 90%, such as more than 95% or more than 99%, with SEQ ID NO: 20, wherein optionally the CDRs are as defined above as SEQ ID NOs: 1 to 3 (according to Kabat) or SEQ ID NOs: 4 to 6 (according to AbM). In particular, the ISVD binding to human serum albumin preferably has the amino acid sequence of SEQ ID NO: 20.

When such an ISVD binding to human serum albumin has 2 or 1 amino acid difference in at least one CDR relative to a corresponding reference CDR sequence (as defined above as SEQ ID NO: 1 to 3 (according to Kabat) or SEQ ID NOs: 4 to 6 (according to AbM)), the ISVD preferably has at least half the binding affinity, and preferably at least the same binding affinity, to human serum albumin as construct ALB23002, wherein the binding affinity is measured using the same method, such as SPR.

When such an ISVD binding to human serum albumin has a C-terminal position it may exhibit a C-terminal alanine (A) or glycine (G) extension and is preferably selected from SEQ ID NOs: 9, 10, 12, 14, 15, 16, 21 (see table A-3). In a preferred embodiment, the ISVD binding to human serum albumin has another position than the C-terminal position (i.e., is not the C-terminal ISVD of the polypeptide of the technology) and is selected from SEQ ID NOs: 17 to 19 (see Table A-3). Hence, in one embodiment, the ISVD binding to human serum albumin is located at the N-terminal of the polypeptide. In other embodiments, the ISVD binding to human serum albumin is located at the C-terminal of the polypeptide.

In particular embodiments, the polypeptides as described herein comprising the ISVD with the one or more CDRs with 1, 2, 3, or 4 amino acid(s) differences, bind to serum albumin with about the same affinity compared to the binding by the amino acid sequence or polypeptide comprising the CDRs without the 4, 3, 2, or 1 amino acid(s) difference, said affinity as measured by surface plasmon resonance.

When comparing two immunoglobulin single variable domains, the term “amino acid difference” refers to an insertion, deletion or substitution of a single amino acid residue on a position of the first sequence, compared to the second sequence; it being understood that two immunoglobulin single variable domains can contain one, two or more such amino acid differences.

In one preferred embodiment, the at least one ISVDs specifically binding to a serum albumin protein comprises or consists of the amino acid sequence of Alb-23002 (SEQ ID NO.: 20). In another preferred embodiment, the at least one ISVDs specifically binding to a serum albumin protein comprises or consists of the amino acid sequence of Alb23002(E1D) (SEQ ID NO.: 61). In another preferred embodiment, the at least one ISVDs specifically binding to a serum albumin protein comprises or consists of the amino acid sequence of ALBX00002 (SEQ ID NO.: 64). In another preferred embodiment, the at least one ISVDs specifically binding to a serum albumin protein comprises or consists of the amino acid sequence of T023500029 (SEQ ID NO.:62). In another preferred embodiment, the at least one ISVDs specifically binding to a serum albumin protein comprises or consists of the amino acid sequence of Alb 82 (SEQ ID NO.:13). In another preferred embodiment, the at least one ISVDs specifically binding to a serum albumin protein comprises or consists of the amino acid sequence of Alb223 (SEQ ID NO.:21). In another preferred embodiment, the at least one ISVDs specifically binding to a serum albumin protein comprises or consists of the amino acid sequence of HSA006A06 (SEQ ID NO.: 65). In another preferred embodiment, the at least one ISVDs specifically binding to a serum albumin protein comprises or consists of the amino acid sequence of HSA006A06-A (SEQ ID NO.: 66). In another embodiment, the at least one ISVDs specifically binding to a serum albumin protein comprises or consists of the amino acid sequence of ALB-1 (SEQ ID NO.: 67).

In other embodiments, the amino acid sequence of the at least one ISVD specifically binding to a serum albumin protein has a sequence identity of more than 90%, such as more than 95%, or more than 99% with T023500029 (SEQ ID NO: 62). In other embodiments, the amino acid sequence of the at least one ISVD specifically binding to a serum albumin protein has a sequence identity of more than 90%, such as more than 95%, or more than 99% with Alb-23002 (SEQ ID NO.: 20). In other embodiments, the amino acid sequence of the at least one ISVD specifically binding to a serum albumin protein has a sequence identity of more than 90%, such as more than 95%, or more than 99% with Alb23002(E1D) (SEQ ID NO.: 61). In other embodiments, the amino acid sequence of the at least one ISVD specifically binding to a serum albumin protein has a sequence identity of more than 90%, such as more than 95%, or more than 99% with ALBX00002 (SEQ ID NO.: 64). In other embodiments, the amino acid sequence of the at least one ISVD specifically binding to a serum albumin protein has a sequence identity of more than 90%, such as more than 95%, or more than 99% with Alb 82 (SEQ ID NO.:13). In other embodiments, the amino acid sequence of the at least one ISVD specifically binding to a serum albumin protein has a sequence identity of more than 90%, such as more than 95%, or more than 99% with Alb223 (SEQ ID NO.:21). In other embodiments, the amino acid sequence of the at least one ISVD specifically binding to a serum albumin protein has a sequence identity of more than 90%, such as more than 95%, or more than 99% with HSA006A06 (SEQ ID NO.: 65). In other embodiments, the amino acid sequence of the at least one ISVD specifically binding to a serum albumin protein has a sequence identity of more than 90%, such as more than 95%, or more than 99% with HSA006A06-A (SEQ ID NO.: 66). In other embodiments, the amino acid sequence of the at least one ISVD specifically binding to a serum albumin protein has a sequence identity of more than 90%, such as more than 95%, or more than 99% with ALB-1 (SEQ ID NO.: 67).

When an ISVD binding to human serum albumin has 2 or 1 amino acid difference in at least one CDR relative to a corresponding reference CDR sequence (e.g., as defined above as SEQ ID NO: 4 to 6, according to AbM numbering) the ISVD preferably has at least half the binding affinity, and preferably at least the same binding affinity, to human serum albumin as construct ALB23002 (SEQ ID NO: 20), wherein the binding affinity is measured using the same method, such as surface plasmon resonance.

When such an ISVD binding to human serum albumin has a C-terminal position in the polypeptide of the present technology, it may exhibit a C-terminal alanine (A) or C-terminal glycine (G) extension, and is preferably selected from SEQ ID NOs: 9, 10, 12, 14, 15, 16, 17, 18, 19 and 21 (see Table A-3).

In particular embodiments, the polypeptides as described herein comprising the ISVD as defined herein with the one or more CDRs with 1, 2, 3, or 4 amino acid(s) differences, bind to serum albumin with about the same affinity compared to the binding by the amino acid sequence or polypeptide comprising the CDRs as defined herein without the 4, 3, 2, or 1 amino acid(s) difference, said affinity as measured by surface plasmon resonance.

Compared to the sequence of SEQ ID NO: 20, the at least one serum albumin binding ISVD comprised in the polypeptides of the present technology preferably also contain (at least): one or more humanizing substitutions;
and/or
one or more mutations (i.e., amino acid substitutions, deletions or additions, and in particular substitutions) that reduce the binding by pre-existing antibodies; and may optionally contain one or more further mutations (e.g., to improve chemical stability of the polypeptide) as described herein.

For suitable humanizing substitutions (and suitable combinations thereof), reference is for example made to WO 09/138519 (or in the prior art cited in WO 09/138519) and WO 08/020079 (or in the prior art cited in WO 08/020079), as well as Tables A-3 to A-8 from WO 08/020079 (which are lists showing possible humanizing substitutions). Some preferred but non-limiting examples of such humanizing substitutions are Q108L and A14P or a suitable combination thereof. Such humanizing substitutions may also be suitably combined with one or more other mutations as described herein (such as with one or more mutations that reduce binding by pre-existing antibodies).

For suitable mutations that can reduce the binding by pre-existing antibodies (and suitable combinations of such mutations), reference is for example made to WO 2012/175741 and WO 2015/173325 and also to for example WO 2013/024059 and WO 2016/118733.

A useful method for identification of certain residues or regions of polypeptides or ISVDs as described herein, that are preferred locations for mutagenesis is called “alanine scanning mutagenesis” as described by Cunningham and Wells 1989 (Science 244: 1081-1085). Here, a residue or group of target residues within the binding molecule is/are identified (e.g., charged residues such as Arg, Asp, His, Lys, and Glu) and replaced by a neutral or negatively charged amino acid (most preferably alanine or polyalanine) to affect the interaction of the amino acids with the epitope. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se needs not to be predetermined. For example, to analyze the performance of a mutation at a given site, ala scanning or random mutagenesis is conducted at a target codon or region and the expressed binding molecule variants are screened for the desired activity.

Preferably, amino acid sequence insertions include amino- and/or carboxyl-terminal fusions ranging in length from 1, 2, 3, 4, 5, 6, 7, 8, 9 or 10 residues to polypeptides containing a hundred or more residues.

Another type of variant is an amino acid substitution variant. These variants have preferably (at least) 1, 2, 3, 4, 5, 6, 7, 8, 9 or 10 amino acid residues in the amino acid sequences, ISVDs or polypeptides replaced by a different residue. The sites of greatest interest for substitution mutagenesis include the CDRs, in particular the hypervariable regions, but FR alterations are also contemplated. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

Generally, if amino acids are substituted in one or more or all of the CDRs, it is preferred that the then-obtained “substituted” sequence is at least 60%, more preferably 65%, even more preferably 70%, particularly preferably 75%, more particularly preferably 80% or even more than 90% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the amino acid sequences, ISVDs or polypeptides may have different degrees of identity to their substituted sequences, e.g., CDR1 may have 80%, while CDR3 may have 90%.

Preferred amino acid substitutions are conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gin; (c) polar, positively charged residues: His, Arg and Lys; (d) large aliphatic, nonpolar residues: Met, Leu, lie, Val and Cys; and (e) aromatic residues: Phe, Tyr and Trp. Further preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gin; lie into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu. However, any substitution (including non-conservative substitution) is envisaged as long as the polypeptide retains its capability to specifically bind to the epitope on FcRn as described herein, specifically bind to FcRn at acidic pH (such as pH 5.0 to 6.8) with an affinity as described herein (e.g., with a K_{D} between 10⁻⁶ and 10⁻¹¹ M) and/or its CDRs have an identity of at least 60%, more preferably 65%, even more preferably 70%, particularly preferably 75%, more particularly preferably 80% to the “original” CDR sequence.

In some embodiments, the ISVD is a (single) domain antibody, a V_{HH}, a humanized V_{HH}, or a camelized V_{H}, preferably a Nanobody®.

Thus, in particular embodiments, the polypeptides according to the present technology comprise at least one ISVD binding to human serum albumin, which is chosen from the group consisting of SEQ ID NOs: 7 to 21, or 61 to 69, and at least one Fc domain of an IgG, as described herein.

According to particular embodiments, the polypeptides of the present technology, such as the FcRn targeting polypeptides of the present technology, are preferably also such that they compete with the polypeptides with the amino acid sequence of SEQ ID NO's: 7 to 21, or 61 to 69, or 102-104 for binding to human serum albumin and/or that they “cross-block” (as defined below) the binding of the polypeptides with the amino acid sequence of SEQ ID NO's: 7 to 21, or 61 to 69, or 102-104 to human serum albumin.

The terms “cross-block”, “cross-blocked” and “cross-blocking” are used interchangeably herein to mean the ability of an immunoglobulin single variable domain or polypeptide to interfere with the binding of a ligand to its target, such as a natural ligand to its receptor(s). The extent to which an immunoglobulin single variable domain or polypeptide of the present technology is able to interfere with the binding of another compound such as the natural ligand to its target and therefore whether it can be said to cross-block according to the present technology, can be determined using competition binding assays. One particularly suitable quantitative cross-blocking assay uses a FACS- or an ELISA-based approach or Alphascreen to measure competition between the labelled (e.g., His tagged or biotinylated) immunoglobulin single variable domain or polypeptide according to the present technology and the other binding agent in terms of their binding to the target. Suitable FACS-, ELISA- or Alphascreen-displacement-based assays for determining whether a binding molecule cross-blocks or is capable of cross-blocking a polypeptide are well known. It will be appreciated that these assays can be used with any of the immunoglobulin single variable domains or other binding agents described herein. Thus, in general, a cross-blocking polypeptide according to the present technology is for example one which will bind to the target in the above cross-blocking assay such that, during the assay and in the presence of a second polypeptide or in the presence of the natural ligand, the recorded displacement of the immunoglobulin single variable domain or polypeptide according to the present technology is between 60% and 100% (e.g., in ELISA/Alphascreen based competition assay) or between 80% to 100% (e.g., in FACS based competition assay) of the maximum theoretical displacement (e.g., displacement by cold (e.g., unlabeled) immunoglobulin single variable domain or polypeptide that needs to be cross-blocked) by the to be tested potentially cross-blocking agent that is present in an amount of 0.01 mM or less.

In particular embodiments, the polypeptides of the present technology are such that they comprise at least one ISVD binding to essentially the same amino acid residues and/or epitope on (human) serum albumin as the amino acid residues and/or epitope bound by the polypeptide with the amino acid sequence of SEQ ID NOs: 7 to 21, or 61 to 69 and even more preferably such that they share essentially the same amino acid interactions as the polypeptide with the amino acid sequence of SEQ ID NOs: 7 to 21, or 61 to 69. For this purpose, according to a specific but non-limiting aspect, the polypeptides according to the present technology comprise at least one ISVD that preferably either has the same CDRs as the sequence of SEQ ID NOs: 7 to 21, or 61 to 69, or compared to the sequence of SEQ ID NOs: 7 to 21, or 61 to 69, preferably contain within their CDRs only such mutations (such as conservative amino acid substitutions) that still allow them to undergo the same or essentially the same amino acid interactions with (human) serum albumin as the polypeptide with the sequence of SEQ ID NOs: 7 to 21, or 61 to 69.

In further particular embodiments, the (i) at least one domain specifically binding to a serum albumin protein comprised in the polypeptide of the present technology is at least one ISVD specifically binding to domain II of serum albumin, such as domain II of human serum albumin.

According to particular embodiments of the present technology, the at least one domain specifically binding to a serum albumin protein that is comprised in the polypeptides of the present technology is at least one ankyrin repeat sequence (DARPin sequence) specifically binding to (human) serum albumin.

In particular embodiments, the polypeptides of the present technology comprise at least one serum albumin binding domain which is an ankyrin repeat sequence, such as for example but not limited to the sequences with SEQ ID NO's 17 to 31 and SEQ ID NOs 43 to 52 as disclosed in and specifically described on pages 15-27 of WO 2012/069654, SEQ ID NO: 50 as disclosed in WO 2016/156596, SEQ ID NO:'s 9 to 11 as disclosed in and specifically described on pages 9-11 of WO 2018/054971 and SEQ ID NO's: 3 and 4 as disclosed and specifically described on pages 5-12 of WO 2020/24517.

Polypeptides comprising at least one of these albumin binding ankyrin repeat sequences are produced and tested for their beneficial PK properties.

Hence, in other embodiments, the (i) at least one domain specifically binding to a serum albumin protein comprised in the polypeptide of the present technology is at least one DARPin specifically binding to serum albumin. For instance, the at least one DARPin may comprise or consist of SEQ ID NO.: 102, or a polypeptide with at least 90%, such as at least 95%, or at least 97%, or at least 99% sequence identity with SEQ ID NO.: 102.

According to particular embodiments of the present technology, the at least one domain specifically binding to albumin that is comprised in the polypeptides of the present technology is at least one affitin (aka Nanofitin®) that specifically binds to (human) serum albumin.

In particular embodiments, the at least one serum albumin binding Affitin is for example but not limited to the sequences with SEQ ID NO's 38 and SEQ ID NO's 45 to 86 as disclosed in and specifically described on pages 6 to 16 of WO 2022/171852.

Polypeptides comprising at least one of these albumin binding affitins are produced and tested for their beneficial PK properties.

Hence, in other embodiments, the (i) at least one domain specifically binding to a serum albumin protein comprised in the polypeptide of the present technology is at least one affitin specifically binding to serum albumin. For instance, the at least one Affitin may comprise or consist of SEQ ID NO.: 103, or a polypeptide with at least 90%, such as at least 95%, or at least 97%, or at least 99% sequence identity with SEQ ID NO.: 103.

According to particular embodiments of the present technology, at least one domain specifically binding to albumin that is comprised in the polypeptides of the present technology is at least one ABD of a bacterial receptor protein that specifically binds to (human) serum albumin.

Streptococcal protein G is a bi-functional receptor present on the surface of certain strains of streptococci and capable of binding to both IgG and serum albumin (Bjorck et al., Mol Immunol 24:11 13, 1987). The structure is highly repetitive with several structurally and functionally different domains (Guss et al., EMBO J 5:1567, 1986), more precisely three Ig-binding motifs and three serum albumin binding domains (Olsson et al., Eur J Biochem 168:319, 1987). The structure of one of the three serum albumin binding domains has been determined, showing a three-helix bundle domain (Kraulis et al., FEBS Lett 378:190, 1996). This motif was named ABD (albumin binding domain) and is 46 amino acid residues in size. In the literature, it has subsequently also been designated G148-GA3. Other bacterial albumin binding proteins than protein G from Streptococcus have also been identified, which contain domains similar to the albumin binding three-helix domains of protein G. Examples of such proteins are the PAB, PPL, MAG and ZAG proteins. Studies of structure and function of such albumin binding proteins have been carried out and reported e.g., by Johansson and co-workers (Johansson et al., J Mol Biol 266:859-865, 1997; Johansson et al., J Biol Chem 277:81 14-8120, 2002), who introduced the designation “GA module” (protein G-related albumin binding module) for the three-helix protein domain responsible for albumin binding. Furthermore, Rozak et al. have reported on the creation of artificial variants of the GA module, which were selected and studied with regard to different species specificity and stability (Rozak et al., Biochemistry 45:3263-3271, 2006; He et al., Protein Science 16:1490-1494, 2007). Recently, variants of the G148-GA3 domain have been developed, with various optimized characteristics. Such variants are for example disclosed in PCT publications WO 2009/016043, WO 2012/004384, WO 2014/04897 and WO 2015/091957.

Polypeptides comprising at least one of these ABD's are produced and tested for their beneficial PK properties.

Hence, in other embodiments, the (i) at least one domain specifically binding to a serum albumin protein comprised in the polypeptide of the present technology is at least one albumin-binding domain (ABD) specifically binding to serum albumin. For instance, the at least one ABD may comprise or consist of SEQ ID NO.: 104, or a polypeptide with at least 90%, such as at least 95%, or at least 97%, or at least 99% sequence identity with SEQ ID NO.: 104.

TABLE A-4
Serum albumin binding protein sequences
(“ID” refers to the SEQ ID NO as used herein)

| Name | ID | Amino acid sequence |
|---|---|---|
| Darpin | 102 | DLGKKLLEAARAGQDDEVRELLKAGADVNAKD |
| | | YFSHTPLHLAARNGHLKIVEVLLKAGADVNAK |
| | | DFAGKTPLHLAANEGHLEIVEVLLKAGADVNA |
| | | QDIFGKTPADIAADAGHEDIAEVLQKAA |
| Affitin | 103 | VKVKFWPRGEEKVVDTSKIAWVLRADKTVMFK |
| (Nanofitin) | | YDDNGKKGYGVVLEKDAPKELLDMLARAEREK |
| ABD | 104 | LKEAKEKAIEELKKAGITSDYYFDLINKAKTV |
| | | EGVNALKDEILKA |

In other embodiments, the (i) at least one domain specifically binding to a serum albumin protein comprised in the polypeptide of the present technology is selected from an albumin binding unit described in any of the following applications, all incorporated by reference: WO 2023/147042, WO 2022/026643, WO 2021/119531, WO 2021/119531, WO 2020/229842, WO 2020/172528, WO 2020/099871, WO 2017/201488, WO 2014/111550, WO 2013/167883, WO 2013/043071, WO 2012/072731, WO 2012/022703, WO 2012/020143, WO 2011/144751, WO 2011/006915, WO 2010/094723, WO 2010/094722, WO 2008/096158, WO 2005/118642, WO 2004/003019, US20140186365 and US20130129727.

### 5.4 Second Domain: A Fc Domain of an IgG or a Fragment Thereof

As described above, the polypeptides of the present technology comprise (i) at least one domain comprising a serum albumin protein and/or a domain, such as a serum albumin binding ISVD, that has high affinity for/binds specifically to serum albumin protein and (ii) at least one Fc domain of an IgG, or a fragment thereof, preferably a FcRn-binding fragment thereof.

As used in the present description, the term “Fc,” “Fc domain,” “Fc region,” or “Fc fragment” is used interchangeably and is defined as the portion of a heavy chain constant region beginning in the hinge region just N-terminal of the papain cleavage site (i.e., residue 216 in IgG, taking the first residue of heavy chain constant region to be 114) and ending at the C-terminus of the heavy chain. Accordingly, a complete Fc, Fc domain, Fc region, or Fc fragment comprises at least a hinge region or a part of it, a CH2 domain, and a CH3 domain. For instance, a Fc domain may comprise at least a hinge region or a part of it, two CH2 domains, and two CH3 domains. Sequence alignment of portions (CH2 and CH3 domains, residues 231 to 447, Eu numbering) of exemplary human IgG1, IgG2, IgG3, and IgG4 Fc domains is shown in FIG. 32 of WO 2021/016571. The term encompasses native/wildtype Fc and Fc variants as described herein and includes molecules in monomeric or multimeric (e.g., dimeric) form, whether digested from whole antibody or produced by other means such as recombinant technology. See, e.g., Ying et al., JBC (2013) 288:25154-164; and Yang et al., JBC (2019) 294:10638-48.

Hence, the term “Fc domain,” “Fc region”, or “Fc” refers to a C-terminal non-antigen binding region of an immunoglobulin heavy chain that contains at least a portion of the constant region. Traditionally, the term Fc domain refers to a protease (e.g., papain) cleavage product encompassing the paired CH2, CH3 and hinge regions of an antibody. In the context of the present disclosure, the term “Fc domain”, “Fc region” or “Fc” refers to any polypeptide (or nucleic acid encoding such a polypeptide), regardless of the means of production, that includes all or a portion of the CH2, CH3 and hinge regions of an immunoglobulin polypeptide. Thus, in some embodiments, the Fc domain includes, from N- to C-terminus, CH2-CH3 and hinge-CH2-CH3. Although the boundaries of the Fc region may vary, the human IgG heavy chain Fc region is usually defined to include residues E216, C226, or A231 to its carboxyl-terminus, wherein the numbering is according to the EU index as in Kabat.

The term includes native (i.e., wild type) Fc regions and variant Fc regions. In certain embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in in Edelman, G M et al. Proc. Natl. Acad. USA, 63, 78-85 (1969) and in Kabat et al., Sequences of Proteins of Immunological Interest, 5th Ed. Public Health Service, National Institutes of Health, Bethesda, MD, 1991.

In certain embodiments, the term “Fc domain”, “Fc region” or “Fc” refers to an immunoglobulin IgG heavy chain constant region comprising a hinge region (starting at Cys226), an IgG CH2 domain, and CH3 domain. In certain embodiments, the Fc region starts at the hinge region and extends to the C-terminus of the IgG heavy chain.

The original immunoglobulin source of the native Fc is typically of human origin and can be any of the immunoglobulins G, such as IgG1, IgG2, IgG3 or IgG4, in particular IgG1 and IgG4. In a preferred embodiment, the Fc domain is of human origin and from IgG1 or IgG4, preferably from IgG4. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e. g., IgG, IgA, and IgE) or subclass (e g., IgG1, IgG2, IgG3, IgG4, IgA1, and IgA2). One example of a native Fc is a disulfide-bonded dimer resulting from papain digestion of an IgG. The term “native Fc”, as used herein, is generic to the monomeric, dimeric, and multimeric forms.

The Fc domain comprised in the polypeptide of the present technology preferably comprises two disulfide-bonded chains (chain 1 and chain 2). The chains may be identical (homodimer) or different (heterodimer). Hence, the polypeptide of the present technology, may comprise two polypeptides bonded by disulfide bridges. The polypeptide of the present technology may comprise more than two polypeptides, such as four polypeptides (more than two chains, such as four chains, see, e.g., Table A-1).

In certain particular embodiments, the Fc region comprises the Fc region of human IgG1, IgG2, IgG3 or IgG4. In certain particular embodiments, the Fc region comprises the CH2 and CH3 domain of IgG, including an Fc domain being one single monomeric Fc chain. In certain particular embodiments, the Fc region comprises the CH2 and CH3 domain of IgG4. In certain other particular embodiments, the Fc region comprises the CH2 and CH3 domain of IgG1. In certain other particular preferred embodiments, the Fc region comprises the hinge region of IgG1 and the CH2 and CH3 domain of IgG4.

In certain embodiments, the IgG CH2 domain starts at Ala 231. In certain other embodiments, the CH3 domain starts at Gly 341. It is understood that the C-terminus Lys residue of human IgG can be optionally absent. It is also understood that conservative amino acid substitutions of the Fc region without affecting the desired structure and/or stability of Fc is contemplated within the scope of the present technology.

In particular embodiments, the polypeptides of the present technology comprise at least one native Fc domain of an immunoglobulin G. In these embodiments, the at least one variant Fc domain of an immunoglobulin G specifically binds to FcRn with a K_{D} value of less than about 550, 525, 500, 475, 450, 425, 400, 375, 350, 325, 300, 275, 250, 225, 200, 175, 150, 125, or 100 nM (e.g., at a pH of between 5.0 and 6.8). In further particular embodiments, the polypeptides of the present technology comprise at least one native Fc domain of an IgG and specifically bind to FcRn, at a pH of between about 5.0 and 6.8, preferably at a pH of about 6.0, with a K_{D} value of less than about 75 nM, such as less than 50 nM, 25 nM, 20 nM, 17 nM, 15 nM, 10 nM, 5 nM, 2.5 nM, 1 nM (at a pH of between 5.0 and 6.8). In yet further particular embodiments, the polypeptides of the present technology comprise at least one native Fc domain of an IgG and bind specifically to FcRn at a pH of between about 5.0 and 6.8, preferably at a pH of about 6.0, with a K_{D} value of between about 250 nM and 1 nM, such as between 100 nM and 1 nM, preferably between 75 nM and 1 nM, such as between 50 nM and 1 nM, most preferably between 25 nM and 1 nM, such as about 20 nM, such as about 17 nM. Preferably, the K_{D} is determined by Kinexa, BLI or SPR, for instance as determined by SPR.

In particular embodiments, the polypeptides of the present technology comprise, in addition to a domain comprising a serum albumin protein and/or a domain specifically binding to a serum albumin protein, one single Fc domain, which is a native Fc domain of an IgG. In these embodiments, the one native Fc domain of an immunoglobulin G specifically binds to FcRn with a K_{D} value of less than about 550, 525, 500, 475, 450, 425, 400, 375, 350, 325, 300, 275, 250, 225, 200, 175, 150, 125, or 100 nM (e.g., at a pH of between 5.0 and 6.8). In further particular embodiments, the one native Fc domain of an IgG specifically binds to FcRn, at a pH of between about 5.0 and 6.8, preferably at a pH of about 6.0, with a K_{D} value of less than about 75 nM, such as less than 50 nM, 25 nM, 20 nM, 17 nM, 15 nM, 10 nM, 5 nM 2.5 nM, 1 nM (at a pH of between 5.0 and 6.8).

In particular embodiments, the polypeptides of the present technology comprise, in addition to a domain comprising a serum albumin protein and/or a domain specifically binding to a serum albumin protein, one single Fc monomeric chain, which is a native Fc monomeric chain of an IgG. In these embodiments, the one native monomeric Fc chain of an immunoglobulin G specifically binds to FcRn with a K_{D} value of less than about 550, 525, 500, 475, 450, 425, 400, 375, 350, 325, 300, 275, 250, 225, 200, 175, 150, 125, or 100 nM (e.g., at a pH of between 5.0 and 6.8). In further particular embodiments, the one native monomeric Fc chain of an IgG specifically binds to FcRn, at a pH of between about 5.0 and 6.8, preferably at a pH of about 6.0, with a K_{D} value of less than about 75 nM, such as less than 50 nM, 25 nM, 20 nM, 17 nM, 15 nM, 10 nM, 5 nM 2.5 nM, 1 nM (at a pH of between 5.0 and 6.8).

In certain embodiments, one or more amino acid modifications may be introduced into a native Fc region comprised in the polypeptides of the technology, thereby generating an Fc variant. Accordingly, in some particular embodiments, the Fc domain is a variant Fc domain.

In certain particular embodiments, the polypeptides of the present technology comprise, in addition to a domain comprising a serum albumin protein and/or a domain specifically binding to a serum albumin protein, at least one variant Fc domain of an immunoglobulin G and specifically bind to FcRn with a K_{D} value of less than about 550, 525, 500, 475, 450, 425, 400, 375, 350, 325, 300, 275, 250, 225, 200, 175, 150, 125, or 100 nM (e.g., at a pH of between 5.0 and 6.8). In yet further particular embodiments, the polypeptides of the present technology comprise at least one variant Fc domain of an IgG and bind to FcRn, i.e., at a pH of between about 5.0 and 6.8, preferably at a pH of about 6.0, with a K_{D} value of between about 250 nM and 1 nM, such as between 100 nM and 1 nM, preferably between 75 nM and 1 nM, such as between 50 nM and 1 nM, most preferably between 25 nM and 1 nM, such as about 20 nM, such as about 17 nM. Preferably, the K_{D} is determined by Kinexa, BLI or SPR, for instance as determined by SPR.

In certain other particular embodiments, the polypeptides of the present technology comprise, in addition to a domain comprising a serum albumin protein and/or a domain specifically binding to a serum albumin protein, at least one variant Fc domain that does not detectably, selectively or specifically bind to FcRn, or that exhibits no or essentially no binding to FcRn neither at a pH of between 5.0 and 6.8 nor at neutral or physiologic pH, such as at a pH of 7.4.

In particular embodiments, the polypeptides of the present technology comprise, in addition to a domain comprising a serum albumin protein and/or a domain specifically binding to a serum albumin protein, one single Fc domain, which is a variant Fc domain of an IgG. In these embodiments, the one variant Fc domain of an immunoglobulin G specifically binds to FcRn with a K_{D} value of less than about 550, 525, 500, 475, 450, 425, 400, 375, 350, 325, 300, 275, 250, 225, 200, 175, 150, 125, or 100 nM (e.g., at a pH of between 5.0 and 6.8). In further particular embodiments, the one variant Fc domain of an IgG specifically binds to FcRn, at a pH of between about 5.0 and 6.8, preferably at a pH of about 6.0, with a K_{D} value of less than about 75 nM, such as less than 50 nM, 25 nM, 20 nM, 17 nM, 15 nM, 10 nM, 5 nM 2.5 nM, 1 nM (at a pH of between 5.0 and 6.8).

In particular embodiments, the polypeptides of the present technology comprise, in addition to a domain comprising a serum albumin protein and/or a domain specifically binding to a serum albumin protein, one single monomeric Fc chain, which is a variant monomeric Fc chain of an IgG. In these embodiments, the one variant monomeric Fc chain of an immunoglobulin G specifically binds to FcRn with a K_{D} value of less than about 550, 525, 500, 475, 450, 425, 400, 375, 350, 325, 300, 275, 250, 225, 200, 175, 150, 125, or 100 nM (e.g., at a pH of between 5.0 and 6.8). In further particular embodiments, the one variant monomeric Fc chain of an IgG specifically binds to FcRn, at a pH of between about 5.0 and 6.8, preferably at a pH of about 6.0, with a K_{D} value of less than about 75 nM, such as less than 50 nM, 25 nM, 20 nM, 17 nM, 15 nM, 10 nM, 5 nM 2.5 nM, 1 nM (at a pH of between 5.0 and 6.8).

The term “Fc,” “Fc domain,” “Fc region,” or “Fc fragment” used in the present description encompasses also “Fc variants” “modified Fc” or “modified Fc domains”, i.e., molecules or sequences that are modified from a native/wildtype Fc but still comprise a binding site for the FcRn. A Fc variant or a modified Fc domain also can be shorter or longer than a native Fc (e.g., shorter or longer than a sequence spanning residues 216 to 447 of human IgG, Eu numbering); for example, the Fc variant or modified Fc may lack certain N-terminal and/or C-terminal amino acid residues of the native Fc, or may contain additional amino acid residues at the N-terminus and/or C-terminus compared to a native Fc. A modified Fc domain itself does not include an antigen-binding domain of an antibody or an antibody variant, or a target-binding domain of an immunoadhesin. The term encompasses a molecule or sequence that is humanized from a non-human native Fc. Furthermore, a native Fc comprises regions that can be removed because they provide structural features or biological activities that are not required for the FcRn antagonists (e.g., antibody-like binding polypeptides) as described in WO 2021/016571. Thus, the term encompasses a molecule or sequence that lacks one or more native Fc sites or residues, or in which one or more Fc sites or residues have been modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor (an FcγR), or (7) antibody-dependent cellular cytotoxicity (ADCC) or complement-dependent cytotoxicity (CDC).

“Fc variant domain”, “Fc variant region”, “Fc variant” or “variant Fc” as used herein is meant a protein comprising at least one amino acid modification in a native Fc domain (as defined herein). The modification can be an addition, deletion, or substitution.

The Fc variant may comprise a human Fc region sequence (e.g., a human IgG1, IgG2, IgG3 or IgG4 Fc region) comprising an amino acid modification (e.g., an addition, deletion and/or substitution) at one or more amino acid positions.

In certain embodiments, the present technology contemplates an Fc variant that possesses some but not all effector functions, which make it a desirable candidate for applications in which the half-life of the polypeptide comprising an Fc region in vivo is important.

Certain effector functions (such as complement dependent cytotoxicity (CDC) and antibody-dependent cellular cytotoxicity (ADCC)) may be unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the Fc lacks FcγR binding (hence likely lacking ADCC activity) but retains FcRn binding ability. Non-limiting examples of in vitro assays to assess ADCC activity of a molecule of interest are described in U.S. Pat. No. 5,500,362 (see e.g., Hellstrom et al., Proc. Nat. Acad. Sci. USA 83:7059-7063 (1986) and Hellstrom et al., Proc. Nat. Acad. Sci. USA 82:1499-1502 (1985); U.S. Pat. No. 5,821,337 (see Bruggemann et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat. Acad. Sci. USA 95:652-656 (1998). C1q binding assays may also be carried out to confirm that the Fc is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg et al., Blood 101:1045-1052 (2003); and Cragg et al., Blood 103:2738-2743 (2004)). FcRn binding and in vivo clearance/half-life determinations can also be performed using methods known in the art (see, e.g., Petkova et al., Int. Immunol. 18(12):1759-1769 (2006)).

Fc regions with reduced effector function include those with substitution of one or more of Fc region residues 238, 265, 269, 270, 297, 327 and 329 (U.S. Pat. No. 6,737,056). Such Fc variants include those with substitutions at two or more of amino acid positions 265, 269, 270, 297 and 327, including the so-called “DANA” Fc variant with substitution of residues 265 and 297 to alanine (U.S. Pat. No. 7,332,581).

Certain Fc variants with improved or diminished binding to FcRs are described for example in U.S. Pat. No. 6,737,056; WO 2004/056312, and Shields et al., J. Biol. Chem. 9(2): 6591-6604 (2001).

In some embodiments, alterations are made in the Fc region that result in diminished C1q binding and/or Complement Dependent Cytotoxicity (CDC), for example as described in U.S. Pat. No. 6,194,551, WO 99/51642, and Idusogie et al. J. Immunol. 164: 4178-4184 (2000).

Fc domains with improved binding to the neonatal Fc receptor (FcRn) according to the present technology include Fc variants with substitutions at for example but not limited to one or more of Fc region residues: 238, 256, 265, 272, 286, 303, 305, 307, 311, 312, 317, 340, 356, 360, 362, 376, 378, 380, 382, 413, 424.

In certain embodiments, it may be desirable to create cysteine engineered Fc fusion protein, in which one or more residues of the Fc region of an antibody are substituted with cysteine residues. In particular embodiments, the substituted residues occur at accessible sites of the Fc. By substituting those residues with cysteine, reactive thiol groups are thereby positioned at accessible sites of the Fc and may be used to conjugate the Fc to other moieties, such as drug moieties or linker-drug moieties, to create an immunoconjugate, as described further herein.

Other variants of suitable Fc domains as well as suitable formats of Fc domain constructs are well known in the art and are inter alia described in published patent applications EP 2654790, U.S. Ser. No. 10/239,944, US 20120251531, U.S. Pat. No. 9,133,274, WO 2014/065945, WO 2015/150447 and WO 2021/016571.

Specific suitable formats of polypeptides according to particular embodiments of the present technology comprising at least one serum albumin protein or binder to a serum albumin protein and at least one Fc domain will become clear from the examples as further described herein.

Hence, in particular embodiments, the polypeptides of the present technology comprise, in addition to at least one domain comprising a serum albumin protein and/or at least one domain specifically binding to a serum albumin protein, at least one Fc domain of an IgG. Accordingly, in particular embodiments, the Fc domain comprised in the polypeptides of the present technology specifically binds to human FcRn (SEQ ID NO: 24) or (polymorphic) variants or isoforms thereof at a pH of between about 5.0 and 6.8. However, in other particular embodiments, the polypeptides of the present technology comprise at least one Fc domain of an IgG that does not specifically bind to FcRn (neither at a pH of between about 5.0 and 6.8 nor at a pH of about 7.4). The Fc domain comprised in the polypeptide of the present technology may also comprise any of the mutations described herein.

In particular embodiments, the polypeptides according to the present technology are preferably such that when these are bound to or otherwise associated with an FcRn molecule, the binding of the FcRn molecule to serum albumin and/or IgG is not (significantly) affected, reduced or inhibited. In these particular embodiments, in a cross-blocking assay (as described herein), when the FcRn binding polypeptide is bound to or otherwise associated with an FcRn molecule, the displacement of serum albumin is less than 40%, such as less than 30%, less than 20%, less than 10% or essentially no displacement is detected (e.g., in ELISA or Alphascreen based competition assay). In this particular embodiment, in a cross-blocking assay (as described herein), when the FcRn binding polypeptide is bound to or otherwise associated with an FcRn molecule, the displacement of IgG is less than 40%, such as less than 30%, less than 20%, less than 10% or essentially no displacement is detected (e.g., in ELISA or Alphascreen based competition assay). It is preferred that the Fc domain comprised in the polypeptide of the present technology is such that when these are bound to or otherwise associated with an FcRn molecule, the binding of the FcRn molecule to serum albumin and/or IgG is not (significantly) affected, reduced or inhibited, as described herein.

In certain embodiments, the Fc domain of an IgG comprised in polypeptide of the present technology may comprise one or more amino acid mutations (e.g., substitutions) which alter the effector functions (e.g., ADCC or CDC function) of the Fc domain, as compared to a corresponding wildtype molecule. In certain embodiments, the Fc domain of an IgG comprised in polypeptide of the present technology may comprise one or more amino acid mutations (e.g., substitutions) which provide one or more desired biochemical characteristics such as the ability to remain monomeric, the ability to noncovalently dimerize, an increased ability to localize at a target site, and glycosylation patterns, as compared to the corresponding wildtype molecule. For example, the modified Fc domain may have reduced glycosylation (e.g., N- or O-linked glycosylation). Exemplary amino acid substitutions which confer reduced or altered glycosylation are disclosed in WO 2005/018572. In some embodiments, the Fc domain is modified to eliminate glycosylation (e.g., “agly” antibodies).

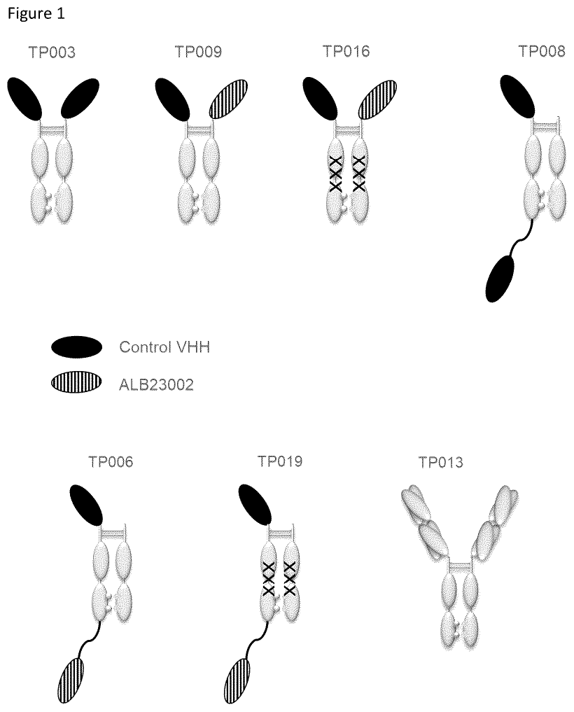

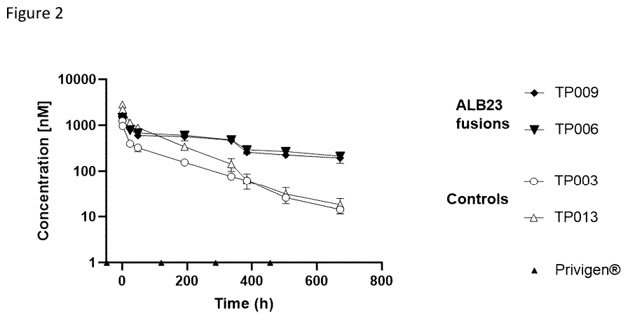

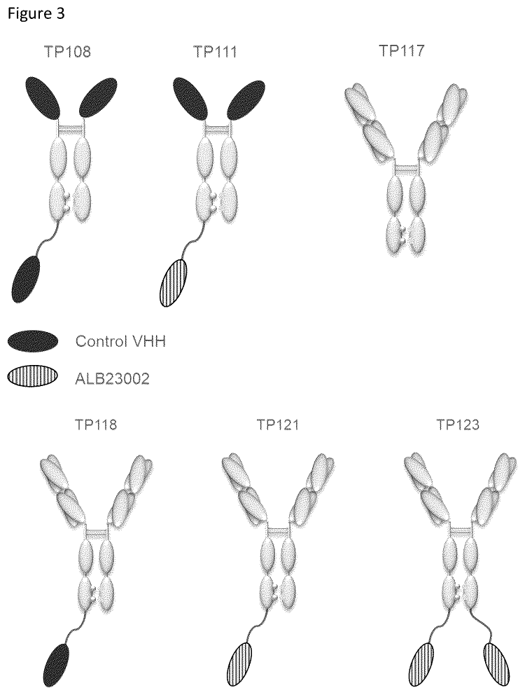

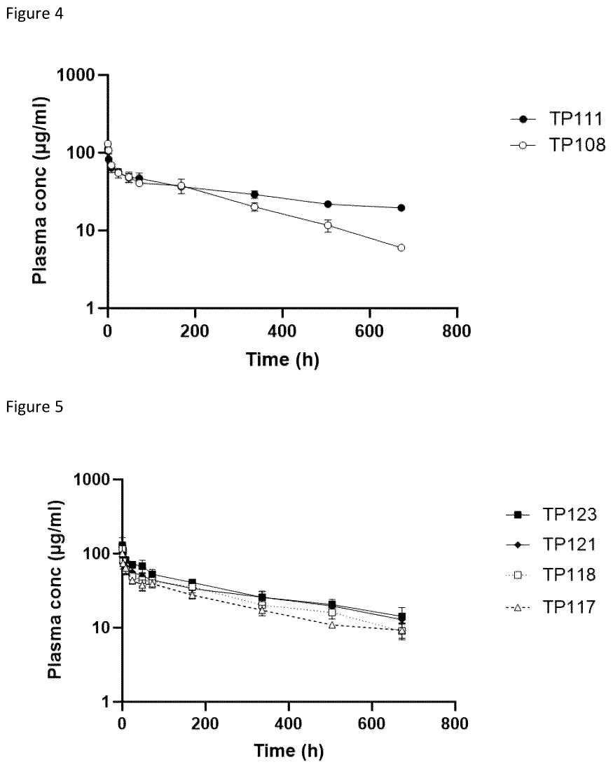

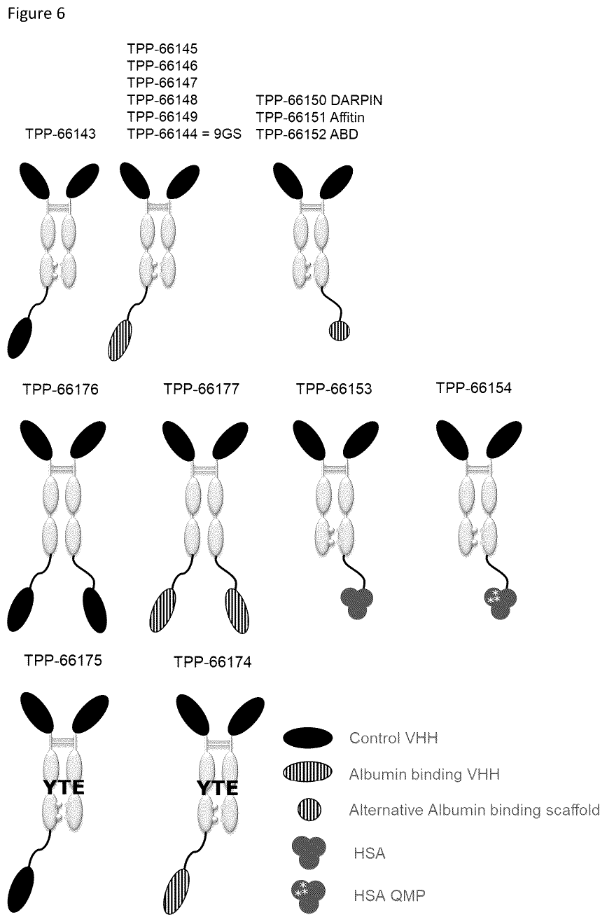

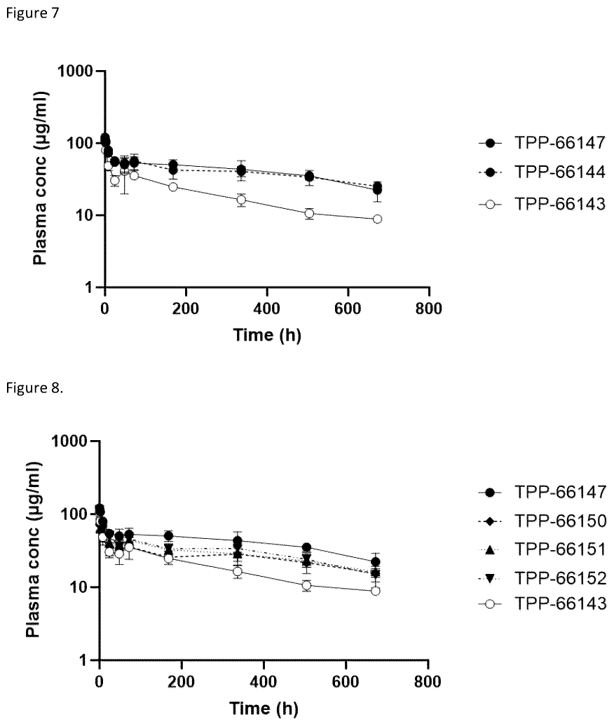

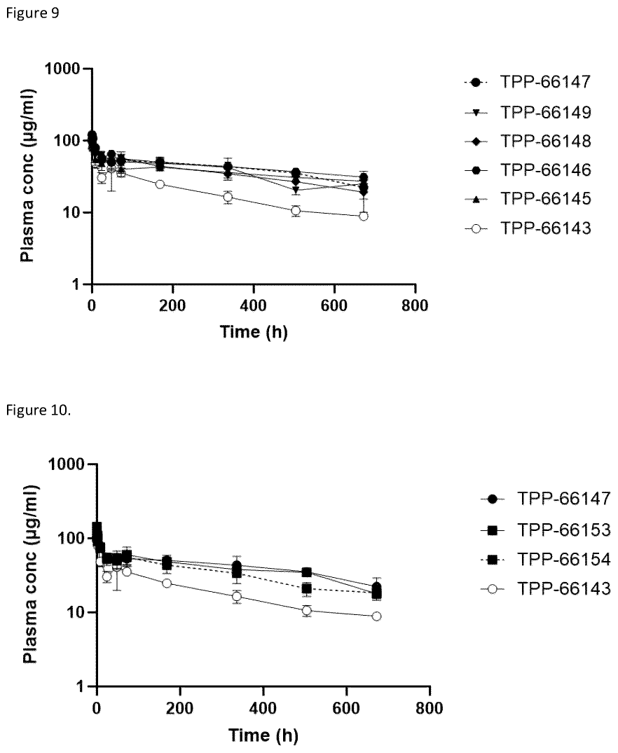

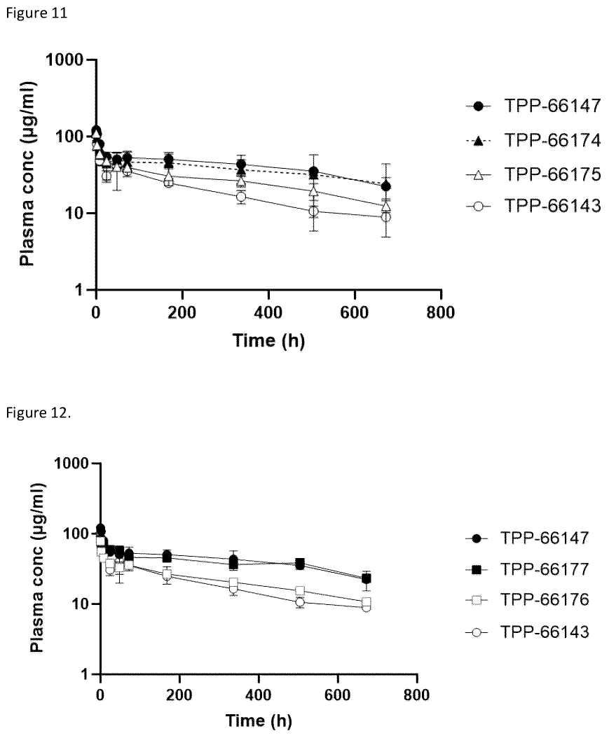

In one preferred embodiment, the Fc domain of an IgG comprised in the polypeptide of the present technology is in a dimeric form and comprises at least a hinge region or a part of it, two CH2 domains, and two CH3 domains, i.e., two polypeptide fragments, see, e.g., FIGS. 1, 3 and 6. The Fc domain of an IgG comprised in the polypeptide of the present technology is preferably dimeric, more preferably heterodimeric. In this preferred embodiment, at least one and preferably both of the CH3 domains comprised in the Fc domain comprise “knob in hole” mutations. Hence, in this preferred embodiment, the Fc domain is heterodimeric. These mutations serve to engineer an interface between a first and second polypeptide (CH3 domains, in this specific case) for heterooligomerization using the “knobs and holes” technology as described in, for example, U.S. Pat. No. 8,216,805, WO 1996/27011, Ridgway, J B et al., “‘Knobs-into-holes’ engineering of antibody CH3 domains for heavy chain heterodimerization”, Protein engineering, 1996, 9, 7:617-21 or in Merchant et al., “An efficient route to human bispecific IgG”, Nature Biotechnology, 1998, 16: 677-681. The preferred interface comprises at least a part of the CH3 domain of the Fc domain. “Protuberances” are constructed by replacing small amino acid side chains from the interface of the first polypeptide (the first CH3 in this case) with larger side chains (e.g., tyrosine or tryptophan). Compensatory “cavities” of identical or similar size to the protuberances are optionally created on the interface of the second polypeptide (the second CH3 in this case) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface. See, e.g., U.S. Pat. No. 8,216,805, WO 1996/27011, Ridgway, J B et al., “‘Knobs-into-holes’ engineering of antibody CH3 domains for heavy chain heterodimerization”, Protein engineering, 1996, 9, 7:617-21 or Merchant et al., “An efficient route to human bispecific IgG”, Nature Biotechnology, 1998, 16: 677-681 for further details. In a preferred embodiment, the amino acids HY in the CH3 domain of Fc domains are mutated into RF, such as at positions 435 and 436, (H435R and Y436F in CH3 domain as described by Jendeberg, L. et al. (1997, J. Immunological Meth., 201:25-34)). Hence, in this preferred embodiment, the Fc domain is heterodimeric.

Hence, in certain embodiments, a polypeptide as described herein comprises a native (i.e., wild type) Fc domain of a human IgG, such as preferably a native Fc of human IgG1 (e.g., Uniprot sequence PODOX5) or a native Fc of human IgG4 (e.g., Uniprot sequence P01861). Polypeptides comprising at least one such native Fc domain were produced and tested for beneficial PK properties as described in the examples below.

In certain specific embodiments, the polypeptides according to the present technology comprise variant Fc domains which have altered binding properties for an Fc ligand relative to an unmodified parent Fc molecule. For example, a polypeptide described herein may comprise an Fc region having one or more of amino acid residues 234, 235, 236, 237, 297, 318, 320 and 322 substituted to a different amino acid residue, such that the variant Fc region has an altered affinity for an effector ligand, e.g., an Fc receptor or the C1 component of complement, as described in U.S. Pat. Nos. 5,624,821 and 5,648,260, both to Winter et al.

In particular embodiments, the polypeptides of the present technology comprise an Fc variant domain with reduced effector function, in particular the so-called “FALA” or “LALA” Fc mutant with substitution of residues 234 and 235 to alanine. Extra optional mutations include the substitution of arginine residue 409 to lysine, deletion of lysine residue 447. Polypeptides comprising at least one Fc domain with the above mutations were produced and tested for beneficial PK properties as described in the examples below.

In particular embodiments, the polypeptides according to the present technology comprise an Fc variant domain showing improved binding to the FcRn receptor compared to the native Fc domain. Such Fc variants include those with substitutions at one or more of Fc region residues 259, 308, 428, and 434. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604).

In certain particular embodiments, the polypeptides according to the present technology comprise an Fc variant domain wherein methionine 428 was substituted to leucine and asparagine 434 was substituted to serine. In certain particular embodiments, the polypeptides according to the present technology comprise an Fc variant domain with the following mutations M252Y, S254T and T256E (YTE, see, e.g., Robbie G J et al., “A novel investigational Fc-modified humanized monoclonal antibody, motavizumab-YTE, has an extended half-life in healthy adults”, Antimicrob Agents Chemother., 2013 December; 57(12):6147-53).

Polypeptides comprising at least one Fc domain with the above mutations are produced and tested for beneficial PK properties.

In particular embodiments, the polypeptides according to the present technology may comprise an Fc variant domain showing reduced or no binding to the FcRn receptor compared to the native Fc domain. Such Fc variants include those with substitutions at one or more of Fc region residues 253, 310 and 453.

In particular embodiments, the polypeptides according to the present technology comprise an Fc variant domain wherein isoleucine 428 was substituted to alanine, histidine 310 was substituted to alanine, and histidine 453 was substituted to alanine, optionally in combination with histidine 453 substituted to alanine.

Polypeptides comprising at least one Fc domain are produced and tested for beneficial PK properties. Examples of Fc domains of an IgG or FcRn-binding fragments thereof which can be comprised in the polypeptide of the present technology is in a dimeric form, i.e., it comprises at least a hinge region or part of it, two CH2 domains, and two CH3 domains, i.e., two polypeptide fragments, see, e.g., FIGS. 1, 3 and 6. Preferably, the Fc domain or FcRn-binding fragments thereof is dimeric, more preferably heterodimeric, as defined above.

In one embodiment, the Fc domain comprised in the polypeptide of the present technology comprises two identical chains, e.g., as described in SEQ ID NO.: 112, 113, 115 or 181, preferably 113 or 181.

As described above, Fc domains from any IgG subtype can be used to generate the Fc domain or FcRn-binding fragment thereof comprised in the polypeptide of the present technology. In some embodiments, the Fc domain or FcRn-binding fragment thereof is derived from a human IgG1, IgG2, IgG3, or IgG4, preferably from IgG1 or IgG4, more preferably from IgG4, and comprise the substitutions described herein relative to the wildtype origin. For instance, the Fc domain comprised in the polypeptide of the present technology comprises or consists of two polypeptides as defined in SEQ ID NO.: 113, 115 or 181, preferably as defined in SEQ ID NO.: 113 or 181, more preferably as defined in SEQ ID NO.: 181 (e.g., two identical chains wherein each chain comprises or consists of SEQ ID NO.: 113, 115 or 181, preferably 181). In other preferred embodiments, the Fc domain comprised in the polypeptide of the present technology comprises or consists of two different polypeptides as defined in SEQ ID NOs.: 116 and 117 or as defined in SEQ ID NOs.: 186 and 187, or as defined in SEQ ID NOs.: 188 and 189, or as defined in SEQ ID NOs.: 198 and 199, or as defined in SEQ ID NO.: 186 and 190. In these cases, both chains forming the Fc domain are different (knob-and-holes).

In certain other embodiments, the Fc domain or FcRn-binding fragment thereof is an artificial Fc derived from more than one IgG subtype. In other embodiments, the Fc domain or FcRn-binding fragment thereof comprises a chimeric hinge (i.e., a hinge comprising hinge portions derived from hinge domains of different antibody isotypes, e.g., an upper hinge domain from an IgG4 molecule and an IgG 1 middle hinge domain). In certain embodiments, the Fc domain or FcRn-binding fragment thereof is an IgG 1 Fc region. In certain embodiments, the Fc domain or FcRn-binding fragment thereof is a human IgG Fc region. In certain embodiments, the Fc domain or FcRn-binding fragment thereof is a human IgG 1 Fc region. In certain embodiments, the Fc domain or FcRn-binding fragment thereof is an IgG 4 Fc region. In certain embodiments, the Fc domain or FcRn-binding fragment thereof is a human IgG 4 Fc region. In certain embodiments, the Fc domain or FcRn-binding fragment thereof is a chimeric Fc region.

In certain embodiments, the Fc domain comprises amino acid alterations, substitutions, insertions and/or deletions that confer the desired characteristics. Useful Fc domains FcRn-binding or fragments thereof being comprised in the FcRn antagonists of the present technology are described in WO 2015/100299 and in WO 2019/110823.

In other embodiments the at least one Fc domain of an IgG or fragment thereof comprised in the polypeptide of the present technology is a Fc domain or fragment thereof which competes with wild-type IgG1 Fc region for binding to FcRn. It is preferred that the Fc domain or fragment thereof which competes with wild-type IgG1 Fc region for binding to FcRn competes with wild-type IgG1 Fc region for binding to FcRn. For instance, the Fc domain or fragment thereof which competes with wild-type IgG1 Fc region for binding to FcRn binds specifically to FcRn with increased affinity relative to wild-type IgG IgG1 Fc region binding to FcRn. In other embodiment, the Fc domain or fragment thereof which competes with wild-type IgG1 Fc region for binding to FcRn has increased FcRn binding affinities at both acidic pH and extracellular physiological pH as compared to wild-type IgG IgG1 Fc region binding to FcRn. In another embodiment, the Fc domain or fragment thereof which competes with wild-type IgG1 Fc region for binding to FcRn specifically binds to FcRn with reduced pH dependence relative to a wild-type IgG1 Fc region. In further embodiments, the Fc domain or fragment thereof which competes with wild-type IgG1 Fc region for binding to FcRn has altered affinity (increased or decreased) for CD16a as compared to a wild-type IgG1 Fc region.

In some embodiments, the Fc domain or fragment thereof comprised in the polypeptide of the present technology comprises at least one, preferably all, of the following amino acids at the following positions:
a) a tyrosine (Y) at amino acid position 252,
b) a threonine (T) at amino acid position 254,
c) a glutamic acid (E) at amino acid position 256,
d) a lysine (K) at amino acid position 433,
e) a phenylalanine (F) at amino acid position 434, and/or
f) a tyrosine (Y) at amino acid position 436;
according to EU numbering.

Non-limiting examples of amino acid sequences that can be used in the Fc domain or fragment thereof are set forth in Table 1 of WO 2015/100299 (SEQ ID NO: 167-169 in the present description). In certain embodiments, the amino acid sequence of the Fc domain or FcRn-binding fragment thereof comprises the amino acid sequence set forth in SEQ ID NO: 167. In certain embodiments, the amino acid sequence of the Fc domain or FcRn-binding fragment thereof comprises or consists of the amino acid sequence set forth in SEQ ID NO: 167, 168, or 169. In one embodiment, if the Fc domain is in a dimeric form, i.e., comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 167. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 168. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 169. In these embodiments, at least one and preferably both of the CH3 domains comprised in the Fc domain comprised in the FcRn antagonists of the present technology may comprise knob in hole mutations, as defined above. Additionally or alternatively, the amino acids HY in the CH3 domains of Fc domain may be mutated into RF, such as at positions 435 and 436, (H435R and Y436F in CH3 domain, as described by Jendeberg, L. et al. (1997, J. Immunological Meth., 201:25-34)).

In certain embodiments, the Fc domain is a homodimer, wherein the amino acid sequence of each of the polypeptides comprised in the Fc domain consists of SEQ ID NO: 167. In certain embodiments, the Fc domain is a homodimer, wherein the amino acid sequence of each of the polypeptides comprised in the Fc domain consists of SEQ ID NO: 168. In certain embodiments, the Fc domain is a homodimer, wherein the amino acid sequence of each of the polypeptides comprised in the Fc domain consists of SEQ ID NO: 169.

In certain embodiments, the amino acid sequence of the Fc domains of the variant Fc region comprises the amino acid sequence set forth in SEQ ID NO: 167.

In certain embodiments, the amino acid sequence of the Fc domains of the variant Fc region comprises the amino acid sequence set forth in SEQ ID NO: 168.

In certain embodiments, the amino acid sequence of the Fc domains of the variant Fc region comprises the amino acid sequence set forth in SEQ ID NO: 169.

In certain embodiments, the polypeptide of the present technology comprises a variant Fc domain or fragment thereof that does not comprise an N-linked glycan at EU position 297. In certain embodiments, the polypeptide of the present technology comprises a variant Fc region that comprises an afucosylated N-linked glycan at EU position 297. In certain embodiments, the polypeptide of the present technology comprises a variant Fc domain or fragment thereof that comprises an N-linked glycan having a bisecting GlcNac at EU position 297.

Further Fc domains or fragments thereof being comprised in the polypeptide of the present technology are described in WO 2021/016571.

In some embodiments, the Fc domain fragment thereof comprised in the polypeptide of the present technology may comprise an amino acid substitution selected from M252, I253, S254, T256, K288, T307, K322, E380, L432, N434, or Y436, and any combinations thereof. Unless otherwise indicated, all Fc residue positions described herein are according to the EU numbering system. In some embodiments, the Fc domain or fragment thereof may comprise a double amino acid substitution at any two amino acid positions selected from M252, I253, S254, T256, K288, T307, K322, E380, L432, N434, and Y436. In some embodiments, the Fc domain or fragment thereof may comprise a triple amino acid substitution at any three amino acid positions selected from M252, I253, S254, T256, K288, T307, K322, E380, L432, N434, and Y436. In some embodiments, the Fc domain or fragment thereof may comprise a quadruple amino acid substitution at any four amino acid positions selected from M252, I253, S254, T256, K288, T307, K322, E380, L432, N434, and Y436. In some embodiments, it may be desirable for a Fc domain or fragment thereof to comprise an amino acid substitution at any of the amino acid positions selected from M252, I253, S254, T256, K288, T307, K322, E380, L432, or Y436, and any combinations thereof, wherein amino acid position N434 is not substituted (i.e., amino acid position N434 is wildtype).

For instance, in other embodiments, the Fc domain or fragment thereof may comprise an amino acid substitution selected from M252Y (i.e., a tyrosine at amino acid position 252), T256D, T256E, K288D, K288N, T307A, T307E, T307F, T307M, T307Q, T307W, E380C, N434F, N434P, N434Y, Y436H, Y436N, or Y436W, and any combinations thereof.

In some embodiments, the Fc domain or fragment thereof may comprise a double amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W. In some embodiments, the Fc domain or fragment thereof may comprise a triple amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W. In some embodiments, the Fc domain or fragment thereof may comprise a quadruple amino acid substitution selected from M252, wherein the substitution is M252Y; T256, wherein the substitution is T256D, or T256E; K288, wherein the substitution is K288D, or K288N; T307, wherein the substitution is T307A, T307E, T307F, T307M, T307Q, or T307W; E380, wherein the substitution is E380C; N434, wherein the substitution is N434F, N434P, or N434Y; Y436, wherein the substitution is Y436H, Y436N, or Y436W. In some embodiments, it may be desirable for a Fc domain or fragment thereof to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted with a phenylalanine (F) or a tyrosine (Y). In some embodiments, it may be desirable for a Fc domain or fragment thereof to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted with a tyrosine (Y). In some embodiments, it may be desirable for a Fc domain or fragment thereof to comprise an amino acid substitution at any of the amino acid positions selected from M252Y, T256D, T256E, K288D, K288N, T307A, T307E, T307F, T307M, T307Q, T307W, E380C, Y436H, Y436N, or Y436W, and any combinations thereof, wherein amino acid position N434 is not substituted (i.e., amino acid position N434 is wildtype).

In certain embodiments, the Fc domain or fragment thereof may comprise an amino acid substitution selected from M252, T256, T307, or N434, and any combinations thereof. In certain embodiments, the Fc domain or fragment thereof may comprise a double amino acid substitution at any two amino acid positions selected from M252, T256, T307, and N434. In certain embodiments, the Fc domain or fragment thereof may comprise a triple amino acid substitution at any three amino acid positions selected from M252, T256, T307, and N434. In certain embodiments, the Fc domain or fragment thereof may comprise a quadruple amino acid substitution at amino acid positions M252, T256, T307, and N434. In some embodiments, it may be desirable for a Fc domain or fragment thereof to comprise an amino acid substitution selected from M252, T256, or T307, and any combinations thereof, wherein amino acid position N434 is not substituted (i.e., amino acid position N434 is wildtype).

In exemplary embodiments, the Fc domain or fragment thereof may comprise an amino acid substitution selected from M252, wherein the substitution is M252Y; T256, wherein the substitution is T256D, or T256E; T307, wherein the substitution is T307Q. or T307W; or N434, wherein the substitution is N434F, or N434Y, and any combinations thereof. In certain embodiments, the Fc domain or fragment thereof may comprise a double amino acid substitution at any two amino acid positions selected from M252, wherein the substitution is M252Y; T256, wherein the substitution is T256D, or T256E; T307, wherein the substitution is T307Q or T307W; or N434, wherein the substitution is N434F, or N434Y. In certain embodiments, the Fc domain or fragment thereof may comprise a triple amino acid substitution at any three amino acid positions selected from M252, wherein the substitution is M252Y; T256, wherein the substitution is T256D, or T256E; T307, wherein the substitution is T307Q, or T307W; or N434, wherein the substitution is N434F, or N434Y. In certain embodiments, the Fc domain or fragment thereof may comprise a quadruple amino acid substitution at amino acid positions selected from M252, wherein the substitution is M252Y; T256, wherein the substitution is T256D, or T256E; T307, wherein the substitution is T307Q or T307W; or N434, wherein the substitution is N434F, or N434Y. In some embodiments, it may be desirable for a Fc domain or fragment thereof to comprise an amino acid substitution selected from M252Y, T256D, T256E, T307Q, or T307W, and any combinations thereof, wherein amino acid position N434 is not substituted with a phenylalanine (F) or a tyrosine (Y). In some embodiments, it may be desirable for a Fc domain or fragment thereof to comprise an amino acid substitution selected from M252Y, T256D, T256E, T307Q, or T307W, and any combinations thereof, wherein amino acid position N434 is not substituted with a tyrosine (Y).

In some embodiments, it may be desirable for a Fc domain or fragment thereof to comprise an amino acid substitution selected from M252Y, T256D, T256E, T307Q, or T307W, and any combinations thereof, wherein amino acid position N434 is not substituted (i.e., amino acid position N434 is wildtype).

In certain embodiments, the Fc domain or fragment thereof may comprise an amino acid substitution selected from T256D, or T256E, and/or T307W, or T307Q, and further comprises an amino acid substitution selected from N434F, or N434Y, or M252Y. In some embodiments, it may be desirable for a Fc domain or fragment thereof to comprise an amino acid substitution selected from T256D, or T256E, and/or T307W, or T307Q, and further comprise the amino acid substitution M252Y, wherein amino acid position N434 is not substituted with a phenylalanine (F) or a tyrosine (Y). In some embodiments, it may be desirable for a Fc domain or fragment thereof to comprise an amino acid substitution selected from T256D, or T256E, and/or T307W, or T307Q, and further comprise the amino acid substitution M252Y, wherein amino acid position N434 is not substituted with a tyrosine (Y). In some embodiments, it may be desirable for a Fc domain or fragment thereof to comprise an amino acid substitution selected from T256D, or T256E, and/or T307W, or T307Q, and further comprise the amino acid substitution M252Y, wherein amino acid position N434 is not substituted (i.e., amino acid position N434 is wildtype).

In some embodiments, the Fc domain or fragment thereof may comprise the amino acid substitutions shown in FIG. 33 of WO 2021/016571. For example, the Fc domain or fragment thereof may comprise double amino acid substitutions M252Y/N434Y (YY); or triple amino acid substitutions selected from M252Y/T307W/N434Y (YWY), M252Y/T256D/N434Y (YDY), and T256D/307W/N434Y (DWY).

In some embodiments, the Fc domain or fragment thereof may comprise a quadruple amino acid substitution selected from M252Y/T256D/T307Q/N434F (YDQF), M252Y/T256D/T307W/N434F (YDWF), M252Y/T256D/T307Q/N434Y (YDQY), M252Y/T256E/T307Q/N434Y (YEQY), M252Y/T256D/T307W/N434Y (YDWY), and M252Y/T256E/T307W/N434Y (YEWY).

In other embodiments, the Fc domain or fragment thereof comprises a combination of the following four amino acid residues:
a) a tyrosine (Y) at amino acid position 252,
b) an aspartic acid (D) or a glutamic acid (E) at amino acid position 256,
c) a tryptophan (W) or a glutamine (Q) at amino acid position 307, and
d) a phenylalanine (F) or a tyrosine (Y) at amino acid position 434;
according to Eu numbering.

In certain embodiments, the amino acid sequence of the Fc domain or fragment thereof comprises or consists of the amino acid sequence set forth in SEQ ID NO: 170 with at least one of the above-recited amino acid substitutions.

In certain embodiments, the amino acid sequence of the Fc domains of the variant Fc region comprises or consists of the amino acid sequence set forth in SEQ ID NO: 171 with the recited amino acid substitutions.

In certain embodiments, the amino acid sequence of the Fc domains of the variant Fc region comprises or consists of the amino acid sequence set forth in SEQ ID NO: 172 with the recited amino acid substitutions.

In certain embodiments, the amino acid sequence of the Fc domains of the variant Fc region comprises or consists of the amino acid sequence set forth in SEQ ID NO: 164 with the recited amino acid substitutions.

Hence, if the Fc domain is in a dimeric form, i.e., comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 170 with at least one of the above-recited amino acid substitutions. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 171 with at least one of the above-recited amino acid substitutions. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 172 with at least one of the above-recited amino acid substitutions. Hence, if the Fc domain comprises at least a hinge region or part of it, two CH2 domains, and two CH3 domains, i.e., two polypeptide fragments, the Fc domain may comprise two polypeptides comprising or consisting of the amino acid sequence set forth in SEQ ID NO: 164 with at least one of the above-recited amino acid substitutions. In these embodiments, at least one and preferably both of the CH3 domains comprised in the Fc domain comprised in the FcRn antagonists of the present technology may comprise knob in hole mutations, as defined above. Additionally or alternatively, the amino acids HY in the CH3 domains of Fc domain may be mutated into RF, such as at positions 435 and 436, (H435R and Y436F in CH3 domain, as described by Jendeberg, L et al. (1997, J. Immunological Meth., 201:25-34)).

In other embodiments, the amino acid sequence of the Fc domain or fragment thereof comprises or consists of the amino acid sequence set forth in any one of SEQ ID NOs: 170, 171, 172 or 164, with at least one of the above-recited amino acid substitutions and further comprising a linker sequence, e.g., as shown in Table A-2, at the N- and/or C-terminal region of the sequence. In a preferred embodiment, the linker comprises or consists of SEQ ID NO: 38, 39, 40, 41, 42 or 200. In a further preferred embodiment, the linker is comprised at the N-terminal region of the Fc domain or FcRn-binding fragment as set forth in any one of SEQ ID NOs: 170, 171, 172 or 164, with at least one of the above-recited amino acid substitutions. In a further preferred embodiment, the linker comprises or consists of SEQ ID NO: 38 or 200.

In other embodiments, the Fc domain or fragment thereof comprises a combination of the following four amino acid residues:
a) a tyrosine (Y) at amino acid position 252,
b) an aspartic acid (D) or a glutamic acid (E) at amino acid position 256,
c) a tryptophan (W) or a glutamine (Q) at amino acid position 307, and
d) a phenylalanine (F) or a tyrosine (Y) at amino acid position 434;
according to Eu numbering.

In other embodiments, the Fc domain or fragment thereof comprises a combination of amino acid residues selected from the group consisting of:
a) a tyrosine (Y) at amino acid position 252, an aspartic acid (D) at amino acid position 256, a glutamine (Q) at amino acid position 307, and a tyrosine (Y) at amino acid position 434;
b) a tyrosine (Y) at amino acid position 252, a glutamic acid (E) at amino acid position 256, a tryptophan (W) at amino acid position 307, and a tyrosine (Y) at amino acid position 434;
c) a tyrosine (Y) at amino acid position 252, a glutamic acid (E) at amino acid position 256, a glutamine (Q) at amino acid position 307, and a tyrosine (Y) at amino acid position 434;
d) a tyrosine (Y) at amino acid position 252, an aspartic acid (D) at amino acid position 256, a glutamine (Q) at amino acid position 307, and a phenylalanine (F) at amino acid position 434;
e) a tyrosine (Y) at amino acid position 252, an aspartic acid (D) at amino acid position 256, a tryptophan (W) at amino acid position 307, and a tyrosine (Y) at amino acid position 434; and
f) a tyrosine (Y) at amino acid position 252, an aspartic acid (D) at amino acid position 256, a tryptophan (W) at amino acid position 307, and a phenylalanine (F) at amino acid position 434.

In certain embodiments, the Fc domain may be mutated to decrease effector function using techniques known in the art. In some embodiments, the modified Fc herein also has altered binding affinity to an Fc-gamma receptors (FcγR). The FcγRs belong to a family that includes several members, e.g., FcγRI, FcγRIIa, FcγRIIb, FcγRIIIa, and FcγRIIb. In some embodiments, the modified Fc herein, while having enhanced FcRn binding affinities, has reduced FcγRIIIa binding affinity, compared to a wildtype Fc domain. In certain embodiments, the variant Fc has increased affinity for FcγRIIIa, called CD 16a in this application.

In certain embodiments of the present technology, the polypeptides disclosed herein comprise (i) at least one domain comprising a serum albumin protein and/or at least one domain specifically binding to a serum albumin protein and (ii) part of or a full-length IgG. In further particular embodiments, the polypeptides disclosed herein comprise (i) at least one domain comprising a serum albumin protein and/or at least one domain specifically binding to a serum albumin protein and (ii) part of or a full length IgG4. In yet further particular embodiments, the polypeptides disclosed herein comprise (i) at least one domain comprising a serum albumin protein and/or at least one domain specifically binding to a serum albumin protein and (ii) part of or a full length IgG1.

In further particular embodiments, the polypeptides disclosed herein comprise (i) at least one domain comprising a serum albumin protein and (ii) a full-length IgG, such as a full length IgG1 or a full length IgG4.

In further particular embodiments, the polypeptides disclosed herein comprise (i) at least one domain specifically binding to a serum albumin protein and (ii) a full-length IgG, such as a full length IgG1 or a full length IgG4.

In further particular embodiments, the polypeptides according to the present technology comprise at least one Fc domain that specifically binds to FcRn at an acidic pH of between 5.0 and 6.8, more preferably at an acidic pH of about 6.0, with an affinity (K_{A}) that is at least ten times higher than the affinity for FcRn of the same polypeptides at neutral or physiologic pH of 7.4. In yet further particular embodiments, at an acidic pH of between 5.0 and 6.8, the polypeptides according to the present technology bind to FcRn with an affinity (K_{A}) that is at least fifty times higher, such as at least hundred times higher than the affinity for FcRn of the same polypeptides at neutral or physiologic pH of 7.4.

In a particular embodiment, at physiologic pH such as at a pH of 7.4, the at least one Fc domain binds to FcRn with a K_{A} value lower than 10⁴ liters/mol.

In certain particular embodiments, the present technology provides polypeptides as described herein characterized in that the at least one Fc domain does not detectably, selectively or specifically bind to FcRn, or that exhibits no or essentially no binding to FcRn at neutral or physiologic pH, such as at a pH of 7.4.

The dissociation constant (K_{D}) of the polypeptides of the present technology for FcRn at acidic pH, preferably at a pH of between 5.0 and 6.8, is at least three times better (i.e., lower value) than the dissociation constant (K_{D}) of the same polypeptides for FcRn at neutral or physiologic pH of about 7.4. In further particular embodiments, the dissociation constant (K_{D}) of the polypeptides of the present technology for FcRn at acidic pH, preferably at a pH of between 5.0 and 6.8, is at least ten times higher/better than the dissociation constant for FcRn of the same polypeptides at neutral or physiologic pH of about 7.4. In yet further particular embodiments, the dissociation constant (K_{D}) of the polypeptides of the present technology for FcRn at acidic pH, preferably at a pH of between 5.0 and 6.8, is at least fifty times higher, such as at least hundred times higher/better than the dissociation constant for FcRn of the same polypeptides at neutral or physiologic pH of about 7.4.

Accordingly, the present technology relates to polypeptides comprising at least one Fc domain that specifically binds to FcRn at acidic pH of between about 6.0 and 7.4, preferably at pH of about 6.0, with an average K_{D} value of between 1 nM and 250 nM, such as at an average K_{D} value of 250 nM or less, even more preferably at an average K_{D} value of 200 nM, 150 nM, 100 nM, 50 nM or even less, such as less than 40, 30, 20, 10, 5, 1 nM such as less than 500, 400, 300, 200, 100, 90, 80, 70, 60, 50, 40, 30, 20 μM, or even less, such as less than 10 μM. Preferably, the K_{D} is determined by Kinexa, BLI or surface plasmon resonance (SPR), for instance as determined by SPR. Preferably, the average K_{D} is measured by SPR on recombinant protein.

In particular embodiments, at an acidic pH of between 5.0 and 6.8, the polypeptides according to the present technology specifically bind to FcRn with an on rate constant (kₒₙ) selected from the group consisting of at least about 10²M⁻¹s⁻¹, of at least about 10³M⁻¹s⁻¹, at least about 10⁴M⁻¹s⁻¹, at least about 10⁵M⁻¹s⁻¹, at least about 10⁶M⁻¹s⁻¹, at least about 10⁷M⁻¹s⁻¹, and at least about 10⁸M⁻¹s⁻¹, preferably as measured by surface plasmon resonance or BLI.

In particular embodiments, at an acidic pH of between 5.0 and 6.8, the polypeptides according to the present technology specifically bind to FcRn with an off rate constant (k_{off}) selected from the group consisting of at most about 10⁻¹s⁻¹, at most about 10⁻²s⁻¹, at most about 10⁻³s⁻¹, of at most about 10⁻⁴s⁻¹, at most about 10⁻⁵s⁻¹, and at most about 10⁻⁶s⁻¹, preferably as measured by surface plasmon resonance or BLI.

In particular embodiments, at an acidic pH of between 5.0 and 6.8, the polypeptides according to the present technology specifically bind to FcRn with an off-rate constant (k_{off}) selected from the group consisting of at most about 10⁻¹s⁻¹, at most about 10⁻²s⁻¹, at most about 10⁻³s, of at most about 10⁻⁴s⁻¹, at most about 10⁻⁵s⁻¹, and at most about 10⁻⁶s⁻¹. The off-rate constant (k_{off}) of these amino acid sequences and polypeptides for FcRn at acidic pH, preferably at a pH of between 5.0 and 6.8 is at least three times lower than the off rate constant (kₒₙ) of the same amino acid sequences and polypeptides for FcRn at neutral or physiologic pH of 7.4. In further particular embodiments, at an acidic pH of between 5.0 and 6.8, the polypeptides according to the present technology bind to FcRn with off rate constant (k_{off}) that is at least ten times lower than the off-rate constant (k_{off}) for FcRn of the same polypeptides at neutral or physiologic pH of 7.4. In yet further particular embodiments, at an acidic pH of between 5.0 and 6.8, the polypeptides according to the present technology bind to FcRn with off rate constant (kₒₙ) that is at least fifty times lower, such as at least hundred times lower than the off-rate constant (kₒₙ) for FcRn of the same polypeptides at neutral or physiologic pH of 7.4.

In certain particular embodiments, the present technology provides polypeptides as described herein characterized in that the at least one Fc domain binds to FcRn at neutral or physiologic pH of 7.4 with an off rate constant (k_{off}) that is at least three times, such as at least ten times, such as at least fifty times, such as at least hundred times higher than the off rate constant (kₒₙ) with which the at least one Fc domain binds to FcRn at acidic pH of between 5.0 and 6.8.

### 5.5 the Polypeptides of the Present Technology

Hence, as explained in detail above, the polypeptides of the present technology comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof.

In particular embodiments, the present technology provides polypeptides comprising at least one domain which is a serum albumin protein or which specifically binds to a serum albumin protein and an Fc domain of an IgG such that the polypeptide has a molecular weight of at least 30 kDa, in particular between about 30 kDa and 250 kDa, more particularly between about 65 kDa and 220 kDa, such as between about 65 kDa and 200 kDa, such as between about 65 kDa and 180 kDa, between about 65 kDa and 170 kDa, such as between about 65 kDa and 160 kDa, particularly between about 65 kDa and 150 kDa, more particularly between about 65 kDa and 130 kDa, most particularly between about 65 kDa and 120 kDa. In particular embodiments, the present technology provides polypeptides comprising at least one domain which is a serum albumin protein or which specifically binds to a serum albumin protein and an Fc domain of an IgG such that the polypeptide has a molecular weight of preferably about 120 kDa, 110 kDa, 100 kDa, 90 kDa, 85 kDa, 80 kD, 75 k Da, 70 k Da, such as about 65 kDa.

The polypeptides of the present technology, by specifically binding to the FcRn receptor and/or otherwise being directed to FcRn, are particularly suitable to be used for extending the in vivo half-life of therapeutic targets or therapeutic molecules of interest to which they are suitably linked, bound or fused (as demonstrated by the Examples described further herein).

The term “half-life” as used here can generally be defined as described in paragraph o) on page 57 of WO 2008/020079 and as mentioned therein refers to the time taken for the serum concentration of the compound or polypeptide to be reduced by 50%, in vivo, for example due to degradation of the sequence or compound and/or clearance or sequestration of the sequence or compound by natural mechanisms. The in vivo half-life of the polypeptide and/or fusion protein of the present technology can be determined in any manner known per se, such as by pharmacokinetic analysis. Suitable techniques will be clear to the person skilled in the art and may for example generally be as described in paragraph o) on page 57 of WO 2008/020079. As also mentioned in paragraph o) on page 57 of WO 2008/020079, the half-life can be expressed using parameters such as the t_{1/2}-alpha, t_{1/2}-beta and the area under the curve (AUC). In this respect it should be noted that the term “half-life” as used herein in particular refers to the t_{1/2}-beta or terminal half-life (in which the t_{1/2}-alpha and/or the AUC or both may be kept out of considerations). Reference is for example made to the standard handbooks, such as Kenneth, A et al: Chemical Stability of Pharmaceuticals: A Handbook for Pharmacists and Peters et al, Pharmacokinetic analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined in paragraph o) on page 57 of WO 2008/020079 and in particular refer to an increase in the t_{1/2}-beta, either with or without an increase in the t_{1/2}-alpha and/or the AUC or both.

The half-life in mammalian species will, among other factors, mainly depend on the binding properties (such as affinity) of the polypeptides and/or fusion proteins of the present technology for the serum albumin from said mammalian species as well on the half-life of the naïve serum albumin in said species.

The half-life of a polypeptide according to the present technology, or a fusion protein, a construct or a compound comprising the same (and further described herein) can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. In particular, half-life may be as defined in WO 2009/068627.

The in vivo half-life of a polypeptide according to the present technology, or a fusion protein, a construct or a compound comprising the same (as further described herein) can be determined in any manner known per se, such as by pharmacokinetic analysis. Suitable techniques will be clear to the person skilled in the art, and may for example generally involve the steps of suitably administering to a warm-blooded animal (i.e., to a human or to another suitable mammal, such as a mouse, rabbit, rat, pig, dog or a primate, for example monkeys from the genus Macaca (such as, and in particular, cynomologus monkeys (Macaca fascicularis) and/or rhesus monkeys (Macaca mulatto)) and baboon (Papio ursinus)) a suitable dose of the amino acid sequence, compound or polypeptide of the present technology; collecting blood samples or other samples from said animal; determining the level or concentration of the amino acid sequence, compound or polypeptide of the present technology in said blood sample; and calculating, from (a plot of) the data thus obtained, the time until the level or concentration of the amino acid sequence, compound or polypeptide of the present technology has been reduced by 50% compared to the initial level upon dosing. Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982). The half-life can be expressed using parameters such as the t_{1/2}-alpha, t_{1/2}-beta and the area under the curve (AUC). Reference is for example made to the Experimental Part below, as well as to the standard handbooks, such as Kenneth et al. 1996 (Chemical Stability of Pharmaceuticals: A Handbook for Pharmacists) and Peters et al. 1996 (Pharmacokinetic Analysis: A Practical Approach). Reference is also made to Gibaldi & Perron 1982 (Pharmacokinetics, Dekker M, 2nd Rev. edition). In the present specification, an “increase in half-life” refers to an increase in any one of these parameters, such as any two of these parameters, or essentially all three these parameters. The terms “increase in half-life” or “increased half-life” in particular refer to an increase in the t_{1/2}-beta, either with or without an increase in the t_{1/2}-alpha and/or the AUC or both.

In the context of the present technology, the term “clearance” or “clearance rate” (systemic plasma or serum clearance) is defined as the rate of drug elimination divided by the plasma concentration of the drug (rate at which a substance is cleared from the plasma compartment of blood.). The clearance of a substance is the volume of plasma that contains the same amount of the substance as has been removed from the plasma per unit time. The clearance or clearance rate can be measured with a timed collection of blood and an analysis of its composition, as described, e.g., in the examples. For instance, blood can be retrieved at different time points and serum can be prepared. Serum samples can be analyzed, e.g., by ELISA, for the presence of the polypeptides. PK parameters such as clearance can be obtained from non-compartmental analysis in Phoenix WinNonlinφ (version 8.2.2.227. Certara) using the Plasma Data Module. See the examples for further details. Clearance may be calculated using the following equation:

$$CL = \frac{Dose \text{?}}{AUC \text{?}}$$

$$\text{?} \text{indicates text missing or illegible when filed}$$

Thus, in particular embodiments, the polypeptides according to the present technology, such as the FcRn binding polypeptides of the present technology, (including fusion proteins, constructs and compounds comprising such polypeptides) will have an increased or extended half-life and/or a decreased or reduced clearance, compared to known polypeptides (described in the prior art) that bind to FcRn and/or are otherwise directed to FcRn, such as an Fc domain as such, or an Fc domain linked to a domain which does not specifically bind serum albumin protein.

Also, the polypeptides according to the present technology, such as the FcRn binding polypeptides of the present technology, if fused to another moiety, such as a therapeutic moiety or moieties, will have an increased half-life and/or a reduced clearance, compared to the other moiety per se, such as the other therapeutic moiety or moieties per se.

Generally, the polypeptides according to the present technology, such as the FcRn binding polypeptides of the present technology (and fusion proteins, constructs and compounds comprising such polypeptides) preferably have a half-life that is at least 1.5 times, preferably at least 2 times, such as at least 5 times, for example at least 10 times or more than 20 times, or greater than the half-life of the known polypeptides (described in the prior art) that bind to FcRn (as measured in either in man or a suitable animal, such as mouse or cynomolgus monkey), such as an Fc domain as such, or an Fc domain linked to a domain which does not specifically bind serum albumin protein.

Also, the polypeptides according to the present technology, such as the FcRn binding polypeptides of the present technology (and fusion proteins, constructs and compounds comprising such polypeptides) preferably have a half-life that is increased at least 30%, at least 50%, at least 75%, for example at least 100%, or increased more than 200%, such as more than 300%, more than 400%, more than 500% or greater compared to the half-life of the known polypeptides (described in the prior art) that bind to FcRn and/or are otherwise directed to FcRn (as measured in either in man or a suitable animal, such as mouse or cynomolgus monkey), such as an Fc domain as such, or an Fc domain linked to a domain which does not specifically bind serum albumin protein.

Also, the polypeptides according to the present technology, such as the FcRn binding polypeptides of the present technology (and fusion proteins, constructs and compounds comprising such polypeptides) preferably have a clearance or clearance rate, as defined herein, that is decreased or reduced at least about 10%, such as at least about 20%, or at least about 25%, or at least about 30%, or at least about 50%, or at least about 75%, or at least about 80%, or at least about 90%, or more, compared to the clearance or clearance rate of the known polypeptides (described in the prior art) that bind to FcRn and/or are otherwise directed to FcRn (as measured in either in man or a suitable animal, such as mouse or cynomolgus monkey), such as an Fc domain as such, or an Fc domain linked to a domain which does not specifically bind serum albumin protein.

Also, the polypeptides according to the present technology, such as the FcRn binding polypeptides of the present technology (and fusion proteins, constructs and compounds comprising such polypeptides) preferably have a clearance or clearance rate, as defined herein, that is decreased or reduced by at least 1.1 fold, such as at least 1.2 fold, or at least 1.3 fold, or at least 1.5 fold, or at least 2 fold, or at least 2.5 fold, or at least 3 fold, or at least 4 fold, or at least 5 fold, or at least 7 fold, or at least 8 fold, or at least 9 fold, or at least 10 fold, or more, compared to the clearance or clearance rate of the known polypeptides (described in the prior art) that bind to FcRn and/or are otherwise directed to FcRn (as measured in either in man or a suitable animal, such as mouse or cynomolgus monkey), such as an Fc domain as such, or an Fc domain linked to a domain which does not specifically bind serum albumin protein.

Also, the polypeptides of the present technology, such as the FcRn binding polypeptides according to the present technology (including fusion proteins, constructs and compounds comprising the same) comprising at least one domain comprising a serum albumin protein or specifically binding to a serum albumin protein and at least one Fc domain of an IgG, if fused to another moiety, such as a therapeutic moiety or moieties, will have an increased half-life, compared to the other moiety per se (as such), such as the other therapeutic moiety or moieties per se (as such).

Generally, the polypeptide, constructs or fusion proteins described herein, comprising a drug and/or a therapeutic moiety preferably have a half-life that is at least 1.1, such as at least 1.2, or at least 1.5 times, preferably at least 2 times, such as at least 3 times, or at least 5 times, for example at least 10 times or more than 20 times, such as more than 50 times, more than 100 times, more than 500 times, preferably more than 1000 times greater than the half-life of the corresponding other moiety per se, such as a drug and/or a therapeutic moiety per se (as measured in either in man or a suitable animal, such as mouse or cynomolgus monkey).

Generally, the polypeptide, constructs or fusion proteins described herein, comprising a drug and/or a therapeutic moiety preferably have a clearance rate, as defined herein, that is at least 1.1, such as at least 1.2, or at least 1.5 times, preferably at least 2 times, such as at least 3 times, or at least 4 times, or at least 5 times, for example at least 10 times or more than 20 times, such as more than 50 times or more than 100 times, or more, lower than the clearance rate of the corresponding other moiety per se, such as a drug and/or a therapeutic moiety per se (as measured in either in man or a suitable animal, such as mouse or cynomolgus monkey).

As mentioned, in one aspect, the polypeptide of the present technology, such as the FcRn binding polypeptide according to the present technology can be used to increase the half-life and/or decrease the clearance rate of (one or more) immunoglobulin single variable domains (ISVDs), such as domain antibodies, single domain antibodies, “dAb's”, VHHs or Nanobody® VHHs (such as VHHs, humanized VHHs or camelized VHs such as camelized human VHs).

Also, the polypeptides provided by the present technology and fusion proteins, constructs and compounds comprising the same (as further described herein) preferably have a half-life (defined as t_{1/2} beta) in man that is more than 1 hour, preferably more than 2 hours, more preferably of more than 6 hours, such as of more than 12 hours, and for example of about one day, two days, one week, about 13 days, about two weeks, about 16 days, or about 17 days, and up to and even beyond the half-life of serum albumin (i.e., in human about 19 days) or up to and beyond the half-life of IgG (i.e., in human about 23 days for wild type IgG and up to 90 days for engineered IgG), such as 3 months, 4 months, 5 months up to 6 months or longer.

Also, the polypeptides provided by the present technology and fusion proteins, constructs and compounds comprising the same (as further described herein) preferably have a half-life (defined as t_{1/2} beta) in mice that is more than 1 hour, preferably more than 2 hours, more preferably of more than 6 hours, such as of more than 12 hours, and for example of about one day, two days, one week, about 13 days, two weeks, about 16 days, or about 17 days, or about 18 days, or about 20 days, or about 23 days, or about 25 days, or about 30 days, or more, or up to and even beyond the half-life of serum albumin or up to and beyond the half-life of IgG, or longer.

Also, the polypeptides provided by the present technology and fusion proteins, constructs and compounds comprising the same (as further described herein) preferably have a clearance rate in mice that is less than 1 mL/hr/kg, preferably less than 0.8 mL/hr/kg, more preferably of less than 0.6 mL/hr/kg, such as of less than 0.5 mi/hr/kg, or less than 0.3 mL/hr/kg, or less than 0.2 mL/hr/kg, such as about 0.16 mL/hr/kg, or about 0.15 mL/hr/kg, or about 0.1 mL/hr/kg, or about 0.09 mL/hr/kg, or about 0.08 mL/hr/kg, or even less than the clearance rate of serum albumin and/or less than the clearance rate of IgG.

The polypeptides according to the different embodiments of the present technology are preferably also such that either:
they have a serum half-life in man (expressed as t₂ beta) that is more than 6 hours, preferably more than 12 hours, more preferably of more than 24 hours, even more preferably more than 72 hours; for example of about one week, two weeks, or about 16 days, or about 17 days, or about 18 days, or about 20 days, or about 23 days, or about 25 days, or about 30 days, or more, and up to and even beyond the half-life of serum albumin (i.e., in human about 19 days) or up to and beyond the half-life of IgG (i.e., in human about 23 days for wild type IgG and up to 90 days for engineered IgG); and/or such that: when they are linked to a therapeutic moiety or entity, they confer to the resulting polypeptide construct of the present technology a serum half-life in man (expressed as t₂ beta) that is more than 6 hours, preferably more than 12 hours, more preferably of more than 24 hours, even more preferably more than 72 hours; for example of about one week, two weeks and up to and even beyond the half-life of serum albumin (i.e., in human about 19 days) or up to and beyond the half-life of IgG (i.e., in human about 23 days for wild type IgG and up to 90 days for engineered IgG), such as 3 months, 4 months, 5 months up to 6 months or longer.

The half-life in mammalian species other than man will, among other factors, mainly depend on the binding properties (such as affinity) of the polypeptide of the present technology for serum albumin and/or FcRn from said mammalian species as well on the half-life of the naive serum albumin and IgG in said species. According to a preferred embodiment of the present technology, when an FcRn binding polypeptide of the present technology is cross-reactive (as defined herein) between human serum albumin and serum albumin from another mammalian species or cross-reactive between human FcRn and FcRn from another mammalian species, then the half-life of the polypeptide of the present technology (and/or of a compound of the present technology comprising said polypeptide) as determined in said species is preferably at least 5%, such as at least 10%, more preferably at least 25%, for example about 50%, about 100%, such as about 125%, about 150% up to about 200% or more of the half-life of serum albumin or IgG, respectively, in said species.

The polypeptides according to the different embodiments of the present technology are preferably also such that either:
they have a serum half-life in mice (expressed as t_{1/2}) that is more than 6 hours, preferably more than 12 hours, more preferably of more than 24 hours, even more preferably more than 72 hours; for example of about one week, two weeks, or about 16 days, or about 17 days, or about 18 days, or about 20 days, or about 23 days, or about 25 days, or about 30 days, or more, and up to and even beyond the half-life of serum albumin or up to and beyond the half-life of IgG; and/or such that: when they are linked to a therapeutic moiety or entity, they confer to the resulting polypeptide construct of the present technology a serum half-life in mice (expressed as t_{1/2}) that is more than 6 hours, preferably more than 12 hours, more preferably of more than 24 hours, even more preferably more than 72 hours; for example of about one week, two weeks or about 16 days, or about 17 days, or about 18 days, or about 20 days, or about 23 days, or about 25 days, or about 30 days, or more and up to and even beyond the half-life of serum albumin or up to and beyond the half-life of IgG, such as 3 months, 4 months, 5 months up to 6 months or longer.

The polypeptides and/or fusion proteins described herein preferably have a half-life that is at 1.1 times, 1.2 times, 1.3 times, 1.4 times, 1.5 times, preferably at least 2 times, preferably at least 3 times, at least 4 times, such as at least 5 times, for example at least 10 times or more than 20 times, greater than the half-life of a therapeutic construct comprising a therapeutic moiety and a known half-life extending moiety as disclosed in the prior art (as measured in either in man or a suitable animal, such as mouse or cynomolgus monkey).

Accordingly, the present technology provides improved polypeptides that can be used for various applications, including but not limited to prolonging the in vivo half-life of (existing or future) therapeutic compounds and/or reducing the clearance rate, as described herein. In particular embodiments, the polypeptides of the present technology have a high affinity for both serum albumin and FcRn.

In particular embodiments, the present technology provides polypeptides as described herein, characterized in that the polypeptides further comprise a therapeutic moiety, which preferably comprises a (single) domain antibody, a Nanobody® VHH, a VHH, a humanized VHH or a camelized VH. As mentioned, in one aspect, a polypeptide according to the present technology can be used to increase the half-life and/or decrease the clearance (i.e., to improve the PK parameters) of (one or more) immunoglobulin single variable domains (ISVDs), such as domain antibodies, single domain antibodies, “dAb's”, V_{HH}s or Nanobody® V_{HH}s (such as V_{HH}s, humanized V_{HH}s or camelized V_{H}s such as camelized human V_{H}s).

In particular embodiments, the polypeptides of the present technology comprise at least one ISVD that has high affinity for/binds specifically to serum albumin, at least one Fc domain of an IgG and at least a second ISVD that has high affinity for/binds specifically to a therapeutically relevant antigen other than FcRn and serum albumin.

It will be appreciated (as is also demonstrated in the Example section) that the albumin binding domain, such as the ISVD binding to serum albumin, and optionally the ISVD binding to a therapeutic target other than FcRn and albumin, can be positioned in any order in the polypeptides of the present technology. More particularly, in one embodiment, the ISVD binding to serum albumin is positioned N-terminally and the ISVD binding another antigen is positioned C-terminally. In another embodiment, the ISVD binding the other antigen is positioned N-terminally and the ISVD binding serum albumin is positioned C-terminally.

Hence, in one embodiment, the albumin and/or albumin binding domain (such as an albumin binding ISVD) comprised in the polypeptide of the present technology can be positioned at the N-terminal part of the polypeptide, e.g., linked (directly or via a linker, as disclosed herein) to the N-terminal part of the Fc domain (i.e., to the N-terminal part of either chain of the Fc-domain, if the Fc domain is a dimer). In another embodiment, the albumin and/or albumin binding domain (such as an albumin binding ISVD) comprised in the polypeptide of the present technology can be positioned at the C-terminal part of the polypeptide, e.g., linked (directly or via a linker, as disclosed herein) to the C-terminal part of the Fc domain (i.e., to the C-terminal part of either chain of the Fc-domain, if the Fc domain is a dimer). The polypeptide of the present technology may also comprise two albumin and/or albumin binding domains, such as two albumin binding ISVDs (see, e.g., SEQ ID NO.: 7-21 or 61-69), one linked (directly or via a linker, as disclosed herein) to the N-terminal part of the Fc domain (i.e., to the N-terminal part of either chain of the Fc-domain, if the Fc domain is a dimer) and another one linked (directly or via a linker, as disclosed herein) to the C-terminal part of the Fc domain (i.e., to the C-terminal part of either chain of the Fc-domain, if the Fc domain is a dimer). The polypeptide of the present technology may also comprise two albumin and/or albumin binding domains, such as two albumin binding ISVDs (see, e.g., SEQ ID NO.: 7-21 or 61-69), both linked (directly or via a linker, as disclosed herein) to the N-terminal part of the Fc domain (i.e., to the N-terminal part of either chain of the Fc-domain, if the Fc domain is a dimer). The polypeptide of the present technology may also comprise two albumin and/or albumin binding domains, such as two albumin binding ISVDs (see, e.g., SEQ ID NO.: 7-21 or 61-69), both linked (directly or via a linker, as disclosed herein) to the C-terminal part of the Fc domain (i.e., to the C-terminal part of either chain of the Fc-domain, if the Fc domain is a dimer). The polypeptide of the present technology may also comprise more than two albumin and/or albumin binding domains, such as two albumin binding ISVDs (see, e.g., SEQ ID NO.: 7-21 or 61-69), such as three or four albumin and/or albumin binding domains, such as two albumin binding ISVDs.

In addition to the albumin and/or albumin binding domain, the polypeptides of the present technology may comprise other groups or moieties, or binding units, as described herein, such as therapeutic moieties, drugs, vaccines and/or imaging agents. Hence, the one or more groups or moieties, or binding units, as described herein, such as therapeutic moieties, drugs, vaccines and/or imaging agents comprised in the polypeptide of the present technology may be linked (directly, or by means of a linker as described herein) to the N-terminal part of the Fc domain (i.e., to the N-terminal part of either chain of the Fc-domain, if the Fc domain is a dimer). In other embodiments, the one or more groups or moieties, or binding units, as described herein, such as therapeutic moieties, drugs, vaccines and/or imaging agents comprised in the polypeptide of the present technology may be linked (directly or via a linker, as disclosed herein) to the C-terminal part of the Fc domain (i.e., to the C-terminal part of either chain of the Fc-domain, if the Fc domain is a dimer). If there are more than one groups or moieties, or binding units, as described herein, such as therapeutic moieties, drugs, vaccines and/or imaging agents comprised in the polypeptide of the present technology, they can be located at the N- and/or C-terminal part of the Fc domain (e.g., at the N- and/or C-terminal part of one or both chains of the Fc domain, if it is a dimer).

For instance, the Fc domain comprised in the polypeptide of the present technology may have one or more albumin or albumin binding domains linked (directly or by means of a linker, as described herein) to the N- and/or C-terminal region of the Fc domain (e.g., to the N- and/or C-terminal region of one or both of the chains of the Fc domain, if it is dimeric) and, in addition, may have one or more groups or moieties, or binding units, as described herein, such as therapeutic moieties, drugs, vaccines and/or imaging agents also linked (directly or by means of a linker, as described herein) to the N- and/or C-terminal region of the Fc domain (e.g., to the N- and/or C-terminal region of one or both of the chains of the Fc domain, if it is dimeric). See FIGS. 1, 3 and 6 and Table A-1 for specific examples.

If the albumin and/or albumin binding domain, or the further group or moiety, or binding unit, as described herein, such as therapeutic moiety, drug, vaccine and/or imaging agent are linked (covalently linked) to the N-terminus of the Fc domain (or to the N-terminus of one or the two chains of the Fc domain, if dimeric), then it may be linked via a peptide linker such as a hinge linker, preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38.

If the albumin and/or albumin binding domain, or the further group or moiety, or binding unit, as described herein, such as therapeutic moiety, drug, vaccine and/or imaging agent are linked (covalently linked) to the C-terminus of the Fc domain (or to the C-terminus of one or the two chains of the Fc domain, if dimeric), then it may be linked via a peptide linker such as a GS linker, preferably comprising or consisting of SEQ ID NO.: 25-37, more preferably comprising or consisting of SEQ ID NO.: 29 or 36.

For instance, the polypeptide of the present technology may comprise a therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the first chain of a heterodimeric Fc region, and an albumin or albumin-binding domain, such as an albumin binding ISVD, affitin, DARPIN or ABC protein, preferably an albumin binding domain selected from SEQ ID NO.: 7-21, 61-69 and 102-104, even more preferably selected from SEQ ID NO.: 7-21 and 61-69 linked, preferably by means of a peptide linker, such as a GS linker, preferably selected from SEQ ID NO.: 25-37, to the N-terminus of the second chain of a heterodimeric Fc region, wherein the Fc domain is preferably a IgG 4 “FALA” Fc region with the knob-and-holes mutation.

For instance, the polypeptide of the present technology may comprise a therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the first chain of a heterodimeric Fc region, and an albumin or albumin-binding domain, such as an albumin binding ISVD, affitin, DARPIN or ABC protein, preferably an albumin binding domain selected from SEQ ID NO.: 7-21, 61-69 and 102-104, even more preferably selected from SEQ ID NO.: 7-21 and 61-69 linked, preferably by means of a peptide linker, such as a GS linker, preferably selected from SEQ ID NO.: 25-37, to the C-terminus of the first chain of a heterodimeric Fc region, wherein the Fc domain is preferably a IgG 4 “FALA” Fc region with the knob-and-holes mutation.

For instance, the polypeptide of the present technology may comprise (i) a therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the first chain of a heterodimeric Fc region, (ii) a second therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the second chain of a heterodimeric Fc region and (iii) an albumin or albumin-binding domain, such as an albumin binding ISVD, affitin, DARPIN or ABC protein, preferably an albumin binding domain selected from SEQ ID NO.: 7-21, 61-69 and 102-104, even more preferably selected from SEQ ID NO.: 7-21 and 61-69, linked, preferably by means of a peptide linker, such as a GS linker, preferably selected from SEQ ID NO.: 25-37, to the C-terminus of the first chain of a heterodimeric Fc region, wherein the Fc domain is preferably a IgG1 Fc region or a IgG4 FALA region with the knob-and-holes mutation.

For instance, the polypeptide of the present technology may comprise (i) a therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the first chain of a heterodimeric Fc region, (ii) a second therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the second chain of a heterodimeric Fc region and (iii) an albumin or albumin-binding domain, such as an albumin binding ISVD, affitin, DARPIN or ABC protein, preferably an albumin binding domain selected from SEQ ID NO.: 7-21, 61-69 and 102-104, even more preferably selected from SEQ ID NO.: 102-104, linked, preferably by means of a peptide linker, such as a GS linker, preferably selected from SEQ ID NO.: 25-37, to the C-terminus of the second chain of a heterodimeric Fc region, wherein the Fc domain is preferably a IgG1 Fc region or a IgG4 FALA region with the knob-and-holes mutation.

For instance, the polypeptide of the present technology may comprise (i) a therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the first chain of a heterodimeric Fc region, (ii) a second therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the second chain of a heterodimeric Fc region, (iii) an albumin or albumin-binding domain, such as an albumin binding ISVD, affitin, DARPIN or ABC protein, preferably an albumin binding domain selected from SEQ ID NO.: 7-21, 61-69 and 102-104, even more preferably selected from SEQ ID NO.: 7-21 and 61-69, linked, preferably by means of a peptide linker, such as a GS linker, preferably selected from SEQ ID NO.: 25-37, to the C-terminus of the first chain of a heterodimeric Fc region and (iv) a second albumin or albumin-binding domain, such as an albumin binding ISVD, affitin, DARPIN or ABC protein, preferably an albumin binding domain selected from SEQ ID NO.: 7-21, 61-69 and 102-104, even more preferably selected from SEQ ID NO.: 7-21 and 61-69, linked, preferably by means of a peptide linker, such as a GS linker, preferably selected from SEQ ID NO.: 25-37, to the C-terminus of the second chain of a heterodimeric Fc region, wherein the Fc domain is preferably a IgG1 Fc region or a IgG4 FALA region with the knob-and-holes mutation.

For instance, the polypeptide of the present technology may comprise (i) a therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the first chain of a heterodimeric Fc region, (ii) a second therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the second chain of a heterodimeric Fc region and (iii) an albumin or albumin-binding domain, preferably an albumin selected from SEQ ID NO.: 22, 23 and 110, even more preferably selected from SEQ ID NO.: 23 and 110, linked, preferably by means of a peptide linker, such as a GS linker, preferably selected from SEQ ID NO.: 25-37, to the C-terminus of the second chain of a heterodimeric Fc region, wherein the Fc domain is preferably a IgG1 Fc region or a IgG4 FALA region with the knob-and-holes mutation.

For instance, the polypeptide of the present technology may comprise (i) a therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the first chain of a heterodimeric Fc region, (ii) a second therapeutic moiety covalently linked, preferably by means of a hinge linker (e.g., preferably a short G1 hinge linker, preferably comprising or consisting of SEQ ID NO.: 38-42 and or 200, more preferably comprising or consisting of SEQ ID NO.: 38 or 200, even more preferably comprising or consisting of SEQ ID NO.: 38) to the N-terminus of the second chain of a heterodimeric Fc region and (iii) an albumin or albumin-binding domain, such as an albumin binding ISVD, affitin, DARPIN or ABC protein, preferably an albumin binding domain selected from SEQ ID NO.: 7-21, 61-69 and 102-104, even more preferably selected from SEQ ID NO.: 7-21 and 61-69, linked, preferably by means of a peptide linker, such as a GS linker, preferably selected from SEQ ID NO.: 25-37, to the C-terminus of the first chain of a heterodimeric Fc region, wherein the Fc domain is preferably a IgG4FALA Fc (YTE) region with the knob-and-holes mutation.

The present technology further provides polypeptides that comprise or essentially consist of (i) at least one domain comprising a serum albumin protein or specifically binding to a serum albumin protein and (ii) an Fc domain of an immunoglobulin G (IgG), and optionally further comprise one or more other groups, residues, moieties or binding units. As will become clear to the skilled person from the further disclosure herein, such further groups, residues, moieties, binding units or amino acid sequences may or may not provide further functionality to the polypeptide of the present technology (and/or to the compound or construct in which it is present) and may or may not modify the properties of the polypeptide of the present technology.

In particular embodiments, the present technology provides polypeptides as described herein, characterized in that the polypeptides further comprise a therapeutic moiety, as described herein in detail.

In particular embodiments, the at least one therapeutic moiety comprises or essentially consists of a therapeutic protein, polypeptide, compound, factor or other entity.

In particular embodiments, the at least one domain comprising a serum albumin protein or at least one domain binding specifically to a serum albumin protein and the at least one Fc domain of an IgG or fragment thereof are directly linked to each other or are linked via linkers or spacers to form a polypeptide according to the present technology. In a preferred embodiment, the at least one domain comprising a serum albumin protein is linked to the at least one Fc domain of an IgG or fragment thereof directly or by means of a linker as defined in the present specification, e.g., selected from the ones depicted in Table A-2. Preferably, the linker is a 9GS linker, or a 35GS linker, or a G1 short hinge or short hinge linker, as defined herein.

In particular embodiments, the (i) at least one domain comprising a serum albumin protein or at least one domain binding specifically to a serum albumin protein and the (ii) at least one Fc domain of an IgG or fragment thereof are directly linked to each other or are linked via linkers or spacers to form a polypeptide according to the present technology. Preferred linkers are depicted in Table A-2. Further preferred linkers are 9GS linkers, or 35GS linkers, or short hinge linker (e.g., SEQ ID NO.: 38 or 200), as defined herein.

For instance, the (i) at least one domain comprising a serum albumin protein or specifically binding to a serum albumin protein may be linked (directly or via a linker) to the N-terminal part of (ii) the Fc domain of an IgG or fragment thereof, e.g., via a hinge region or part thereof (e.g., SEQ ID NOs: 38-42 and 200). For instance, the (i) at least one domain comprising a serum albumin protein or specifically binding to a serum albumin protein may be linked (directly or via a linker) to the C-terminal part of (ii) the Fc domain of an IgG or fragment thereof, e.g., via a peptide linker (see, e.g., SEQ ID NO: 25-37, preferably a 9GS or 35GS linker).

In the context of the present application, “linked via a linker” or “covalently linked via a linker” means that the linker is directly attached to the N-terminal or C-terminal region of the Fc domain or fragment thereof, such as the Fc domains defined by SEQ ID NOs: 112-113, 115-117, 164, 167-172, 181, 186-190 and 198-199 (ii), and the N-terminal or C-terminal region of the at least one domain comprising a serum albumin protein or specifically binding to a serum albumin protein (i). For instance, the C-terminal region of (i) at least one domain comprising a serum albumin protein or specifically binding to a serum albumin protein (as defined by e.g., SEQ ID Nos: 22, 23, 109, 110, 7-21, 61-69) may be linked to the N-terminal region of the Fc domain of an IgG or fragment thereof (as defined by e.g., SEQ ID Nos: 112-113, 115-117, 164, 167-172, 181, 186-190 and 198-199) (ii), wherein the linker (as defined by e.g., SEQ ID NOs: 38-42 and 200) is directly linked to the C-terminus of the at least one domain comprising a serum albumin protein or specifically binding to a serum albumin protein (i) and directly linked to the N-terminus of the Fc domain or fragment thereof (ii). For instance, the N-terminal region of (i) at least one domain comprising a serum albumin protein or specifically binding to a serum albumin protein (as defined by e.g., SEQ ID Nos: 22, 23, 109, 110, 7-21, 61-69) may be linked to the C-terminal region of the Fc domain of an IgG, or fragment thereof (as defined by e.g., SEQ ID Nos: 112-113, 115-117, 164, 167-172, 181, 186-190 and 198-199) (ii), wherein the linker (as defined by e.g., SEQ ID NOs: 25-37) is directly linked to the N-terminus of the at least one domain comprising a serum albumin protein or specifically binding to a serum albumin protein (i) and directly linked to the C-terminus of the Fc domain or fragment thereof (ii).

In another embodiment, the linker is comprised at the N-terminal region of the Fc domain (e.g., SEQ ID NO.: 112).

In other preferred embodiments, the polypeptide comprising (i) at least one domain comprising a serum albumin protein or specifically binding to a serum albumin protein and (ii) a Fc domain of an IgG, or fragment thereof, further comprises further groups, residues, moieties or binding units, as described below, such as a therapeutic moiety (iii), as defined in detail below. In particular embodiments, the at least one Fc domain of an IgG or fragment thereof (ii) and the at least one domain comprising a serum albumin protein or at least one domain binding specifically to a serum albumin protein (i) and the further group, residue, moiety or binding units (iii) are directly linked to each other or are linked via linkers or spacers to form a polypeptide according to the present technology, as defined in detain herein.

The further group, residue, moiety or binding units may be linked via a linker means that the linker is directly attached to the N-terminal or C-terminal region of the Fc domain or fragment thereof. For instance, the further group, residue, moiety or binding units may be linked (directly or via a linker) to the N-terminal part of ii) the Fc domain of an IgG or fragment thereof, e.g., via a hinge region or part thereof, see, e.g., SEQ ID NOs: 38-42 or 200. For instance, the further group, residue, moiety or binding units may be linked (directly or via a linker) to the C-terminal part of ii) the Fc domain of an immunoglobulin G (IgG), e.g., via a peptide linker, see, e.g., SEQ ID NO: 25-37. In certain instances, the further group, residue, moiety or binding units is not directly linked to the at least one domain comprising a serum albumin protein or specifically binding to a serum albumin protein (i). In certain instances, the further group, residue, moiety or binding units is not linked to the at least one domain comprising a serum albumin protein or specifically binding to a serum albumin protein (i) via a linker (e.g., as shown in Table A-2).

FIGS. 1, 3 and 6 shows various orientations of the polypeptides of the present application comprising (i) at least one domain comprising a serum albumin protein and (ii) the Fc domain of an IgG or fragment thereof. See also Table A-1. For instance, in compound TP009 (FIG. 1), the at least one domain comprising a serum albumin protein or specifically binding to a serum albumin protein (i) is located at the N-terminal part of the Fc domain of an IgG or fragment thereof (ii) (chain 2), and it is linked to the dimeric Fc domain through the hinge region e.g., see SEQ ID NO: 38. For instance, in construct TP006 (FIG. 1), the at least one domain specifically binding to a serum albumin protein (i) is located at the C-terminal part of the Fc domain of an IgG or fragment thereof (ii) (chain 1), and it is linked to the Fc domain (polypeptide (ii)) through a peptide linker (35GS (SEQ ID NO.: 36)). For instance, in construct TPP-66153 (FIG. 6), the at least one domain comprising a serum albumin protein (i) is located at the C-terminal part of the Fc domain of an IgG or fragment thereof (ii) (chain 2), and it is linked to the Fc domain (polypeptide (ii)) through a peptide linker (35GS (SEQ ID NO.: 36)).

In some embodiments, the polypeptides of the present technology do not comprise protease-cleavable linkers, such as mouse or human matrix metalloproteinase (MMP) linkers. In particular, the polypeptides of the present technology do not comprise matrix metalloproteinase (MMP) linkers such as GPLGMWSR (SEQ ID NO: 125) or GPLGVR (SEQ ID NO: 126). In some embodiments, the polypeptides of the present technology do not comprise a mouse lower hinge sequence, such as CPPCKCPAPNLLGGP (SEQ ID NO: 131).

In some embodiments, the polypeptide of the present technology does not comprise or consist of one of the polypeptides of Table A-5, as disclosed in Table S1 of Fu-Yao Jiang: “A lesion-selective albumin-CTLA4Ig as a safe and effective treatment for collagen-induced arthritis”, Inflammation and Regeneration, vol. 43, no. 13 16, February 2023.

TABLE A-5
Polypeptides which are not and/or are not comprised
in the polypeptide of the present technology.

| Polypeptide | Masking domain | Linker | CTLA4 ECD | Ig |
|---|---|---|---|---|
| mAlb- | Albumin Glu25- | Core hinge-lower | CTLA4 Ile38- | IgG₂ₐ Hinge- |
| CTLA4Ig | Ala609 | hinge/upper CH2: | Ser160 | CH2—CH3 |
| | (Protein ID: | CPPCKCPAPNLLGGP | (Protein ID: | (IMGT Accnum: |
| | NP_033784.2) | | NP_033973.2) | V00825) |
| hAlb-CTLA4Ig | Albumin Asp25- | Core hinge-lower | CTLA4 Ala37- | IgG₁ Hinge- |
| | Leu609 | hinge/upper CH2: | Asp161 | CH2 CH3 |
| | (Protein ID: | CPPCPAPELLGGP | (Protein ID: | (IMGT Accnum: |
| | NP_000468.1) | | NP_005205.2) | J00228) |
| mAlb-MMP- | Albumin Glu25- | MMP substrate: | CTLA4 Ile38- | IgG₂ₐ Hinge- |
| CTLA4Ig | Ala609 | GPLGMWSRAAQPA | Ser160 | CH2 CH3 |
| | (Protein ID: | | (Protein ID: | (IMGT Accnum: |
| | NP_033784.2) | | NP.033973.2) | V00825) |

-IMGT, the international ImMunoGeneTics information system (https://www.imgt.org/).
-N/A, not applicable.

Preferably, the polypeptide of the present technology does not comprise or consist of a polypeptide as depicted in Table A-5a.

TABLE A-5a
Polypeptides which are not and/or are not comprised
in the polypeptide of the present technology.

| Polypeptide | Masking domain | Linker | CTLA4 ECD | Ig |
|---|---|---|---|---|
| mCTLA4Ig | N/A | N/A | CTLA4 Ile38- | IgG₂ₐ Hinge- |
| | | | Ser160 (Protein ID: | CH2 CH3 |
| | | | NP_033973.2) | (IMGT Accnum: |
| | | | | V00825) |
| mAlb- | Albumin Glu25- | Core hinge-lower | CTLA4 Ile38- | IgG₂ₐ Hinge- |
| CTLA4Ig | Ala609 | hinge/upper CH2: | Ser160 | CH2 CH3 |
| | (Protein ID: | CPPCKCPAPNLLGGP | (Protein ID: | (IMGT Accnum: |
| | NP_033784.2) | | NP_033973.2) | V00825) |
| hCTLA4Ig | N/A | N/A | CTLA4 Ala37- | IgG₁ Hinge- |
| | | | Asp161 | CH2 CH3 |
| | | | (Protein ID: | (IMGT Accnum: |
| | | | NP_005205.2) | J00228) |
| hAlb-CTLA4Ig | Albumin Asp25- | Core hinge-lower | CTLA4 Ala37- | IgG₁ Hinge- |
| | Leu609 | hinge/upper CH2: | Asp161 | CH2 CH3 |
| | (Protein ID: | CPPCPAPELLGGP | (Protein ID: | (IMGT Accnum: |
| | NP_000468.1) | | NP_005205.2) | J00228) |
| mAlb-CTLA4 | Albumin Glu25 | Core hinge-lower | CTLA4 Ile38- | N/A |
| ECD | Ala609 | hinge/upper CH2: | Ser160 | |
| | (Protein ID: | CPPCKCPAPNLLGGP | (Protein ID: | |
| | NP_033784.2) | | NP_033973.2) | |
| mIg-CTLA4 | IgG2a CH2 CH3 | Core hinge-lower | CTLA4 Ile38- | N/A |
| ECD | (IMGT Accnum: | hinge/upper CH2: | Ser160 | |
| | V00825) | CPPCKCPAPNLLGGP | (Protein ID: | |
| | | | NP_033973.2) | |
| mAlb-MMP- | Albumin Glu25- | MMP substrate: | CTLA4 Ile38- | IgG₂ₐ Hinge- |
| CTLA4Ig | Ala609 | GPLGMWSRAAQPA | Ser160 | CH2 CH3 |
| | (Protein ID: | | (Protein ID: | (IMGT Accnum: |
| | NP_033784.2) | | NP.033973.2) | V00825) |
| Ab lock- | PRGPTIKPCPPCKCP | MMP substrate: | CTLA4 Ile38- | IgG₂ₐ Hinge- |
| mCTLA4Ig | | GPLGMWSRGAQPA | Ser160 | CH2 CH3 |
| | | | (Protein ID: | (IMGT Accnum: |
| | | | NP_033973.2) | V00825) |
| VpreB- | VpreB Gln20- | MMP substrate: | CTLA4 Ile38- | IgG₂ₐ Hinge- |
| mCTLA4Ig | Ser121 | GPLGMWSR | Ser160 | CH2 CH3 |
| | (Protein ID: | | (Protein ID: | (IMGT Accnum: |
| | NP_058679.1) | | NP_033973.2) | V00825) |

-IMGT, the international ImMunoGeneTics information system (https://www.imgt.org/).
-N/A, not applicable.

Hence, in one embodiment, the present technology provides a polypeptide that comprises (i) at least one domain comprising a serum albumin protein or specifically binding to a serum albumin protein and (ii) an Fc domain of an immunoglobulin G (IgG) or fragment thereof, wherein the polypeptide does not comprise or consist of a polypeptide disclosed in Fu-Yao Jiang: “A lesion-selective album in-CTLA4Ig as a safe and effective treatment for collagen-induced arthritis”, Inflammation and Regeneration, vol. 43, no. 13 16 Feb. 2023, in particular in Table S1 (Supplementary information).

Hence, in one embodiment, the present technology provides a polypeptide that comprises (i) at least one domain comprising a serum albumin protein or specifically binding to a serum albumin protein and (ii) an Fc domain of an immunoglobulin G (IgG) or fragment thereof, wherein the polypeptide does not comprise or consist of a polypeptide selected form mCTLA4Ig, mAlb-CTLA4Ig, hCTLA4Ig, hAlb-CTLA4Ig, mAlb-CTLA4 ECD, mlg-CTLA4 ECD, mAlb-MMP-CTLA4Ig, Ab lock-mCTLA4Ig, VpreB-mCTLA4Ig as described in Table A-5.

In one embodiment, the present technology provides a polypeptide that comprises (i) at least one domain comprising a serum albumin protein or specifically binding to a serum albumin protein and (ii) an Fc domain of an immunoglobulin G (IgG) or fragment thereof, wherein the polypeptide does not comprise the protein CTLA4 Ile38-Ser160 (Protein ID: NP_033973.2) and/or the protein CTLA4 Ala37-Asp161 (Protein ID: NP_005205.2). In another embodiment, the present technology provides a polypeptide that comprises (i) at least one domain comprising a serum albumin protein or specifically binding to a serum albumin protein and (ii) an Fc domain of an immunoglobulin G (IgG) or fragment thereof, wherein the polypeptide does not comprise a CTLA4 protein.

In another embodiment, the present technology provides a polypeptide that comprises (i) at least one domain comprising a serum albumin protein or specifically binding to a serum albumin protein and (ii) an Fc domain of an immunoglobulin G (IgG) or fragment thereof, wherein the polypeptide does not comprise the protein Albumin Glu25 Ala609 (Protein ID: NP_033784.2) linked by a linker to the protein CTLA4 Ile38-Ser160 (Protein ID: NP_033973.2) and/or linked by a linker to the protein CTLA4 Ala37-Asp161 (Protein ID: NP_005205.2), wherein the linker is selected from CPPCKCPAPNLLGGP (SEQ ID NO.: 131), CPPCPAPELLGGP (SEQ ID NO.: 132), GPLGMWSRAAQPA (SEQ ID NO.: 111), GPLGMWSRGAQPA (SEQ ID NO.: 135) and GPLGMWSR (SEQ ID NO.: 125). In another embodiment, the polypeptide of the present technology does not comprise the protein Albumin Glu25 Ala609 (Protein ID: NP_033784.2) directly linked to the protein CTLA4 Ile38-Ser160 (Protein ID: NP_033973.2) and/or directly linked to the protein CTLA4 Ala37-Asp161 (Protein ID: NP_005205.2). In another embodiment, the polypeptide of the present technology does not comprise a serum albumin protein linked to the protein CTLA4 Ile38-Ser160 (Protein ID: NP_033973.2) and/or linked to the protein CTLA4 Ala37-Asp161 (Protein ID: NP_005205.2) by means of a linker selected from CPPCKCPAPNLLGGP (SEQ ID NO.: 131), CPPCPAPELLGGP (SEQ ID NO.: 132), GPLGMWSRAAQPA (SEQ ID NO.: 111), GPLGMWSRGAQPA (SEQ ID NO.: 135) and GPLGMWSR (SEQ ID NO.: 125). In another embodiment, the polypeptide of the present technology does not comprise a serum albumin protein directly linked to the protein CTLA4 Ile38-Ser160 (Protein ID: NP_033973.2) and/or linked to the protein CTLA4 Ala37-Asp161 (Protein ID: NP_005205.2). In another further embodiment, the polypeptide of the present technology does not comprise a serum albumin protein linked to a protein CTLA4 by means of a linker selected from CPPCKCPAPNLLGGP (SEQ ID NO.: 131), CPPCPAPELLGGP (SEQ ID NO.: 132), GPLGMWSRAAQPA (SEQ ID NO.: 111), GPLGMWSRGAQPA (SEQ ID NO.: 135) and GPLGMWSR (SEQ ID NO.: 125). In another further embodiment, the polypeptide of the present technology does not comprise a serum albumin protein directly linked to a protein CTLA4.

In another embodiment, the polypeptide of the present technology does not comprise:
The protein Albumin Glu25-Ala609 (Protein ID: NP_033784.2) linked to the protein CTLA4 Ile38-Ser160 (Protein ID: NP_033973.2) by means of the linker CPPCKCPAPNLLGGP (SEQ ID NO.: 131);
The protein Albumin Asp25-Leu609 (Protein ID: NP_000468.1) linked to the protein CTLA4 Ala37-Asp161 (Protein ID: NP_005205.2) by means of the linker CPPCPAPELLGGP (SEQ ID NO.: 132);
The protein Albumin Glu25 Ala609 (Protein ID: NP_033784.2) linked to the protein CTLA4 Ilee38-Ser160 (Protein ID: NP_033973.2) by means of the linker CPPCKCPAPNLLGGP (SEQ ID NO.: 131); and/or
The protein Albumin Glu25-Ala609 (Protein ID: NP_033 784.2) linked to the protein CTLA4 Ile38-Ser160 (Protein ID: NP.033973.2) by means of the linker GPLGMWSRAAQPA (SEQ ID NO.: 111).

In another embodiment, the polypeptide of the present technology does not comprise protein Albumin Glu25-Ala609 (Protein ID: NP_033784.2) (amino acids 25-609 of SEQ ID NO.: 180). In another embodiment, the polypeptide of the present technology does not comprise or consist of polypeptide hAlb-CTLA4Ig, which consists of: Albumin Asp25-Leu609 (Protein ID: NP_000468.1), Core hinge-lower hinge/upper CH2: CPPCPAPELLGGP, CTLA4 Ala37-Asp161 (Protein ID: NP_005205.2) and IgG, Hinge-CH2-CH3 (IMGT Accnum: J00228) (IMGT, the international ImMunoGeneTics information system (https://www.imgt.org/)).

In a preferred embodiment, the polypeptides of the present technology comprises (i) at least one domain comprising a serum albumin protein or specifically binding to a serum albumin protein and (ii) an Fc domain of an IgG or a fragment thereof, wherein the albumin, if present, is a human albumin, and wherein the polypeptide does not comprise a polypeptide comprising or consisting of CTLA4 Ala37-Aspl61 (Protein ID: NP_005205.2) and/or a polypeptide comprising or consisting of IgG₁ Hinge-CH2-CH3 (IMGT Accnum: J00228, wherein IMGT refers to the international ImMunoGeneTics information system (https://www.imgt.org/)).

In another embodiment, the polypeptides of the present technology comprises (i) at least one domain comprising a serum albumin protein or specifically binding to a serum albumin protein, (ii) an Fc domain of an IgG or a fragment thereof and (iii) a therapeutic moiety, wherein the albumin, if present, is a human albumin, and wherein the therapeutic moiety is not linked to the albumin, either directly or by means of a peptide linker such as CPPCPAPELLGGP (SEQ ID NO.: 132).

In another embodiment, the polypeptides of the present technology comprises (i) at least one domain comprising a serum albumin protein or specifically binding to a serum albumin protein, (ii) an Fc domain of an IgG or a fragment thereof and (iii) a therapeutic moiety, wherein the therapeutic moiety is not linked to the albumin, either directly or by means of a peptide linker, wherein the linker is preferably not a cleavable linker, such as a MMP cleavable linker, e.g., GPLGMWSRAAQPA (SEQ ID NO.: 111) or, and/or wherein the linker is not CPPCKCPAPNLLGGP (SEQ ID NO.: 131) and/or CPPCPAPELLGGP (SEQ ID NO.: 132).

In one embodiment, the polypeptide of the present technology does not comprise or consist of a polypeptide comprising, from the N- to the C-terminal, the following components:

Albumin-hinge-eCTLA4-Fc

As depicted in FIG. 2B of Fu-Yao Jiang: “A lesion-selective albumin-CTLA4Ig as a safe and effective treatment for collagen-induced arthritis”, Inflammation and Regeneration, vol. 43, no. 13 16, February 2023.

In another embodiment, the polypeptide of the present technology does not comprise or consist of an albumin protein linked (directly or by means of a peptide linker, as described herein) to the N-terminus of a CTLA4 protein.

In another embodiment, the polypeptide of the present technology does not comprise a mouse (Mus musculus) CTLA4 protein. In another embodiment, the polypeptide of the present technology does not comprise any of the following proteins:
CTLA4 Ile38-Ser160 (Protein ID: NP_033973.2); and
CTLA4 Ala37-Asp161 (Protein ID: NP_005205.2).

In one embodiment, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise an Fc region which comprises or consists of efgartigimod (CAS Registry No. 1821402-21-4). In particular, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise an Fc region which comprises or consists of SEQ ID NO.: 167, SEQ ID NO.: 168 and/or SEQ ID NO.: 169. Hence, the polypeptide of the present technology does not comprise a polypeptide comprising or consisting of SEQ ID NO.: 167, SEQ ID NO.: 168 and/or SEQ ID NO.: 169. In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise an Fc region which comprises or consists of SEQ ID NO.: 170, SEQ ID NO.: 171, SEQ ID NO.: 172 and/or SEQ ID NO.: 164.

In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise a Fc domain which comprises amino acid W at EU position 366.

In particular, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise an Fc region which comprises or consists of SEQ ID NO.: 163, SEQ ID NO.: 179 and/or SEQ ID NO.: 183. Hence, the polypeptide of the present technology does not comprise a polypeptide comprising or consisting of SEQ ID NO.: 163, SEQ ID NO.: 179 and/or SEQ ID NO.: 183.

In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise a Fc domain which comprises amino acids S, A, and V at EU positions 366, 368, and 407, respectively.

In particular, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise an Fc region which comprises or consists of SEQ ID NO.: 184, SEQ ID NO.: 185 and/or SEQ ID NO.: 211. Hence, the polypeptide of the present technology does not comprise a polypeptide comprising or consisting of SEQ ID NO.: 184, SEQ ID NO.: 185 and/or SEQ ID NO.: 211.

In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise a Fc domain which comprises amino acids Y, T, E, K, and F at EU positions 252, 254, 256, 433, and 434, respectively. In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise a Fc domain which comprises amino acids Y, T, E, K, F, and Y at EU positions 252, 254, 256, 433, 434, and 436, respectively.

In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise an IgG1 Fc domain which comprises amino acids Y, T, E, K, and F at EU positions 252, 254, 256, 433, and 434, respectively. In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise an IgG1 Fc domain which comprises amino acids Y, T, E, K, F, and Y at EU positions 252, 254, 256, 433, 434, and 436, respectively.

In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise a human IgG1 Fc domain which comprises amino acids Y, T, E, K, and F at EU positions 252, 254, 256, 433, and 434, respectively. In other embodiments, the present technology provides a polypeptide comprise or consist of (i) at least one domain comprising a serum albumin protein and/or a domain, such as a serum albumin binding ISVD, that has high affinity for/binds specifically to serum albumin protein and (ii) at least one Fc domain of an IgG or fragment thereof, preferably a FcRn-binding fragment thereof, wherein the polypeptide does not comprise a human IgG1 Fc domain which comprises amino acids Y, T, E, K, F, and Y at EU positions 252, 254, 256, 433, 434, and 436, respectively.

In other embodiments, the polypeptide of the present technology does not comprise a variant IgG Fc region which comprises a first Fc domain and a second Fc domain which form a dimer, wherein the first Fc domain and/or the second Fc domain comprise amino acids Y, T, E, K, and F at EU positions 252, 254, 256, 433, and 434, respectively.

In other embodiments, the polypeptide of the present technology does not comprise a variant IgG Fc region which comprises a first Fc domain and a second Fc domain which form a dimer, wherein the first Fc domain and/or the second Fc domain amino acids Y, T, E, K, F, and Y at EU positions 252, 254, 256, 433, 434, and 436 respectively.

In other embodiments, the polypeptide of the present technology does not comprise a variant IgG Fc region which comprises a first Fc domain and a second Fc domain which form a dimer, wherein both the first Fc domain and the second Fc domain comprise amino acids Y, T, E, K, and F at EU positions 252, 254, 256, 433, and 434, respectively.

In other embodiments, the polypeptide of the present technology does not comprise a variant IgG Fc region which comprises a first Fc domain and a second Fc domain which form a dimer, wherein both the first Fc domain and the second Fc domain comprise amino acids Y, T, E, K, F, and Y at EU positions 252, 254, 256, 433, 434, and 436, respectively.

In other embodiments, the polypeptide of the present technology does not comprise a variant IgG Fc region which comprises a first Fc domain and a second Fc domain which form a dimer, wherein the first Fc domain and/or the second Fc domain comprise amino acids Y, T, E, K, and F at EU positions 252, 254, 256, 433, and 434, respectively and wherein the first Fc domain and/or the second Fc domain is an IgG1 Fc domain.

In other embodiments, the polypeptide of the present technology does not comprise a variant IgG Fc region which comprises a first Fc domain and a second Fc domain which form a dimer, wherein the first Fc domain and/or the second Fc domain comprise amino acids Y, T, E, K, and F at EU positions 252, 254, 256, 433, and 434, respectively and wherein the first Fc domain and/or the second Fc domain is a human IgG Fc domain.

In other embodiments, the polypeptide of the present technology does not comprise a variant IgG Fc region which comprises a first Fc domain and a second Fc domain which form a dimer, wherein the first Fc domain and/or the second Fc domain comprise amino acids Y, T, E, K, and F at EU positions 252, 254, 256, 433, and 434, respectively and wherein the first Fc domain and/or the second Fc domain is a human IgG1 Fc domain.

In other embodiments, the polypeptide of the present technology does not comprise a variant IgG Fc region which comprises a first Fc domain and a second Fc domain which form a dimer, wherein the first Fc domain and/or the second Fc domain comprise amino acids Y, T, E, K, and F at EU positions 252, 254, 256, 433, and 434, respectively and wherein both the first Fc domain and the second Fc domain are IgG1 Fc domains.

In other embodiments, the polypeptide of the present technology does not comprise a variant IgG Fc region which comprises a first Fc domain and a second Fc domain which form a dimer, wherein the first Fc domain and/or the second Fc domain comprise amino acids Y, T, E, K, and F at EU positions 252, 254, 256, 433, and 434, respectively and wherein both the first Fc domain and the second Fc domain are human IgG Fc domains.

In other embodiments, the polypeptide of the present technology does not comprise a variant IgG Fc region which comprises a first Fc domain and a second Fc domain which form a dimer, wherein the first Fc domain and/or the second Fc domain comprise amino acids Y, T, E, K, and F at EU positions 252, 254, 256, 433, and 434, respectively and wherein both the first Fc domain and the second Fc domain 5 are human IgG1 Fc domains.

In one embodiment, the polypeptide of the present technology does not comprise an IgG1 Fc domain. In a preferred embodiment, the polypeptide of the present technology comprises an IgG4 Fc domain.

In other embodiments, the Fc domain comprised in the polypeptide of the present technology is a variant Fc domain which does not bind to FcRn with a higher affinity at pH 6.0 and/or at pH 7.4 as compared to a corresponding wild-type Fc region. In other embodiments, the polypeptide of the present technology does not comprise a Fc domain which comprises a combination of amino acids selected from the following:
(i) Q and L at EU positions 250 and 428, respectively;
(ii) P and A at EU positions 308 and 434, respectively;
(iii) P and Y at EU positions 308 and 434, respectively; or
(iv) Y, E and Y at EU positions 252, 286 and 434, respectively.

In other embodiments, the polypeptide of the present technology does not comprise a Fc domain which comprises a combination of amino acid substitutions selected from the following:
(i) M252Y, S254T, T256E, H433K and N434F;
(ii) T250Q and M428L;
(iii) V308P and N434A;
(iv) V308P and N434Y; or
(v) M252Y, N286E and N434Y.

In one embodiment, the polypeptide of the present technology does not comprise an Fc region comprising a methionine (M) to tyrosine (Y) substitution in position 252, a serine (S) to threonine (T) substitution in position 254, and a threonine (T) to glutamic acid (E) substitution in position 256, numbered according to the EU numbering system. See U.S. Pat. No. 7,658,921. This type of mutant Fc domain is referred to as “YTE mutant”. Hence, in one embodiment, the polypeptide of the present technology does not comprise a YTE mutant Fc domain. In an embodiment, the polypeptide of the present technology does not comprise Fc domain comprising one, two, three, or more amino acid substitutions of amino acid residues at positions 251-257, 285-290, 308-314, 385-389, and 428-436, numbered according to the EU numbering system.

In one particular embodiment, the polypeptide of the present technology does not comprise a full-length antibody. As used herein, the terms “antibody” and “antibodies” include full-length antibodies. Full-length antibodies comprise four polypeptide chains; two heavy chains and two light chains, usually connected by disulfide bonds. Light chains consist of one variable domain V_{L} and one constant domain C_{L}, while heavy chains contain one variable domain V_{H} and three to four constant domains (C_{H}). In one embodiment, the polypeptide of the present technology does not comprise or consists of rozanolixizumab (UCB7665), nipocalimab (M281), orilanolimab (ALXN1830/SYNT001), or batoclimab (IMVT-1401/RVT1401/HBM9161), which are all anti-FcRn antibodies. Nipocalimab comprises the light chain (SEQ ID NO: 218) and heavy chain (SEQ ID NO: 219) sequences. Rozanolixizumab comprises the light chain (SEQ ID NO: 212) and heavy chain (SEQ ID NO: 213) sequences. Orilanolimab comprises the light chain (SEQ ID NO: 214) and heavy chain (SEQ ID NO: 215) sequences. Batoclimab comprises the light chain (SEQ ID NO: 216) and heavy chain (SEQ ID NO: 217) sequences.

In some embodiments, the polypeptide of the present technology does not comprise at least one polypeptide as defined in any one of SEQ ID NOs.: 220-260.

In one embodiment, the polypeptide of the present technology comprises (i) at least one domain comprising a serum albumin protein or specifically binding to a serum albumin protein and (ii) an Fc domain of an IgG or a fragment thereof, wherein the Fc domain is a native Fc domain. In some embodiments, the Fc region comprises or consists of the Fc region of human IgG1 or IgG4. Preferably, the Fc domain is a variant Fc domain, as described herein, such as the so-called “FALA” or “LA” Fc mutant with substitution of residues 234 and 235 to alanine. In other embodiments, the Fc variant domain comprises the following mutations M252Y, S254T and T256E (YTE, see, e.g., Robbie G J et al., “A novel investigational Fc-modified humanized monoclonal antibody, motavizumab-YTE, has an extended half-life in healthy adults”, Antimicrob Agents Chemother., 2013 December; 57(12):6147-53).

Preferably, the polypeptide of the present technology comprises (i) at least one domain comprising a serum albumin protein and (ii) an Fc domain of an IgG or a fragment thereof, wherein the Fc domain comprises or consists of two identical polypeptides as defined in SEQ ID NO.: 113, 115 or 181, preferably 113 or 181. In another preferred embodiment, the polypeptide of the present technology comprises (i) at least one domain comprising a serum albumin protein and (ii) an Fc domain of an IgG or a fragment thereof, wherein the Fc domain comprises or consists of two different polypeptides selected from:
SEQ ID NOs.: 116 and 117;
SEQ ID NOs.: 186 and 187;
SEQ ID NOs.:188 and 189;
SEQ ID NOs.: 198 and 199; and
SEQ ID NO.: 186 and 190.

In one embodiment, the polypeptide of the present technology comprises (i) at least one domain specifically binding to a serum albumin protein and (ii) an Fc domain of an IgG or a fragment thereof, wherein the Fc domain comprises or consists of two identical polypeptides as defined in SEQ ID NO.: 113, 115 or 181, preferably 113 or 181. In another preferred embodiment, the polypeptide of the present technology comprises (i) at least one domain specifically binding to a serum albumin protein and (ii) an Fc domain of an IgG or a fragment thereof, wherein the Fc domain comprises or consists of two different polypeptides selected from:
SEQ ID NOs.: 116 and 117;
SEQ ID NOs.: 186 and 187;
SEQ ID NOs.: 188 and 189;
SEQ ID NOs.: 198 and 199; and
SEQ ID NO.: 186 and 190.

In another embodiment, the polypeptide of the present technology comprises (i) at least one domain specifically binding to a serum albumin protein and (ii) an Fc domain of an IgG or a fragment thereof, wherein the at least one domain specifically binding to a serum albumin protein is an albumin-binding ISVD, preferably selected from SEQ ID NO.: 7-21 and 61-69, more preferably wherein the albumin binding domain is selected from a polypeptide comprising or consisting of: ALB23002 (SEQ ID NO.: 20), Alb23002-A (SEQ ID NO.: 21), HSA006A06 (SEQ ID NO.: 65), ALBX00002 (SEQ ID NO.: 64), ALB11002 (SEQ ID NO.: 13) and T023500029 (SEQ ID NO.: 69), even more preferably selected from HSA006A06 (SEQ ID NO.: 65), ALB11002 (SEQ ID NO.: 13) and ALB23002 (SEQ ID NO.: 20).

In another embodiment, the polypeptide of the present technology comprises (i) at least one domain specifically binding to a serum albumin protein and (ii) an Fc domain of an IgG or a fragment thereof, wherein the at least one domain specifically binding to a serum albumin protein is an albumin-binding ISVD, preferably selected from SEQ ID NO.: 7-21 and 61-69, more preferably wherein the albumin binding domain is selected from a polypeptide comprising or consisting of: ALB23002 (SEQ ID NO.: 20), Alb23002-A (SEQ ID NO.: 21), HSA006A06 (SEQ ID NO.: 65), ALBX00002 (SEQ ID NO.: 64), ALB11002 (SEQ ID NO.: 13) and T023500029 (SEQ ID NO.: 69), even more preferably selected from HSA006A06 (SEQ ID NO.: 65), ALB11002 (SEQ ID NO.: 13) and ALB23002 (SEQ ID NO.: 20), and wherein the Fc domain comprises or consists of two identical polypeptide as defined in SEQ ID NO.: 113, 115 or 181, preferably 113 or 181 or wherein the Fc domain comprises or consists of two different polypeptides selected from:
SEQ ID NOs.: 116 and 117;
SEQ ID NOs.: 186 and 187;
SEQ ID NOs.: 188 and 189;
SEQ ID NOs.: 198 and 199; and
SEQ ID NO.: 186 and 190.

In one embodiment, the (i) at least one domain comprising a serum albumin protein or specifically binding to a serum albumin protein and (ii) an Fc domain of an IgG comprised in the polypeptide of the present technology are linked by means of a non-cleavable peptide linker, preferably a GS linker as defined herein, even more preferably a linker selected from SEQ ID NO.: 25 to 37, even more preferably a 9GS linker (SEQ ID NO.: 29) or a 35GS linker (SEQ ID NO.: 36).

In one embodiment, the polypeptide of the present technology comprises a single albumin binding ISVD, preferably selected from SEQ ID NO.: 7-21 and 61-69, more preferably wherein the albumin binding domain is selected from a polypeptide comprising or consisting of: ALB23002 (SEQ ID NO.: 20), Alb23002-A (SEQ ID NO.: 21), HSA006A06 (SEQ ID NO.: 65), ALBX00002 (SEQ ID NO.: 64), ALB11002 (SEQ ID NO.: 13) and T023500029 (SEQ ID NO.: 69), even more preferably selected from HSA006A06 (SEQ ID NO.: 65), ALB11002 (SEQ ID NO.: 13) and ALB23002 (SEQ ID NO.: 20).

In another embodiment, the polypeptide of the present technology comprises (i) at least one domain specifically binding to a serum albumin protein and (ii) an Fc domain of an IgG or a fragment thereof, wherein the at least one domain specifically binding to a serum albumin protein is a DARPin, an affitin or protein ABD, preferably selected from SEQ ID NO.: 102-104. In another embodiment, the polypeptide of the present technology comprises (i) at least one domain specifically binding to a serum albumin protein and (ii) an Fc domain of an IgG or a fragment thereof, wherein the at least one domain specifically binding to a serum albumin protein is a DARPin, an affitin or protein ABD, preferably selected from SEQ ID NO.: 102-104, and wherein the Fc domain comprises or consists of two identical polypeptide as defined in SEQ ID NO.: 113, 115 or 181, preferably 113 or 181 or wherein the Fc domain comprises or consists of two different polypeptides selected from:
SEQ ID NOs.: 116 and 117;
SEQ ID NOs.: 186 and 187;
SEQ ID NOs.: 188 and 189;
SEQ ID NOs.: 198 and 199; and
SEQ ID NO.: 186 and 190.

In another embodiment, the Fc region comprised in the polypeptide of the present technology comprises or consists of two identical polypeptides as defined in SEQ ID NO.: 115 or 181, preferably 181 or comprises or consists of two different polypeptides selected from:
SEQ ID NOs.: 186 and 187;
SEQ ID NOs.: 188 and 189;
SEQ ID NOs.: 198 and 199; and
SEQ ID NO.: 186 and 190.

In another embodiment, the polypeptide of the present technology comprises (i) at least one domain specifically binding to a serum albumin protein and (ii) an Fc domain of an IgG or a fragment thereof, wherein the at least one domain specifically binding to a serum albumin protein is an albumin-binding ISVD, preferably selected from SEQ ID NO.: 7-21 and 61-69, more preferably wherein the albumin binding domain is selected from a polypeptide comprising or consisting of: ALB23002 (SEQ ID NO.: 20), Alb23002-A (SEQ ID NO.: 21), HSA006A06 (SEQ ID NO.: 65), ALBX00002 (SEQ ID NO.: 64), ALB11002 (SEQ ID NO.: 13) and T023500029 (SEQ ID NO.: 69), even more preferably selected from HSA006A06 (SEQ ID NO.: 65), ALB11002 (SEQ ID NO.: 13) and ALB23002 (SEQ ID NO.: 20), and wherein the Fc domain comprises or consists of two identical polypeptide as defined in SEQ ID NO.: 115 or 181, preferably 181 or comprises or consists of two different polypeptides selected from:
SEQ ID NOs.: 186 and 187;
SEQ ID NOs.: 188 and 189;
SEQ ID NOs.: 198 and 199; and
SEQ ID NO.: 186 and 190.

In another embodiment, the polypeptide of the present technology comprises (i) at least one domain specifically binding to a serum albumin protein and (ii) an Fc domain of an IgG or a fragment thereof, wherein the at least one domain specifically binding to a serum albumin protein is a DARPin, an affitin or protein ABD, preferably selected from SEQ ID NO.: 102-104. In another embodiment, the polypeptide of the present technology comprises (i) at least one domain specifically binding to a serum albumin protein and (ii) an Fc domain of an IgG or a fragment thereof, wherein the at least one domain specifically binding to a serum albumin protein is a DARPin, an affitin or protein ABD, preferably selected from SEQ ID NO.: 102-104, and wherein the Fc domain comprises or consists of two identical polypeptide as defined in SEQ ID NO.: 115 or 181, preferably 181 or comprises or consists of two different polypeptides selected from:
SEQ ID NOs.: 186 and 187;
SEQ ID NOs.: 188 and 189;
SEQ ID NOs.: 198 and 199; and
SEQ ID NO.: 186 and 190.

In a preferred embodiment, the polypeptide of the present technology comprises or consists of a polypeptide as described in Table A-1. In another preferred embodiment, the polypeptide of the present invention comprises or consists of a polypeptide as described in Table A-11.

### 5.6 Further Groups, Residues, Moieties or Binding Units

Thus, the polypeptides of the present technology can generally be prepared by a method which comprises at least one step of suitably linking (directly or by means of a linker, as described herein) the one or more domains, i.e., the domain comprising a serum albumin protein or the serum albumin binding domain and the Fc domain of an IgG or fragment thereof, to each other and optionally in addition to one or more further groups, residues, moieties or binding units, as mentioned above, either directly or via one or more suitable linkers.

For example, such further groups, residues, moieties or binding units may be one or more additional immunoglobulins, so as to form a (fusion) protein or (fusion) polypeptide. In a preferred but non-limiting aspect, the one or more other groups, residues, moieties or binding units are immunoglobulin single variable domains. Even more preferably, the one or more other groups, residues, moieties or binding units are chosen from the group consisting of domain antibodies, immunoglobulin single variable domains that are suitable for use as a domain antibody, single domain antibodies, immunoglobulin single variable domains (ISVDs) that are suitable for use as a single domain antibody, “dAb” 's, immunoglobulin single variable domains that are suitable for use as a dAb, VHHs, humanized VHHs, camelized VHs, or Nanobody® VHHs. Alternatively, such groups, residues, moieties or binding units may for example be chemical groups, residues, moieties, which may or may not by themselves be biologically and/or pharmacologically active. For example, and without limitation, such groups may be linked to the one or more domains in the polypeptides of the present technology so as to provide a “derivative” of a polypeptide of the present technology, as further described herein. A polypeptide of the present technology may also include additional groups with certain functionalities, such as a label, a toxin, one or more linkers, a binding sequence, etc. These additional functionalities include both amino acid-based and non-amino acid-based groups.

It should be appreciated that the terms “fusion protein”, “fusion polypeptide construct”, “compound of the present technology”, “polypeptide construct” and “polypeptide” can be used interchangeably herein (unless the context clearly dictates otherwise).

In some embodiments, the domains comprised in the polypeptides of the present technology are antibody-based scaffolds and/or non-antibody-based scaffolds as disclosed herein.

Polypeptides of the present technology can also be prepared by a method which generally comprises at least the steps of providing a nucleic acid that encodes a polypeptide of the present technology, expressing said nucleic acid in a suitable manner, and recovering the expressed polypeptide of the present technology. Such methods can be performed in a manner known per se, which will be clear to the skilled person, for example on the basis of the methods and techniques further described herein. The process of designing/selecting and/or preparing a polypeptide of the present technology, starting from a polypeptide comprising at least one domain of the present technology, is also referred to herein as “formatting” said polypeptide of the present technology. Examples of ways in which a polypeptide of the present technology can be formatted, and examples of such formats will be clear to the skilled person based on the disclosure herein.

Hence, the present technology is also directed to a polypeptide or fusion protein comprising the polypeptide of the present technology, as defined herein (comprising (i) at least one domain comprising a serum albumin protein or specifically binding to a serum albumin protein and (ii) an Fc domain of an IgG, or a fragment thereof) and one or more further groups, residues, moieties or binding units, either directly or via one or more suitable linkers. PK parameters (such as half-life or clearance) are improved by the presence of the polypeptide of the present technology in the fusion protein, as described herein. The half-life of the one or more further groups, residues, moieties or binding units is increased by the presence of the polypeptide of the present technology in the fusion protein, as described herein, as compared with the half-life of the one or more further groups, residues, moieties or binding units per se (as such). The clearance rate of the one or more further groups, residues, moieties or binding units is decreased or reduced by the presence of the polypeptide of the present technology in the fusion protein, as described herein, as compared with the clearance rate of the one or more further groups, residues, moieties or binding units per se (as such).

Suitable linkers for use in the molecule of the present technology will be clear to the skilled person and may generally be any linker used in the art to link amino acid sequences or any other molecule comprised in the fusion protein. Preferably, said linker is suitable for use in constructing proteins or polypeptides that are intended for pharmaceutical use. Some particularly preferred linkers include the linkers that are used in the art to link antibody fragments or antibody domains. For example, a linker may be a suitable amino acid or amino acid sequence, and in particular amino acid sequences of between 1 and 50, preferably between 1 and 30, such as between 1 and 10 amino acid residues. Some preferred examples of such amino acid sequences include Gly-Ser linkers, for example of the type (GlyₓSer_{y})_{z}, such as (for example (Gly₄Ser)₃ or (Gly₃Ser₂)₃, as described in WO 1999/42077 and the GS30, GS15, GS9 and GS7 linkers described in the applications by Ablynx mentioned herein (see for example WO 2006/040153 and WO 2006/122825), as well as hinge-like regions, such as the hinge regions of naturally occurring heavy chain antibodies or similar sequences (such as described in WO 1994/04678). Examples of linkers are also provided in Table A-2. Polyethylene glycol (PEG), in any of the variants described below, may also be used as a linker in the fusion protein of the present technology. Other suitable linkers for use in the molecule of the present technology are described, e.g., in Kjeldsen T. et al. (“Dually reactive long recombinant linkers for bioconjugations as an alternative to PEG”, ACS Omega, 2020, 5:19827-19833). As described therein polar protein sequences with PEG-like properties, sometimes called “recombinant PEG”, have in recent years been described by Alvarez (“Improving protein pharmacokinetics by genetic fusion to simple amino acid sequences”, J. Biol. Chem., 2004, 279:3375-3381), Amunix (mixed sequences of GEDSTAP residues, termed “ELNN polypeptides”, see, e.g., US 2014/0301974 A1), XL-protein (PAS repeats), Novo Nordisk (GQAP-like repeats), SOBI and others. Further suitable linkers for use in the molecule of the present technology are, e.g., cleavable linkers, i.e., linkers which have a trigger in its structure that can be efficiently cleaved. For instance, Su, Z. et al. (“Antibody-drug conjugates: Recent advances in linker chemistry”, Acta Pharmaceutica Sinica B, 2021, 11(12): 3889-3907) reviews linkers that may be comprised in antibody-drug conjugates and which may also be used in the molecule of the present technology. For example, suitable linkers for use in the molecule of the present technology are APN-maleimide linker (3-(4-(2,5-dioxo-2,5-dihydro-1H-pyrrol-1-yl)phenyl)propiolonitrile, MAPN) or bis-maleimido-PEG3 (BM(PEG)3) linker (BM(PEG)3 (1,11-bismaleimido-triethyleneglycol)). In addition, bifunctional linkers may be used. For instance, the APN-Maleimide linker (806536, Sigma-Aldrich) can be used. Some other particularly preferred linkers are poly-alanine (such as AAA), as well as the linkers GS30 (SEQ ID NO: 85 in WO 06/122825) and GS9 (SEQ ID NO: 84 in WO 06/122825).

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the present technology that the length, the degree of flexibility and/or other properties of the linker(s) used (although not critical, as it usually is for linkers used in ScFv fragments) may have some influence on the properties of the final polypeptide of the present technology, including but not limited to the affinity, specificity or avidity for FcRn, or for one or more of the other antigens. Based on the disclosure herein, the skilled person will be able to determine the optimal linker(s) for use in a specific polypeptide of the present technology, optionally after some limited routine experiments.

It is also within the scope of the present technology that the linker(s) used confer one or more other favorable properties or functionality to the polypeptides of the present technology, and/or provide one or more sites for the formation of derivatives and/or for the attachment of functional groups (e.g., as described herein for the derivatives of the ISVDs, Nanobody® VHHs, or polypeptides of the present technology). For example, linkers containing one or more charged amino acid residues can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the present technology, optionally after some limited routine experiments.

Finally, when two or more linkers are used in the polypeptides of the present technology, these linkers may be the same or different. Again, based on the disclosure herein, the skilled person will be able to determine the optimal linkers for use in a specific polypeptide of the present technology, optionally after some limited routine experiments.

It will be appreciated that the order of the domains in the polypeptides of the present technology, such as, e.g., a first domain (e.g., serum albumin protein or domain binding specifically to a serum albumin protein), a second binding domain (e.g., an Fc domain of an IgG or fragment thereof), a third binding domain (e.g., one or more further groups, residues, moieties or binding units, or a domain binding to a therapeutically relevant target, or a therapeutic moiety) etc., in the polypeptide (i.e., the orientation or configuration of the binding domains or building blocks) can be chosen according to the needs of the person skilled in the art, as well as the relative affinities which may depend on the location of these binding domains in the polypeptide. Whether the polypeptide comprises one or more linkers to interconnect the binding domains and optionally further groups, residues or moieties is a matter of design choice. However, some orientations, with or without linkers, may provide preferred binding characteristics in comparison to other orientations. However, all different possible orientations are encompassed by the present technology. In a preferred embodiment, the third binding domain (e.g., one or more further groups, residues, moieties or binding units, or a domain binding to a therapeutically relevant target, or a therapeutic moiety) is not directly linked or is not linked by means of a linker, as defined herein, to the first domain as defined herein (e.g., a serum albumin protein or a domain binding specifically to a serum albumin protein).

In one embodiment, the present technology provides a polypeptide or fusion protein that comprises (i) at least one domain comprising a serum albumin protein or specifically binding to a serum albumin protein, (ii) an Fc domain of an IgG and (ii) one or more groups, residues, moieties or binding units, optionally attached via one or more linkers, in which said one or more other groups, residues, moieties or binding units target the molecule of the present technology to target molecules on cells, organs or tissues (“targeting moiety”). A targeting moiety, as defined herein, is any group, residue, moiety, or binding unit which is capable of being directed through its binding to a target. An amino acid sequence (such as an ISVD, an antibody, antigen-binding domains or fragments such as VHH domains or VH/VL domains, or generally an antigen binding protein or polypeptide or a fragment thereof) that “(specifically) binds”, that “can (specifically) bind to”, that “has affinity for” and/or that “has specificity for” a specific antigenic determinant, epitope, antigen or protein, or for a specific non-protein molecule, such as nucleic acids (such as DNA or RNA) or glycans (or for at least one part, fragment or epitope thereof) is said to be “against” or “directed against” said antigenic determinant, epitope, antigen, protein or non-protein molecule. Specific binding of an antigen-binding protein to an antigen or antigenic determinant can be determined in any suitable manner known per se, including, for example, Scatchard analysis and/or competitive binding assays, such as radio-immunoassays (RIA), enzyme immunoassays (EIA) and sandwich competition assays, and the different variants thereof known per se in the art; as well as the other techniques mentioned herein.

In one embodiment, the present technology provides a polypeptide or fusion protein that comprises (i) at least one domain comprising a serum albumin protein or specifically binding to a serum albumin protein, (ii) an Fc domain of an IgG and (ii) one or more groups, residues, moieties or binding units, optionally attached via one or more linkers, in which said one or more other groups, residues, moieties or binding units are capable of exerting a therapeutic activity in the animal or human body (“therapeutic moiety or precursor therefrom”). A therapeutic moiety, as defined herein, is any group, residue, moiety, or binding unit which is capable of exerting a therapeutic activity in the animal and/or human body. The therapeutic moiety may also be in the form of a precursor, which then gets activated to exert its therapeutic activity. For instance, a therapeutic moiety according to the present technology may be any therapeutic agent such as a drug, protein, peptide, gene, compound or any other pharmaceutically active ingredient which may be used for the treatment and/or prevention of a certain disease condition. For instance, a therapeutic moiety may be a therapeutic antibody, or a therapeutic ISVD. Non-limiting examples of therapeutic moieties which may be present in the polypeptide or fusion protein of the present technology are the following:
Epidermal growth factor receptor (EGFR)-binding molecules, as described, e.g., in WO 2005/044858, WO 2007/042289 or WO 2016/097313.
Von Willebrand factor (vWF)-binding molecules, as described in, e.g., WO 2006/122825.
Human epidermal growth factor receptor 2 (HER-2 or receptor tyrosine-protein kinase erbB-2)-binding molecules, as described, e.g., in WO 2009/068625.
Interleukin-6 receptor (IL-6R)-binding molecules, as described, e.g., in WO 2010/115995 or WO 2010/115998.
Neonatal Fc receptor (FcRn)-binding molecules, as described, e.g., in WO 2008/074867 or WO 2009/080764.
Polymeric immunoglobulin receptor (pigR)-binding molecules, as described, e.g., also in WO 2008/074867 or WO 2009/080764.
Vascular endothelial growth factor receptor 1 (VEGF-R1, also called Flt-I)-binding molecules, as described, e.g., in WO 2008/142165.
Platelet derived growth factor receptor beta (PDGF-Rp)-binding molecules, as described, e.g., in WO 2008/142165.
Fibroblast growth factor receptor 4 (FGF-R4)-binding molecules, as described, e.g., in WO 2008/142165.
Tumor necrosis factor-α (TNF-αt)-binding molecules, as described, e.g., in WO 2006/122786, WO 2015/173325, WO 2017/081320, WO 2021/110816, WO 2021/110817, or WO 2022/129572.
Insulin-like growth factor 1 receptor (IGF-IR)-binding molecules, as described in, e.g., WO 2007/042289.
Vascular endothelial growth factor (VEGF)-binding molecules, as described, e.g., in WO 2008/101985.
Receptor activator of nuclear factor kappa-β ligand (RANK-L)-binding molecules, as described, e.g., in WO 2008/142164 or WO 2015/173325.
Interleukin 23 (IL-23)-binding molecules, as described, e.g., in WO 2009/068627, WO 2011/135026, WO 2011/161263, WO 2015/173325, WO 2017/072299 or WO 2021/110816.
Respiratory syncytial virus (RSV) fusion (F) protein binding molecules, as described, e.g., in WO 2009/147248 or WO 2010/139808.
Influenza HSN1 hemagglutinin (HA)-binding molecules, as described, e.g., in WO 2009/147248.
Rabies virus 6-protein-binding molecules, as described, e.g., in WO 2009/147248.
CXC chemokine receptor type 4 (CXCR4)-binding molecules, as described, e.g., in WO 2009/138519, WO 2011/083141, WO 2011/161266, WO 2015/044386, WO 2015/173325 or WO 2016/156570.
CXC chemokine receptor type 7 (CXCR7)-binding molecules, as described, e.g., in WO 2009/138519, WO 2011/117423, WO 2012/130874 or WO 2015/173325.
Sclerostin-binding molecules, as described, e.g., in WO 2010/130830
Dickkopf-1 (Dkk-1)-binding molecules, as described, e.g., in WO 2010/130832.
HER-3-binding molecules, as described, e.g., in WO 2011/144749 or WO 2015/173325.
c-Met-binding molecules, as described, e.g., in WO 2012/042026, WO 2013/045707 or WO 2015/173325.
Amyloid beta (Aβ or A-beta)-binding molecules, as described, e.g., in WO 2011/107507 or WO 2015/173325.
CXC chemokine receptor type 2 (CXCR2)-binding molecules, as described, e.g., in WO 2012/062713 or WO 2013/168108.
Immunoglobulin E (IgE)-binding molecules, as described, e.g., in WO 2012/175740, WO 2014/087010 or WO 2015/173325.
Interleukin-17 (IL-17)A, IL-17F and/or IL-17A/F-binding molecules, as described, e.g., in WO 2012/156219.
Hepatocyte growth factor (HGF)-binding molecules, as described, e.g., in WO 2013/110531.
Pseudomonas aeruglnosa PcrV-binding molecules, as described, e.g., in WO 2013/128031.
P2X7-binding molecules, as described, e.g., in WO 2013/178783.
Interleukin-3 receptor (CD123)-binding molecules, as described, e.g., in WO 2015/044386 or WO 2018/091606.
Interleukin-3 receptor alpha (IL-3Rα)-binding molecules, as described, e.g., in WO 2015/044386.
Potassium voltage-gated channel, shaker-related subfamily, member 3 (Kᵥ1.3)-binding molecules, as described, e.g., in WO 2015/193452 or WO 2015/173325.
OX40L-binding molecules, as described, e.g., in WO 2011/073180, WO 2015/173325, WO 2021/110817 or WO 2022/063984.
CD40L-binding molecules, as described, e.g., in WO 2017/089618.
T-cell receptor (TCR)-binding molecules, as described, e.g., in WO 2016/180969, WO 2018/091606 or WO 2022/129637.
CD-4-binding molecules, as described, e.g., in WO 2016/156570.
CD-3-binding molecules, as described, e.g., in WO 2016/180982.
Glucocorticoid-induced Tumor Necrosis Factor (TNF) receptor-related protein (GITR)-binding molecules, as described, e.g., in WO 2017/068186.
P2X purinoceptor 7 (P2X7)-binding molecules, as described, e.g., in WO 2017/081265.
CD38-binding molecules, as described, e.g., in WO 2017/081211.
Macrophage migration inhibitory factor (MIF)-binding molecules, as described, e.g., in WO 2018/050833.
Matrix Metallopeptidase 13 (MMP13)-binding molecules, as described, e.g., in WO 2018/220235 or WO 2018/220236.
A disintegrin and metalloproteinase with thrombospondin motifs (ADAMTS)-binding molecules, as described, e.g., in WO 2018/220236.
Interleukin 13 (IL-13)-binding molecules, as described, e.g., in WO 2021/116182 or WO 2022/063984.
Thymic stromal lymphopoietin (TSLP)-binding molecules, as described, e.g., in WO 2021/116182.
Interleukin 6 (IL-6)-binding molecules, as described, e.g., in WO 2022/129572.

In a preferred embodiment the therapeutic moiety is directed against a desired antigen or target, is capable of binding to a desired antigen (and in particular capable of specifically binding to a desired antigen), and/or is capable of interacting with a desired target. In another embodiment, the at least one therapeutic moiety comprises or essentially consists of a therapeutic protein or polypeptide. In a further embodiment, the at least one therapeutic moiety comprises or essentially consists of a binding domain or binding unit, such as an immunoglobulin or immunoglobulin sequence (including but not limited to a fragment of an immunoglobulin), such as an antibody or an antibody fragment (including but not limited to an ScFv fragment), or of another suitable protein scaffold, such as protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23: 1257), and binding moieties based on DNA or RNA including but not limited to DNA or RNA aptamers (Ulrich et al., Comb Chem High Throughput Screen 2006 9(8):619-32). In one embodiment, the therapeutic moiety is not CTLA-4.

In yet another aspect, the at least one therapeutic moiety comprises or essentially consists of an antibody variable domain, such as a heavy chain variable domain or a light chain variable domain.

In a preferred aspect, the at least one therapeutic moiety comprises or essentially consists of at least one immunoglobulin single variable domain, such as a domain antibody, single domain antibody, “dAb” or a VHH (such as a Nanobody® VHH, a humanized VHH or a camelized VH) or an IgNAR domain.

For example, and without limitation, such polypeptides of the present technology may comprise in addition to the at least one domain comprising a serum albumin protein or specifically binding to a serum albumin protein and the at least one Fc domain of an IgG additionally at least one, such as two or three ISVD's (and preferably Nanobody® VHH's) against a therapeutic target. In these polypeptides, the at least one serum albumin protein or binding domain specific for a serum albumin protein and the Fc domain of an IgG and the additional one or more other groups, drugs, agents, residues, moieties or binding units may be directly linked to each other (as for example described in WO 99/23221) and/or may be linked to each other via one or more suitable spacers or linkers, or any combination thereof.

In one embodiment, the therapeutic moiety is not CTLA4 Ile38-Ser160 (Protein ID: NP_033973.2) or is not CTLA4 Ala37-Asp161 (Protein ID: NP_005205.2). In other embodiment, the therapeutic moiety is not VpreB Gln20-Ser121 (Protein ID: NP_058679.1].

In one embodiment, the present technology provides a polypeptide or fusion protein that comprises (i) at least one domain comprising a serum albumin protein or specifically binding to a serum albumin protein, (ii) an Fc domain of an IgG and (ii) one or more groups, residues, moieties or binding units, optionally attached via one or more linkers, in which said one or more other groups, residues, moieties or binding units are used for imaging purposes (“imaging moiety”). Examples of imaging moieties are provided in Agdeppa E D, Spilker M E. A review of imaging agent development. AAPS J. 2009 June; 11(2):286-99. For instance, the imaging moiety present in the molecule of the present technology may be suitable for radiotherapy and for radio/fluorescence-guided cancer surgery. For instance, the imaging moiety may comprise radioactive isotopes that can be used for diagnostic and therapeutic proposes. For instance, the imaging moiety may be a contrast agent. For instance, the imaging moiety may be a non-radioactive medical isotope. For instance, the imaging moiety may include desferrioxamine (DFO), such as used for ⁸⁹Zirconium-DFO-labeling. For instance, the imaging moiety may be a fluorophore such as Alexa 647 or pHAb.

In one embodiment, the present technology provides a polypeptide or fusion protein that comprises (i) at least one domain comprising a serum albumin protein or specifically binding to a serum albumin protein, (ii) an Fc domain of an IgG and (ii) one or more groups, residues, moieties or binding units, optionally attached via one or more linkers, in which said one or more other groups, residues, moieties or binding units are able to impart certain toxicity to cells and/or tissues (“toxic moiety” or “drug”). A toxic moiety which may be attached or conjugated to the protein-based carrier building block may belong to the “tubulin inhibitor” family (e.g., maytansinoids, auristatins, taxol derivates) or to the “DNA-modifying agents” family (e.g., calicheamicins, duocarymycins). They can also be antibiotics or enzymes. For a review, see Criscitiello C. et al., “Antibody-drug conjugates in solid tumors: a look into novel targets”, Hematol Oncol, 2021, 14:20.

In one embodiment, the present technology provides a polypeptide or fusion protein that comprises (i) at least one domain comprising a serum albumin protein or specifically binding to a serum albumin protein, (ii) an Fc domain of an IgG and (ii) one or more groups, residues, moieties or binding units, optionally attached via one or more linkers, in which said one or more other groups, residues, moieties or binding units have a therapeutic and/or prophylactic effect, i.e., is a “vaccine”. A vaccine is a biological preparation that provides active acquired immunity to a particular antigen. Vaccines may be prophylactic or therapeutic.

### 5.7 Methods for Preparing the Polypeptides of the Present Technology

The present technology also relates to methods for preparing the polypeptides, ISVDs, compounds, fusion proteins and constructs described herein. The polypeptides, ISVDs, compounds, fusion proteins and constructs of the present technology can be prepared in a manner known per se, as will be clear to the skilled person from the further description herein. For example, polypeptides, ISVDs, compounds, fusion proteins and constructs of the present technology can be prepared in any manner known per se for the preparation of antibodies and in particular for the preparation of antibody fragments (including but not limited to (single) domain antibodies and ScFv fragments). Some preferred, but non-limiting methods for preparing the polypeptides, fusion proteins and constructs include the methods and techniques described herein.

Hence, another embodiment of the present technology relates to a method for producing the polypeptides binding to FcRn of the present technology. As described in detail above, the polypeptides according to the present technology comprise (i) at least one domain comprising a serum albumin protein and/or at least one domain specifically binding to a serum albumin protein and (ii) an Fc domain of an immunoglobulin G (IgG) or a fragment thereof, preferably a FcRn-binding fragment thereof. The skilled person is aware of means of linking two polypeptides (i) and (ii), or more, if any, to prepare the polypeptides binding to FcRn of the present technology. For instance, the method may comprise the steps of:
a) Providing at least a first (i) and a second (ii) polypeptide, or more, as described above;
b) Covalently linking the polypeptides together, directly or by means of a linker, as described below.

For instance, the method may comprise the steps of:
a) Selecting at least a first (i) and a second (ii) polypeptide, or more, as described above;
b) Designing a genetic construct which encodes a protein sequence comprising the first (i) and second (ii) polypeptides, or more; and
c) Introducing said genetic construct in an expression system to obtain the FcRn antagonists of the present technology, as described above in the present specification.

The methods for producing a polypeptide, ISVD, compound, fusion protein and construct of the present technology may comprise the following steps:
the expression, in a suitable host cell or (non-human) host organism (also referred to herein as a “(non-human) host of the present technology”) or in another suitable expression system of a nucleic acid that encodes said ISVD, polypeptide or protein construct of the present technology, optionally followed by:
isolating and/or purifying the polypeptide, ISVD, compound and construct of the present technology thus obtained.

In particular, such a method may comprise the steps of:
cultivating and/or maintaining a host cell or (non-human) host organism of the present technology under conditions that are such that said host cell or (non-human) host organism of the present technology expresses and/or produces at least one polypeptide, ISVD, fusion protein compound and/or construct of the present technology; optionally followed by:
isolating and/or purifying the polypeptide, ISVD, fusion protein, compound and/or construct of the present technology thus obtained.

Usually, for ease of expression and production, a polypeptide of the present technology will be a linear polypeptide. However, the present technology in its broadest sense is not limited thereto. For example, when a polypeptide of the present technology comprises three or more domains and/or ISVDs and/or Nanobody® VHHs, it is possible to link them by use of a linker with three or more “arms”, with each “arm” being linked to a domain, ISVD or Nanobody® VHH, so as to provide a “star-shaped” construct. It is also possible, although usually less preferred, to use circular constructs.

In the context of the present technology, the position of each of the polypeptides ((i) and (ii), or more, if present) in the polypeptides of the present technology is not limited. For instance, the first polypeptide (i) may be located in the N-terminal part of the polypeptides, whereas the second polypeptide (ii) may be located in the C-terminal part of the polypeptides. In addition, the first polypeptide (i) may be located in the C-terminal part of the polypeptides, whereas the second polypeptide (ii) may be located in the N-terminal part of the polypeptides. In addition, the at least one first polypeptide (i) and the at least one second polypeptide (ii) may be directly linked to each other or linked via a linker, such as peptide linkers.

The use of linkers to connect two or more (poly)peptides is well known in the art and is described below in this description, see also Table A-2.

For instance, the at least one second polypeptide (ii), the Fc domain or a fragment thereof may comprise, in its N-terminal part, a sequence comprising or consisting of part of the hinge region. The “hinge region” is s a short sequence of the heavy chains (H) of antibodies linking the Fab (Fragment antigen binding) region to the Fc (Fragment crystallizable) region. For instance, the Fc domain or a fragment thereof may comprise, in its N-terminal part, a sequence comprising or consisting of a sequence selected from SEQ ID NO: 38-42 and 200. In a preferred embodiment, the Fc domain or a fragment thereof may comprise, in its N-terminal part, a sequence comprising or consisting of SEQ ID NO: 38 or 200, more preferably SEQ ID NO.: 38.

When the Fc domain or a fragment thereof is located at the C-terminal part of the polypeptide, the other polypeptide (e.g., the at least one first polypeptide (i)) will be located at the N-terminal part of the polypeptide. In this case, both polypeptides may be linked directly or by means of a linker, as described above. If they are linked by means of a linker, in a preferred embodiment, they are linked by means of the hinge region comprised in the Fc domain or fragment thereof, preferably comprising or consisting of a polypeptide as described in Table A-2, such as SEQ ID NO: 25-42 or 200, as described above.

In the specific embodiment where the at least one second (ii) polypeptide comprised in the polypeptide of the present technology is a dimeric Fc domain (i.e., a Fc domain comprising two polypeptides, each comprising at least one CH2 and at least one CH3 domains), the other polypeptide (i) comprised in the polypeptide may be linked (directly or via a linker, as described below) to the N- or C-terminal part of one of the polypeptides (chains) comprised in the dimeric Fc domain. For instance, if the at least one second polypeptide (ii) is a dimeric Fc domain, the at least one first polypeptide (i) may be linked (directly or via a linker, as described below) to the N-terminal part of one of the polypeptides comprised in the dimeric Fc domain, e.g., to the hinge region or part thereof of one of the polypeptides, see, e.g., SEQ ID NO: 38-42 and 200, preferably 38 or 200. For instance, if the at least one second polypeptide (ii) is a dimeric Fc domain, the at least one first polypeptide (i) may be linked (directly or via a linker, as described below) to the C-terminal part of one of the polypeptides comprised in the dimeric Fc domain, e.g., via a peptide linker, see, e.g., SEQ ID NO: 25 to 37, preferably SEQ ID NO: 29 or 36, see Table A-2. See FIGS. 1, 3 and 6.

### 5.8 Linkers

In the polypeptides according to the present technology the at least one serum albumin protein or the at least one domain binding to serum albumin protein and the at least one Fc domain of an IgG or fragment thereof (and the further groups, residues, moieties or binding units, if any) are directly (covalently) linked to each other or are (covalently) linked via a linker, such as a peptidic linker. The use of linkers to connect two or more (poly)peptides is well known in the art. One frequently used class of peptidic linkers are known as the “Gly-Ser” or “GS” linkers. These are linkers that essentially consist of glycine (G) and serine (S) residues, and usually comprise one or more repeats of a peptide motif such as the GGGGS (SEQ ID NO: 26) motif (for example, exhibiting the formula (Gly-Gly-Gly-Gly-Ser)ₙ in which n may be 1, 2, 3, 4, 5, 6, 7 or more). Some often used examples of such GS linkers are 9GS linkers (e.g., GGGGSGGGS, SEQ ID NO: 29), 15GS linkers (n=3) (e.g., SEQ ID NO.: 31) and 35GS linkers (n=7) (e.g., SEQ ID NO.: 36). Reference is for example made to Chen et al., Adv. Drug Deliv. Rev. 2013 Oct. 15; 65(10): 1357-1369; and Klein et al., Protein Eng. Des. Sel. (2014) 27 (10): 325-330. In particular but non-limiting embodiments, the linker is chosen from the group consisting of linkers of 3A, 3GS, 5GS, 7GS, 9GS, 10GS, 15GS, 18GS, 20GS, 25GS, 30GS and 35GS (SEQ ID NOs: 25 to 42).

TABLE A-2
Linker sequences
(“ID” refers to the SEQ ID NO as used herein)

| Name | ID | Amino acid sequence |
|---|---|---|
| 3A linker | 25 | AAA |
| 5GS linker | 26 | GGGGS |
| 7GS linker | 27 | SGGSGGS |
| 8GS linker | 28 | GGGGSGGS |
| 9GS linker | 29 | GGGGSGGGS |
| 10GS linker | 30 | GGGGSGGGGS |
| 15GS linker | 31 | GGGGSGGGGSGGGGS |
| 18GS linker | 32 | GGGGSGGGGSGGGGSGGS |
| 20GS linker | 33 | GGGGSGGGGSGGGGSGGGGS |
| 25GS linker | 34 | GGGGSGGGGSGGGGSGGGGSGGGGS |
| 30GS linker | 35 | GGGGSGGGGSGGGGSGGGGSGGGGS |
| | | GGGGS |
| 35GS linker | 36 | GGGGSGGGGSGGGGSGGGGSGGGGS |
| | | GGGGSGGGGS |
| 40GS linker | 37 | GGGGSGGGGSGGGGSGGGGSGGGGS |
| | | GGGGSGGGGSGGGGS |
| Short G1 hinge | 38 | DKTHTCPPCP |
| G1 hinge | 39 | EPKSCDKTHTCPPCP |
| 9GS-G1 hinge | 40 | GGGGSGGGSEPKSCDKTHTCPPCP |
| Llama upper long | 41 | EPKTPKPQPAAA |
| hinge region | | |
| G3 hinge | 42 | ELKTPLGDTTHTCPRCPEPKSCDTP |
| | | PPCPRCPEPKSCDTPPPCPRCPEPK |
| | | SCDTPPPCPRCP |
| Short hinge | 200 | DKTHTCPSCP |
| linker | | |

### 5.9 Nucleic Acid Sequences and Genetic Constructs

Accordingly, the present technology also relates to a nucleic acid or nucleotide sequence that encodes an ISVD, polypeptide, compound, (fusion) protein or (multispecific) construct of the present technology (also referred to as “nucleic acid of the present technology” or “nucleotide sequence of the present technology”). A nucleic acid of the present technology can be in the form of single or double stranded DNA or RNA and is preferably in the form of double stranded DNA. For example, the nucleotide sequences of the present technology may be genomic DNA, cDNA or synthetic DNA (such as DNA with a codon usage that has been specifically adapted for expression in the intended host cell or host organism).

According to one embodiment of the present technology, the nucleic acid of the present technology is in essentially isolated from, as defined herein. The nucleic acid of the present technology may also be in the form of, be present in and/or be part of a vector, such as for example a plasmid, cosmid or YAC, which again may be in essentially isolated form. A nucleic acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence that is “in essentially isolated form” is preferably essentially homogeneous, as determined using a suitable technique, such as a suitable chromatographical technique, such as polyacrylamide-gel electrophoresis.

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).

Alternatively, the degree of sequence identity between two or more nucleotide sequences may be calculated using a known computer algorithm for sequence alignment such as NCBI Blast v2.0, using standard settings. Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A. Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence.

The nucleic acids of the present technology can be prepared or obtained in a manner known per se, based on the information on the polypeptides or protein constructs of the present technology given herein, and/or can be isolated from a suitable natural source. Also, as will be clear to the skilled person, to prepare a nucleic acid of the present technology, also several nucleotide sequences, such as at least one nucleotide sequence encoding an immunoglobulin single variable domain of the present technology and for example nucleic acids encoding one or more linkers can be linked together in a suitable manner.

Techniques for generating the nucleic acids of the present technology will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g., to create cassettes and/or regions that may easily be digested and/or ligated using suitable restriction enzymes), and/or the introduction of mutations by means of a PCR reaction using one or more “mismatched” primers. These and other techniques will be clear to the skilled person, and reference is again made to the standard handbooks, such as Sambrook et al. and Ausubel et al., mentioned herein, as well as the Examples below.

The nucleic acid of the present technology may also be in the form of, be present in and/or be part of a genetic construct, as will be clear to the person skilled in the art. Such genetic constructs generally comprise at least one nucleic acid of the present technology that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the present technology will also be referred to herein as “genetic constructs of the present technology”.

The genetic constructs of the present technology may be DNA or RNA and are preferably double-stranded DNA. The genetic constructs of the present technology may also be in a form suitable for transformation of the intended host cell or (non-human) host organism, in a form suitable for integration into the genomic DNA of the intended host cell or in a form suitable for independent replication, maintenance and/or inheritance in the intended host organism. For instance, the genetic constructs of the present technology may be in the form of a vector, such as for example a plasmid, cosmid, YAC, a viral vector or transposon. In particular, the vector may be an expression vector, i.e., a vector that can provide for expression in vitro and/or in vivo (e.g., in a suitable host cell, host organism and/or expression system).

In a preferred but non-limiting embodiment, a genetic construct of the present technology comprises
a) at least one nucleic acid of the present technology; operably connected to
b) one or more regulatory elements, such as a promoter and optionally a suitable terminator; and optionally also c) one or more further elements of genetic constructs known per se; in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person and may for instance depend upon the type of construct used; the intended host cell or host organism; the manner in which the nucleotide sequences of the present technology of interest are to be expressed (e.g., via constitutive, transient or inducible expression); and/or the transformation technique to be used. For example, regulatory sequences, promoters and terminators known per se for the expression and production of antibodies and antibody fragments (including but not limited to (single) domain antibodies and ScFv fragments) may be used in an essentially analogous manner.

Preferably, in the genetic constructs of the present technology, said at least one nucleic acid of the present technology and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promoter). Generally, when two nucleotide sequences are operably linked, they will be in the same orientation and usually also in the same reading frame. They will usually also be essentially contiguous, although this may also not be required.

### 5.10 (Non-Human) Hosts and Host Cells

The nucleic acids of the present technology and/or the genetic constructs of the present technology may be used to transform a host cell or (non-human) host organism, i.e., for expression and/or production of the polypeptide or protein construct of the present technology. The host is preferably a non-human host. Suitable (non-human) hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividons; of Staphylococcus, for example of Staphylococcus carnosus; and of Lactococcus, for example of Loctococcus lactis; a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojoe; or from other filamentous fungi; a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanalica; of Hansenula, for example of Hansenula polymorpho; of Kluyveromyces, for example of Kluyveromyces lactis; of Arxula, for example of Arxula adeninivorans; of Yarrowia, for example of Yarrowia lipolytica; an amphibian cell or cell line, such as Xenopus oocytes; an insect-derived cell or cell line, such as cells/cell lines derived from lepidoptera, including but not limited to Spodoptera SF9 and Sf21 cells or cells/cell lines derived from Drosophila, such as Schneider and Kc cells; a plant or plant cell, for example in tobacco plants; and/or a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells; as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al. 1998 (Res. Immunol. 149: 589-99); Riechmann and Muyldermans 1999 (J. Immunol. Met. 231: 25-38); van der Linden 2000 (J. Biotechnol. 80: 261-70); Joosten et al. 2003 (Microb. Cell Fact. 2: 1); Joosten et al. 2005 (Appl. Microbiol. Biotechnol. 66: 384-92); and the further references cited herein.

For expression of the polypeptides, ISVDs, compounds or constructs in a cell, they may also be expressed as so-called “intrabodies”, as for example described in WO 94/02610, WO 95/22618 and U.S. Pat. No. 7,004,940; WO 03/014960; in Cattaneo and Biocca 1997 (Intracellular Antibodies: Development and Applications. Landes and Springer-Verlag); and in Kontermann 2004 (Methods 34: 163-170).

According to one preferred, but non-limiting embodiment of the present technology, the polypeptide, ISVD, (fusion)protein or construct of the present technology is produced in a bacterial cell, in particular a bacterial cell suitable for large scale pharmaceutical production, such as cells of the strains mentioned above.

According to another preferred, but non-limiting embodiment of the present technology, polypeptide, ISVD, (fusion)protein, or construct of the present technology is produced in a yeast cell, in particular a yeast cell suitable for large scale pharmaceutical production, such as cells of the species mentioned above.

According to yet another preferred, but non-limiting embodiment of the present technology, the polypeptide, ISVD, (fusion)protein or construct of the present technology is produced in a mammalian cell, in particular in a human cell or in a cell of a human cell line, and more in particular in a human cell or in a cell of a human cell line that is suitable for large scale pharmaceutical production, such as the cell lines mentioned hereinabove.

Suitable techniques for transforming a host or host cell of the present technology will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the present technology may be performed. This may for instance be a selection step based on a selectable marker present in the genetic construct of the present technology or a step involving the detection of the polypeptide of the present technology, e.g., using specific antibodies.

The transformed host cell (which may be in the form or a stable cell line) or host organisms (which may be in the form of a stable mutant line or strain) form further aspects of the present technology.

Preferably, these host cells or host organisms are such that they express or are (at least) capable of expressing (e.g., under suitable conditions), the ISVD, polypeptide, compound, (fusion)protein or construct of the present technology (and in case of a host organism: in at least one cell, part, tissue or organ thereof). The present technology also includes further generations, progeny and/or offspring of the host cell or host organism of the present technology, for instance obtained by cell division or by sexual or asexual reproduction.

Accordingly, in another aspect, the present technology relates to a (non-human) host or host cell that expresses (or that under suitable circumstances is capable of expressing) an ISVD, polypeptide, (fusion)protein or construct of the present technology; and/or that contains a nucleic acid encoding the same. Some preferred but non-limiting examples of such hosts or host cells can be as generally described in WO 04/041867, WO 04/041865 or WO 09/068627. For example, ISVDs, polypeptides, (fusion)proteins and constructs of the present technology may with advantage be expressed, produced or manufactured in a yeast strain, such as a strain of Pichia pastoris. Reference is also made to WO 04/25591, WO 10/125187, WO 11/003622, and WO 12/056000 which also describes the expression/production in Pichia and other hosts/host cells of immunoglobulin single variable domains and polypeptides comprising the same.

To produce/obtain expression of the polypeptides, ISVDs, (fusion)proteins or constructs of the present technology, the transformed host cell or transformed host organism may generally be kept, maintained and/or cultured under conditions such that the (desired) ISVD, polypeptide, (fusion)protein or construct of the present technology is expressed/produced. Suitable conditions will be clear to the skilled person and will usually depend upon the host cell/host organism used, as well as on the regulatory elements that control the expression of the (relevant) nucleotide sequence of the present technology. Again, reference is made to the handbooks and patent applications mentioned above in the paragraphs on the genetic constructs of the present technology.

Generally, suitable conditions may include the use of a suitable medium, the presence of a suitable source of food and/or suitable nutrients, the use of a suitable temperature, and optionally the presence of a suitable inducing factor or compound (e.g., when the nucleotide sequences of the present technology are under the control of an inducible promoter); all of which may be selected by the skilled person. Again, under such conditions, the ISVDs, polypeptides, (fusion)proteins or constructs of the present technology may be expressed in a constitutive manner, in a transient manner, or only when suitably induced.

It will also be clear to the skilled person that the polypeptide, ISVD, (fusion)protein or construct of the present technology may (first) be generated in an immature form (as mentioned above), which may then be subjected to post-translational modification, depending on the host cell/host organism used. Also, the ISVD, polypeptide, (fusion)protein or construct of the present technology may be glycosylated, again depending on the host cell/host organism used.

The polypeptide, ISVD, (fusion)protein or construct of the present technology may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g., using a specific, cleavable amino acid sequence fused with the polypeptide or construct of the present technology) and/or preparative immunological techniques (i.e., using antibodies against the amino acid sequence to be isolated).

An polypeptide or protein is considered to be “(in) essentially isolated (form)”—for example, compared to its native biological source and/or the reaction medium or cultivation medium from which it has been obtained—when it has been separated from at least one other component with which it is usually associated in said source or medium, such as another protein/polypeptide, another biological component or macromolecule or at least one contaminant, impurity or minor component. In particular, a polypeptide or protein is considered “essentially isolated” when it has been purified at least 2-fold, in particular at least 10-fold, more in particular at least 100-fold, and up to 1000-fold or more. A polypeptide or protein that is “in essentially isolated form” is preferably essentially homogeneous, as determined using a suitable technique, such as a suitable chromatographical technique, such as polyacrylamide-gel electrophoresis.

### 5.11 Pharmaceutical Compositions and Use in Therapy

The present technology also provides a composition comprising the polypeptide and/or fusion protein of the present technology. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

Hence, the present technology also relates to a pharmaceutical composition comprising the polypeptide, ISVD, fusion protein, compound or construct of the present technology.

The present technology thus provides the polypeptide and/or fusion protein or a composition comprising the same of the present technology for use as a medicament. Also provided is the polypeptide and/or fusion protein or a composition comprising the same of the present technology for use in the (prophylactic and/or therapeutic) treatment. Hence, the present technology provides a method of prophylactic and/or therapeutic treatment which comprises the administration of the the polypeptide and/or fusion protein or a composition comprising the same of the present technology to a subject in need thereof. The present technology further provides the use of the polypeptide and/or fusion protein or a composition comprising the same of the present technology for the manufacture of a medicament. The present technology further provides the use of the polypeptide and/or fusion protein or a composition comprising the same of the present technology in therapy/as a medicament. Also provided is the molecule of the present technology or a composition comprising the polypeptide and/or fusion protein or a composition comprising the same of the present technology for use in the (prophylactic and/or therapeutic) treatment of an autoimmune/inflammatory disease and/or a proliferative disease, such as cancer, such as hematological (blood) and solid tumor cancer disease. Hence, the present technology provides a method of prophylactic and/or therapeutic treatment of an autoimmune/inflammatory disease and/or a proliferative disease, such as cancer, such as hematological (blood) and solid tumor cancer disease, wherein the method comprises the administration of the the polypeptide and/or fusion protein or a composition comprising the same of the present technology to a subject in need thereof. The present technology provides a method for treating an autoimmune/inflammatory disease and/or a proliferative disease, such as cancer, such as hematological (blood) and solid tumor cancer disease, wherein the method comprises the administration of the the polypeptide and/or fusion protein or a composition comprising the same of the present technology to a subject in need thereof. The present technology further provides the use of the polypeptide and/or fusion protein or a composition comprising the same of the present technology for the manufacture of a medicament for the (prophylactic and/or therapeutic) treatment of an autoimmune/inflammatory disease and/or a proliferative disease, such as cancer, such as hematological (blood) and solid tumor cancer disease. The present technology further provides the use of the polypeptide and/or fusion protein or a composition comprising the same of the present technology in a method for treating an autoimmune/inflammatory disease and/or a proliferative disease, such as cancer, such as hematological (blood) and solid tumor cancer disease.

Also provided is polypeptide and/or fusion protein or a composition comprising the same of the present technology for use in the (prophylactic and/or therapeutic) treatment of an infectious disease. Hence, the present technology provides a method of prophylactic and/or therapeutic treatment of an infectious disease, wherein the method comprises the administration of the the polypeptide and/or fusion protein or a composition comprising the same of the present technology to a subject in need thereof. The present technology provides a method for treating an infectious disease, wherein the method comprises the administration of the the polypeptide and/or fusion protein or a composition comprising the same of the present technology to a subject in need thereof. The present technology further provides the use of the polypeptide and/or fusion protein or a composition comprising the same of the present technology for the manufacture of a medicament for the (prophylactic and/or therapeutic) treatment of an infectious disease. The present technology further provides the use of the polypeptide and/or fusion protein or a composition comprising the same of the present technology in a method for treating an infectious disease.

Also provided is the polypeptide and/or fusion protein or a composition comprising the same of the present technology for use as a vaccine. Hence, the present technology provides a vaccine comprising the polypeptide and/or fusion protein or a composition comprising the same of the present technology, optionally further comprising further components such as pharmaceutically acceptable carriers and/or adjuvants.

A “subject” as referred to in the context of the present technology can be any animal. In one embodiment, the subject is a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal. In one embodiment, the subject is a human subject.

In the above methods, the polypeptides, ISVDs, compounds or constructs, fusion proteins or compositions comprising the same of the present technology can be administered in any suitable manner, depending on the specific pharmaceutical formulation or composition to be used. Thus, the polypeptides, ISVDs, compounds or constructs of the present technology and/or the compositions comprising the same can for example be administered orally, intraperitoneally, intravenously, subcutaneously, intramuscularly, or via any other route of administration that circumvents the gastrointestinal tract, intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

As used herein, the term “therapeutic agent” or “therapeutic moiety” refers to any agent or moiety that can be used in the treatment and/or management of a disease or disorder, such as a hyperproliferative cell disorder, e.g., cancer, or one or more symptoms thereof, or such as an inflammatory disease, infectious disease and/or autoimmune disease. In certain embodiments, the term “therapeutic agent” refers to a multispecific polypeptide of the present technology. Preferably, a therapeutic agent is an agent which is known to be useful for, or has been or is currently being used for the treatment, prevention and/or management of a disease or disorder, or one or more symptoms thereof.

As used herein, a “therapeutically effective amount” in the present context refers to the amount of a therapy alone, or in combination with other therapies, that provides a therapeutic benefit in the treatment and/or management of a disease and/or disorder. In one aspect, a therapeutically effective amount refers to the amount of a therapy sufficient to cure, modify, stabilize or control a disease and/or disorder, or one or more symptoms thereof. In another aspect, a therapeutically effective amount refers to the amount of a therapy sufficient to reduce the symptoms of a disease and/or disorder. In another aspect, a therapeutically effective amount refers to the amount of a therapy sufficient to delay or minimize the spread of a disease and/or disorder. Used in connection with an amount of a multispecific polypeptide of the present technology, the term can encompass an amount that improves overall therapy, reduces or avoids unwanted effects, or enhances the therapeutic efficacy of or synergies with another therapy. In one embodiment, a therapeutically effective amount of a therapy reduces or avoids unwanted effects, or enhances the therapeutic efficacy of or synergies with another therapy by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline) in an assay known in the art or described herein.

As used herein, the term “therapy” refers to any protocol, method and/or agent that can be used in the treatment, prevention and/or management of a disease and/or disorder, or symptoms thereof. In certain embodiments, the terms “therapies” and “therapy” refer to a biological therapy, supportive therapy, and/or other therapies useful in the treatment, prevention and/or management of a disease and/or disorder, or one or more symptoms thereof known to one of skill in the art, such as medical personnel.

As used herein, the terms “treat”, “treatment” and “treating” in the context of administering (a) therapy(ies) to a subject refer to the reduction or amelioration of the progression, severity, and/or duration of a diseases or disorder, and/or the amelioration of one or more symptoms thereof resulting from the administration of one or more therapies (including, but not limited to, the administration of one or more prophylactic or therapeutic agents).

The polypeptides, ISVDs, compounds, fusion proteins or constructs of the present technology and/or the compositions comprising the same are administered according to a regime of treatment that is suitable for preventing and/or treating the disease and/or disorder to be prevented or treated. The clinician will generally be able to determine a suitable treatment regimen, depending on factors such as the stage of the disease and/or disorder to be treated, the severity of the disease and/or disorder to be treated and/or the severity of the symptoms thereof, the specific polypeptide, ISVD, compound or construct of the present technology to be used, the specific route of administration and pharmaceutical formulation or composition to be used, the age, gender, weight, diet, general condition of the patient, and similar factors well known to the clinician.

Generally, the treatment regimen will comprise the administration of one or more polypeptides, ISVDs, compounds or constructs of the present technology, or of one or more compositions comprising the same, in one or more pharmaceutically effective amounts or doses. The specific amount(s) or doses to be administered can be determined by the clinician, again based on the factors cited above.

Usually, in the above method, a single polypeptide, ISVD, compound or construct of the present technology will be used. It is however within the scope of the present technology to use two or more polypeptides, ISVDs, compounds and/or constructs of the present technology in combination.

The polypeptides, ISVDs, compounds or constructs of the present technology may also be used in combination with one or more further pharmaceutically active compounds or principles, i.e., as a combined treatment regimen, which may or may not lead to a synergistic effect. Again, the clinician will be able to select such further compounds or principles, as well as a suitable combined treatment regimen, based on the factors cited above and his expert judgement.

In particular, the polypeptides, ISVDs, compounds or constructs of the present technology may be used in combination with other pharmaceutically active compounds or principles that are or can be used for the prevention and/or treatment of the disease and/or disorder cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g., essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

In one aspect, the disclosure provides methods for the administration of immunoglobulin single variable domains and polypeptide constructs thereof comprising one or more immunoglobulin single variable domains, polypeptides, compounds and/or constructs. In some embodiments, the immunoglobulin single variable domain, polypeptide, compound and/or construct is administered as a pharmaceutical composition. The pharmaceutical composition, in addition to the immunoglobulin single variable domains and polypeptide constructs thereof includes a pharmaceutically acceptable carrier.

Since the compounds or polypeptides of the present technology have an increased half-life and/or a decreased clearance, they are preferably administered to the circulation. As such, they can be administered in any suitable manner that allows the compound or polypeptide of the present technology to enter the circulation, such as intravenously, via injection or infusion, or in any other suitable manner (including oral administration, subcutaneous administration, intramuscular administration, administration through the skin, intranasal administration, administration via the lungs, etc.). Suitable methods and routes of administration will be clear to the skilled person, again for example also from the teaching of the published patent applications of Ablynx N.V., such as for example WO 04/041862, WO 2006/122786, WO 2008/020079, WO 2008/142164 or WO 2009/068627.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Methods of preparing these formulations or compositions include the step of bringing into association an immunoglobulin single variable domain or polypeptide construct with the carrier and, optionally, one or more accessory ingredients. In general, the formulations are prepared by uniformly and intimately bringing into association an immunoglobulin single variable domain or polypeptide construct with liquid carriers, or finely divided solid carriers, or both, and then, if necessary, shaping the product.

5.12 Methods for Increasing or Extending the Serum Half-Life (Ta/n) and/or Reducing Clearance

Hence, the pharmacokinetic (PK) parameters of any group, residue, moiety or binding unit, as described above, may be improved by linking, (directly or by means of a linker) the group, residue, moiety or binding unit to a polypeptide that comprises (i) at least one domain comprising a serum albumin protein or specifically binding to a serum albumin protein and (ii) an Fc domain of an IgG (the polypeptide of the present technology), as described herein.

Pharmacokinetic (PK) parameters describe drug/therapeutic moiety/moiety absorption, distribution, metabolism, and elimination and how these processes define plasma (serum) drug/moiety concentrations. For instance, the serum half-life (t_{1/2}) is the time (e.g., in hours) taken for concentration of a molecule (such as a drug or a therapeutic moiety) to decrease from its maximum concentration (Cₘₐₓ) to half of Cₘₐₓ in the blood plasma or serum. The term “half-life” has been described in the present specification. For instance, the clearance is a pharmacokinetic parameter representing the efficiency of drug elimination. Clearance is defined as the volume of plasma cleared of a drug over a specified time period. Clearance is equal to the rate at which a drug is removed from plasma(mg/min) divided by the concentration of that drug in the plasma (mg/mL). Clearance can be calculated as described herein. A reduction in drug clearance is associated with an increase in the half-life of a drug/therapeutic moiety/moiety, and an increase in clearance is associated with a decrease in the half-life of the drug/therapeutic moiety/moiety.

Hence, the pharmacokinetic (PK) parameters of any group, residue, moiety or binding unit, as described above, which is linked to the polypeptide of the present technology (i.e., comprised in the fusion protein or polypeptide of the present technology), as described herein, may be improved as compared with the PK parameters of the group, residue, moiety or binding unit as such (i.e., without the polypeptide of the present technology). For instance, the serum half-life (t_{1/2}) of any group, residue, moiety or binding unit, as described above, which is linked to the polypeptide of the present technology may be increased or extended as compared with the serum half-life (t_{1/2}) of the group, residue, moiety or binding unit as such (i.e., without the polypeptide of the present technology). For instance, the clearance of any group, residue, moiety or binding unit, as described above, which is linked to the polypeptide of the present technology may be reduced or decreased as compared with the clearance of the group, residue, moiety or binding unit as such (i.e., without the polypeptide of the present technology). The present technology thus provides a method for improving at least one PK parameter, such as serum half-life (t_{1/2}) and/or clearance, of any group, residue, moiety or binding unit, as described above, the method comprising:
a. Providing a group, residue, moiety or binding unit, as described above;
b. Linking (directly or by means of a linker) the group, residue, moiety or binding unit provided in a. to a polypeptide that comprises (i) at least one domain comprising a serum albumin protein or specifically binding to a serum albumin protein and (ii) an Fc domain of an IgG, as described herein.

Hence, the present technology provides a method for increasing or extending the serum half-life (t_{1/2}) of any group, residue, moiety or binding unit, as described above, the method comprising:
a. Providing a group, residue, moiety or binding unit, as described above;
b. Linking (directly or by means of a linker) the group, residue, moiety or binding unit provided in a. to a polypeptide that comprises (i) at least one domain comprising a serum albumin protein or specifically binding to a serum albumin protein and (ii) an Fc domain of an IgG, as described herein.

Hence, the present technology provides a method for decreasing or reducing the clearance of any group, residue, moiety or binding unit, as described above, the method comprising:
a. Providing a group, residue, moiety or binding unit, as described above;
b. Linking (directly or by means of a linker) the group, residue, moiety or binding unit provided in a. to a polypeptide that comprises (i) at least one domain comprising a serum albumin protein or specifically binding to a serum albumin protein and (ii) an Fc domain of an IgG, as described herein.

The present technology thus provides the polypeptide of the present technology for improving the PK parameters such as serum half-life (t_{1/2}) and/or clearance of any group, residue, moiety or binding unit, as described above (as compared with the PK parameters of the group, residue, moiety or binding unit as such, not linked to the polypeptide of the present technology), by linking (directly or by means of a linker) the group, residue, moiety or binding unit to a polypeptide that comprises (i) at least one domain comprising a serum albumin protein or specifically binding to a serum albumin protein and (ii) an Fc domain of an IgG, as described herein.

The present technology further provides the use of the polypeptides of the present technology for improving the PK parameters such as serum half-life (t_{1/2}) and/or clearance of any group, residue, moiety or binding unit, as described above (as compared with the PK parameters of the group, residue, moiety or binding unit as such, not linked to the polypeptide of the present technology), by linking (directly or by means of a linker) the group, residue, moiety or binding unit to a polypeptide that comprises (i) at least one domain comprising a serum albumin protein or specifically binding to a serum albumin protein and (ii) an Fc domain of an IgG, as described herein.

If the polypeptide of the present technology is linked (directly or by means of a linker, as defined herein) to one or more drugs and/or therapeutic moieties and/or vaccines, pharmacokinetic (PK) parameters of that drug and/or therapeutic moiety and/or vaccine which is linked to the polypeptide of the present technology (i.e., comprised in the fusion protein or polypeptide of the present technology), as described herein, may be improved as compared with the PK parameters of the drug and/or therapeutic moiety and/or vaccine as such (i.e., without the polypeptide of the present technology). For instance, the serum half-life (t_{1/2}) of a drug and/or therapeutic moiety and/or vaccine which is linked to the polypeptide of the present technology may be increased or extended as compared with the serum half-life (t_{1/2}) of the drug and/or therapeutic moiety and/or vaccine as such (i.e., without the polypeptide of the present technology). For instance, the clearance of a drug and/or therapeutic moiety and/or vaccine which is linked to the polypeptide of the present technology may be reduced or decreased as compared with the clearance of the drug and/or therapeutic moiety and/or vaccine as such (i.e., without the polypeptide of the present technology). The present technology thus provides a method for improving at least one PK parameter, such as serum half-life (t_{1/2}) and/or clearance, of a drug and/or therapeutic molecule and/or vaccine, the method comprising:
a. Providing a drug and/or a therapeutic moiety and/or vaccine;
b. Linking (directly or by means of a linker) the drug and/or therapeutic moiety and/or vaccine provided in a. to a polypeptide that comprises (i) at least one domain comprising a serum albumin protein or specifically binding to a serum albumin protein and (ii) an Fc domain of an IgG, as described herein.

Hence, the present technology provides a method for increasing or extending the serum half-life (t_{1/2}) of a drug and/or a therapeutic molecule and/or vaccine, the method comprising:
a. Providing a drug and/or a therapeutic moiety and/or vaccine;
b. Linking (directly or by means of a linker) the drug and/or therapeutic moiety and/or vaccine provided in a. to a polypeptide that comprises (i) at least one domain comprising a serum albumin protein or specifically binding to a serum albumin protein and (ii) an Fc domain of an IgG, as described herein.

Hence, the present technology provides a method for decreasing or reducing the clearance of a drug and/or a therapeutic molecule and/or vaccine, the method comprising:
a. Providing a drug and/or a therapeutic moiety and/or vaccine;
b. Linking (directly or by means of a linker) the drug and/or therapeutic moiety and/or vaccine provided in a. to a polypeptide that comprises (i) at least one domain comprising a serum albumin protein or specifically binding to a serum albumin protein and (ii) an Fc domain of an IgG, as described herein.

The present technology thus provides the polypeptide of the present technology for improving the PK parameters such as serum half-life (t_{1/2}) and/or clearance of a drug and/or a therapeutic molecule and/or vaccine (as compared with the PK parameters of the drug and/or therapeutic molecule and/or vaccine as such, not linked to the polypeptide of the present technology) by linking (directly or by means of a linker) the drug and/or therapeutic moiety and/or vaccine to a polypeptide that comprises (i) at least one domain comprising a serum albumin protein or specifically binding to a serum albumin protein and (ii) an Fc domain of an IgG, as described herein.

Further, the present technology provides the use of the polypeptides of the present technology for improving the PK parameters such as serum half-life (t_{1/2}) and/or clearance of a drug and/or a therapeutic molecule and/or vaccine (as compared with the PK parameters of the drug and/or therapeutic and/or vaccine molecule as such, not linked to the polypeptide of the present technology), by linking (directly or by means of a linker) the drug and/or therapeutic molecule and/or vaccine to a polypeptide that comprises (i) at least one domain comprising a serum albumin protein or specifically binding to a serum albumin protein and (ii) an Fc domain of an IgG, as described herein.

In the context of the present technology, “improving the PK parameters” may refer to increasing or extending the serum half-life (t_{1/2}) and/or to reducing or decreasing the clearance of a molecule, such as a drug or therapeutic moiety.

The figures, sequence listing, and the experimental part/examples are only given to further illustrate the present technology and should not be interpreted or construed as limiting the scope of the present technology and/or of the appended claims in any way, unless explicitly indicated otherwise herein.

The embodiments illustrated and discussed in this specification are intended only to teach those skilled in the art the best way known to the inventors to make and use the present technology. Modifications and variation of the above-described embodiments of the present technology are possible without departing from the present technology, as appreciated by those skilled in the art in light of the above teachings. It is therefore understood that, within the scope of the claims and their equivalents, the present technology may be practiced otherwise than as specifically described.

The present technology will now be further described by means of the following non-limiting preferred aspects, examples and figures.

The entire contents of all of the references (including literature references, issued patents, published patent applications, and co-pending patent applications) cited throughout this application are hereby expressly incorporated by reference, in particular for the teaching that is referenced hereinabove.

Hence, in one embodiment, the polypeptide of the present technology comprises or consists of a polypeptide selected from: TP006, TP009, TP121, TP123, TP111, TPP-66144, TPP-66145, TPP-66146, TPP-66147, TPP-66148, TPP-66149, TPP-66150, TPP-66151, TPP-66152, TPP-66153, TPP-66154, TPP-66174 and TPP-66177. These preferred polypeptides are defined in Table A-1.

### EXAMPLES

### A. Composition of the Polypeptide Constructs of the Present Technology

### 1. Albumin Proteins in the Polypeptides According to Particular Embodiments of the Present Technology

According to particular embodiments, the polypeptides of the present technology comprise at least one domain that comprises a serum albumin protein.

Human serum albumin (HSA) has been well characterized as a polypeptide of 585 amino acids, the sequence of which can be found in Peters, T., Jr. (1996) All about Albumin: Biochemistry, Genetics and Medical, Applications pp 10, Academic Press, Inc., Orlando (ISBN 0-12-552110-3). It has a characteristic binding to its receptor FcRn, where it binds at pH 6.0 but not at pH 7.4.

The plasma half-life of HSA has been found to be approximately 19 days. A natural variant having lower plasma half-life has been identified (Peach, R. J. and Brennan, S. O. (1991) Biochim Biophys Acta. 1097:49-54) having the substitution D494N. This substitution generated an N-glycosylation site in this variant, which is not present in the wild-type albumin. It is not known whether the glycosylation or the amino acid change is responsible for the change in plasma half-life.

Otagiri et al., (2009), Biol. Pharm, Bull. 32(4), 527-534, discloses that 77 albumin variants are known. Of these, 25 are found to be variants in domain III. A natural variant lacking the last 175 amino acids at the carboxy termini has been shown to have reduced half-life (Andersen et al (2010), Clinical Biochemistry 43, 367-372). Iwao et al. (2007) studied the half-life of naturally occurring human albumin variants using a mouse model, and found that K541E and K560E had reduced half-life, E501K and E570K had increased half-life and K573E had almost no effect on half-life (Iwao, et. al. (2007) B.B.A. Proteins and Proteomics 1774, 1582-1590).

Galliano et al. (1993) Biochim. Biophys. Acta 1225, 27-32 discloses a natural variant E505K. Minchiotti et al. (1990) discloses a natural variant K536E. Minchiotti et al (1987) Biochim. Biophys. Acta 916, 411-418 discloses a natural variant K574N. Takahashi et al (1987) Proc. Natl. Acad. Sci. USA 84, 4413-4417, discloses a natural variant D550G. Carlson et al (1992). Proc. Nat. Acad. Sci. USA 89, 8225-8229, discloses a natural variant D550A.

In particular embodiments, the polypeptides of the present technology comprise at least one serum albumin protein, or a fragment or variant thereof, such as for example but not limited to the albumin proteins, fragments and variants disclosed in WO 2011/124718, WO 2011/051489, WO 2013/075066, WO 2013/135896 and WO 2014/072481.

Polypeptides according to particular embodiments of the present technology comprising at least one serum albumin protein and at least one Fc domain are produced and tested for their beneficial PK properties.

### 2. Albumin Binders in the Polypeptides According to Particular Embodiments of the Present Technology

### 2 a Immunoglobulin Single Variable Domains (ISVDs) Specifically Binding to Serum Albumin

According to particular embodiments of the present technology, the at least one domain specifically binding to albumin that is comprised in the polypeptides of the present technology is at least one ISVD, specifically binding to (human) serum albumin.

The international publication WO 2006/122787 (in the name of Applicant) describes a number of ISVDs binding to (human) serum albumin. These ISVDs include the Nanobody® VHH called Alb-1 (SEQ ID NO: 52 in WO 2006/122787) and humanized variants thereof, such as Alb-8 (SEQ ID NO: 62 in WO 2006/122787).

Moreover, WO 2012/175400 (in the name of Applicant) describes a further improved version of Alb-1, called Alb-23.

In particular embodiments, the polypeptides of the present technology comprise at least one serum albumin binding moiety selected from Alb-1, Alb-3, Alb-4, Alb-5, Alb-6, Alb-7, Alb-8, Alb-9, Alb-10 and Alb-23, preferably Alb-8 or Alb-23 or its variants, as shown on pages 7-9 of WO 2012/175400 and the albumin binders described in WO 2012/175741, WO 2015/173325, WO 2017/080850, 30 WO 2017/085172, WO 2018/104444, WO 2018/134235, WO 2018/134234 (all in the name of Applicant).

Some preferred serum albumin binders for the polypeptides of the present technology are shown in Table A-3. Polypeptides comprising at least one of these albumin binding ISVDs were produced and tested for their beneficial PK properties, as described in Examples 2 and 4 below.

### 2 b) DARPin® Sequences Specifically Binding to Serum Albumin

According to particular embodiments of the present technology, the at least one domain specifically binding to a serum albumin protein that is comprised in the polypeptides of the present technology is at least one ankyrin repeat sequence (DARPin sequence) specifically binding to (human) serum albumin.

In particular embodiments, the polypeptides of the present technology comprise at least one serum albumin binding domain which is an ankyrin repeat sequence, such as for example but not limited to the sequences with SEQ ID NO's 17 to 31 and SEQ ID NO's 43 to 52 as disclosed in and specifically described on pages 15-27 of WO 2012/069654, SEQ ID NO: 50 as disclosed in WO 2016/156596, SEQ ID NO:'s 9 to 11 as disclosed in and specifically described on pages 9-11 of WO 2018/054971 and SEQ ID NO's: 3 and 4 as disclosed and specifically described on pages 5-12 of WO 2020/24517.

Polypeptides comprising at least one of these albumin binding ankyrin repeat sequences are produced and tested for their beneficial PK properties.

### 2 c) ABD's (Albumin Binding Domains) of Bacterial Receptor Proteins

According to particular embodiments of the present technology, at least one domain specifically binding to albumin that is comprised in the polypeptides of the present technology is at least one ABD of a bacterial receptor protein that specifically binds to (human) serum albumin.

Streptococcal protein G is a bi-functional receptor present on the surface of certain strains of streptococci and capable of binding to both IgG and serum albumin (Bjorck et al., Mol Immunol 24:11 13, 1987). The structure is highly repetitive with several structurally and functionally different domains (Guss et al., EMBO J 5:1567, 1986), more precisely three Ig-binding motifs and three serum albumin binding domains (Olsson et al., Eur J Biochem 168:319, 1987). The structure of one of the three serum albumin binding domains has been determined, showing a three-helix bundle domain (Kraulis et al., FEBS Lett 378:190, 1996). This motif was named ABD (albumin binding domain) and is 46 amino acid residues in size. In the literature, it has subsequently also been designated G148-GA3. Other bacterial albumin binding proteins than protein G from Streptococcus have also been identified, which contain domains similar to the albumin binding three-helix domains of protein G. Examples of such proteins are the PAB, PPL, MAG and ZAG proteins. Studies of structure and function of such albumin binding proteins have been carried out and reported e.g., by Johansson and co-workers (Johansson et al., J Mol Biol 266:859-865, 1997; Johansson et al., J Biol Chem 277:81 14-8120, 2002), who introduced the designation “GA module” (protein G-related albumin binding module) for the three-helix protein domain responsible for albumin binding. Furthermore, Rozak et al. have reported on the creation of artificial variants of the GA module, which were selected and studied with regard to different species specificity and stability (Rozak et al., Biochemistry 45:3263-3271, 2006; He et al, Protein Science 16:1490-1494, 2007). Recently, variants of the G148-GA3 domain have been developed, with various optimized characteristics. Such variants are for example disclosed in WO publications WO 2009/016043, WO 2012/004384, WO 2014/04897 and WO 2015/091957.

Polypeptides comprising at least one of these ABD's are produced and tested for their beneficial PK properties.

### 2 d) Affitins (Commercialized as Nanoftin®) Specifically Binding to Serum Albumin

According to particular embodiments of the present technology, the at least one domain specifically binding to albumin that is comprised in the polypeptides of the present technology is at least one Affitin (aka Nanofitin®) that specifically binds to (human) serum albumin.

In particular embodiments, the at least one serum albumin binding Affitin is for example but not limited to the sequences with SEQ ID NOs 38 and SEQ ID NO's 45 to 86 as disclosed in and specifically described on pages 6 to 16 of WO 2022/171852.

Polypeptides comprising at least one of these albumin binding affitins are produced and tested for their beneficial PK properties.

### 3. Fc Domains in the Polypeptides According to Particular Embodiments of the Present Technology

The polypeptides according to the present technology further comprise an Fc domain of an IgG. Fc domain of an IgG refers to the C-terminal non-antigen binding region of an immunoglobulin G heavy chain that contains at least a portion of the constant region. In particular embodiments, the Fc domain can be a native Fc region, i.e., as it occurs in natural antibodies, or it can be a variant Fc region, comprising one or more alterations, mutations or variations as compared to the native Fc domain. In particular embodiments, an Fc domain of an IgG can also be a fragment of a native Fc domain or a fragment of a variant Fc domain.

### 3 a) Native (i.e., Wild-Type) Fc Domain of Immunoglobulin G (IgG)

In certain embodiments, a polypeptide as described herein comprises a native Fc domain of a human IgG, such as preferably a native Fc of human IgG1 (e.g., Uniprot sequence PODOX5) or a native Fc of human IgG4 (e.g., Uniprot sequence P01861). Polypeptides comprising at least one such native Fc domain were produced and tested for beneficial PK properties as described in Examples 1, 2 and 4 below.

3 b) Variant Fc Domain with Reduced Effector Function

In certain specific embodiments, the polypeptides according to the present technology comprise variant Fc domains which have altered binding properties for an Fc ligand relative to an unmodified parent Fc molecule. For example, a polypeptide described herein may comprise an Fc region having one or more of amino acid residues 234, 235, 236, 237, 297, 318, 320 and 322 substituted to a different amino acid residue, such that the variant Fc region has an altered affinity for an effector ligand, e.g., an Fc receptor or the C1 component of complement, as described in U.S. Pat. Nos. 5,624,821 and 5,648,260, both to Winter et al.

In particular embodiments, the polypeptides of the present technology comprise an Fc variant domain with reduced effector function, in particular the so-called “FALA” or “LALA” Fc mutant with substitution of residues 234 and 235 to alanine. Extra optional mutations include the substitution of arginine residue 409 to lysine, deletion of lysine residue 447.

Polypeptides comprising at least one Fc domain with the above mutations were produced and tested for beneficial PK properties as described in Examples 1, 2 and 4 below.

3 c) Variant Fc Domains of IgG with Improved Binding Affinity for the FcRn Receptor

In particular embodiments, the polypeptides according to the present technology comprise an Fc variant domain showing improved binding to the FcRn receptor compared to the native Fc domain. Such Fc variants include those with substitutions at one or more of Fc region residues 259, 308, 428, and 434. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 3074 311A, 312A, 376A, 3780, 380A, 382A, 434A (Shields et al., Journal of Biological Chemistry, 2001, 276(9):6591-6604).

In certain particular embodiments, the polypeptides according to the present technology comprise an Fc variant domain wherein methionine 428 was substituted to leucine and asparagine 434 was substituted to serine.

Polypeptides comprising at least one Fc domain with the above mutations are produced and tested for beneficial PK properties.

3 d) Variant Fc Domains of IgG with Reduced or No Binding to the FcRn Receptor

In particular embodiments, the polypeptides according to the present technology comprise an Fc variant domain showing reduced or no binding to the FcRn receptor compared to the native Fc domain. Such Fc variants include those with substitutions at one or more of Fc region residues 253, 310 and 453.

In particular embodiments, the polypeptides according to the present technology comprise an Fc variant domain wherein isoleucine 428 was substituted to alanine, histidine 310 was substituted to alanine, and histidine 453 was substituted to alanine, optionally in combination with histidine 453 substituted to alanine.

Polypeptides comprising at least one Fc domain are produced and tested for beneficial PK properties.

The constructs used in the examples are described in Table A-1.

### B. Production, Binding and PK Properties of the Polypeptide Constructs of the Present Technology

### Example 1

### Generation and Expression of Fusion Protein Constructs Comprising an Albumin Binding ISVD and an Fc Domain

Asymmetrical fusion proteins of an albumin binding Nanobody® VHH (ISVD) linked to an Fc domain of an IgG were generated using the Knob-in-Hole technology as commonly known in the art (and as described for instance in patent publication WO 1996/27011 by Genentech as well as scientific publications by Ridgway, J B et al. “‘Knobs-into-holes’ engineering of antibody CH3 domains for heavy chain heterodimerization”, Protein engineering 9,7 (1996): 617-21 and Merchant et al., “An efficient route to human bispecific IgG”, Nature Biotechnology 16, (1998): 677-681).

DNA fragments encoding an albumin binding Nanobody® VHH (ISVD) and/or control Nanobody® VHH (ISVD) and an Fc domain of an IgG, obtained by PCR with specific combinations of forward and reverse primers each carrying a specific Bpil restriction site, were cloned in the appropriate expression vector via Golden Gate cloning (Engler C, Marillonnet S. Golden Gate cloning. Methods Mol Biol. 2014; 1116:119-31). After Sanger sequence confirmation, the plasmid DNA was then transfected into CHOEBNALT85 cells (QMCF Technology) for protein production. The Nanobody® VHH-Fc fusion proteins were purified from the cell supernatants using a protein A capture step followed by an ion exchange and/or size exclusion chromatography purification step.

### Example 2

### Binding Studies of Albumin Binding Nanobody® VHH-Fc Fusion Polypeptide Constructs

A set of Nanobody® VHH-Fc fusion proteins was generated that typically consisted of an Fc domain linked to (i) a Nanobody® VHH specifically binding to serum albumin and (ii) a Nanobody® VHH (ISVD) not binding to serum albumin or any other envisaged target but solely included in the polypeptide construct so as to create a similar size (i.e., molecular weight) as the corresponding test construct (also referred to as “control” or “irrelevant” ISVD, see Tables A-1 and A-8). The Fc domains in the constructs were IgG4 FALA Fc backbone sequence variants with knob in hole mutations as described herein whereas the albumin binding Nanobody® VHH (ISVD) used, was in each case the Alb23002 sequence as described herein. The Nanobody® VHH sequences in these fusion proteins were fused via a linker (as described in detail herein) to the N- and/or C-terminus of the Fc chain, i.e., via an IgG1 hinge and/or a GS linker, respectively (see FIG. 1). As is clear from FIG. 1, some of these constructs comprise additional amino acid differences or variations in the Fc backbone sequence (i.e., I253A, H310A, H435A). These Fc sequence variants were made to test constructs that showed no binding to FcRn and will be referred to further herein as non-binding Fc-variants. As a control, Nanobody® VHH-Fc fusion proteins were generated, comprising the same composition of the test constructs, except that the Nanobody® VHH binding to serum albumin was replaced by a Nanobody® VHH not binding to serum albumin or any other envisaged target (i.e., variants of the IgG4 FALA Fc backbone sequence linked to two Nanobody® VHH's not binding to serum albumin, see e.g., FIG. 1, construct TP003). As second control, a monoclonal antibody (TP013) was generated containing the same IgG4 FALA Fc backbone containing knob in hole mutations.

### i) Binding to Human Serum Albumin (HSA) and Mouse Serum Albumin (MSA)

The affinities of the purified Nanobody® VHH-Fc fusion proteins for human and mouse serum albumin (HSA and MSA, respectively) at pH 6.0 and pH 7.4 were determined on a Biacore 8K+ instrument. HSA or MSA (HSA: Sigma-Aldrich—Sigma, Cat No. A8763; MSA: Albumin Bioscience, Cat No. 2601) was immobilized on a Series S Sensor Chip CM5. The Nanobody®VHH-Fc fusion proteins were injected at 9 different concentrations (between 0.6 and 2000 nM) and allowed to associate for 120 s at 30 μL/min and dissociate for 600 s. Evaluation of the sensorgrams was based on the 1:1 Langmuir dissociation model simultaneously fitting on and off-rates. The affinities are shown in Table 1.

A 5 to 10-fold higher affinity for HSA was observed when ALB23002 was present at the N-terminus (for TP009 and TP016) compared to the C-terminus (TP006 and TP019). No significant (>3-fold) difference in affinity was observed at pH 6.0 and pH 7.4.

TABLE 1
HSA and MSA affinities of Nanobody ®VHH-Fc fusion constructs

| | | pH 6.0 | | | pH 7.4 | | |
|---|---|---|---|---|---|---|---|
| Target | Construct | ka (1/Ms) | kd (1/s) | KD (M) | ka (1/Ms) | kd (1/s) | KD (M) |
| HSA | TP006 | 5.4E+04 | 1.7E−03 | 3.2E−08 | 2.6E+04 | 1.7E−03 | 6.7E−08 |
| | TP009 | 1.4E+05 | 3.7E−04 | 2.7E−09 | 7.5E+04 | 5.0E−04 | 6.7E−09 |
| | TP016 | 1.1E+05 | 4.0E−04 | 3.7E−09 | 6.1E+04 | 5.7E−04 | 9.3E−09 |
| | TP019 | 6.1E+04 | 1.6E−03 | 2.6E−08 | 3.6E+04 | 1.9E−03 | 5.1E−08 |
| MSA | TP006 | 3.1E+04 | 2.6E−03 | 8.4E−08 | 2.3E+04 | 2.8E−03 | 1.2E−07 |
| | TP009 | 1.6E+05 | 2.0E−03 | 1.3E−08 | 1.3E+05 | 2.6E−03 | 2.1E−08 |
| | TP016 | 1.7E+05 | 3.8E−03 | 2.2E−08 | 1.2E+05 | 4.2E−03 | 3.5E−08 |
| | TP019 | 1.0E+07 | 5.0E−01 | 5.0E−08 | 1.0E+07 | 5.0E−01 | 5.0E−08 |

Italics and underlined: indicative values

### ii) Binding to Human FcRn

The Nanobody®VHH-Fc proteins were characterized by affinity determination for human FcRn at pH 6.0 on the Biacore 8K+ instrument. For affinity measurements, ˜1000-2000 RU of biotinylated human FcRn was captured on a Series S Sensor Chip SA. The Nanobody®VHH-Fc fusion proteins were injected at 9 different concentrations (between 0.5 and 1500 nM) in the absence or presence of 30 μM HSA or MSA and allowed to associate for 120 s at 30 μL/min and dissociate for 600 s. Evaluation of the sensorgrams was based on the 1:1 Langmuir dissociation model simultaneously fitting on and off-rates. The affinity for human FcRn at pH 6.0 in the absence of HSA is shown in Table 2.

Almost no FcRn binding was detected for the mutated Fc (TP016 and TP019). All Nanobody®VHH-Fc constructs with an Fc domain not mutated at positions 253, 310 and 435 (i.e., having I253, H310, H435) showed specific binding to FcRn at pH 6.0. For the Fc-fusion constructs that contained the albumin binding Nanobody®VHH (ISVD) ALB23002 (constructs TP006 and TP009), the off-rates were slower in the presence of MSA or HSA, suggesting an avidity effect through simultaneous direct and indirect FcRn binding.

TABLE 2
FcRn affinities of Nanobody ®VHH-Fc fusion constructs

| | Human FcRn | | |
|---|---|---|---|
| Construct | ka (1/Ms) | kd (1/s) | KD (M) |
| TP003 | 4.6E+05 | 1.7E−03 | 3.6E−09 |
| TP006 | 4.5E+06 | 8.3E−02 | 1.8E−08 |
| TP008 | 2.0E+06 | 8.6E−03 | 4.3E−09 |
| TP009 | 2.3E+06 | 6.7E−03 | 3.0E−09 |
| TP013 | 9.3E+05 | 2.9E−02 | 3.1E−08 |
| TP016 | 3.9E+04 | 3.9E−02 | 1.0E−06 |
| TP019 | 3.8E+04 | 6.9E−02 | 1.8E−06 |

Italics and underlined: indicative values

For the constructs demonstrating specific binding to FcRn, the data was reanalyzed, and a bivalent analyte fit was used to fit the data. The affinity for human FcRn at pH 6.0 in the absence and presence of HSA is shown in Table 3. For the Fc-fusion constructs that contained the albumin binding domain (Nanobody®VHH ALB23002 (ISVD), constructs TP006 and TP009), the off-rates were slower in the presence of HSA, suggesting an avidity effect through simultaneous direct and indirect FcRn binding.

TABLE 3
FcRn affinities of Nanobody ®VHH-Fc fusion constructs (bivalent analyte fit)

| | | Human FcRn | | | | | | |
|---|---|---|---|---|---|---|---|---|
| | | ka1 | kd1 | KD1 | ka2 | kd2 | KD2 | |
| | Construct | (1/Ms) | (1/s) | (M) | (1/RUs) | (1/s) | (M) | Remark |
| no | TP003 | 2.5E+05 | 9.8E−03 | 3.9E−08 | 1.5E−01 | 2.4E+00 | 1.6E+01 | |
| HSA | TP006 | 1.4E+05 | 4.2E−02 | 2.9E−07 | 1.3E−04 | 2.0E−03 | 1.5E+01 | |
| | TP008 | 2.7E+05 | 2.0E−02 | 7.5E−08 | 6.2E−05 | 2.1E−03 | 3.3E+01 | |
| | TP009 | 2.4E+05 | 3.7E−02 | 1.5E−07 | 1.3E−03 | 1.8E−02 | 1.4E+01 | |
| | TP013 | 2.1E+05 | 4.0E−02 | 1.9E−07 | 7.1E−04 | 7.0E−02 | 9.8E+01 | |
| with | TP003 | 4.7E+05 | 3.6E−02 | 7.7E−08 | 1.0E+02 | 1.9E+03 | 1.9E+01 | |
| HSA | TP006 | | 1.6E−03 | | | 5.9E−03 | | no good fit |
| | | | | | | | | possible |
| | TP008 | 2.6E+05 | 4.7E−02 | 1.9E−07 | 7.1E+00 | 1.2E+02 | 1.7E+01 | |
| | TP009 | 7.5E+04 | 1.7E−03 | 2.3E−08 | 7.4E−03 | 9.5E−02 | 1.3E+01 | |
| | TP013 | | | | | | | no good fit |
| | | | | | | | | possible |

For the constructs demonstrating specific binding to FcRn, SPR analysis was repeated under different conditions. The FcRn affinities at pH 6.0 of the Nanobody®VHH-Fc proteins were repeated with a slightly altered experimental set-up (lower FcRn coating density) and altered fit. For this, ˜600 RU of biotinylated human FcRn was captured on a Series S Sensor Chip SA. The Nanobody®VHH-Fc fusion proteins were injected at 9 different concentrations (between 1 and 7500 nM) and allowed to associate for 120s at 30 μL/min and dissociate for 600s. Evaluation of the sensorgrams was based on the Bivalent Analyte fit. The affinity for human FcRn at pH 6.0 is shown in Table 4. TP016 and TP019 were not included, as they hardly showed any FcRn binding in the previous experiment.

TABLE 4
FcRn affinities of Nanobody ®VHH-Fc fusion constructs (bivalent analyte fit)

| Construct | ka1 (1/Ms) | kd1 (1/s) | KD1 (M) | ka2 (1/RUs) | kd2 (1/s) | KD2 (M) |
|---|---|---|---|---|---|---|
| TP003 | 7.70E+04 | 2.38E−04 | 3.09E−09 | 1.10E−02 | 3.29E−02 | 2.99E+00 |
| TP006 | 9.44E+03 | 4.02E−03 | 4.26E−07 | 1.07E+01 | 7.19E+01 | 6.73E+00 |
| TP008 | 3.25E+06 | 1.98E−02 | 6.10E−09 | 2.87E+01 | 5.11E+01 | 1.78E+00 |
| TP009 | 2.28E+04 | 3.44E−04 | 1.51E−08 | 9.17E−01 | 1.02E+01 | 1.11E+01 |
| TP013 | 1.68E+05 | 1.22E−01 | 7.27E−07 | 1.54E−03 | 6.22E−02 | 4.03E+01 |

Italics and underlined: indicative values

### Example 3

### Development and Optimization of Serum PK Assays

### i) Serum PK Assay for Test Constructs TP003, TP006, TP009, TP016

Pharmacokinetic experiments were initiated in TG32 (B6.Cg-FcgrttmlDcr Tg(FCGRT) 32Dcr/Dcr) mice to evaluate half-life of albumin-binding ISVD genetically fused to IgG-Fc domain sequences. A specific and sensitive ligand binding assay was developed to measure concentrations of all constructs in mouse serum.

A streptavidin-coated MSD GOLD 96-well SMALLSPOT® plate (Meso Scale Discovery) was blocked with Superblock T20™ (Thermo Scientific) for 30 minutes at RT. The plate was then washed and incubated for 1 hour at RT and at 600 rpm with 2.0 μg/mL biotinylated generic mAb directed against the frameworks of the ISVD moiety used in each construct. Calibrators and QCs were prepared in pooled mouse serum. After washing the plate, calibrators, QCs and samples were applied to the plate at an MRD of 20 to 100 (depending on the construct) in PBS 0.1% casein and incubated for 1 hour at RT and at 600 rpm. After washing, the plate was incubated for 1 hour at RT and at 600 rpm with 2.0 μg/mL sulfo-labelled mAb directed against a specific ISVD moiety, depending on the format under evaluation. After the plate was washed, 2×MSD Read buffer (Meso Scale Discovery) was added and the plate was read on a Sector Imager Quickplex SQ 120 (Meso scale Discovery).

For in vivo experiments in which a mixture of human IgGs (hIVIG; Privigen®) was used to mimic endogenous IgG competition, hIgG assay interference was evaluated.

### ii) Serum PK Assay for Control TP013

A Nunc-Immuno™MaxiSorp™ flat bottom 96-well solid plate (Sigma-Aldrich) was coated overnight at 4° C. with 1 μg/mL of an anti-idiotype Fab. The plate was washed and blocked with Superblock T20™ (Thermo Scientific) for 1 hour at RT. Calibrators and QCs were prepared in pooled mouse serum. After washing the plate, calibrators, QCs and samples were applied at an MRD of 10 in PBS 0.1% casein and incubated for 1 hour at RT and at 600 rpm. Next, the plate was washed and incubated for 1 hour at RT and at 600 rpm with 1.0 μg/mL of an HRP-conjugated anti-idiotype mAb. After washing, the plate was incubated with TMB for 20 minutes at RT after which the reaction was stopped by the addition of 1M HCl and a colorimetric read-out was performed on a Tecan Sunrise Microplate reader. For in vivo experiments in which a mixture of human IgGs (hIVIG; Privigen®) was used to mimic endogenous IgG competition, hIgG assay interference was evaluated.

### Example 4

### Pharmacokinetics of Polypeptide Constructs in Transgenic Mice

### i) Pharmacokinetics of IgG4 FALA Fc-ISVD Polypeptide Constructs in Transgenic Mice (TP003, TP009, TP013, TP016)

Six Tg32 mice (B6.Cg-FcgrttmlDcr Tg(FCGRT) 32Dcr/DcrJ) mice were injected intravenously in the tail with either 5 mg/kg ISVD-Fc constructs (TP003, TP009, TP016) or 8 mg/kg monoclonal antibody (TP013). ISVD-Fc constructs consisted of identical IgG Fc, except for TP016, which had mutations I253A, H310A, H435A (IHH) to abrogate FcRn binding. Fc constructs were genetically fused to 2 ISVD domains, either 2 non-targeting ISVD N-terminally (CNB: negative control; TP003) or 1 non-targeting ISVD and 1 albumin-targeting VHH (ALB23002) (TP009 and TP016). TP009 and TP016 were evaluated in one study, while TP003 and TP013 were evaluated in another study, under identical conditions including polypeptide constructs to bridge between both studies. All animal studies were conducted according to Sanofi's standards regarding animal welfare.

Blood was retrieved at different time points (composite sampling, 2 mice per time point) and serum was prepared. Serum samples were analyzed by ELISA for the presence of ISVD-Fc constructs or monoclonal antibody construct as described in Example 3. Half-lives values were obtained by estimating the in vivo endosomal FcRn affinities in a mechanistic model and reported in Table 5. PK parameters were also obtained from the same dataset, from non-compartmental analysis in Phoenix WinNonlin® (version 8.2.2.227. Certara) using the Plasma Data Module. When applicable, sampling times with steep concentration decline of compound due to suspected ADA impact were excluded from analysis. These PK parameters are reported in Table 6. We can conclude from the results that the clearance (Cl) and half-life (t_{1/2}) of an ISVD-Fc construct comprising a ISVD specifically binding to albumin linked to an Fc domain binding to FcRn is significantly improved compared to constructs of similar size (molecular weight) but comprising either only an Fc domain binding to FcRn (but no serum albumin protein binding domain) such as a full IgG construct, or only an albumin-binding ISVD (linked to a non-FcRn binding Fc domain).

TABLE 5
Calculated half-lives (hr) obtained by mechanistic
modeling of ISVD-Fc constructs and mAb

| | TP003 | TP009 | TP013 | TP016 |
|---|---|---|---|---|
| t_{1/2} (hours) | 165 | 412 | 201 | 46 |

TABLE 6
PK parameters of ISVD-Fc constructs
and mAb obtained by NCA analysis

| Test | | Dose | Cl | t_{1/2} |
|---|---|---|---|---|
| compound | HLE domain | (mg/kg) | (mL/hr/kg) | (hours) |
| TP003 | IgG4 FALA Fc | 5 | 0.796 | 144 |
| TP009 | ALB23002, IgG4 FALA Fc | 5 | 0.196 | 390 |
| TP013 | IgG4 FALA Fc | 8 | 0.365 | 207 |
| TP016 | ALB23002 | 5 | 2.34 | 43.9 |

ii) Pharmacokinetics of IgG4 FALA Fc-ISVD Polypeptide Constructs in Transgenic Mice with IgG Competition (TP003, TP006, TP009, TP013)

To mimic relevant competition with hIgG, Tg32 mice (B6.Cg-FcgrttmlDcr Tg(FCGRT) 32Dcr/DcrJ) were preloaded with a mixture of purified hIgG (hIVIG; Privigen®). Privigen® was administered intravenously once weekly, with the first administration 2 days prior to initiation of the PK study. In total, 4 Privigen® injections of 250 mg/kg were administered, yielding physiologically relevant hIgG serum concentrations for the duration of the study (data not shown). All groups received Privigen® treatment, and for some compounds a group was included where Privigen® was not administered. These groups allowed evaluation of the impact of hIgG on PK, which could be expected for compounds that bind to the Fc epitope on FcRn (data not shown).

Two days after the first Privigen® administration, 6 Tg32 mice (B6.Cg-FcgrttmlDcr Tg(FCGRT) 32Dcr/DcrJ) were injected intravenously in the tail with 5 to 8 mg/kg ISVD-Fc constructs (TP003, TP006 or TP009) or monoclonal antibody (TP013), respectively. ISVD-Fc constructs consisted of identical IgG Fc, genetically fused to 2 VHH domains, either 2 non-targeting (“irrelevant”) ISVDs N-terminally (negative control; TP003), or one non-targeting (“Irrelevant”)N-terminally ISVDs and one albumin-targeting ISVD (ALB23002, either C- or N-terminally; TP006 and TP009, respectively) (see FIG. 1).

Blood was retrieved at different time points (composite sampling, 2 mice per time point) and serum was prepared. Serum samples were analyzed by ELISA for the presence of ISVD-Fc constructs or monoclonal antibody construct as described in Example 3. Results are shown in FIG. 2.

PK parameters were obtained from non-compartmental analysis in Phoenix WinNonlin® (version 8.2.2.227, Certara) using the Plasma Data Module. When applicable, sampling times with steep concentration decline of compound due to suspected ADA impact were excluded from analysis. PK parameters are reported in Table 7.

We can conclude from the results that the clearance and half-life of ISVD-Fc constructs comprising an albumin-binding ISVD is significantly improved compared to constructs comprising non-targeting ISVD or compared to a monoclonal antibody. Clearance values of 0.163-0.186 mL/hr/kg and half-lives (t_{1/2}) of 291-310 hours (hrs or h or hr) were observed for constructs containing ALB23002 (TP006 and TP009, respectively), compared to clearance of 0.520-0.741 mL/hr/kg and half-life of 110-134 hrs for the controls (TP013 and TP003, respectively). In conclusion, Alb23002 in N- or C-terminal fusion to Fc reduces clearance by approximately 3.5-4-fold and prolongs half-life (2-fold) in Tg32 mice compared to control ISVD-Fc fusion.

Impact of IgG competition on the clearance of the test items was considered limited, as evidenced by the comparison test items between both studies as well as the analysis of the same test article with and without Privigen® addition in the second study (data not shown).

TABLE 7
PK parameters of ISVD-Fc constructs
and mAb obtained by NCA analysis

| Test | | Dose | Cl | t_{1/2} |
|---|---|---|---|---|
| compound | HLE domain | (mg/kg) | (mL/hr/kg) | (hr) |
| TP003 | IgG4 FALA Fc | 5 | 0.741 | 134 |
| TP006 | IgG4 FALA Fc, ALB23002 | 5 | 0.163 | 310 |
| TP009 | ALB23002, IgG4 FALA Fc | 5 | 0.186 | 291 |
| TP013 | IgG4 FALA Fc | 8 | 0.520 | 110 |

### Example 5

### Generation and Expression of Fusion Protein Constructs Comprising an Albumin Binding ISVD and an Fc Domain or Full-Length Antibody

Fusion proteins of an albumin binding Nanobody®VHH (ISVD) linked to (i) an Fc domain of an IgG or (ii) a full-length IgG were generated using the Knob-in-Hole technology as commonly known in the art (and as described for instance in patent publication WO 1996/27011 by Genentech as well as scientific publications by Ridgway, J B et al. “‘Knobs-into-holes’ engineering of antibody CH3 domains for heavy chain heterodimerization”, Protein engineering 9,7 (1996): 617-21 and Merchant et al. “An efficient route to human bispecific IgG”, Nature Biotechnology 16, (1998): 677-681).

DNA fragments of (i) a FcRn binding Nanobody® VHH (ISVD) and/or control Nanobody® VHH (ISVD) and (ii) an Fc domain of an IgG or the full-length heavy chain of the IgG, obtained by PCR with specific combinations of forward and reverse primers each carrying a specific Bpil restriction site, were combined and cloned in the appropriate expression vector via Golden Gate cloning (Engler C, Marillonnet S. Golden Gate cloning. Methods Mol Biol. 2014; 1116:119-31). For the generation of the Nanobody®-IgG fusions (ISVD-IgG fusions), the light chain of the IgG was cloned in a separate expression vector. After Sanger sequence confirmation, the plasmid DNA was then transfected into CHOEBNALT85 cells (QMCF Technology) for protein production. The Nanobody®VHH-Fc-IgG fusion proteins were purified from the cell supernatants using a protein A capture step followed by an ion exchange and/or size exclusion chromatography purification step.

### Example 6

### Binding Studies of Albumin Binding Nanobody®VHH (ISVD)—IgG1 Fc/IgG1 Fusion Polypeptide Constructs

A set of Nanobody®VHH-IgG1 Fc/IgG1 fusion proteins was generated that typically consisted of (i) an Fc domain or (ii) a full length IgG1 linked to (i) one or two Nanobody® VHHs (ISVDs) specifically binding to serum albumin and/or (ii) to one or two Nanobody® VHHs (ISVDs) not binding to serum albumin or any other envisaged target but solely included in the polypeptide construct so as to create a similar size (i.e., molecular weight) as the corresponding test construct. The Fc domains in the constructs were IgG1 Fc backbone sequence variants with knob in hole mutations as described herein and the hole chain also contained two additional mutations (i.e., H435R, Y436F) to ease the purification of the final protein. The IgG1 Fc backbone was the native Fc. The albumin binding Nanobody®VHH (albumin binding ISVD) used was in each case the Alb23002 sequence as described herein (SEQ ID NO.: 20). The Nanobody® VHH (ISVD) sequences in these fusion proteins were fused via a linker (as described in detail herein) to the C-terminus of the Fc chain, i.e., via a 9GS linker. As controls, Nanobody®VHH-IgG1 Fc/IgG1 fusion proteins were generated, comprising the same composition of the test constructs, except that the Nanobody® VHH (ISVD) binding to serum albumin was replaced by a Nanobody® VHH (ISVD) not binding to serum albumin or any other envisaged target (e.g., constructs TP111 and TP121).

### i) Binding to Human Serum Albumin (HSA) and Mouse Serum Albumin (MSA)

The affinities of the purified Nanobody®VHH-IgG1 Fc/IgG1 fusion proteins for human and mouse serum albumin (HSA and MSA, respectively) at pH 7.4 were determined on a Biacore 8K+ instrument. HSA or MSA (HSA: Sigma-Aldrich—Sigma, Cat No. A8763; MSA: Albumin Bioscience, Cat No. 2601) was immobilized on a Series S Sensor Chip C1. The Nanobody®VHH-Fc fusion proteins were injected at 9 different concentrations (between 1.6 and 2500 nM) and allowed to associate for 120s at 30 μL/min and dissociate for 600 s. Evaluation of the sensorgrams was based on the 1:1 Langmuir dissociation model simultaneously fitting on and off-rates. For MSA, only the off-rates are shown because the on-rates could not be fitted properly. The data is shown in Table 8. All constructs display a similar HSA affinity and MSA off-rate. For the IgG fusion with 2 albumin binding Nanobody®VHHs (i.e., TP123), the data was fitted with the bivalent analyte fit and the data is shown in Table 9.

TABLE 8
HSA affinities and MSA off-rates of Nanobody ®VHH-
IgG1 Fc/IgG1 fusion constructs

| | HSA | | | MSA |
|---|---|---|---|---|
| Construct | ka (1/Ms) | kd (1/s) | KD (M) | kd (1/s) |
| TP111 | 5.12E+04 | 1.88E−03 | 3.66E−08 | 4.20E−02 |
| TP121 | 2.67E+04 | 2.04E−03 | 7.64E−08 | 3.85E−02 |

TABLE 9
HSA and MSA affinity of IgG fusion with two albumin binding Nanobody ®VHHs (ISVDs)

| | | ka1 | | | ka2 | | |
|---|---|---|---|---|---|---|---|
| Target | Construct | (1/Ms) | kd1 (1/s) | KD1 (M) | (1/RUs) | kd2 (1/s) | KD2 (M) |
| HSA | TP123 | 3.75E+04 | 2.53E−03 | 6.74E−08 | 1.47E−04 | 3.30E−03 | 2.24E+01 |
| MSA | TP123 | 6.14E+04 | 3.44E−02 | 5.60E−07 | 6.57E−05 | 4.69E−04 | 7.13E+00 |

ii) Binding to human FcRn

The Nanobody®VHH-IgG1 Fc/IgG1 proteins were characterized by affinity determination for human FcRn at pH 6.0 on the Biacore 8K+ instrument. For affinity measurements, ˜700 RU of biotinylated human FcRn was captured on a Series S Sensor Chip SA. The Nanobody®VHH-Fc fusion proteins were injected at 9 different concentrations (between 1 and 7500 nM) and allowed to associate for 120 s at 30 μL/min and dissociate for 600 s. Evaluation of the sensorgrams was based on the Bivalent Analyte fit. The affinity for human FcRn at pH 6.0 is shown in Table 10. The presence of an ALB23002 building block at the C-terminus has no impact on the FcRn affinity (e.g., TP111 vs TP108).

TABLE 10
FcRn affinities of Nanobody ®VHH-IgG1 Fc/IgG1 fusion constructs

| | ka1 | kd1 | KD1 | ka2 | kd2 | |
|---|---|---|---|---|---|---|
| Construct | (1/Ms) | (1/s) | (M) | (1/RUs) | (1/s) | KD2 (M) |
| TP108 | 2.7E+05 | 1.4E−01 | 5.1E−07 | 7.6E−05 | 5.3E−04 | 7.0E+00 |
| TP111 | 2.3E+05 | 1.5E−01 | 6.5E−07 | 6.4E−05 | 5.5E−04 | 8.5E+00 |
| TP117 | 1.2E+05 | 1.6E−01 | 1.3E−06 | 3.1E−05 | 1.0E−03 | 3.3E+01 |
| TP118 | 1.2E+05 | 1.5E−01 | 1.2E−06 | 2.3E−05 | 5.4E−04 | 2.3E+01 |
| TP121 | 7.1E+04 | 1.6E−01 | 2.3E−06 | 1.8E−05 | 3.6E−04 | 2.0E+01 |
| TP123 | 6.3E+04 | 1.5E−01 | 2.4E−06 | 1.7E−05 | 5.9E−04 | 3.4E+01 |

### Example 7

### Generation and Expression of Fusion Protein Constructs Comprising an Albumin Protein or Albumin Binding Protein and an Fc Domain

Asymmetrical fusion proteins of an albumin protein, albumin binder or albumin binding Nanobody®VHH (ISVD) linked to an Fc domain of an IgG were generated using the Knob-in-Hole technology as commonly known in the art (and as described for instance in patent publication WO 1996/27011 by Genentech as well as scientific publications by Ridgway, J B et al. “‘Knobs-into-holes’ engineering of antibody CH3 domains for heavy chain heterodimerization”, Protein engineering 9,7 (1996):617-21 and Merchant et al. “An efficient route to human bispecific IgG”, Nature Biotechnology 16, (1998): 677-681).

The protein productions were externalized to a CRO, with production in CHO cells, followed by purification from the cell supernatants using a protein A capture step followed by a size exclusion chromatography purification step.

### Example 8

### Binding Studies of Albumin-Fc or Albumin Binder-Fc Fusion Polypeptide Constructs

A set of Fc fusion proteins was generated that typically consisted of an Fc domain linked to (i) an albumin protein or (ii) an albumin binder (i.e., DARPin®, ABD or Affitin) or (iii) a Nanobody® VHH (ISVD) that specifically binds to serum albumin and (iv) to two or three Nanobody® VHHs (ISVDs) not binding to serum albumin or any other envisaged target but solely included in the polypeptide construct so as to create a similar size (i.e., molecular weight) as the corresponding test construct. The asymmetrical Fc domains in the constructs were IgG4 FALA Fc backbone sequence variants with knob in hole mutations as described herein and the hole chain also contained two additional mutations (i.e., H435R, Y436F) to ease the purification of the final protein. The symmetrical Fc fusions were generated without the knob in hole mutations. The Fc backbone was an IgG4 FALA Fc or an IgG4 FALA Fc with improved binding affinity for the FcRn receptor (i.e., YTE). The human albumin protein was the wild-type HSA protein (containing amino acids 25 until 609 from uniprot ID P02768 (i.e., HSA(25-609)), or a mutated version with increased (i.e., HSA(QMP)=HSA(25-609)(E529Q, T551M, K597P)) FcRn binding, see SEQ ID NO.: 23 and 110, respectively). The albumin binders were the DARPin®, ABD or Affitin (SEQ ID NO.: 102, 103 and 104, respectively) or an albumin binding Nanobody®VHH (ISVD) (ALB23002 (SEQ ID NO.: 20), HSA006A06 (SEQ ID NO.: 65), ALB11002 (SEQ ID NO.: 13), ALBX00002 (SEQ ID NO.: 64), T0235002C06 (L11V, T14P, D74S, K83R, V89L) (T023500029-A, SEQ ID NO.: 69)). The albumin protein or the albumin binders in these fusion proteins were fused (as described in detail herein) to the C-terminus of the Fc chain via a GS linker, generally 35GS, but 9GS was also used once, TPP-66144 (see, e.g., FIG. 6 and Table A-1). As a control, Nanobody®VHH-Fc fusion proteins were generated, comprising the same composition of the test constructs, except that the Nanobody® VHH (ISVD) binding to serum albumin was replaced by a Nanobody® VHH (ISVD) not binding to serum albumin or any other envisaged target (“irrelevant ISVD”) (i.e., variants of the IgG4 FALA Fc backbone sequence linked to three Nanobody®VHHs (ISVDs) not binding to serum albumin (“irrelevant”), see e.g., FIG. 6 and Table A-1, e.g., construct TPP-66143 or TPP-66176, which are variants of the IgG4 FALA Fc backbone sequence linked to three or four Nanobody®VHHs (ISVD)).

### i) Binding to Human Serum Albumin (HSA) and Mouse Serum Albumin (MSA)

The binding of the purified Fc fusion proteins for human and mouse serum albumin (HSA, as described above and MSA, as described below) at pH 7.4 was determined on a Biacore 8K+ instrument. HSA or MSA (HSA: Sigma-Aldrich—Sigma, Cat No. A8763; MSA: Albumin Bioscience, Cat No. 2601) was immobilized on a Series S Sensor Chip C1. The Fc fusion proteins were injected at 9 different concentrations (between 1.6 and 2500 nM) and allowed to associate for 120 s at 30 μL/min and dissociate for 600s. Evaluation of the sensorgrams was based on the 1:1 Langmuir dissociation model simultaneously fitting on and off-rates. For MSA, only the off-rates are shown because the on-rates could not be fitted properly. The data is shown in Table 11. For the symmetrical Fc fusions with 2 albumin binding Nanobody®VHHs (ISVDs), the data was fitted with the bivalent analyte fit and the data is shown in Table 12.

TABLE 11
HSA affinities and MSA off-rates of asymmetrical Fc fusion constructs

| | Albumin binding | HSA | | | MSA |
|---|---|---|---|---|---|
| Construct | protein | ka (1/Ms) | kd (1/s) | KD (M) | kd (1/s) |
| TPP-66144 | ALB23002 | 2.40E+04 | 1.24E−03 | 5.16E−08 | 8.42E−02 |
| TPP-66145 | HSA006A06 | 4.41E+04 | 1.62E−03 | 3.67E−08 | 5.60E−02 |
| TPP-66146 | ALB11002 | 5.72E+04 | 4.86E−03 | 8.49E−08 | similar to |
| | | | | | TPP-66145 |
| TPP-66147 | ALB23002 | 4.38E+04 | 1.19E−03 | 2.71E−08 | 7.50E−02 |
| TPP-66148 | ALBX00002 | 4.85E+04 | 2.47E−04 | 5.10E−09 | 1.87E−03 |
| TPP-66149 | T023500029 | 6.23E+04 | 5.11E−04 | 8.20E−09 | 5.03E−04 |
| TPP-66150 | DARPin ® | | significantly | | significantly |
| | | | faster off- | | faster off- |
| | | | rate | | rate |
| TPP-66151 | Affitin | 1.19E+05 | 3.10E−04 | 2.61E−09 | 1.75E−03 |
| TPP-66152 | ABD | 8.95E+04 | 2.01E−04 | 2.24E−09 | 1.11E−03 |
| TPP-66174 | ALB23002 | 4.84E+04 | 1.04E−03 | 2.15E−08 | 8.19E−02 |

Italics and underlined: indicative values

TABLE 12
HSA and MSA affinities of symmetrical Fc fusion constructs

| | | ka1 | | | ka2 | | |
|---|---|---|---|---|---|---|---|
| Target | Construct | (1/Ms) | kd1 (1/s) | KD1 (M) | (1/RUs) | kd2 (1/s) | KD2 (M) |
| HSA | TPP-66177 | 5.43E+04 | 7.76E−04 | 1.43E−08 | 4.94E−01 | 5.69E+00 | 1.15E+01 |
| MSA | TPP-66177 | 4.59E+04 | 4.04E−04 | 8.80E−09 | 3.18E+00 | 2.70E+01 | 8.48E+00 |

Italics and underlined: indicative values

### ii) Binding to Human FcRn

The Fc proteins were characterized by affinity determination for human FcRn at pH 6.0 on the Biacore 8K+ instrument. For affinity measurements, ˜500-600 RU of biotinylated human FcRn was captured on a Series S Sensor Chip SA. The Fc fusion proteins were injected at 9 different concentrations (between 1 and 7500 nM) and allowed to associate for 120 s at 30 μL/min and dissociate for 600 s. Evaluation of the sensorgrams was based on the Bivalent Analyte fit. The affinity for human FcRn at pH 6.0 is shown in Table 13. The constructs with the engineered IgG4 FALA Fc (YTE) variant showed an increased FcRn binding compared to the parental IgG4 FALA Fc (e.g., TPP-66175 versus TPP-66143). FcRn binding was also increased for the HSA fusion constructs, e.g. TPP-66153 and TPP-66154 versus TPP-66143.

TABLE 13
FcRn affinities of Fc fusion constructs

| | FcRn binding | ka1 | | | ka2 | | |
|---|---|---|---|---|---|---|---|
| Construct | via | (1/Ms) | kd1 (1/s) | KD1 (M) | (1/RUs) | kd2 (1/s) | KD2 (M) |
| TPP-66143 | IgG4 FALA Fc | 8.28E+04 | 7.79E−02 | 9.41E−07 | 3.22E−05 | 1.36E−04 | 4.24E+00 |
| TPP-66144 | IgG4 FALA Fc | 8.08E+05 | 8.34E−02 | 1.03E−07 | 1.70E−02 | 4.85E−02 | 2.86E+00 |
| TPP-66145 | IgG4 FALA Fc | 8.03E+04 | 8.93E−02 | 1.11E−06 | 3.12E−05 | 1.15E−04 | 3.68E+00 |
| TPP-66146 | IgG4 FALA Fc | 4.34E+04 | 9.11E−02 | 2.10E−06 | 3.49E−05 | 5.08E−05 | 1.45E+00 |
| TPP-66147 | IgG4 FALA Fc | 1.01E+05 | 1.07E−01 | 1.06E−06 | 3.30E−05 | 1.05E−04 | 3.17E+00 |
| TPP-66148 | IgG4 FALA Fc | 1.07E+05 | 1.10E−01 | 1.03E−06 | 3.33E−05 | 7.12E−05 | 2.14E+00 |
| TPP-66149 | IgG4 FALA Fc | 8.70E+04 | 1.04E−01 | 1.20E−06 | 4.37E−05 | 1.48E−04 | 3.39E+00 |
| TPP-66150 | IgG4 FALA Fc | 7.39E+04 | 8.59E−02 | 1.16E−06 | 3.02E−05 | 1.92E−04 | 6.37E+00 |
| TPP-66151 | IgG4 FALA Fc | 1.02E+05 | 1.48E−01 | 1.46E−06 | 6.98E−05 | 1.45E−04 | 2.08E+00 |
| TPP-66152 | IgG4 FALA Fc | 5.75E+04 | 1.07E−01 | 1.86E−06 | 5.00E−05 | 1.79E−04 | 3.58E+00 |
| TPP-66153 | IgG4 FALA | 3.14E+04 | 7.80E−03 | 2.48E−07 | 2.32E+01 | 8.70E+01 | 3.74E+00 |
| | Fc, HSA | | | | | | |
| TPP-66154 | IgG4 FALA | 6.48E+03 | 2.97E−04 | 4.58E−08 | 5.45E−03 | 4.00E−06 | 7.33E−04 |
| | Fc, | | | | | | |
| | HSA(QMP) | | | | | | |
| TPP-66174 | IgG4 FALA Fc | 6.50E+04 | 7.86E−03 | 1.21E−07 | 5.58E+01 | 1.14E+02 | 2.04E+00 |
| | (YTE) | | | | | | |
| TPP-66175 | IgG4 FALA Fc | 9.33E+04 | 1.67E−02 | 1.79E−07 | 2.32E−02 | 9.22E−02 | 3.98E+00 |
| | (YTE) | | | | | | |
| TPP-66176 | IgG4 FALA Fc | 6.29E+04 | 7.94E−02 | 1.26E−06 | 3.79E−05 | 1.72E−04 | 4.53E+00 |
| TPP-66177 | IgG4 FALA Fc | 5.15E+04 | 8.72E−02 | 1.69E−06 | 4.07E−05 | 1.48E−04 | 3.64E+00 |

### Example 9

Development and Optimization of Plasma PKC Assays for with Albumin Binding VHH (ISVD)—IgG1 Fc Fusions (TP108, TP111, TP117, TP118, TP121 and TP123)

A specific plasma PK assay for test constructs TP108, TP111, TP117, TP118, TP121 and TP123 displayed in Table A-1 was developed to support the pharmacokinetic experiments performed in Tg32 mice. Briefly, the concentration of each compound at each timepoint were determined by a bottom-up LC-MS2 assay. Plasma samples were immunocaptured with a goat anti-human IgG biotinylated antibody. After elution, an isotopically labelled peptide was spiked as internal standard. Subsequently, samples were digested with trypsin and the resulting surrogate peptides were analysed by LC-MS/MS. Calibration standards and QC samples were prepared by spiking each compound in blank plasma.

Analysis of the peptides was performed in a Nexera UHPLC (Shimazdu) with an autosampler Exion multiplate (Sciex) hyphenated to a Sciex 6500+ mass spectrometer. For separation, a column Ascentis Express Peptide ES-C18 75×2.1 mm (Thermo Fisher Scientific) was flushed at room temperature with a stepwise gradient of water/formic acid (100/0.1; v/v) and acetonitrile/DMSO (98/2; v/v/v) with a 0.50 mL min-1 flow. The mass spectrometer was operated in positive mode according manufacturer's instructions with the ion source at 5500 V and 500° C. Dwell times were 5 ms. One unique surrogate peptide in the Fc domain was used for quantification. Chromatographic peak areas were calculated with Analyst (Sciex). Concentrations were calculated by using the ratio area of the analyte to the area of the internal standard in the same sample and interpolating the results in the calibration curve obtained with the calibration standards.

Pharmacokinetics in Mice with Albumin Binding VHH (ISVD)—IgG1 Fc Fusions

A PK experiment was designed to evaluate the PK properties of ISVD-IgG1 Fc and ISVD-IgG1 constructs (represented in FIG. 3 and Table A-1). These constructs were designed with KiH mutations. Fc-fusion constructs had 2 non-targeting ISVD fused N-terminally. Constructs either had no C-terminal fusions (TP117), a non-targeting or albumin-targeting ISVD domain fused C-terminally or 2 albumin-binding ISVD fused C-terminally (TP123). All animal studies were conducted according to Sanofi's standards regarding animal welfare.

Three Tg32 mice (B6.Cg-Fcgrttm1Dcr Tg(FCGRT) 32Dcr/DcrJ) were injected intravenously in the tail with 5 mg/kg ISVD-Fc or ISVD-IgG fusions.

Blood samples were collected into K2EDTA tubes at different time points (3 mice per time point) and processed to plasma by centrifugation (3000 g at 5° C. for 10 minutes). Plasma samples were frozen on dry ice within 90 minutes of collection. All Plasma samples were stored at −70° C. until shipping for analysis.

PK parameters were obtained from non-compartmental analysis in Phoenix WinNonlin® (version 8.2.2.227. Certara) using the Plasma Data Module. When applicable, sampling times with steep concentration decline of compound due to suspected ADA impact were excluded from analysis. Results are shown in FIGS. 4 and 5 and PK parameters are reported in Table 14.

TABLE 14
PK parameters of ISVD-IgG1 Fc fusions obtained by NCA analysis

| | | Dose | Cl | t_{1/2} |
|---|---|---|---|---|
| Test compound | HLE domain | (mg/kg) | (mL/hr/kg) | (hr) |
| TP108 | IgG1 Fc | 5 | 0.272 | 212 |
| TP111 | IgG1 Fc, ALB23002 | 5 | 0.161 | 429 |
| TP117 | IgG1 Fc | 5 | 0.283 | 290 |
| TP118 | IgG1 Fc | 5 | 0.237 | 289 |
| TP121 | IgG1 Fc, ALB23002 | 5 | 0.198 | 341 |
| TP123 | lgG1 Fc, 2x ALB23002 | 5 | 0.200 | 262 |

We can conclude from the results that an albumin-binding ISVD, fused to an IgG1 Fc domain significantly extends the half-life of the Fc domain (Table 14 and FIGS. 4-5). Improved half-lives and reduced clearance were observed for all constructs with a C-terminal albumin-binding ISVD compared to their respective control (Table 14). A single Albumin-binding ISVD is sufficient to achieve significant clearance reduction and improvement in half-life.

### Example 10

### Development and Optimization of Plasma PK Assays for IgG4 FALA Fc-Fusion Constructs

A specific plasma pharmacokinetic (PK) assay for test constructs displayed in FIG. 6 was developed to support the PK experiments performed in Tg32 mice. Briefly, the concentration of each compound at each timepoint were determined by a bottom-up LC-MS2 assay. Plasma samples were diluted, reduced, carbamidomethylated and digested with trypsin. After spiking with an internal standard peptide, solid phase extraction was performed (Thermo Scientific SOLAμ™ SPE). The resulting eluates were analysed by LC-MS/MS. Calibration standards and QC samples were prepared by spiking the compounds in blank plasma.

Quantification of the peptides was performed in an Sciex Exion UHPLC hyphenated to a Sciex 6500+ mass spectrometer. For separation, a column Kinetex XB C18, 130 Å 1.7 μm 100×2.1 mm (Phenomenex) was flushed at 50° C. with a stepwise gradient of water/formic acid (100/0.1; v/v) and acetonitrile/formic acid (100/0.1; v) with a 0.40 mL min-1 flow. The mass spectrometer was operated in positive mode according manufacturer's instructions with the ion source at 5500 V and 500° C. Dwell times were 50 ms. One multiple reaction transition corresponding to a unique peptide in the Fc domain was used as surrogate for quantification. Chromatographic peak areas were determined with the algorithm Analyst (Sciex). Concentrations were calculated by using the ratio area of the analyte to the area of the internal standard in the same sample and interpolating the results in the calibration curve obtained with the calibration standards.

Pharmacokinetics in Mice with IgG4 FALA Fc Fused to Albumin-Binding Moieties or Albumin (Variants)

A PK experiment was designed to evaluate the PK properties of an IgG4 FALA antibody Fc domain fused C-terminally to either an albumin binder (ISVD or other albumin-binding scaffolds) or human albumin variants. Fc-fusion constructs had 2 non-targeting ISVD fused N-terminally and the Fc consisted of either IgG4 FALA Fc, or IgG4 FALA Fc engineered for improved FcRn binding (i.e., YTE mutation). Control constructs consisted of the same Fc domains, but with C-terminal control ISVD fusion. Symmetrical constructs had on their C-terminus either 2 fused Albumin-binding ISVD or 2 control ISVD. All C-terminal ISVD were fused via a 35 GS-linker except for TPP66144. All constructs are listed in Table A-1/FIG. 6. All animal studies were conducted according to Sanofi's standards regarding animal welfare.

Three Tg32 mice (B6.Cg-FcgrttmlDcr Tg(FCGRT) 32Dcr/DcrJ) were injected intravenously in the tail vein with 5 mg/kg of Fc fusion construct. Blood samples were collected into K2EDTA tubes at different time points (3 mice per time point) and processed to plasma by centrifugation (3000 g at 5° C. for 10 minutes). Plasma samples were frozen on dry ice within 90 minutes of collection. All plasma samples were be stored at −70° C. until shipping for analysis.

PK parameters were obtained from non-compartmental analysis in Phoenix WinNonlin® (version 8.2.2.227. Certara) using the Plasma Data Module. When applicable, sampling times with steep concentration decline of compound due to suspected ADA impact were excluded from analysis. Results are shown in FIGS. 7-12 and PK parameters are reported in Table 15.

TABLE 15
PK parameters of IgG4 FALA Fc fusions
obtained by NCA analysis

| Test | | Dose | Cl | t_{1/2} |
|---|---|---|---|---|
| compound | HLE domain | (mg/kg) | (mL/hr/kg) | (hr) |
| TPP66143 | IgG4 FALA Fc | 5 | 0.306 | 288 |
| TPP66144 | IgG4 FALA Fc, ALB23002 | 5 | 0.105 | 552 |
| | (9GS) | | | |
| TPP66147 | IgG4 FALA Fc, ALB23002 | 5 | 0.111 | 434 |
| TPP66145 | IgG4 FALA Fc, HSA006A06 | 5 | 0.0909 | 782 |
| TPP66146 | IgG4 FALA Fc, ALB11002 | 5 | 0.0777 | 603 |
| TPP66148 | IgG4 FALA Fc, ALBX00002 | 5 | 0.143 | 431 |
| TPP66149 | IgG4 FALA Fc, T023500029 | 5 | 0.115 | 492 |
| TPP66150 | IgG4 FALA Fc, DARPIN | 5 | 0.160 | 627 |
| TPP66151 | IgG4 FALA Fc, Affitin | 5 | 0.158 | 498 |
| TPP66152 | IgG4 FALA Fc, ABD | 5 | 0.156 | 471 |
| TPP66153 | IgG4 FALA Fc, HSA | 5 | 0.116 | 467 |
| TPP66154 | IgG4 FALA Fc, HSA (QMP) | 5 | 0.145 | 390 |
| TPP66174 | IgG4 FALA Fc(YTE), | 5 | 0.136 | 635 |
| | ALB23002 | | | |
| TPP66175 | IgG4 FALA Fc(YTE) | 5 | 0.199 | 387 |
| TPP66176 | IgG4 FALA Fc | 5 | 0.236 | 399 |
| TPP66177 | IgG4 FALA Fc, 2x | 5 | 0.106 | 552 |
| | ALB23002 | | | |

We can conclude from the results that an albumin-binding moiety, fused to an IgG4 FALA Fc domain significantly reduces the clearance and extends the half-life of the polypeptide (FIGS. 7-12). Largest improvement in pharmacokinetic properties was observed for those cases where the albumin-binding moiety was an albumin-binding ISVD, such as ALB23002, ALB11002, HSA006A06, ALBX00002 or T023500029 as compared to other albumin-binding scaffolds such as a DARPIN®, affitin or a bacterial derived ABD. In addition, direct fusion of a human albumin (variant) to Fc was also shown to significantly improve pharmacokinetic properties of the polypeptide. Maximal effects in half-life extension were reached with a single albumin-binding ISVD, regardless whether the Fc domain was WT or engineered for improved FcRn binding (YTE).

TABLE 16
Summary of PK parameters of different tested compounds.

| | | Dose | Cl | t_{1/2} |
|---|---|---|---|---|
| Test compounds | HLE domain | (mg/kg) | (mL/hr/kg) | (hr) |
| TP003* | IgG4 FALA Fc | 5 | 0.796 | 144 |
| TP013* | IgG4 FALA Fc | 8 | 0.365 | 207 |
| TP009 | ALB23002, IgG4 FALA Fc | 5 | 0.196 | 390 |
| TP006 | IgG4 FALA Fc, ALB23002 | 5 | 0.163 | 310 |
| TP108* | IgG1 Fc | 5 | 0.272 | 212 |
| TP111 | IgG1 Fc, ALB23002 | 5 | 0.161 | 429 |
| TP117* | IgG1 Fc | 5 | 0.283 | 290 |
| TP118* | IgG1 Fc | 5 | 0.237 | 289 |
| TP121 | IgG1 Fc, ALB23002 | 5 | 0.198 | 341 |
| TP123 | IgG1 Fc, 2x ALB23002 | 5 | 0.200 | 262 |
| TPP66143* | IgG4 FALA Fc | 5 | 0.306 | 288 |
| TPP66144 | IgG4 FALA Fc, ALB23002 | 5 | 0.105 | 552 |
| TPP66147 | IgG4 FALA Fc, ALB23002 | 5 | 0.111 | 434 |
| TPP66145 | IgG4 FALA Fc, HSA006A06 | 5 | 0.0909 | 782 |
| TPP66146 | IgG4 FALA Fc, ALB11002 | 5 | 0.0777 | 603 |
| TPP66148 | IgG4 FALA Fc, ALBX00002 | 5 | 0.143 | 431 |
| TPP66149 | IgG4 FALA Fc, T023500029 | 5 | 0.115 | 492 |
| TPP66150 | IgG4 FALA Fc, DARPIN | 5 | 0.160 | 627 |
| TPP66151 | IgG4 FALA Fc, Affitin | 5 | 0.158 | 498 |
| TPP66152 | IgG4 FALA Fc, ABD | 5 | 0.156 | 471 |
| TPP66153 | IgG4 FALA Fc, HSA | 5 | 0.116 | 467 |
| TPP66154 | IgG4 FALA Fc, HSA(QMP) | 5 | 0.145 | 390 |
| TPP66174 | IgG4 FALA Fc(YTE), | 5 | 0.136 | 635 |
| | ALB23002 | | | |
| TPP66175* | IgG4 FALA Fc(YTE) | 5 | 0.199 | 387 |
| TPP66176* | IgG4 FALA Fc, 2x CNB | 5 | 0.236 | 399 |
| TPP66177 | IgG4 FALA Fc, 2x ALB23002 | 5 | 0.106 | 552 |

Control polypeptides are bold and marked by*. “Cl” stands for clearance, as in the other tables.

### SEQUENCE LISTING

TABLE A-6
CDRs and FR sequences of Alb-binding ISVDs
(“ID” refers to the SEQ ID NO as used herein)

| Name | ID | Sequence |
|---|---|---|
| CDR1 Alb 23002 Kabat | 1 | SFGMS |
| CDR2 Alb 23002Kabat | 2 | SISGSGSDTLYADSVKG |
| CDR3 Alb 23002Kabat | 3 | GGSLSR |
| CDR1 Alb 23002AbM | 4 | GFTFRSFGMS |
| CDR2 Alb 23002AbM | 5 | SISGSGSDTL |
| CDR3 Alb 23002AbM | 6 | GGSLSR |
| ALB23002 FR1 (AbM) | 70 | EVQLVESGGGVVQPGGSLRLSCAAS |
| ALB23002 FR2 (AbM) | 71 | WVRQAPGKGPEWVS |
| ALB23002 FR3 (AbM) | 72 | YADSVKGRFTISRDNSKNTLYLQMNSLRPEDTALYYCTI |
| ALB23002 FR4 (AbM/Kabat) | 73 | SSQGTLVTVSS |
| ALB23002 FR1 (Kabat) | 74 | EVQLVESGGGVVQPGGSLRLSCAASGFTFR |
| ALB23002 FR2 (Kabat) | 70 | WVRQAPGKGPEWVS |
| ALB23002 FR3 (Kabat) | 76 | RFTISRDNSKNTLYLQMNSLRPEDTALYYCTI |
| CDR1 Alb-binding ISVDs (AbM) | 77 | GFTFSSFGMS |
| T023500029 CDR1 (Kabat) | 78 | TYVMG |
| T023500029 CDR2 (Kabat) | 79 | AISQNSIHTYYANSVKG |
| T023500029 CDR1 (Abm) | 80 | GGTFSTYVMG |
| T023500029 CDR2 (Abm) | 81 | AISQNSIHTY |
| T023500029 CDR3 (Kabat/Abm) | 82 | SRFTSWYTADYEYDY |
| T023500029 FR1 (Abm) | 83 | EVQLVESGGGVVQPGDSLRLSCAAS |
| T023500029 FR2 (Abm) | 84 | WFRQAPGKEREFVS |
| T023500029 FR3 (Abm) | 85 | YANSVKGRFTISRDNSKNTVYLQLNSLRPEDTALYYCAA |
| T023500029 FR4 (Abm and Kabat) | 86 | WGQGTLVTVSS |
| T023500029 FR1 (Kabat) | 87 | EVQLVESGGGVVQPGDSLRLSCAASGGTFS |
| T023500029 FR2 (Kabat) | 84 | WFRQAPGKEREFVS |
| T023500029 FR3 (Kabat) | 89 | RFTISRDNSKNTVYLQLNSLRPEDTALYYCAA |
| AlbX00001 and ALBX00002 CDR1 (AbM) | 90 | GLTFSSYAMG |
| AlbX00001 and ALBX00002 CDR2 (AbM) | 91 | SISRGGGYTY |
| AlbX00001 and ALBX00002 CDR3 (AbM and Kabat) | 92 | ARYWATGSEYEFDY |
| AlbX00001 and ALBX00002 CDR1 (Kabat) | 93 | SYAMG |
| AlbX00001 and ALBX00002 CDR2 (Kabat) | 94 | SISRGGGYTYYADSVKG |
| AlbX00001 and ALBX00002 FR1 (AbM) | 70 | EVQLVESGGGVVQPGGSLRLSCAAS |
| AlbX00001 and ALBX00002 FR2 (AbM) | 96 | WFRQAPGKERERVV |
| ALBX00002 FR3 (AbM) | 97 | YADSVKGRFTISRDNSKNTVYLQMNSLRPEDTALYYCAA |
| ALBX00002 FR4 (AbM and Kabat) | 86 | WGQGTLVTVSS |
| ALBX00002 FR1 (Kabat) | 99 | EVQLVESGGGVVQPGGSLRLSCAASGLTFS |
| ALBX00002 FR2 (Kabat) | 96 | WFRQAPGKERERVV |
| ALBX00002 FR3 (Kabat) | 101 | RFTISRDNSKNTVYLQMNSLRPEDTALYYCAA |
| AlbX00001 FR3 (AbM) | 118 | YADSVKGRFTISRDNSENTVYLQMNSLRPEDTALYYCAA |
| AlbX00001 FR3 (Kabat) | 138 | RFTISRDNSENTVYLQMNSLRPEDTALYYCAA |

TABLE A-3
Serum albumin binding ISVD sequences
(“ID” refers to the SEQ ID NO as used herein)

| Name | ID | Amino acid sequence |
|---|---|---|
| Alb8 | 7 | EVQLVESGGGLVQPGNSLRLSCAASGFTFSSFGMSWVRQAPGK |
| | | GLEWVSSISGSGSDTLYADSVKGRFTISRDNAKTTLYLQMNSLRP |
| | | EDTAVYYCTIGGSLSRSSQGTLVTVSS |
| Alb23 | 8 | EVQLLESGGGLVQPGGSLRLSCAASGFTFRSFGMSWVRQAPGK |
| | | GPEWVSSISGSGSDTLYADSVKGRFTISRDNSKNTLYLQMNSLR |
| | | PEDTAVYYCTIGGSLSRSSQGTLVTVSS |
| Alb129 | 9 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTATYYCTIGGSLSRSSQGTLVTVSSA |
| Alb132 | 10 | EVQLVESGGGVVQPGGSLRLSCAASGFTFRSFGMSWVRQAPG |
| | | KGPEWVSSISGSGSDTLYADSVKGRFTISRDNSKNTLYLQMNSL |
| | | RPEDTATYYCTIGGSLSRSSQGTLVTVSSA |
| Alb11 | 11 | EVQLVESGGGLVQPGNSLRLSCAASGFTFSSFGMSWVRQAPGK |
| | | GLEWVSSISGSGSDTLYADSVKGRFTISRDNAKTTLYLQMNSLRP |
| | | EDTAVYYCTIGGSLSRSSQGTLVTVSS |
| Alb11 (S112K)-A | 12 | EVQLVESGGGLVQPGNSLRLSCAASGFTFSSFGMSWVRQAPGK |
| | | GLEWVSSISGSGSDTLYADSVKGRFTISRDNAKTTLYLQMNSLRP |
| | | EDTAVYYCTIGGSLSRSSQGTLVKVSSA |
| Alb82 (Alb11002) | 13 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTALYYCTIGGSLSRSSQGTLVTVSS |
| Alb82-A (ALB11002-A) | 14 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTALYYCTIGGSLSRSSQGTLVTVSSA |
| Alb82-AA | 15 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTALYYCTIGGSLSRSSQGTLVTVSSAA |
| Alb82-AAA | 16 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTALYYCTIGGSLSRSSQGTLVTVSSAAA |
| Alb82-G | 17 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTALYYCTIGGSLSRSSQGTLVTVSSG |
| Alb82-GG | 18 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTALYYCTIGGSLSRSSQGTLVTVSSGG |
| Alb82-GGG | 19 | EVQLVESGGGVVQPGNSLRLSCAASGFTFSSFGMSWVRQAPG |
| | | KGLEWVSSISGSGSDTLYADSVKGRFTISRDNAKTTLYLQMNSLR |
| | | PEDTALYYCTIGGSLSRSSQGTLVTVSSGGG |
| Alb23002 | 20 | EVQLVESGGGVVQPGGSLRLSCAASGFTFRSFGMSWVRQAPG |
| | | KGPEWVSSISGSGSDTLYADSVKGRFTISRDNSKNTLYLQMNSL |
| | | RPEDTALYYCTIGGSLSRSSQGTLVTVSS |
| Alb223/Alb23002-A | 21 | EVQLVESGGGVVQPGGSLRLSCAASGFTFRSFGMSWVRQAPG |
| | | KGPEWVSSISGSGSDTLYADSVKGRFTISRDNSKNTLYLQMNSL |
| | | RPEDTALYYCTIGGSLSRSSQGTLVTVSSA |
| Alb23002(E1D) | 61 | DVQLVESGGGVVQPGGSLRLSCAASGFTFRSFGMSWVRQAPG |
| | | KGPEWVSSISGSGSDTLYADSVKGRFTISRDNSKNTLYLQMNSL |
| | | RPEDTALYYCTIGGSLSRSSQGTLVTVSS |
| T023500029 | 62 | EVQLVESGGGVVQPGDSLRLSCAASGGTFSTYVMGWFRAPGK |
| | | EREFVSAISQNSIHTYYANSVKGRFTISRDNSKNTVYLQLNSLRPE |
| | | DTALYYCAASRFTSWYTADYEYDYWGQGTLVTVSS |
| AlbX00001 | 63 | EVQLVESGGGVVQPGGSLRLSCAASGLTFSSYAMGWFRQAPG |
| | | KERERVVSISRGGGYTYYADSVKGRFTISRDNSENTVYLQMNSL |
| | | RPEDTALYYCAAARYWATGSEYEFDYWGQGTLVTVSS |
| ALBX00002 | 64 | EVQLVESGGGVVQPGGSLRLSCAASGLTFSSYAMGWFRQAPG |
| | | KERERVVSISRGGGYTYYADSVKGRFTISRDNSKNTVYLQMNSL |
| | | RPEDTALYYCAAARYWATGSEYEFDYWGQGTLVTVSS |
| HSA006A06 | 65 | EVQLVESGGGLVQPGNSLRLSCAASGFTFRSFGMSWVRQAPG |
| | | KEPEWVSSISGSGSDTLYADSVKGRFTISRDNAKTTLYLQMNSLK |
| | | PEDTAVYYCTIGGSLSRSSQGTQVTVSS |
| HSA006A06-A | 66 | EVQLVESGGGLVQPGNSLRLSCAASGFTFRSFGMSWVRQAPG |
| | | KEPEWVSSISGSGSDTLYADSVKGRFTISRDNAKTTLYLQMNSLK |
| | | PEDTAVYYCTIGGSLSRSSQGTQVTVSSA |
| ALB-1 | 67 | AVQLVESGGGLVQPGNSLRLSCAASGFTFRSFGMSWVRQAPG |
| | | KEPEWVSSISGSGSDTLYADSVKGRFTISRDNAKTTLYLQMNSLK |
| | | PEDTAVYYCTIGGSLSRSSQGTQVTVSS |
| ALBX00002-A | 68 | EVQLVESGGGVVQPGGSLRLSCAASGLTFSSYAMGWFRQAPG |
| | | KERERVVSISRGGGYTYYADSVKGRFTISRDNSKNTVYLQMNSL |
| | | RPEDTALYYCAAARYWATGSEYEFDYWGQGTLVTVSSA |
| T0235002C06(L11V, T14P, D74S, | 69 | EVQLVESGGGVVQPGDSLRLSCAASGGTFSTYVMGWFRQAPG |
| K83R, V89L)-A (T023500029-A) | | KEREFVSAISQNSIHTYYANSVKGRFTISRDNSKNTVYLQLNSLRP |
| | | EDTALYYCAASRFTSWYTADYEYDYWGQGTLVTVSSA |

TABLE A-4
Serum albumin binding protein sequences
(“ID” refers to the SEQ ID NO as used herein)

| Name | ID | Amino acid sequence |
|---|---|---|
| Darpin | 102 | DLGKKLLEAARAGQDDEVRELLKAGADVNAKDYF |
| | | SHTPLHLAARNGHLKIVEVLLKAGADVNAKDFAG |
| | | KTPLHLAANEGHLEIVEVLLKAGADVNAQDIFGK |
| | | TPADIAADAGHEDIAEVLQKAA |
| Affitin | 103 | VKVKFWPRGEEKVVDTSKIAWVLRADKTVMFKYD |
| (Nanofitin) | | DNGKKGYGVVLEKDAPKELLDMLARAEREK |
| ABD | 104 | LKEAKEKAIEELKKAGITSDYYFDLINKAKTVEG |
| | | VNALKDEILKA |

TABLE A-8
“Irrelevant” or “Control” ISVD sequences
(“ID” refers to the SEQ ID NO as used herein)

| Name | ID | Amino acid sequence |
|---|---|---|
| RSV001A04(E1D, L11V, | 105 | DVQLVESGGGVVQAGGSLSISCAASGGSLSNYVLGWFRQAPGKEREFVA |
| V89L, Q108L) | | AINWRGDITIGPPNVEGRFTISRDNAKNTGYLQMNSLAPDDTALYYCGAG |
| | | TPLNPGAYIYDWSYDYWGRGTLVTVSS |
| HER2005F07(E1D, Q108L) | 106 | DVQLVESGGGLVQAGGSLRLSCAASGITFSINTMGWYRQAPGKQRELVA |
| | | LISSIGDTYYADSVKGRFTISRDNAKNTVYLQMNSLKPEDTAVYYCKRFRTA |
| | | AQGTDYWGQGTLVTVSS |
| Non-binding ISVD | 182 | EVQLVESGGGVVQAGGSLSISCAASGGSLSNYVLGWFRQAPGKEREFVA |
| (RSV001A04+) | | AINWRGDITIGPPNVEGRFTISRDNAKNTGYLQMNSLAPDDTALYYCGAG |
| (RSV001A04(L11V, V89L, | | TPLNPGAYIYDWSYDYWGRGTLVTVSSA |
| Q108L)-A) | | |
| Trastuzumab(VH)- | 127 | EVQLVESGGGLVQPGGSLRLSCAASGFNIRDTYIHWVRQAPGKGLEWVA |
| IgG1 | | RIYPTQGYTRYADSVKGRFTISADTSKNTAYLQMNSLRAEDTAVYYCSRW |
| | | GGEGFYAMDYWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQT |
| | | YICNVNHKPSNTKVDKKVEPKSCDKTHTCPPCPAPELLGGPSVFLFPPKPK |
| | | DTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQV |
| | | YTLPPCRDELTKNQVSLWCLVKGFYPSDIAVEWESNGQPENNYKTTPPVL |
| | | DSDGSFFLYSKLTVDKSRWQQGNVFSCSVMHEALHNHYTQKSLSLSPG |
| Trastuzumab VH | 107 | EVQLVESGGGLVQPGGSLRLSCAASGFNIRDTYIHWVRQAPGKGLEWVA |
| | | RIYPTQGYTRYADSVKGRFTISADTSKNTAYLQMNSLRAEDTAVYYCSRW |
| | | GGEGFYAMDYWGQGTLVTVSS |
| Trastuzumab | 129 | DIQMTQSPSSLSASVGDRVTITCRASQDVNTAVAWYQQKPGKAPKLLIYS |
| complete light chain | | ASFLYSGVPSRFSGSRSGTDFTLTISSLQPEDFATYYCQQHYTTPPTFGQGT |
| | | KVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSP |
| | | VTKSFNRGEC |
| Trastuzumab (VL) | 108 | DIQMTQSPSSLSASVGDRVTITCRASQDVNTAVAWYQQKPGKAPKLLIYS |
| | | ASFLYSGVPSRFSGSRSGTDFTLTISSLQPEDFATYYCQQHYTTPPTFGQGT |
| | | KVEIK |
| Palivizumab_IgG4FALA_ | 191 | QVTLRESGPALVKPTQTLTLTCTFSGFSLSTSGMSVGWIRQPPGKALEWLA |
| knobs | | DIWWDDKKDYNPSLKSRLTISKDTSKNQVVLKVTNMDPADTATYYCARS |
| | | MITNWYFDVWGAGTTVTVSSASTKGPSVFPLAPCSRSTSESTAALGCLVK |
| | | DYFPEPVTVSWNSGALTSGVHTFPAVLQSSGLYSLSSVVTVPSSSLGTKTYT |
| | | CNVDHKPSNTKVDKRVESKYGPPCPPCPAPEAAGGPSVFLFPPKPKDTLM |
| | | ISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLP |
| | | PCQEEMTKNQVSLWCLVKGFYPSDIAVEWESNGQPENNYKTTPPVLDSD |
| | | GSFFLYSKLTVDKSRWQEGNVFSCSVMHEALHNHYTQKSLSLSLG |
| Palivizumab | 192 | QVTLRESGPALVKPTQTLTLTCTFSGFSLSTSGMSVGWIRQPPGKALEWLA |
| IgG4FALA_hole | | DIWWDDKKDYNPSLKSRLTISKDTSKNQVVLKVTNMDPADTATYYCARS |
| | | MITNWYFDVWGAGTTVTVSSASTKGPSVFPLAPCSRSTSESTAALGCLVK |
| | | DYFPEPVTVSWNSGALTSGVHTFPAVLQSSGLYSLSSVVTVPSSSLGTKTYT |
| | | CNVDHKPSNTKVDKRVESKYGPPCPPCPAPEAAGGPSVFLFPPKPKDTLM |
| | | ISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVCTLP |
| | | PSQEEMTKNQVSLSCAVKGFYPSDIAVEWESNGQPENNYKTTPPVLDSD |
| | | GSFFLVSKLTVDKSRWQEGNVFSCSVMHEALHNHYTQKSLSLSLG |
| Palivizumab(VH) | 193 | QVTLRESGPALVKPTQTLTLTCTFSGFSLSTSGMSVGWIRQPPGKALEWLA |
| | | DIWWDDKKDYNPSLKSRLTISKDTSKNQVVLKVTNMDPADTATYYCARS |
| | | MITNWYFDVWGAGTTVTVSS |
| Palivizumab complete | 194 | DIQMTQSPSTLSASVGDRVTITCKCQLSVGYMHWYQQKPGKAPKLLIYDT |
| light chain | | SKLASGVPSRFSGSGSGTEFTLTISSLQPDDFATYYCFQGSGYPFTFGGGTK |
| | | LEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPV |
| | | TKSFNRGEC |
| Palivizumab(VL) | 195 | DIQMTQSPSTLSASVGDRVTITCKCQLSVGYMHWYQQKPGKAPKLLIYDT |
| | | SKLASGVPSRFSGSGSGTEFTLTISSLQPDDFATYYCFQGSGYPFTFGGGTK |
| | | LEIK |
| 13F07(E1D) | 196 | DVQLVESGGGLVQPGGSLRLSCAASGLTFSTNPMYWYRQAPGKQRELVA |
| | | SISSRGITNYADSVKGRFTISRDNSKNTVYLQMNSLRPEDTAVYYCRLASLS |
| | | SGTVYWGQGTLVTVSS |
| 13F07 | 197 | EVQLVESGGGLVQPGGSLRLSCAASGLTFSTNPMYWYRQAPGKQRELVA |
| | | SISSRGITNYADSVKGRFTISRDNSKNTVYLQMNSLRPEDTAVYYCRLASLS |
| | | SGTVYWGQGTLVTVSS |

TABLE A-9
HSA, Fc and FcRn sequences (“ID” refers to the SEQ ID NO as used herein)

| Name | ID | Amino acid sequence |
|---|---|---|
| Sequence of HSA | 109 | MKWVTFISLLFLFSSAYSRGVFRRDAHKSEVAHRFKDLGEENFKALVLIA |
| uniprot ID P02768 | | FAQYLQQCPFEDHVKLVNEVTEFAKTCVADESAENCDKSLHTLFGDKLC |
| | | TVATLRETYGEMADCCAKQEPERNECFLQHKDDNPNLPRLVRPEVDV |
| | | MCTAFHDNEETFLKKYLYEIARRHPYFYAPELLFFAKRYKAAFTECCQAA |
| | | DKAACLLPKLDELRDEGKASSAKQRLKCASLQKFGERAFKAWAVARLSQ |
| | | RFPKAEFAEVSKLVTDLTKVHTECCHGDLLECADDRADLAKYICENQDSI |
| | | SSKLKECCEKPLLEKSHCIAEVENDEMPADLPSLAADFVESKDVCKNYAE |
| | | AKDVFLGMFLYEYARRHPDYSVVLLLRLAKTYETTLEKCCAAADPHECYA |
| | | KVFDEFKPLVEEPQNLIKQNCELFEQLGEYKFQNALLVRYTKKVPQVSTP |
| | | TLVEVSRNLGKVGSKCCKHPEAKRMPCAEDYLSVVLNQLCVLHEKTPVS |
| | | DRVTKCCTESLVNRRPCFSALEVDETYVPKEFNAETFTFHADICTLSEKER |
| | | QIKKQTALVELVKHKPKATKEQLKAVMDDFAAFVEKCCKADDKETCFA |
| | | EEGKKLVAASQAALGL |
| Human serum albumin | 22 | DAHKSEVAHRFKDLGEENFKALVLIAFAQYLQQCPFEDHVKLVNEVTEF |
| (1) | | AKTCVADESAENCDKSLHTLFGDKLCTVATLRETYGEMADCCAKQEPE |
| | | RNECFLQHKDDNPNLPRLVRPEVDVMCTAFHDNEETFLKKYLYEIARRH |
| | | PYFYAPELLFFAKRYKAAFTECCQAADKAACLLPKLDELRDEGKASSAKQ |
| | | RLKCASLQKFGERAFKAWAVARLSQRFPKAEFAEVSKLVTDLTKVHTEC |
| | | CHGDLLECADDRADLAKYICENQDSISSKLKECCEKPLLEKSHCIAEVEND |
| | | EMPADLPSLAADFVESKDVCKNYAEAKDVFLGMFLYEYARRHPDYSVV |
| | | LLLRLAKTYETTLEKCCAAADPHECYAKVFDEFKPLVEEPQNLIKQNCELF |
| | | KQLGEYKFQNALLVRYTKKVPQVSTPTLVEVSRNLGKVGSKCCKHPEAK |
| | | RMPCAEDYLSVVLNQLCVLHEKTPVSDRVTKCCTESLVNRRPCFSALEV |
| | | DETYVPKEFNAETFTFHADICTLSEKERQIKKQTALVELVKHKPKATKEQL |
| | | KAVMDDFAAFVEKCCKADDKETCFAEEGKKLVAASQAALGL |
| Human serum albumin | 23 | DAHKSEVAHRFKDLGEENFKALVLIAFAQYLQQCPFEDHVKLVNEVTEF |
| (2) (HSA(25-609)) | | AKTCVADESAENCDKSLHTLFGDKLCTVATLRETYGEMADCCAKQEPE |
| | | RNECFLQHKDDNPNLPRLVRPEVDVMCTAFHDNEETFLKKYLYEIARRH |
| | | PYFYAPELLFFAKRYKAAFTECCQAADKAACLLPKLDELRDEGKASSAKQ |
| | | RLKCASLQKFGERAFKAWAVARLSQRFPKAEFAEVSKLVTDLTKVHTEC |
| | | CHGDLLECADDRADLAKYICENQDSISSKLKECCEKPLLEKSHCIAEVEND |
| | | EMPADLPSLAADFVESKDVCKNYAEAKDVFLGMFLYEYARRHPDYSVV |
| | | LLLRLAKTYETTLEKCCAAADPHECYAKVFDEFKPLVEEPQNLIKQNCELF |
| | | EQLGEYKFQNALLVRYTKKVPQVSTPTLVEVSRNLGKVGSKCCKHPEAK |
| | | RMPCAEDYLSVVLNQLCVLHEKTPVSDRVTKCCTESLVNRRPCFSALEV |
| | | DETYVPKEFNAETFTFHADICTLSEKERQIKKQTALVELVKHKPKATKEQL |
| | | KAVMDDFAAFVEKCCKADDKETCFAEEGKKLVAASQAALGL |
| Sequence of HSA | 180 | MKWVTFLLLLFVSGSAFSRGVFRREAHKSEIAHRYNDLGEQHFKGLVLI |
| Uniprot ID: | | AFSQYLQKCSYDEHAKLVQEVTDFAKTCVADESAANCDKSLHTLFGDKL |
| NP_033784.2 (Mouse | | CAIPNLRENYGELADCCTKQEPERNECFLQHKDDNPSLPPFERPEAEAM |
| serum albumin, MSA) | | CTSFKENPTTFMGHYLHEVARRHPYFYAPELLYYAEQYNEILTQCCAEA |
| | | DKESCLTPKLDGVKEKALVSSVRQRMKCSSMQKFGERAFKAWAVARLS |
| | | QTFPNADFAEITKLATDLTKVNKECCHGDLLECADDRAELAKYMCENQ |
| | | ATISSKLQTCCDKPLLKKAHCLSEVEHDTMPADLPAIAADFVEDQEVCK |
| | | NYAEAKDVFLGTFLYEYSRRHPDYSVSLLLRLAKKYEATLEKCCAEANPP |
| | | ACYGTVLAEFQPLVEEPKNLVKTNCDLYEKLGEYGFQNAILVRYTQKAP |
| | | QVSTPTLVEAARNLGRVGTKCCTLPEDQRLPCVEDYLSAILNRVCLLHEK |
| | | TPVSEHVTKCCSGSLVERRPCFSALTVDETYVPKEFKAETFTFHSDICTLP |
| | | EKEKQIKKQTALAELVKHKPKATAEQLKTVMDDFAQFLDTCCKAADKD |
| | | TCFSTEGPNLVTRCKDALA |
| Human FcRn | 24 | AESHLSLLYHLTAVSSPAPGTPAFWVSGWLGPQQYLSYNSLRGEAEPC |
| | | GAWVWENQVSWYWEKETTDLRIKEKLFLEAFKALGGKGPYTLQGLLG |
| | | CELGPDNTSVPTAKFALNGEEFMNFDLKQGTWGGDWPEALAISQRW |
| | | QQQDKAANKELTFLLFSCPHRLREHLERGRGNLEWKEPPSMRLKARPS |
| | | SPGFSVLTCSAFSFYPPELQLRFLRNGLAAGTGQGDFGPNSDGSFHASS |
| | | SLTVKSGDEHHYCCIVQHAGLAQPLRVELESPAKSS |
| HSA(25- | 110 | DAHKSEVAHRFKDLGEENFKALVLIAFAQYLQQCPFEDHVKLVNEVTEF |
| 609)(E529Q, T551M, | | AKTCVADESAENCDKSLHTLFGDKLCTVATLRETYGEMADCCAKQEPE |
| K597P) | | RNECFLQHKDDNPNLPRLVRPEVDVMCTAFHDNEETFLKKYLYEIARRH |
| | | PYFYAPELLFFAKRYKAAFTECCQAADKAACLLPKLDELRDEGKASSAKQ |
| | | RLKCASLQKFGERAFKAWAVARLSQRFPKAEFAEVSKLVTDLTKVHTEC |
| | | CHGDLLECADDRADLAKYICENQDSISSKLKECCEKPLLEKSHCIAEVEND |
| | | EMPADLPSLAADFVESKDVCKNYAEAKDVFLGMFLYEYARRHPDYSVV |
| | | LLLRLAKTYETTLEKCCAAADPHECYAKVFDEFKPLVEEPQNLIKQNCELF |
| | | EQLGEYKFQNALLVRYTKKVPQVSTPTLVEVSRNLGKVGSKCCKHPEAK |
| | | RMPCAEDYLSVVLNQLCVLHEKTPVSDRVTKCCTESLVNRRPCFSALEV |
| | | DETYVPKEFNAQTFTFHADICTLSEKERQIKKQMALVELVKHKPKATKE |
| | | QLKAVMDDFAAFVEKCCKADDKETCFAEEGPKLVAASQAALGL |
| Human IgG1 fragment | 112 | DKTHTCPPCPAPELLGGPSVFLFPPKPKDTLMISRTPEVTCVVVDVSHED |
| with DKTHTCPPCP | | PEVKFNWYVDGVEVHNAKTKPREEQYNSTYRVVSVLTVLHQDWLNGK |
| linker | | EYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRW |
| | | QQGNVFSCSVMHEALHNHYTQKSLSLSPG |
| Human IgG1 fragment | 113 | APELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFS |
| | | CSVMHEALHNHYTQKSLSLSPG |
| Human IgG4 fragment | 115 | APEFLGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNK |
| | | GLPSSIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFSC |
| | | SVMHEALHNHYTQKSLSLSLG |
| IgG4 FALA Fc domain | 181 | APEAAGGPSVFLFPPKPKDTLMISRTPEVTCVVVDVSQEDPEVQFNWY |
| | | VDGVEVHNAKTKPREEQFNSTYRVVSVLTVLHQDWLNGKEYKCKVSN |
| | | KGLPSSIEKTISKAKGQPREPQVYTLPPSQEEMTKNQVSLTCLVKGFYPS |
| | | DIAVEWESNGQPENNYKTTPPVLDSDGSFFLYSKLTVDKSRWQEGNVF |
| | | SCSVMHEALHNHYTQKSLSLSLG |
| human IgG1 | 116 | APELLGGPSVFLFPPKPKDTLMISRTPEVTCVVVDVSHEDPEVKFNWYV |
| Fc_dk_knobs | | DGVEVHNAKTKPREEQYNSTYRVVSVLTVLHQDWLNGKEYKCKVSNK |
| | | ALPAPIEKTISKAKGQPREPQVYTLPPCRDELTKNQVSLWCLVKGFYPSD |
| | | IAVEWESNGQPENNYKTTPPVLDSDGSFFLYSKLTVDKSRWQQGNVFS |
| | | CSVMHEALHNHYTQKSLSLSPG |
| human IgG1 | 117 | APELLGGPSVFLFPPKPKDTLMISRTPEVTCVVVDVSHEDPEVKFNWYV |
| Fc_dk_holes_RF | | DGVEVHNAKTKPREEQYNSTYRVVSVLTVLHQDWLNGKEYKCKVSNK |
| | | ALPAPIEKTISKAKGQPREPQVCTLPPSRDELTKNQVSLSCAVKGFYPSDI |
| | | AVEWESNGQPENNYKTTPPVLDSDGSFFLVSKLTVDKSRWQQGNVFS |
| | | CSVMHEALHNRFTQKSLSLSPG |
| IgG4 FALA Fc domain | 186 | APEAAGGPSVFLFPPKPKDTLMISRTPEVTCVVVDVSQEDPEVQFNWY |
| (knobs) | | VDGVEVHNAKTKPREEQFNSTYRVVSVLTVLHQDWLNGKEYKCKVSN |
| | | KGLPSSIEKTISKAKGQPREPQVYTLPPCQEEMTKNQVSLWCLVKGFYP |
| | | SDIAVEWESNGQPENNYKTTPPVLDSDGSFFLYSKLTVDKSRWQEGNV |
| | | FSCSVMHEALHNHYTQKSLSLSLG |
| IgG4 FALA Fc domain | 187 | APEAAGGPSVFLFPPKPKDTLMISRTPEVTCVVVDVSQEDPEVQFNWY |
| (holes) | | VDGVEVHNAKTKPREEQFNSTYRVVSVLTVLHQDWLNGKEYKCKVSN |
| | | KGLPSSIEKTISKAKGQPREPQVCTLPPSQEEMTKNQVSLSCAVKGFYPS |
| | | DIAVEWESNGQPENNYKTTPPVLDSDGSFFLVSKLTVDKSRWQEGNVF |
| | | SCSVMHEALHNRFTQKSLSLSLG |
| IgG4 FALA Fc domain | 188 | APEAAGGPSVFLFPPKPKDTLYITREPEVTCVVVDVSQEDPEVQFNWYV |
| (knobs YTE) | | DGVEVHNAKTKPREEQFNSTYRVVSVLTVLHQDWLNGKEYKCKVSNK |
| | | GLPSSIEKTISKAKGQPREPQVYTLPPCQEEMTKNQVSLWCLVKGFYPS |
| | | DIAVEWESNGQPENNYKTTPPVLDSDGSFFLYSKLTVDKSRWQEGNVF |
| | | SCSVMHEALHNHYTQKSLSLSLG |
| IgG4 FALA Fc domain | 189 | APEAAGGPSVFLFPPKPKDTLYITREPEVTCVVVDVSQEDPEVQFNWYV |
| (holes YTE) | | DGVEVHNAKTKPREEQFNSTYRVVSVLTVLHQDWLNGKEYKCKVSNK |
| | | GLPSSIEKTISKAKGQPREPQVCTLPPSQEEMTKNQVSLSCAVKGFYPSD |
| | | IAVEWESNGQPENNYKTTPPVLDSDGSFFLVSKLTVDKSRWQEGNVFS |
| | | CSVMHEALHNRFTQKSLSLSLG |
| IgG4 FALA Fc domain | 190 | APEAAGGPSVFLFPPKPKDTLMISRTPEVTCVVVDVSQEDPEVQFNWY |
| (holes) | | VDGVEVHNAKTKPREEQFNSTYRVVSVLTVLHQDWLNGKEYKCKVSN |
| | | KGLPSSIEKTISKAKGQPREPQVCTLPPSQEEMTKNQVSLSCAVKGFYPS |
| | | DIAVEWESNGQPENNYKTTPPVLDSDGSFFLVSKLTVDKSRWQEGNVF |
| | | SCSVMHEALHNHYTQKSLSLSLG |
| IgG4 FALA Fc domain | 198 | APEAAGGPSVFLFPPKPKDTLMASRTPEVTCVVVDVSQEDPEVQFNWY |
| (knobs_AAA) | | VDGVEVHNAKTKPREEQFNSTYRVVSVLTVLAQDWLNGKEYKCKVSN |
| | | KGLPSSIEKTISKAKGQPREPQVYTLPPCQEEMTKNQVSLWCLVKGFYP |
| | | SDIAVEWESNGQPENNYKTTPPVLDSDGSFFLYSKLTVDKSRWQEGNV |
| | | FSCSVMHEALHNAYTQKSLSLSLG |
| IgG4 FALA Fc domain | 199 | APEAAGGPSVFLFPPKPKDTLMASRTPEVTCVVVDVSQEDPEVQFNWY |
| (holes_AAA) | | VDGVEVHNAKTKPREEQFNSTYRVVSVLTVLAQDWLNGKEYKCKVSN |
| | | KGLPSSIEKTISKAKGQPREPQVCTLPPSQEEMTKNQVSLSCAVKGFYPS |
| | | DIAVEWESNGQPENNYKTTPPVLDSDGSFFLVSKLTVDKSRWQEGNVF |
| | | SCSVMHEALHNAYTQKSLSLSLG |
| Amino acid sequences | 167 | CPPCPAPELLGGPSVFLFPPKPKDTLYITREPEVTCVVVDVSHEDPEVKF |
| of Fc regions | | NWYVDGVEVHNAKTKPREEQYNSTYRVVSVLTVLHQDWLNGKEYKCK |
| (WO 2015/100299) | | VSNKALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGN |
| | | VFSCSVMHEALKFHYTQKSLSLSPG |
| Amino acid sequences | 168 | DKTHTCPPCPAPELLGGPSVFLFPPKPKDTLYITREPEVTCVVVDVSHED |
| of Fc regions | | PEVKFNWYVDGVEVHNAKTKPREEQYNSTYRVVSVLTVLHQDWLNGK |
| (WO 2015/100299) | | EYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRW |
| | | QQGNVFSCSVMHEALKFHYTQKSLSLSPGK |
| Amino acid | 169 | DKTHTCPPCPAPELLGGPSVFLFPPKPKDTLYITREPEVTCVVVDVSHED |
| sequences of Fc | | PEVKFNWYVDGVEVHNAKTKPREEQYNSTYRVVSVLTVLHQDWLNGK |
| regions | | EYKCKVSNKALPAPIEKTISKAKGQPREPQVYTLPPSRDELTKNQVSLTCL |
| (WO 2015/100299) | | VKGFYPSDIAVEWESNGQPENNYKTTPPVLDSDGSFFLYSKLTVDKSRW |
| | | QQGNVFSCSVMHEALKFHYTQKSLSLSPG |
| Amino acid sequences | 170 | APELLGGPSVFLFPPKPKDTLMISRTPEVTCVVVDVSHEDPEVKFNWYV |
| of Fc regions | | DGVEVHNAKTKPREEQYNSTYRVVSVLTVLHQDWLNGKEYKCKVSNK |
| (WO 2021/016571) | | ALPAPIEKTISKAKGQPREPQVYTLPPSRDELTKNQVSLTCLVKGFYPSDI |
| | | AVEWESNGQPENNYKTTPPVLDSDGSFFLYSKLTVDKSRWQQGNVFS |
| | | CSVMHEALHNHYTQKSLSLSPGK |
| Amino acid sequences | 171 | APEFLGGPSVFLFPPKPKDTLMISRTPEVTCVVVDVSQEDPEVQFNWYV |
| of Fc regions | | DGVEVHNAKTKPREEQFNSTYRVVSVLTVLHQDWLNGKEYKCKVSNK |
| (WO 2021/016571) | | GLPSSIEKTISKAKGQPREPQVYTLPPSQEEMTKNQVSLTCLVKGFYPSDI |
| | | AVEWESNGQPENNYKTTPPVLDSDGSFFLYSRLTVDKSRWQEGNVFSC |
| | | SVMHEALHNHYTQKSLSLSLGK |
| Amino acid sequences | 172 | APPVAGPSVFLFPPKPKDTLMISRTPEVTCVVVDVSHEDPEVQFNWYV |
| of Fc regions | | DGVEVHNAKTKPREEQFNSTFRVVSVLTVVHQDWLNGKEYKCKVSNK |
| (WO 2021/016571) | | GLPAPIEKTISKTKGQPREPQVYTLPPSREEMTKNQVSLTCLVKGFYPSDI |
| | | AVEWESNGQPENNYKTTPPMLDSDGSFFLYSKLTVDKSRWQQGNVFS |
| | | CSVMHEALHNHYTQKSLSLSPGK |
| Amino acid sequences | 164 | APELLGGPSVFLFPPKPKDTLMISRTPEVTCVVVDVSHEDPEVQFKWYV |
| of Fc regions | | DGVEVHNAKTKPREEQYNSTFRVVSVLTVLHQDWLNGKEYKCKVSNK |
| (WO 2021/016571) | | ALPAPIEKTISKTKGQPREPQVYTLPPSREEMTKNQVSLTCLVKGFYPSDI |
| | | AVEWESSGQPENNYNTTPPMLDSDGSFFLYSKLTVDKSRWQQGNIFSC |
| | | SVMHEALHNRFTQKSLSLSPGK |
| Amino acid sequences | 163 | CPPCPAPELLGGPSVFLFPPKPKDTLYITREPEVTCVVVDVSHEDPEVKF |
| of Fc region | | NWYVDGVEVHNAKTKPREEQYNSTYRVVSVLTVLHQDWLNGKEYKCK |
| | | VSNKALPAPIEKTISKAKGQPREPQVYTLPPSRDELTKNQVSLWCLVKGF |
| | | YPSDIAVEWESNGQPENNYKTTPPVLDSDGSFFLYSKLTVDKSRWQQG |
| | | NVFSCSVMHEALKFHYTQKSLSLSPG |
| Amino acid sequences | 179 | DKTHTCPPCPAPELLGGPSVFLFPPKPKDTLYITREPEVTCVVVDVSHED |
| of Fc region | | PEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLWC |
| | | LVKGFYPSDIAVEWESNGQPENNYKTTPPVLDSDGSFFLYSKLTVDKSR |
| | | WQQGNVFSCSVMHEALKFHYTQKSLSLSPG |
| Amino acid sequences | 183 | DKTHTCPPCPAPELLGGPSVFLFPPKPKDTLYITREPEVTCVVVDVSHED |
| of Fc region | | PEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLWC |
| | | LVKGFYPSDIAVEWESNGQPENNYKTTPPVLDSDGSFFLYSKLTVDKSR |
| | | WQQGNVFSCSVMHEALKFHYTQKSLSLSPGK |
| Amino acid sequences | 184 | CPPCPAPELLGGPSVFLFPPKPKDTLYITREPEVTCVVVDVSHEDPEVKF |
| of Fc region | | NWYVDGVEVHNAKTKPREEQYNSTYRVVSVLTVLHQDWLNGKEYKCK |
| | | VSNKALPAPIEKTISKAKGQPREPQVYTLPPSRDELTKNQVSLSCAVKGF |
| | | YPSDIAVEWESNGQPENNYKTTPPVLDSDGSFFLVSKLTVDKSRWQQG |
| | | NVFSCSVMHEALKFHYTQKSLSLSPG |
| Amino acid sequences | 185 | DKTHTCPPCPAPELLGGPSVFLFPPKPKDTLYITREPEVTCVVVDVSHED |
| of Fc region | | PEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLSCA |
| | | VKGFYPSDIAVEWESNGQPENNYKTTPPVLDSDGSFFLVSKLTVDKSR |
| | | WQQGNVFSCSVMHEALKFHYTQKSLSLSPG |
| Amino acid sequences | 211 | DKTHTCPPCPAPELLGGPSVFLFPPKPKDTLYITREPEVTCVVVDVSHED |
| of Fc region | | PEVKFNWYVDGVEVHNAKTKPREEQYNSTYRVVSVLTVLHQDWLNGK |
| | | EYKCKVSNKALPAPIEKTISKAKGQPREPQVYTLPPSRDELTKNQVSLSCA |
| | | VKGFYPSDIAVEWESNGQPENNYKTTPPVLDSDGSFFLVSKLTVDKSR |
| | | WQQGNVFSCSVMHEALKFHYTQKSLSLSPGK |

TABLE A-10
Antibodies and FcRn/antigen-binding molecules
(“ID” refers to the SEQ ID NO as used herein)

| Name | ID | Amino acid sequence |
|---|---|---|
| Heavy chain | 212 | DIQMTQSPSSLSASVGDRVTITCKSSQSLVGASGKTYLYWLFQKPGK |
| sequence of | | APKRLIYLVSTLDSGIPSRFSGSGSGTEFTLTISSLQPEDFATYYCLQGT |
| rozanolixizumab | | HFPHTFGQGTKLEIKRTVAAPSVFIFPPSDEQLKSGTASVVCLLNNFY |
| | | PREAKVQWKVDNALQSGNSQESVTEQDSKDSTYSLSSTLTLSKADY |
| | | EKHKVYACEVTHQGLSSPVTKSFNRGEC |
| Light chain | 213 | EVPLVESGGGLVQPGGSLRLSCAVSGFTFSNYGMVWVRQAPGKGL |
| sequence of | | EWVAYIDSDGDNTYYRDSVKGRFTISRDNAKSSLYLQMNSLRAEDT |
| rozanolixizumab | | AVYYCTTGIVRPFLYWGQGTLVTVSSASTKGPSVFPLAPCSRSTSEST |
| | | AALGCLVKDYFPEPVTVSWNSGALTSGVHTFPAVLQSSGLYSLSSVV |
| | | TVPSSSLGTKTYTCNVDHKPSNTKVDKRVESKYGPPCPPCPAPEFLG |
| | | GPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPS |
| | | DIAVEWESNGQPENNYKTTPPVLDSDGSFFLYSRLTVDKSRWQEG |
| | | NVFSCSVMHEALHNHYTQKSLSLSLGK |
| Heavy chain | 214 | DIQMTQSPSSLSASVGDRVTITCKASDHINNWLAWYQQKPGQAPR |
| sequence of | | LLISGATSLETGVPSRFSGSGTGKDYTLTISSLQPEDFATYYCQQYWS |
| orilanolimab | | TPYTFGGGTKVEIKRTVAAPSVFIFPPSDEQLKSGTASVVCLLNNFYP |
| | | REAKVQWKVDNALQSGNSQESVTEQDSKDSTYSLSSTLTLSKADYE |
| | | KHKVYACEVTHQGLSSPVTKSFNRGEC |
| Light chain | 215 | QVQLVQSGAELKKPGASVKLSCKASGYTFTSYGISWVKQATGQGLE |
| sequence of | | WIGEIYPRSGNTYYNEKFKGRATLTADKSTSTAYMELRSLRSEDSAV |
| orilanolimab | | YFCARSTTVRPPGIWGTGTTVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGG |
| | | PSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGL |
| | | PSSIEKTISKAKGQPREPQVYTLPPSQEEMTKNQVSLTCLVKGFYPSD |
| | | IAVEWESNGQPENNYKTTPPVLDSDGSFFLYSRLTVDKSRWQEGN |
| | | VFSCSVMHEALHNHYTQKSLSLSLG |
| Heavy chain | 216 | SYVLTQSPSVSVAPGQTARITCGGNNIGSKSVHWYQQKPGQAPVL |
| sequence of | | VVYDDSDRPSGIPERFSASNSGNTATLTISRVEAGDEADYYCQVWD |
| batoclimab | | SSSDHVVFGGGTKLTVLGQPKAAPSVTLFPPSSEELQANKATLVCLIS |
| | | DFYPGAVTVAWKADSSPVKAGVETTTPSKQSNNKYAASSYLSLTPE |
| | | QWKSHRSYSCQVTHEGSTVEKTVAPTECS |
| Light chain | 21 | QLLLQESGPGLVKPSETLSLTCTVSGGSLSSSFSYWVWIRQPPGKGL |
| sequence of | | EWIGTIYYSGNTYYNPSLKSRLTISVDTSKNHFSLKLSSVTAADTAVYY |
| batoclimab | | CARRAGILTGYLDSWGQGTLVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKRVEPKSCDKTHTCPPCPAPE |
| | | AAGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRW |
| | | QQGNVFSCSVMHEALHNHYTQKSLSLSPG |
| Heavy chain | 218 | QSALTQPASVSGSPGQSITISCTGTGSDVGSYNLVSWYQQHPGKAP |
| sequence of | | KLMIYGDSERPSGVSNRFSGSKSGNTASLTISGLQAEDEADYYCSSY |
| nipocalimab | | AGSGIYVFGTGTKVTVLGQPKAAPSVTLFPPSSEELQANKATLVCLIS |
| | | DFYPGAVTVAWKADSSPVKAGVETTTPSKQSNNKYAASSYLSLTPE |
| | | QWKSHKSYSCQVTHEGSTVEKTVAPTECS |
| Light chain | 219 | EVQLLESGGGLVQPGGSLRLSCAASGFTFSTYAMGWVRQAPGKGL |
| sequence of | | EWVSSIGASGSQTRYADSVKGRFTISRDNSKNTLYLQMNSLRAEDT |
| nipocalimab | | AVYYCARLAIGDSYWGQGTMVTVSSASTKGPSVFPLAPSSKSTSGG |
| | | TAALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCPAP |
| | | ELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVS |
| | | NKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRW |
| | | QQGNVFSCSVMHEALHNHYTQKSLSLSPG |
| FcRn/antigen- | 220 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RAFGMSWVRQAPGKGPEWVSSISGSGSDTLYADSVKGRFTISRDN |
| | | SKNTLYLQMNSLRPEDTAVYYCTIGGSLSRSSQGTLVTVSS |
| FcRn/antigen- | 221 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSAGMSWVRQAPGKGPEWVSSISGSGSDTLYADSVKGRFTISRDN |
| | | SKNTLYLQMNSLRPEDTAVYYCTIGGSLSRSSQGTLVTVSS |
| FcRn/antigen- | 222 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFAMSWVRQAPGKGPEWVSSISGSGSDTLYADSVKGRFTISRDNS |
| | | KNTLYLQMNSLRPEDTAVYYCTIGGSLSRSSQGTLVTVSS |
| FcRn/antigen- | 223 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGASWVRQAPGKGPEWVSSISGSGSDTLYADSVKGRFTISRDNS |
| | | KNTLYLQMNSLRPEDTAVYYCTIGGSLSRSSQGTLVTVSS |
| FcRn/antigen- | 224 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMAWVRQAPGKGPEWVSSISGSGSDTLYADSVKGRFTISRDN |
| | | SKNTLYLQMNSLRPEDTAVYYCTIGGSLSRSSQGTLVTVSS |
| FcRn/antigen- | 225 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSAISGSGSDTLYADSVKGRFTISRDN |
| | | SKNTLYLQMNSLRPEDTAVYYCTIGGSLSRSSQGTLVTVSS |
| FcRn/antigen- | 226 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSASGSGSDTLYADSVKGRFTISRDN |
| | | SKNTLYLQMNSLRPEDTAVYYCTIGGSLSRSSQGTLVTVSS |
| FcRn/antigen- | 227 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSIAGSGSDTLYADSVKGRFTISRDN |
| | | SKNTLYLQMNSLRPEDTAVYYCTIGGSLSRSSQGTLVTVSS |
| FcRn/antigen- | 228 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISASGSDTLYADSVKGRFTISRDNS |
| | | KNTLYLQMNSLRPEDTAVYYCTIGGSLSRSSQGTLVTVSS |
| FcRn/antigen- | 229 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGAGSDTLYADSVKGRFTISRDN |
| | | SKNTLYLQMNSLRPEDTAVYYCTIGGSLSRSSQGTLVTVSS |
| FcRn/antigen- | 230 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSASDTLYADSVKGRFTISRDNS |
| | | KNTLYLQMNSLRPEDTAVYYCTIGGSLSRSSQGTLVTVSS |
| FcRn/antigen- | 231 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGADTLYADSVKGRFTISRDN |
| | | SKNTLYLQMNSLRPEDTAVYYCTIGGSLSRSSQGTLVTVSS |
| FcRn/antigen- | 232 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSATLYADSVKGRFTISRDNS |
| | | KNTLYLQMNSLRPEDTAVYYCTIGGSLSRSSQGTLVTVSS |
| FcRn/antigen- | 233 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDALYADSVKGRFTISRDN |
| | | SKNTLYLQMNSLRPEDTAVYYCTIGGSLSRSSQGTLVTVSS |
| FcRn/antigen- | 234 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTAYADSVKGRFTISRDN |
| | | SKNTLYLQMNSLRPEDTAVYYCTIGGSLSRSSQGTLVTVSS |
| FcRn/antigen- | 235 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLAADSVKGRFTISRDN |
| | | SKNTLYLQMNSLRPEDTAVYYCTIGGSLSRSSQGTLVTVSS |
| FcRn/antigen- | 236 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYAASVKGRFTISRDNS |
| | | KNTLYLQMNSLRPEDTAVYYCTIGGSLSRSSQGTLVTVSS |
| FcRn/antigen- | 237 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADAVKGRFTISRDN |
| | | SKNTLYLQMNSLRPEDTAVYYCTIGGSLSRSSQGTLVTVSS |
| FcRn/antigen- | 238 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSAKGRFTISRDN |
| | | SKNTLYLQMNSLRPEDTAVYYCTIGGSLSRSSQGTLVTVSS |
| FcRn/antigen- | 239 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVAGRFTISRDN |
| | | SKNTLYLQMNSLRPEDTAVYYCTIGGSLSRSSQGTLVTVSS |
| FcRn/antigen- | 240 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKARFTISRDNS |
| | | KNTLYLQMNSLRPEDTAVYYCTIGGSLSRSSQGTLVTVSS |
| FcRn/antigen- | 241 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIAGSLSRSSQGTLVTVSS |
| FcRn/antigen- | 242 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGASLSRSSQGTLVTVSS |
| FcRn/antigen- | 243 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGGALSRSSQGTLVTVSS |
| FcRn/antigen- | 244 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGGSASRSSQGTLVTVSS |
| FcRn/antigen- | 245 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGGSLARSSQGTLVTVSS |
| FcRn/antigen- | 246 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGGSLSASSQGTLVTVSS |
| FcRn/antigen- | 247 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGGTLSRSSQGTLVTVSS |
| FcRn/antigen- | 248 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFAMSWVRQAPGKGPEWVSSISGSGSDTLYADSVKGRFTISRDNS |
| | | KNTLYLQMNSLRPEDTAVYYCTIGGALSRSSQGTLVTVSS |
| FcRn/antigen- | 249 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFAMSWVRQAPGKGPEWVSSISASGSDTLYADSVKGRFTISRDNS |
| | | KNTLYLQMNSLRPEDTAVYYCTIGGSLSRSSQGTLVTVSS |
| FcRn/antigen- | 250 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSIAASGSDTLYADSVKGRFTISRDN |
| | | SKNTLYLQMNSLRPEDTAVYYCTIGGSLSRSSQGTLVTVSS |
| FcRn/antigen- | 251 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSIAGSGSDTLYADSVKGRFTISRDN |
| | | SKNTLYLQMNSLRPEDTAVYYCTIGGALSRSSQGTLVTVSS |
| FcRn/antigen- | 252 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSHGMSWVRQAPGKGPEWVSSISGSGSDTLYADSVKGRFTISRDN |
| | | SKNTLYLQMNSLRPEDTAVYYCTIGGSLSRSSQGTLVTVSS |
| FcRn/antigen- | 253 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGHSWVRQAPGKGPEWVSSISGSGSDTLYADSVKGRFTISRDNS |
| | | KNTLYLQMNSLRPEDTAVYYCTIGGSLSRSSQGTLVTVSS |
| FcRn/antigen- | 254 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSHSDTLYADSVKGRFTISRDNS |
| | | KNTLYLQMNSLRPEDTAVYYCTIGGSLSRSSQGTLVTVSS |
| FcRn/antigen- | 255 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTHYADSVKGRFTISRDN |
| | | SKNTLYLQMNSLRPEDTAVYYCTIGGSLSRSSQGTLVTVSS |
| FcRn/antigen- | 256 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIHGSLSRSSQGTLVTVSS |
| FcRn/antigen- | 257 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGHSLSRSSQGTLVTVSS |
| FcRn/antigen- | 258 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFGMSWVRQAPGKGPEWVSSISGSGSDTLYADSVKGRFTISRDN |
| | | SKNTLYLQMNSLRPEDTAVYYCTIGGSHSRSSQGTLVTVSS |
| FcRn/antigen- | 259 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSEVQLLESGGGLVQPGGSLRLSCAASGFTF |
| | | RSFHMSWVRQAPGKGPEWVSSISGSGSDTLYADSVKGRFTISRDNS |
| | | KNTLYLQMNSLRPEDTAVYYCTIGGHLSRSSQGTLVTVSS |
| FcRn/antigen- | 260 | DKTHTCPPCPAPELLGGPSVFLFPPKPKDTLYITREPEVTCVVVDVSH |
| binding molecule | | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFF |
| | | LYSKLTVDKSRWQQGNVFSCSVMHEALKFHYTQKSLSLSPGGGGG |
| | | SGGGGSGGGGSGGGGSGGGGSGGGGSEVQLLESGGGLVQPGGS |
| | | LRLSCAASGFTFRSAGMSWVRQAPGKGPEWVSSISGSGSDTLYADS |
| | | VKGRFTISRDNSKNTLYLQMNSLRPEDTAVYYCTIGGSLSRSSQGTL |
| | | VTVSSA |

TABLE A-7
Linker sequences
(“ID” refers to the SEQ ID NO as used herein)

| Name | ID | Amino acid sequence |
|---|---|---|
| 3A linker | 25 | AAA |
| 5GS linker | 26 | GGGGS |
| 7GS linker | 27 | SGGSGGS |
| 8GS linker | 28 | GGGGSGGS |
| 9GS linker | 29 | GGGGSGGGS |
| 10GS linker | 30 | GGGGSGGGGS |
| 15GS linker | 31 | GGGGSGGGGSGGGGS |
| 18GS linker | 32 | GGGGSGGGGSGGGGSGGS |
| 20GS linker | 33 | GGGGSGGGGSGGGGSGGGGS |
| 25GS linker | 34 | GGGGSGGGGSGGGGSGGGGSGGGGS |
| 30GS linker | 35 | GGGGSGGGGSGGGGSGGGGSGGGGS |
| | | GGGGS |
| 35GS linker | 36 | GGGGSGGGGSGGGGSGGGGSGGGGS |
| | | GGGGSGGGGS |
| 40GS linker | 37 | GGGGGGGGSGGGGSGGGGSGGGGSG |
| | | GGGSGGGGSGGGGS |
| Short G1 hinge | 38 | DKTHTCPPCP |
| G1 hinge | 39 | EPKSCDKTHTCPPCP |
| 9GS-G1 hinge | 40 | GGGGSGGGSEPKSCDKTHTCPPCP |
| Llama upper long | 41 | EPKTPKPQPAAA |
| hinge region | | |
| G3 hinge | 42 | ELKTPLGDTTHTCPRCPEPKSCDTP |
| | | PPCPRCPEPKSCDTPPPCPRCPEPK |
| | | SCDTPPPCPRCP |
| Short hinge linker | 200 | DKTHTCPSCP |
| MMP linkers | 125 | GPLGMWSR |
| MMP linkers | 126 | GPLGVR |
| Mouse lower hinge | 131 | CPPCKCPAPNLLGGP |
| sequence | | |
| Core hinge-lower | 132 | CPPCPAPELLGGP |
| hinge/upper CH | | |
| MMP substrate | 111 | GPLGMWSRAAQPA |
| Masking domain | 134 | PRGPTIKPCPPCKCP |
| MMP substrate | 135 | GPLGMWSRGAQPA |

TABLE A-11
Construct sequences (“ID” refers to the SEQ ID NO as used herein)

| Name | ID | Amino acid sequence |
|---|---|---|
| T-0263-00_TP108 | 119 | DVQLVESGGGLVQAGGSLRLSCAASGITFSINTMGWYRQAPGKQRELVALISSI |
| (Chain 1) | | GDTYYADSVKGRFTISRDNAKNTVYLQMNSLKPEDTAVYYCKRFRTAAQGTDY |
| | | WGQGTLVTVSSDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLN |
| | | GKEYKCKVSNKALPAPIEKTISKAKGQPREPQVYTLPPCRDELTKNQVSLWCLVK |
| | | GFYPSDIAVEWESNGQPENNYKTTPPVLDSDGSFFLYSKLTVDKSRWQQGNVF |
| | | SCSVMHEALHNHYTQKSLSLSPGGGGGSGGGSEVQLVESGGGVVQAGGSLSIS |
| | | CAASGGSLSNYVLGWFRQAPGKEREFVAAINWRGDITIGPPNVEGRFTISRDNA |
| | | KNTGYLQMNSLAPDDTALYYCGAGTPLNPGAYIYDWSYDYWGRGTLVTVSSA |
| T-0263-00_TP108 | 120 | DVQLVESGGGVVQAGGSLSISCAASGGSLSNYVLGWFRQAPGKEREFVAAIN |
| (Chain 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPELLGGPSVFLFPPKPKDTLMIS |
| | | RTPEVTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVCTLPPSRDELTKN |
| | | QVSLSCAVKGFYPSDIAVEWESNGQPENNYKTTPPVLDSDGSFFLVSKLTVDKS |
| | | RWQQGNVFSCSVMHEALHNRFTQKSLSLSPG |
| T-0263-00_TP111 | 121 | DVQLVESGGGLVQAGGSLRLSCAASGITFSINTMGWYRQAPGKQRELVALISSI |
| (Chain 1) | | GDTYYADSVKGRFTISRDNAKNTVYLQMNSLKPEDTAVYYCKRFRTAAQGTDY |
| | | WGQGTLVTVSSDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLN |
| | | GKEYKCKVSNKALPAPIEKTISKAKGQPREPQVYTLPPCRDELTKNQVSLWCLVK |
| | | GFYPSDIAVEWESNGQPENNYKTTPPVLDSDGSFFLYSKLTVDKSRWQQGNVF |
| | | SCSVMHEALHNHYTQKSLSLSPGGGGGSGGGSEVQLVESGGGVVQPGGSLRL |
| | | SCAASGFTFRSFGMSWVRQAPGKGPEWVSSISGSGSDTLYADSVKGRFTISRD |
| | | NSKNTLYLQMNSLRPEDTALYYCTIGGSLSRSSQGTLVTVSSA |
| T-0263-00_TP111 | 122 | DVQLVESGGGVVQAGGSLSISCAASGGSLSNYVLGWFRQAPGKEREFVAAIN |
| (Chain 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPELLGGPSVFLFPPKPKDTLMIS |
| | | RTPEVTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVCTLPPSRDELTKN |
| | | QVSLSCAVKGFYPSDIAVEWESNGQPENNYKTTPPVLDSDGSFFLVSKLTVDKS |
| | | RWQQGNVFSCSVMHEALHNRFTQKSLSLSPG |
| T-0263-00_TP113 | 123 | DVQLVESGGGLVQAGGSLRLSCAASGITFSINTMGWYRQAPGKQRELVALISSI |
| (Chain 1) | | GDTYYADSVKGRFTISRDNAKNTVYLQMNSLKPEDTAVYYCKRFRTAAQGTDY |
| | | WGQGTLVTVSSDKTHTCPPCPAPELLGGPSVFLFPPKPKDTLMISRDPEVTCVV |
| | | VDVSHEDPEVKFNWYVDGVEVHNAKTKPREEQYNSTYRVVSVLQVLHQDWL |
| | | NGKEYKCKVSNKALPAPIEKTISKAKGQPREPQVYTLPPCRDELTKNQVSLWCLV |
| | | KGFYPSDIAVEWESNGQPENNYKTTPPVLDSDGSFFLYSKLTVDKSRWQQGNV |
| | | FSCSVMHEALHNHYTQKSLSLSPGGGGGSGGGSEVQLVESGGGVVQAGGSLSI |
| | | SCAASGGSLSNYVLGWFRQAPGKEREFVAAINWRGDITIGPPNVEGRFTISRDN |
| | | AKNTGYLQMNSLAPDDTALYYCGAGTPLNPGAYIYDWSYDYWGRGTLVTVSS |
| | | A |
| T-0263-00_TP113 | 124 | DVQLVESGGGVVQAGGSLSISCAASGGSLSNYVLGWFRQAPGKEREFVAAIN |
| (Chain 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPELLGGPSVFLFPPKPKDTLMIS |
| | | RDPEVTCVVVDVSHEDPEVKFNWYVDGVEVHNAKTKPREEQYNSTYRVVSVL |
| | | QVLHQDWLNGKEYKCKVSNKALPAPIEKTISKAKGQPREPQVCTLPPSRDELTK |
| | | NQVSLSCAVKGFYPSDIAVEWESNGQPENNYKTTPPVLDSDGSFFLVSKLTVDK |
| | | SRWQQGNVFSCSVMHEALHNRFTQKSLSLSPG |
| T-0263-00_TP117 | 127 | EVQLVESGGGLVQPGGSLRLSCAASGFNIRDTYIHWVRQAPGKGLEWVARIYP |
| (Chain 1) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVYTLPPCRDELTKNQVSLWCLVKGFY |
| | | PSDIAVEWESNGQPENNYKTTPPVLDSDGSFFLYSKLTVDKSRWQQGNVFSCS |
| | | VMHEALHNHYTQKSLSLSPG |
| T-0263-00_TP117 | 128 | EVQLVESGGGLVQPGGSLRLSCAASGFNIRDTYIHWVRQAPGKGLEWVARIYP |
| (Chain 2) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVCTLPPSRDELTKNQVSLSCAVKGFYP |
| | | SDIAVEWESNGQPENNYKTTPPVLDSDGSFFLVSKLTVDKSRWQQGNVFSCSV |
| | | MHEALHNRFTQKSLSLSPG |
| T-0263-00_TP117 | 129 | DIQMTQSPSSLSASVGDRVTITCRASQDVNTAVAWYQQKPGKAPKLLIYSASFL |
| (Chain 3) | | YSGVPSRFSGSRSGTDFTLTISSLQPEDFATYYCQQHYTTPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| T-0263-00_TP118 | 130 | EVQLVESGGGLVQPGGSLRLSCAASGFNIRDTYIHWVRQAPGKGLEWVARIYP |
| (Chain 1) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVYTLPPCRDELTKNQVSLWCLVKGFY |
| | | PSDIAVEWESNGQPENNYKTTPPVLDSDGSFFLYSKLTVDKSRWQQGNVFSCS |
| | | VMHEALHNHYTQKSLSLSPGGGGGSGGGSEVQLVESGGGVVQAGGSLSISCA |
| | | ASGGSLSNYVLGWFRQAPGKEREFVAAINWRGDITIGPPNVEGRFTISRDNAK |
| | | NTGYLQMNSLAPDDTALYYCGAGTPLNPGAYIYDWSYDYWGRGTLVTVSSA |
| T-0263-00_TP118 | 128 | EVQLVESGGGLVQPGGSLRLSCAASGFNIRDTYIHWVRQAPGKGLEWVARIYP |
| (Chain 2) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVCTLPPSRDELTKNQVSLSCAVKGFYP |
| | | SDIAVEWESNGQPENNYKTTPPVLDSDGSFFLVSKLTVDKSRWQQGNVFSCSV |
| | | MHEALHNRFTQKSLSLSPG |
| T-0263-00_TP118 | 129 | DIQMTQSPSSLSASVGDRVTITCRASQDVNTAVAWYQQKPGKAPKLLIYSASFL |
| (Chain 3) | | YSGVPSRFSGSRSGTDFTLTISSLQPEDFATYYCQQHYTTPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| T-0263-00_TP121 | 133 | EVQLVESGGGLVQPGGSLRLSCAASGFNIRDTYIHWVRQAPGKGLEWVARIYP |
| (Chain 1) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVYTLPPCRDELTKNQVSLWCLVKGFY |
| | | PSDIAVEWESNGQPENNYKTTPPVLDSDGSFFLYSKLTVDKSRWQQGNVFSCS |
| | | VMHEALHNHYTQKSLSLSPGGGGGSGGGSEVQLVESGGGVVQPGGSLRLSCA |
| | | ASGFTFRSFGMSWVRQAPGKGPEWVSSISGSGSDTLYADSVKGRFTISRDNSK |
| | | NTLYLQMNSLRPEDTALYYCTIGGSLSRSSQGTLVTVSSA |
| T-0263-00_TP121 | 128 | EVQLVESGGGLVQPGGSLRLSCAASGFNIRDTYIHWVRQAPGKGLEWVARIYP |
| (Chain 2) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVCTLPPSRDELTKNQVSLSCAVKGFYP |
| | | SDIAVEWESNGQPENNYKTTPPVLDSDGSFFLVSKLTVDKSRWQQGNVFSCSV |
| | | MHEALHNRFTQKSLSLSPG |
| T-0263-00_TP121 | 129 | DIQMTQSPSSLSASVGDRVTITCRASQDVNTAVAWYQQKPGKAPKLLIYSASFL |
| (Chain 3) | | YSGVPSRFSGSRSGTDFTLTISSLQPEDFATYYCQQHYTTPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| T-0263-00_TP123 | 136 | EVQLVESGGGLVQPGGSLRLSCAASGFNIRDTYIHWVRQAPGKGLEWVARIYP |
| (Chain 1) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVYTLPPCRDELTKNQVSLWCLVKGFY |
| | | PSDIAVEWESNGQPENNYKTTPPVLDSDGSFFLYSKLTVDKSRWQQGNVFSCS |
| | | VMHEALHNHYTQKSLSLSPGGGGGSGGGSEVQLVESGGGVVQPGGSLRLSCA |
| | | ASGFTFRSFGMSWVRQAPGKGPEWVSSISGSGSDTLYADSVKGRFTISRDNSK |
| | | NTLYLQMNSLRPEDTALYYCTIGGSLSRSSQGTLVTVSSA |
| T-0263-00_TP123 | 137 | EVQLVESGGGLVQPGGSLRLSCAASGFNIRDTYIHWVRQAPGKGLEWVARIYP |
| (Chain 2) | | TQGYTRYADSVKGRFTISADTSKNTAYLQMNSLRAEDTAVYYCSRWGGEGFYA |
| | | MDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKKVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKE |
| | | YKCKVSNKALPAPIEKTISKAKGQPREPQVCTLPPSRDELTKNQVSLSCAVKGFYP |
| | | SDIAVEWESNGQPENNYKTTPPVLDSDGSFFLVSKLTVDKSRWQQGNVFSCSV |
| | | MHEALHNRFTQKSLSLSPGGGGGSGGGSEVQLVESGGGVVQPGGSLRLSCAA |
| | | SGFTFRSFGMSWVRQAPGKGPEWVSSISGSGSDTLYADSVKGRFTISRDNSKN |
| | | TLYLQMNSLRPEDTALYYCTIGGSLSRSSQGTLVTVSSA |
| T-0263-00_TP123 | 129 | DIQMTQSPSSLSASVGDRVTITCRASQDVNTAVAWYQQKPGKAPKLLIYSASFL |
| (Chain 3) | | YSGVPSRFSGSRSGTDFTLTISSLQPEDFATYYCQQHYTTPPTFGQGTKVEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| TPP-66143 (Chain | 139 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLGGGGGSGGGGSGGGGSGGGG |
| | | SGGGGSGGGGSGGGGSEVQLVESGGGVVQAGGSLSISCAASGGSLSNYVLGW |
| | | FRQAPGKEREFVAAINWRGDITIGPPNVEGRFTISRDNAKNTGYLQMNSLAPD |
| | | DTALYYCGAGTPLNPGAYIYDWSYDYWGRGTLVTVSSA |
| TPP-66143 (Chain | 140 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLG |
| TPP-66144 (Chain | 141 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLGGGGGSGGGSEVQLVESGGGV |
| | | VQPGGSLRLSCAASGFTFRSFGMSWVRQAPGKGPEWVSSISGSGSDTLYADSV |
| | | KGRFTISRDNSKNTLYLQMNSLRPEDTALYYCTIGGSLSRSSQGTLVTVSSA |
| TPP-66144 (Chain | 140 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLG |
| TPP-66145 (Chain | 143 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLGGGGGSGGGGSGGGGSGGGG |
| | | SGGGGSGGGGSGGGGSEVQLVESGGGLVQPGNSLRLSCAASGFTFRSFGMS |
| | | WVRQAPGKEPEWVSSISGSGSDTLYADSVKGRFTISRDNAKTTLYLQMNSLKPE |
| | | DTAVYYCTIGGSLSRSSQGTQVTVSSA |
| TPP-66145 (Chain | 140 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLG |
| TPP-66146 (Chain | 145 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLGGGGGSGGGGSGGGGSGGGG |
| | | SGGGGSGGGGSGGGGSEVQLVESGGGVVQPGNSLRLSCAASGFTFSSFGMS |
| | | WVRQAPGKGLEWVSSISGSGSDTLYADSVKGRFTISRDNAKTTLYLQMNSLRPE |
| | | DTALYYCTIGGSLSRSSQGTLVTVSSA |
| TPP-66146 (Chain | 140 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLG |
| TPP-66147 (Chain | 147 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLGGGGGSGGGGSGGGGSGGGG |
| | | SGGGGSGGGGSGGGGSEVQLVESGGGVVQPGGSLRLSCAASGFTFRSFGMS |
| | | WVRQAPGKGPEWVSSISGSGSDTLYADSVKGRFTISRDNSKNTLYLQMNSLRP |
| | | EDTALYYCTIGGSLSRSSQGTLVTVSSA |
| TPP-66147 (Chain | 140 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLG |
| TPP-66148 (Chain | 149 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLGGGGGSGGGGSGGGGSGGGG |
| | | SGGGGSGGGGSGGGGSEVQLVESGGGVVQPGGSLRLSCAASGLTFSSYAMG |
| | | WFRQAPGKERERVVSISRGGGYTYYADSVKGRFTISRDNSKNTVYLQMNSLRPE |
| | | DTALYYCAAARYWATGSEYEFDYWGQGTLVTVSSA |
| TPP-66148 (Chain | 140 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLG |
| TPP-66149 (Chain | 151 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLGGGGGSGGGGSGGGGSGGGG |
| | | SGGGGSGGGGSGGGGSEVQLVESGGGVVQPGDSLRLSCAASGGTFSTYVMG |
| | | WFRQAPGKEREFVSAISQNSIHTYYANSVKGRFTISRDNSKNTVYLQLNSLRPED |
| | | TALYYCAASRFTSWYTADYEYDYWGQGTLVTVSSA |
| TPP-66149 (Chain | 140 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLG |
| TPP-66150 (Chain | 153 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLG |
| TPP-66150 (Chain | 154 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLGGGGGSGGGGSGGGGSGGGG |
| | | SGGGGSGGGGSGGGGSDLGKKLLEAARAGQDDEVRELLKAGADVNAKDYFSH |
| | | TPLHLAARNGHLKIVEVLLKAGADVNAKDFAGKTPLHLAANEGHLEIVEVLLKAG |
| | | ADVNAQDIFGKTPADIAADAGHEDIAEVLQKAA |
| TPP-66151 (Chain | 153 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLG |
| TPP-66151 (Chain | 156 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLGGGGGSGGGGSGGGGSGGGG |
| | | SGGGGSGGGGSGGGGSVKVKFWPRGEEKVVDTSKIAWVLRADKTVMFKYDD |
| | | NGKKGYGVVLEKDAPKELLDMLARAEREK |
| TPP-66152 (Chain | 153 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLG |
| TPP-66152 (Chain | 158 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLGGGGGSGGGGSGGGGSGGGG |
| | | SGGGGSGGGGSGGGGSLKEAKEKAIEELKKAGITSDYYFDLINKAKTVEGVNALK |
| | | DEILKA |
| TPP-66153 (Chain | 153 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLG |
| TPP-66153 (Chain | 160 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLGGGGGSGGGGSGGGGSGGGG |
| | | SGGGGSGGGGSGGGGSDAHKSEVAHRFKDLGEENFKALVLIAFAQYLQQCPFE |
| | | DHVKLVNEVTEFAKTCVADESAENCDKSLHTLFGDKLCTVATLRETYGEMADCC |
| | | AKQEPERNECFLQHKDDNPNLPRLVRPEVDVMCTAFHDNEETFLKKYLYEIARR |
| | | HPYFYAPELLFFAKRYKAAFTECCQAADKAACLLPKLDELRDEGKASSAKQRLKC |
| | | ASLQKFGERAFKAWAVARLSQRFPKAEFAEVSKLVTDLTKVHTECCHGDLLECA |
| | | DDRADLAKYICENQDSISSKLKECCEKPLLEKSHCIAEVENDEMPADLPSLAADFV |
| | | ESKDVCKNYAEAKDVFLGMFLYEYARRHPDYSVVLLLRLAKTYETTLEKCCAAAD |
| | | PHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVT |
| | | KCCTESLVNRRPCFSALEVDETYVPKEFNAETFTFHADICTLSEKERQIKKQTALV |
| | | ELVKHKPKATKEQLKAVMDDFAAFVEKCCKADDKETCFAEEGKKLVAASQAAL |
| | | GL |
| TPP-66154 (Chain | 153 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPCQEEMTK |
| | | NQVSLWCLVKGFYPSDIAVEWESNGQPENNYKTTPPVLDSDGSFFLYSKLTVDK |
| | | SRWQEGNVFSCSVMHEALHNHYTQKSLSLSLG |
| TPP-66154 (Chain | 162 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVCTLPPSQEEMTK |
| | | NQVSLSCAVKGFYPSDIAVEWESNGQPENNYKTTPPVLDSDGSFFLVSKLTVDK |
| | | SRWQEGNVFSCSVMHEALHNRFTQKSLSLSLGGGGGSGGGGSGGGGSGGGG |
| | | SGGGGSGGGGSGGGGSDAHKSEVAHRFKDLGEENFKALVLIAFAQYLQQCPFE |
| | | DHVKLVNEVTEFAKTCVADESAENCDKSLHTLFGDKLCTVATLRETYGEMADCC |
| | | AKQEPERNECFLQHKDDNPNLPRLVRPEVDVMCTAFHDNEETFLKKYLYEIARR |
| | | HPYFYAPELLFFAKRYKAAFTECCQAADKAACLLPKLDELRDEGKASSAKQRLKC |
| | | ASLQKFGERAFKAWAVARLSQRFPKAEFAEVSKLVTDLTKVHTECCHGDLLECA |
| | | DDRADLAKYICENQDSISSKLKECCEKPLLEKSHCIAEVENDEMPADLPSLAADFV |
| | | ESKDVCKNYAEAKDVFLGMFLYEYARRHPDYSVVLLLRLAKTYETTLEKCCAAAD |
| | | PHECYAKVFDEFKPLVEEPQNLIKQNCELFEQLGEYKFQNALLVRYTKKVPQVST |
| | | PTLVEVSRNLGKVGSKCCKHPEAKRMPCAEDYLSVVLNQLCVLHEKTPVSDRVT |
| | | KCCTESLVNRRPCFSALEVDETYVPKEFNAQTFTFHADICTLSEKERQIKKQMAL |
| | | VELVKHKPKATKEQLKAVMDDFAAFVEKCCKADDKETCFAEEGPKLVAASQAA |
| | | LGL |
| TPP-66156 (Chain | 165 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRDPEVTCVVVDVSQEDPEVQFNWYVDGVEVHNAKTKPREEQFNSTYRVVSV |
| | | LQVLHQDWLNGKEYKCKVSNKGLPSSIEKTISKAKGQPREPQVYTLPPCQEEMT |
| | | KNQVSLWCLVKGFYPSDIAVEWESNGQPENNYKTTPPVLDSDGSFFLYSKLTVD |
| | | KSRWQEGNVFSCSVMHEALHNHYTQKSLSLSLGGGGGSGGGGSGGGGSGGG |
| | | GSGGGGGGGGSGGGGSEVQLVESGGGVVQPGGSLRLSCAASGFTFRSFGMS |
| | | WVRQAPGKGPEWVSSISGSGSDTLYADSVKGRFTISRDNSKNTLYLQMNSLRP |
| | | EDTALYYCTIGGSLSRSSQGTLVTVSSA |
| TPP-66156 (Chain | 166 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLMI |
| | | SRDPEVTCVVVDVSQEDPEVQFNWYVDGVEVHNAKTKPREEQFNSTYRVVSV |
| | | LQVLHQDWLNGKEYKCKVSNKGLPSSIEKTISKAKGQPREPQVCTLPPSQEEMT |
| | | KNQVSLSCAVKGFYPSDIAVEWESNGQPENNYKTTPPVLDSDGSFFLVSKLTVD |
| | | KSRWQEGNVFSCSVMHEALHNRFTQKSLSLSLG |
| TPP-66174 (Chain | 173 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLYIT |
| | | REPEVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPCQEEMTKN |
| | | QVSLWCLVKGFYPSDIAVEWESNGQPENNYKTTPPVLDSDGSFFLYSKLTVDKS |
| | | RWQEGNVFSCSVMHEALHNHYTQKSLSLSLGGGGGSGGGGSGGGGSGGGGS |
| | | GGGGSGGGGSGGGGSEVQLVESGGGVVQPGGSLRLSCAASGFTFRSFGMSW |
| | | VRQAPGKGPEWVSSISGSGSDTLYADSVKGRFTISRDNSKNTLYLQMNSLRPED |
| | | TALYYCTIGGSLSRSSQGTLVTVSSA |
| TPP-66174 (Chain | 174 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLYIT |
| | | REPEVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVCTLPPSQEEMTKN |
| | | QVSLSCAVKGFYPSDIAVEWESNGQPENNYKTTPPVLDSDGSFFLVSKLTVDKS |
| | | RWQEGNVFSCSVMHEALHNRFTQKSLSLSLG |
| TPP-66175 (Chain | 175 | DVQLVESGGGVVQAGGSLSISCAASGGSLSNYVLGWFRQAPGKEREFVAAIN |
| 1) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLYIT |
| | | REPEVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPCQEEMTKN |
| | | QVSLWCLVKGFYPSDIAVEWESNGQPENNYKTTPPVLDSDGSFFLYSKLTVDKS |
| | | RWQEGNVFSCSVMHEALHNHYTQKSLSLSLGGGGGSGGGGSGGGGGGGGS |
| | | GGGGSGGGGSGGGGSEVQLVESGGGVVQAGGSLSISCAASGGSLSNYVLGWF |
| | | RQAPGKEREFVAAINWRGDITIGPPNVEGRFTISRDNAKNTGYLQMNSLAPDD |
| | | TALYYCGAGTPLNPGAYIYDWSYDYWGRGTLVTVSSA |
| TPP-66175(Chain | 174 | DVQLVESGGGVVQAGGSLSISCAASGGSLSNYVLGWFRQAPGKEREFVAAIN |
| 2) | | WRGDITIGPPNVEGRFTISRDNAKNTGYLQMNSLAPDDTALYYCGAGTPLNPG |
| | | AYIYDWSYDYWGRGTLVTVSSDKTHTCPPCPAPEAAGGPSVFLFPPKPKDTLYIT |
| | | REPEVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVCTLPPSQEEMTKN |
| | | QVSLSCAVKGFYPSDIAVEWESNGQPENNYKTTPPVLDSDGSFFLVSKLTVDKS |
| | | RWQEGNVFSCSVMHEALHNRFTQKSLSLSLG |
| TPP-66176 | 177 | DVQLVESGGGVVQAGGSLSISCAASGGSLSNYVLGWFRQAPGKEREFVAAIN |
| | | WRGDITIGPPNVEGRFTISRDNAKNTGYLQMNSLAPDDTALYYCGAGTPLNPG |
| | | AYIYDWSYDYWGRGTLVTVSSDKTHTCPSCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSKLTVDKS |
| | | RWQEGNVFSCSVMHEALHNHYTQKSLSLSLGGGGGSGGGGSGGGGSGGGGS |
| | | GGGGSGGGGSGGGGSEVQLVESGGGVVQAGGSLSISCAASGGSLSNYVLGWF |
| | | RQAPGKEREFVAAINWRGDITIGPPNVEGRFTISRDNAKNTGYLQMNSLAPDD |
| | | TALYYCGAGTPLNPGAYIYDWSYDYWGRGTLVTVSSA |
| TPP-66177 | 178 | DVQLVESGGGVVQAGGSLSISCAASGGSLSNYVLGWFRQAPGKEREFVAAIN |
| | | WRGDITIGPPNVEGRFTISRDNAKNTGYLQMNSLAPDDTALYYCGAGTPLNPG |
| | | AYIYDWSYDYWGRGTLVTVSSDKTHTCPSCPAPEAAGGPSVFLFPPKPKDTLMI |
| | | SRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSKLTVDKS |
| | | RWQEGNVFSCSVMHEALHNHYTQKSLSLSLGGGGGSGGGGSGGGGSGGGGS |
| | | GGGGSGGGGSGGGGSEVQLVESGGGVVQPGGSLRLSCAASGFTFRSFGMSW |
| | | VRQAPGKGPEWVSSISGSGSDTLYADSVKGRFTISRDNSKNTLYLQMNSLRPED |
| | | TALYYCTIGGSLSRSSQGTLVTVSSA |
| TP003 Chain 1 | 201 | DVQLVESGGGLVQPGGSLRLSCAASGLTFSTNPMYWYRQAPGKQRELVASISS |
| | | RGITNYADSVKGRFTISRDNSKNTVYLQMNSLRPEDTAVYYCRLASLSSGTVYW |
| | | GQGTLVTVSSDKTHTCPPCPAPEAAGGPSVFLFPPKPKDTLMISRTPEVTCVVV |
| | | DVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPCQEEMTKNQVSLWCLV |
| | | KGFYPSDIAVEWESNGQPENNYKTTPPVLDSDGSFFLYSKLTVDKSRWQEGNV |
| | | FSCSVMHEALHNHYTQKSLSLSLG |
| TP003 Chain 2 | 202 | DVQLVESGGGLVQPGGSLRLSCAASGLTFSTNPMYWYRQAPGKQRELVASISS |
| | | RGITNYADSVKGRFTISRDNSKNTVYLQMNSLRPEDTAVYYCRLASLSSGTVYW |
| | | GQGTLVTVSSDKTHTCPPCPAPEAAGGPSVFLFPPKPKDTLMISRTPEVTCVVV |
| | | DVSQEDPEVQFNWYVDGVEVHNAKTKPREEQFNSTYRVVSVLTVLHQDWLN |
| | | GKEYKCKVSNKGLPSSIEKTISKAKGQPREPQVCTLPPSQEEMTKNQVSLSCAVK |
| | | GFYPSDIAVEWESNGQPENNYKTTPPVLDSDGSFFLVSKLTVDKSRWQEGNVF |
| | | SCSVMHEALHNHYTQKSLSLSLG |
| TP006 Chain 1 | 203 | DVQLVESGGGLVQPGGSLRLSCAASGLTFSTNPMYWYRQAPGKQRELVASISS |
| | | RGITNYADSVKGRFTISRDNSKNTVYLQMNSLRPEDTAVYYCRLASLSSGTVYW |
| | | GQGTLVTVSSDKTHTCPPCPAPEAAGGPSVFLFPPKPKDTLMISRTPEVTCVVV |
| | | DVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPCQEEMTKNQVSLWCLV |
| | | KGFYPSDIAVEWESNGQPENNYKTTPPVLDSDGSFFLYSKLTVDKSRWQEGNV |
| | | FSCSVMHEALHNHYTQKSLSLSLGGGGGSGGGGSGGGGSGGGGSGGGGSGG |
| | | GGSGGGGSEVQLVESGGGVVQPGGSLRLSCAASGFTFRSFGMSWVRQAPGK |
| | | GPEWVSSISGSGSDTLYADSVKGRFTISRDNSKNTLYLQMNSLRPEDTALYYCTI |
| | | GGSLSRSSQGTLVTVSS |
| TP006 Chain 2 | 204 | DKTHTCPPCPAPEAAGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCKVSNK |
| | | GLPSSIEKTISKAKGQPREPQVCTLPPSQEEMTKNQVSLSCAVKGFYPSDIAVEW |
| | | ESNGQPENNYKTTPPVLDSDGSFFLVSKLTVDKSRWQEGNVFSCSVMHEALHN |
| | | HYTQKSLSLSLG |
| TP008 | 205 | DVQLVESGGGLVQPGGSLRLSCAASGLTFSTNPMYWYRQAPGKQRELVASISS |
| Chain 1 | | RGITNYADSVKGRFTISRDNSKNTVYLQMNSLRPEDTAVYYCRLASLSSGTVYW |
| | | GQGTLVTVSSDKTHTCPPCPAPEAAGGPSVFLFPPKPKDTLMISRTPEVTCVVV |
| | | DVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPCQEEMTKNQVSLWCLV |
| | | KGFYPSDIAVEWESNGQPENNYKTTPPVLDSDGSFFLYSKLTVDKSRWQEGNV |
| | | FSCSVMHEALHNHYTQKSLSLSLGGGGGSGGGGSGGGGSGGGGSGGGGSGG |
| | | GGSGGGGSEVQLVESGGGLVQPGGSLRLSCAASGLTFSTNPMYWYRQAPGKQ |
| | | RELVASISSRGITNYADSVKGRFTISRDNSKNTVYLQMNSLRPEDTAVYYCRLASL |
| | | SSGTVYWGQGTLVTVSS |
| TP008 | 204 | DKTHTCPPCPAPEAAGGPSVFLFPPKPKDTLMISRTPEVTCVVVDVSQEDPEVQ |
| Chain 2 | | FNWYVDGVEVHNAKTKPREEQFNSTYRVVSVLTVLHQDWLNGKEYKCKVSNK |
| | | GLPSSIEKTISKAKGQPREPQVCTLPPSQEEMTKNQVSLSCAVKGFYPSDIAVEW |
| | | ESNGQPENNYKTTPPVLDSDGSFFLVSKLTVDKSRWQEGNVFSCSVMHEALHN |
| | | HYTQKSLSLSLG |
| TP009 | 201 | DVQLVESGGGLVQPGGSLRLSCAASGLTFSTNPMYWYRQAPGKQRELVASISS |
| Chain 1 | | RGITNYADSVKGRFTISRDNSKNTVYLQMNSLRPEDTAVYYCRLASLSSGTVYW |
| | | GQGTLVTVSSDKTHTCPPCPAPEAAGGPSVFLFPPKPKDTLMISRTPEVTCVVV |
| | | DVSQEDPEVQFNWYVDGVEVHNAKTKPREEQFNSTYRVVSVLTVLHQDWLN |
| | | GKEYKCKVSNKGLPSSIEKTISKAKGQPREPQVYTLPPCQEEMTKNQVSLWCLV |
| | | KGFYPSDIAVEWESNGQPENNYKTTPPVLDSDGSFFLYSKLTVDKSRWQEGNV |
| | | FSCSVMHEALHNHYTQKSLSLSLG |
| TP009 | 206 | DVQLVESGGGVVQPGGSLRLSCAASGFTFRSFGMSWVRQAPGKGPEWVSSIS |
| Chain 2 | | GSGSDTLYADSVKGRFTISRDNSKNTLYLQMNSLRPEDTALYYCTIGGSLSRSSQ |
| | | GTLVTVSSDKTHTCPPCPAPEAAGGPSVFLFPPKPKDTLMISRTPEVTCVVVDVS |
| | | QEDPEVQFNWYVDGVEVHNAKTKPREEQFNSTYRVVSVLTVLHQDWLNGKEY |
| | | KCKVSNKGLPSSIEKTISKAKGQPREPQVCTLPPSQEEMTKNQVSLSCAVKGFYP |
| | | SDIAVEWESNGQPENNYKTTPPVLDSDGSFFLVSKLTVDKSRWQEGNVFSCSV |
| | | MHEALHNHYTQKSLSLSLG |
| TP016 | 207 | DVQLVESGGGLVQPGGSLRLSCAASGLTFSTNPMYWYRQAPGKQRELVASISS |
| Chain 1 | | RGITNYADSVKGRFTISRDNSKNTVYLQMNSLRPEDTAVYYCRLASLSSGTVYW |
| | | GQGTLVTVSSDKTHTCPPCPAPEAAGGPSVFLFPPKPKDTLMASRTPEVTCVVV |
| | | DVSQEDPEVQFNWYVDGVEVHNAKTKPREEQFNSTYRVVSVLTVLAQDWLN |
| | | GKEYKCKVSNKGLPSSIEKTISKAKGQPREPQVYTLPPCQEEMTKNQVSLWCLV |
| | | KGFYPSDIAVEWESNGQPENNYKTTPPVLDSDGSFFLYSKLTVDKSRWQEGNV |
| | | FSCSVMHEALHNAYTQKSLSLSLG |
| TP016 | 208 | DVQLVESGGGVVQPGGSLRLSCAASGFTFRSFGMSWVRQAPGKGPEWVSSIS |
| Chain 2 | | GSGSDTLYADSVKGRFTISRDNSKNTLYLQMNSLRPEDTALYYCTIGGSLSRSSQ |
| | | GTLVTVSSDKTHTCPPCPAPEAAGGPSVFLFPPKPKDTLMASRTPEVTCVVVDV |
| | | SQEDPEVQFNWYVDGVEVHNAKTKPREEQFNSTYRVVSVLTVLAQDWLNGKE |
| | | YKCKVSNKGLPSSIEKTISKAKGQPREPQVCTLPPSQEEMTKNQVSLSCAVKGFY |
| | | PSDIAVEWESNGQPENNYKTTPPVLDSDGSFFLVSKLTVDKSRWQEGNVFSCS |
| | | VMHEALHNAYTQKSLSLSLG |
| TP019 | 209 | DVQLVESGGGLVQPGGSLRLSCAASGLTFSTNPMYWYRQAPGKQRELVASISS |
| Chain 1 | | RGITNYADSVKGRFTISRDNSKNTVYLQMNSLRPEDTAVYYCRLASLSSGTVYW |
| | | GQGTLVTVSSDKTHTCPPCPAPEAAGGPSVFLFPPKPKDTLMASRTPEVTCVVV |
| | | DVSQEDPEVQFNWYVDGVEVHNAKTKPREEQFNSTYRVVSVLTVLAQDWLN |
| | | GKEYKCKVSNKGLPSSIEKTISKAKGQPREPQVYTLPPCQEEMTKNQVSLWCLV |
| | | KGFYPSDIAVEWESNGQPENNYKTTPPVLDSDGSFFLYSKLTVDKSRWQEGNV |
| | | FSCSVMHEALHNAYTQKSLSLSLGGGGGSGGGGSGGGGSGGGGSGGGGSGG |
| | | GGSGGGGSEVQLVESGGGVVQPGGSLRLSCAASGFTFRSFGMSWVRQAPGK |
| | | GPEWVSSISGSGSDTLYADSVKGRFTISRDNSKNTLYLQMNSLRPEDTALYYCTI |
| | | GGSLSRSSQGTLVTVSS |
| TP019 | 210 | DKTHTCPPCPAPEAAGGPSVFLFPPKPKDTLMASRTPEVTCVVVDVSQEDPEV |
| Chain 2 | | QFNWYVDGVEVHNAKTKPREEQFNSTYRVVSVLTVLAQDWLNGKEYKCKVSN |
| | | KGLPSSIEKTISKAKGQPREPQVCTLPPSQEEMTKNQVSLSCAVKGFYPSDIAVE |
| | | WESNGQPENNYKTTPPVLDSDGSFFLVSKLTVDKSRWQEGNVFSCSVMHEAL |
| | | HNAYTQKSLSLSLG |

### ITEMS OF THE PRESENT TECHNOLOGY

1. Polypeptide that comprises (i) at least one domain comprising a serum albumin protein or specifically binding to a serum albumin protein and (ii) an Fc domain of an immunoglobulin G (IgG).
2. Polypeptide according to item 1, characterized in that said at least one domain that comprises a serum albumin protein is human serum albumin or a part or variant of human serum albumin.
3. Polypeptide according to item 1, characterized in that the at least one domain specifically binding to a serum albumin protein specifically binds to amino acid residues on said serum albumin protein that are not involved in binding of serum albumin to FcRn.
4. Polypeptide according to items 1 and 3, characterized in that said at least one domain specifically binding to a serum albumin protein specifically binds to domain II of human serum albumin.
5. Polypeptide according to items 1, 3 and 4 characterized in that said at least one domain specifically binding to a serum albumin protein is chosen from the group consisting of an Affibody®, a scFv, a Fab, a Designed Ankyrin Repeat Protein (DARPin®), an Albumin Binding Domain (ABD), a Nanofitin® (aka affitin) and an immunoglobulin variable domain sequence (ISVD).
6. Polypeptide according to items 1 and 3 to 5, characterized in that said at least one domain specifically binding to a serum albumin protein is at least one ISVD specifically binding to serum albumin.
7. Polypeptide according to items 1 and 3 to 6, characterized in that said at least one domain specifically binding to a serum albumin protein is at least one ISVD specifically binding to human serum albumin, wherein the ISVD is a (single) domain antibody, a Nanobody® VHH, a VHH, a humanized VHH, or a camelized VH.
8. Polypeptide according to any of items 1 to 7, wherein said Fc domain of an IgG is an Fc region of an immunoglobulin G type 1, (IgG1), an immunoglobulin G type 2 (IgG2), an immunoglobulin G type 3 (IgG3), or an immunoglobulin G type 4 (IgG4).
9. Polypeptide according to any of items 1 to 8, wherein said Fc domain is a native Fc domain of an immunoglobulin G or is a variant Fc domain of an IgG or a fragment thereof.
10. Polypeptide according to any of items 1 to 9, further comprising a therapeutic moiety.
11. Polypeptide according to any of items 1 to 10, in which the therapeutic moiety comprises at least one ISVD specifically binding to a therapeutic target.
12. Polypeptide according to according to any of items 1 to 11, characterized in that it has a serum half-life in man that is at least 5%, such as at least 10%, at least 25%, at least 50%, at least 100%, at least 200%, at least 300%, at least 400% or at least 500% of the half-life of serum albumin in man.
13. Pharmaceutical composition comprising a polypeptide according to any one of items 1 to 12.
14. Nucleic acid or nucleic acid sequence encoding a polypeptide according to any of items 1 to 12.
15. Vector comprising a nucleic acid or nucleic acid sequence according to item 14.
16. Host cell transformed or transfected with the nucleic acid or nucleic acid sequence according to item 14 or with the vector according to item 15.
17. A method or process for producing the polypeptides according to any of items 1 to 12, said method at least comprising the steps of:
   a. expressing, in a suitable (non-human) host cell or host organism or in another suitable expression system, a nucleic acid sequence; optionally followed by:
   b. isolating and/or purifying the polypeptide according to any of items 1 to 12.
18. Polypeptide according to any of items 1 to 12 for use in treating a subject in need thereof.
19. Polypeptide according to any of items 1 to 12 for use in therapy.
20. Kit comprising a polypeptide according to any of items 1 to 12, a nucleic acid or nucleic acid sequence according to item 14, a vector according to item 15, or a host cell according to item 16.

1. Polypeptide that comprises (i) at least one domain comprising a serum albumin protein or specifically binding to a serum albumin protein and (ii) an Fc domain of an immunoglobulin G (IgG), wherein the polypeptide does not comprise or consist of one of the polypeptides as depicted in the below table:

| | Masking | | | |
|---|---|---|---|---|
| Polypeptide | domain | Linker | CTLA4 ECD | Ig |
| mAlb- | Albumin Glu25- | Core hinge-lower | CTLA4 Ile38- | IgG₂ₐ Hinge- |
| CTLA4Ig | Ala609 | hinge/upper CH2: | Ser160 | CH2—CH3 |
| | (Protein ID: | CPPCKCPAPNLLGGP | (Protein ID: | (IMGT |
| | NP_033784.2) | | NP_033973.2) | Accnum: |
| | | | | V00825) |
| hAlb- | Albumin Asp25- | Core hinge-lower | CTLA4 Ala37- | IgG₁ Hinge- |
| CTLA4Ig | Leu609 | hinge/upper CH2: | Asp161 | CH2—CH3 |
| | (Protein ID: | CPPCPAPELLGGP | (Protein ID: | (IMGT |
| | NP_000468.1) | | NP_005205.2) | Accnum: |
| | | | | J00228) |
| mAlb- | Albumin Glu25- | MMP substrate: | CTLA4 Ile38- | IgG₂ₐ Hinge- |
| MMP- | Ala609 | GPLGMWSRAAQPA | Ser160 | CH2—CH3 |
| CTLA4Ig | (Protein ID: | | (Protein ID: | (IMGT |
| | NP_033784.2) | | NP.033973.2) | Accnum: |
| | | | | V00825) |

-IMGT, the international ImMunoGeneTics information system (https://www.imgt.org/).
-N/A, not applicable.

2. Polypeptide according to claim 1, characterized in that said at least one domain that comprises a serum albumin protein is human serum albumin or a part or variant of human serum albumin.

3. Polypeptide according to claim 1, characterized in that the at least one domain specifically binding to a serum albumin protein specifically binds to amino acid residues on said serum albumin protein that are not involved in binding of serum albumin to FcRn.

4. Polypeptide according to claim 1, characterized in that said at least one domain specifically binding to a serum albumin protein specifically binds to domain II of human serum albumin.

5. Polypeptide according to claim 1 characterized in that said at least one domain specifically binding to a serum albumin protein is chosen from the group consisting of an affibody molecule, a scFv, a Fab, a Designed Ankyrin Repeat Protein (DARPin), an Albumin Binding Domain (ABD), an affitin and an immunoglobulin variable domain sequence (ISVD).

6. Polypeptide according to claim 1, characterized in that said at least one domain specifically binding to a serum albumin protein is at least one ISVD specifically binding to serum albumin.

7. Polypeptide according to claim 1, characterized in that said at least one domain specifically binding to a serum albumin protein is at least one ISVD specifically binding to human serum albumin, wherein the ISVD is a (single) domain antibody, a VHH, a humanized VHH, or a camelized VH.

8. Polypeptide according to claim 1, wherein said Fc domain of an IgG is an Fc region of an immunoglobulin G type 1, (IgG 1), an immunoglobulin G type 2 (IgG2), an immunoglobulin G type 3 (IgG3), or an immunoglobulin G type 4 (IgG4).

9. Polypeptide according to claim 1, wherein said Fc domain is a native Fc domain of an immunoglobulin G or is a variant Fc domain of an IgG or a fragment thereof.

10. Polypeptide according to claim 1, further comprising a therapeutic moiety.

11. Polypeptide according to claim 1, in which the therapeutic moiety comprises at least one ISVD specifically binding to a therapeutic target.

12. Polypeptide according to claim 1, characterized in that the polypeptide has a serum half-life in man that is at least 5%, such as at least 10%, at least 25%, at least 50%, at least 100%, at least 200%, at least 300%, at least 400% or at least 500% of the half-life of serum albumin in man.

13. Pharmaceutical composition comprising a polypeptide according to claim 1.

14. Nucleic acid or nucleic acid sequence encoding a polypeptide according to claim 1.

15. Vector comprising a nucleic acid or nucleic acid sequence according to claim 14.

16. Host cell or (non-human) host organism transformed or transfected with the nucleic acid or nucleic acid sequence according to claim 14 or with a vector comprising a nucleic acid or nucleic acid sequence according to claim 14.

17. A method or process for producing a polypeptide, said method at least comprising the steps of:
a. expressing, in a suitable host cell or (non-human) host organism or in another suitable expression system, a nucleic acid sequence encoding the polypeptide according to claim 1; optionally followed by:
b. isolating and/or purifying the polypeptide.

18. A method for treating a subject in need thereof, comprising administering to the subject a polypeptide according to claim 1.

19. Kit comprising a polypeptide according to claim 1.


SEQUENCE LISTING (260 sequences)

SEQ ID NO: 1 - protein, 5 aa, synthetic construct
  REGION 1..5: note = CDR1 Alb 23 Kabat
SFGMS

SEQ ID NO: 2 - protein, 17 aa, synthetic construct
  REGION 1..17: note = CDR2 Alb 23 Kabat
SISGSGSDTLYADSVKG

SEQ ID NO: 3 - protein, 6 aa, synthetic construct
  REGION 1..6: note = CDR3 Alb 23 Kabat
GGSLSR

SEQ ID NO: 4 - protein, 10 aa, synthetic construct
  REGION 1..10: note = CDR1 Alb 23 AbM
GFTFRSFGMS

SEQ ID NO: 5 - protein, 10 aa, synthetic construct
  REGION 1..10: note = CDR2 Alb 23 AbM
SISGSGSDTL

SEQ ID NO: 6 - protein, 6 aa, synthetic construct
  REGION 1..6: note = CDR3 Alb 23 AbM
GGSLSR

SEQ ID NO: 7 - protein, 115 aa, synthetic construct [sequence omitted]

SEQ ID NO: 8 - protein, 115 aa, synthetic construct [sequence omitted]

SEQ ID NO: 9 - protein, 116 aa, synthetic construct
  REGION 1..116: note = Alb129
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 10 - protein, 116 aa, synthetic construct
  REGION 1..116: note = Alb132
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 11 - protein, 115 aa, synthetic construct [sequence omitted]

SEQ ID NO: 12 - protein, 116 aa, synthetic construct
  REGION 1..116: note = Alb11 (S112K)-A
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 13 - protein, 115 aa, synthetic construct
  REGION 1..115: note = Alb82
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 14 - protein, 116 aa, synthetic construct
  REGION 1..116: note = Alb82-A
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 15 - protein, 117 aa, synthetic construct
  REGION 1..117: note = Alb82-AA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 16 - protein, 118 aa, synthetic construct
  REGION 1..118: note = Alb82-AAA
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 17 - protein, 116 aa, synthetic construct
  REGION 1..116: note = Alb82-G
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 18 - protein, 117 aa, synthetic construct
  REGION 1..117: note = Alb82-GG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 19 - protein, 118 aa, synthetic construct
  REGION 1..118: note = Alb82-GGG
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 20 - protein, 115 aa, synthetic construct
  REGION 1..115: note = Alb23002
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 21 - protein, 116 aa, synthetic construct
  REGION 1..116: note = Alb223
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 22 - protein, 585 aa, Homo sapiens
  REGION 1..585: note = Human serum albumin (1)
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 23 - protein, 585 aa, Homo sapiens [sequence omitted]

SEQ ID NO: 24 - protein, 274 aa, Homo sapiens
  REGION 1..274: note = Human FcRn
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 25 - skipped

SEQ ID NO: 26 - protein, 5 aa, synthetic construct
  REGION 1..5: note = 5GS linker
GGGGS

SEQ ID NO: 27 - protein, 7 aa, synthetic construct
  REGION 1..7: note = 7GS linker
SGGSGGS

SEQ ID NO: 28 - protein, 8 aa, synthetic construct
  REGION 1..8: note = 8GS linker
GGGGSGGS

SEQ ID NO: 29 - protein, 9 aa, synthetic construct
  REGION 1..9: note = 9GS linker
GGGGSGGGS

SEQ ID NO: 30 - protein, 10 aa, synthetic construct
  REGION 1..10: note = 10GS linker
GGGGSGGGGS

SEQ ID NO: 31 - protein, 15 aa, synthetic construct
  REGION 1..15: note = 15GS linker
GGGGSGGGGSGGGGS

SEQ ID NO: 32 - protein, 18 aa, synthetic construct
  REGION 1..18: note = 18GS linker
GGGGSGGGGSGGGGSGGS

SEQ ID NO: 33 - protein, 20 aa, synthetic construct
  REGION 1..20: note = 20GS linker
GGGGSGGGGSGGGGSGGGGS

SEQ ID NO: 34 - protein, 25 aa, synthetic construct
  REGION 1..25: note = 25GS linker
GGGGSGGGGSGGGGSGGGGSGGGGS

SEQ ID NO: 35 - protein, 30 aa, synthetic construct
  REGION 1..30: note = 30GS linker
GGGGSGGGGSGGGGSGGGGSGGGGSGGGGS

SEQ ID NO: 36 - protein, 35 aa, synthetic construct
  REGION 1..35: note = 35GS linker
GGGGSGGGGSGGGGSGGGGSGGGGSGGGGSGGGGS

SEQ ID NO: 37 - protein, 40 aa, synthetic construct
  REGION 1..40: note = 40GS linker
GGGGSGGGGSGGGGSGGGGSGGGGSGGGGSGGGGSGGGGS

SEQ ID NO: 38 - protein, 10 aa, synthetic construct
  REGION 1..10: note = Short G1 hinge
DKTHTCPPCP

SEQ ID NO: 39 - protein, 15 aa, synthetic construct
  REGION 1..15: note = G1 hinge
EPKSCDKTHTCPPCP

SEQ ID NO: 40 - protein, 24 aa, synthetic construct
  REGION 1..24: note = 9GS-G1 hinge
GGGGSGGGSEPKSCDKTHTCPPCP

SEQ ID NO: 41 - protein, 12 aa, synthetic construct
  REGION 1..12: note = Llama upper long hinge region
EPKTPKPQPAAA

SEQ ID NO: 42 - protein, 62 aa, synthetic construct
  REGION 1..62: note = G3 hinge
ELKTPLGDTTHTCPRCPEPKSCDTPPPCPRCPEPKSCDTPPPCPRCPEPKSCDTPPPCPRCP

SEQ ID NO: 43 - protein, 5 aa, synthetic construct
  REGION 1..5: note = Hallmark Residues at positions 43-47
KEREL

SEQ ID NO: 44 - protein, 5 aa, synthetic construct
  REGION 1..5: note = Hallmark Residues at positions 43-47
KEREF

SEQ ID NO: 45 - protein, 5 aa, synthetic construct
KQREL

SEQ ID NO: 46 - protein, 5 aa, synthetic construct
  REGION 1..5: note = Hallmark Residues at positions 43-47
KQREF

SEQ ID NO: 47 - protein, 5 aa, synthetic construct
  REGION 1..5: note = Hallmark Residues at positions 43-47
KEREG

SEQ ID NO: 48 - protein, 5 aa, synthetic construct
  REGION 1..5: note = Hallmark Residues at positions 43-47
KQREW

SEQ ID NO: 49 - protein, 5 aa, synthetic construct
  REGION 1..5: note = Hallmark Residues positions 43-47
KQREG

SEQ ID NO: 50 - protein, 5 aa, synthetic construct
TEREL

SEQ ID NO: 51 - protein, 5 aa, synthetic construct
  REGION 1..5: note = Hallmark Residues at positions 43-47
TQREL

SEQ ID NO: 52 - protein, 5 aa, synthetic construct
  REGION 1..5: note = Hallmark Residues positions 43-47
DECKL

SEQ ID NO: 53 - protein, 5 aa, synthetic construct
  REGION 1..5: note = Hallmark Residues positions 43-47
  VARIANT 5: note = X can be L or R
KECEX

SEQ ID NO: 54 - protein, 5 aa, synthetic construct
  REGION 1..5: note = Hallmark Residues positions 43-47
KQCEL

SEQ ID NO: 55 - protein, 5 aa, synthetic construct
  REGION 1..5: note = Hallmark Residues positions 43-47
REREG

SEQ ID NO: 56 - protein, 5 aa, synthetic construct
  REGION 1..5: note = Hallmark Residues positions 43-47
  VARIANT 5: note = X can be L,F or W
RQREX

SEQ ID NO: 57 - protein, 5 aa, synthetic construct
  REGION 1..5: note = Hallmark Residues positions 43-47.
QEREG

SEQ ID NO: 58 - protein, 5 aa, synthetic construct
  REGION 1..5: note = Hallmark Residues positions 43-47
  VARIANT 5: note = X can be W, L or F
QQREX

SEQ ID NO: 59 - protein, 5 aa, synthetic construct
  REGION 1..5: note = KGREG
KGREG

SEQ ID NO: 60 - protein, 5 aa, synthetic construct
  REGION 1..5: note = Hallmark Residues positions 43-47
KDREV

SEQ ID NO: 61 - protein, 115 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 62 - protein, 123 aa, synthetic construct
EVQLVESGGGVVQPGDSLRLSCAASGGTFSTYVMGWFRAPGKEREFVSAISQNSIHTYYANSVKGRFTISRDNSKNTVYLQLNSLRPEDTALYYCAASRFTSWYTADYEYDYWGQGTLVTVSS

SEQ ID NO: 63 - protein, 123 aa, synthetic construct
EVQLVESGGGVVQPGGSLRLSCAASGLTFSSYAMGWFRQAPGKERERVVSISRGGGYTYYADSVKGRFTISRDNSENTVYLQMNSLRPEDTALYYCAAARYWATGSEYEFDYWGQGTLVTVSS

SEQ ID NO: 64 - protein, 123 aa, synthetic construct
EVQLVESGGGVVQPGGSLRLSCAASGLTFSSYAMGWFRQAPGKERERVVSISRGGGYTYYADSVKGRFTISRDNSKNTVYLQMNSLRPEDTALYYCAAARYWATGSEYEFDYWGQGTLVTVSS

SEQ ID NO: 65 - protein, 115 aa, synthetic construct [sequence omitted]

SEQ ID NO: 66 - protein, 116 aa, synthetic construct
EVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSA

SEQ ID NO: 67 - protein, 115 aa, synthetic construct [sequence omitted]

SEQ ID NO: 68 - protein, 124 aa, synthetic construct
EVQLVESGGGVVQPGGSLRLSCAASGLTFSSYAMGWFRQAPGKERERVVSISRGGGYTYYADSVKGRFTISRDNSKNTVYLQMNSLRPEDTALYYCAAARYWATGSEYEFDYWGQGTLVTVSSA

SEQ ID NO: 69 - protein, 125 aa, synthetic construct
EVQLVESGGGVVQPGDSLRLSCAASGGTFSTYVMGWFRQAPGKEREFVSAISQNSIHTYYANSVKGRFTISRDNSKNTVYLQLNSLRPEDTALYYCAASRFTSWYTADYEYDYWGQGTLVTVSSA

SEQ ID NO: 70 - protein, 25 aa, synthetic construct
EVQLVESGGGVVQPGGSLRLSCAAS

SEQ ID NO: 71 - protein, 14 aa, synthetic construct
WVRQAPGKGPEWVS

SEQ ID NO: 72 - protein, 39 aa, synthetic construct
YADSVKGRFTISRDNSKNTLYLQMNSLRPEDTALYYCTI

SEQ ID NO: 73 - protein, 11 aa, synthetic construct
SSQGTLVTVSS

SEQ ID NO: 74 - protein, 30 aa, synthetic construct
EVQLVESGGGVVQPGGSLRLSCAASGFTFR

SEQ ID NO: 75 - protein, 4 aa, synthetic construct
KERE

SEQ ID NO: 76 - protein, 32 aa, synthetic construct
RFTISRDNSKNTLYLQMNSLRPEDTALYYCTI

SEQ ID NO: 77 - protein, 10 aa, synthetic construct
GFTFSSFGMS

SEQ ID NO: 78 - protein, 5 aa, synthetic construct
TYVMG

SEQ ID NO: 79 - protein, 17 aa, synthetic construct
AISQNSIHTYYANSVKG

SEQ ID NO: 80 - protein, 10 aa, synthetic construct
GGTFSTYVMG

SEQ ID NO: 81 - protein, 10 aa, synthetic construct
AISQNSIHTY

SEQ ID NO: 82 - protein, 15 aa, synthetic construct
SRFTSWYTADYEYDY

SEQ ID NO: 83 - protein, 25 aa, synthetic construct
EVQLVESGGGVVQPGDSLRLSCAAS

SEQ ID NO: 84 - protein, 14 aa, synthetic construct
WFRQAPGKEREFVS

SEQ ID NO: 85 - protein, 39 aa, synthetic construct
YANSVKGRFTISRDNSKNTVYLQLNSLRPEDTALYYCAA

SEQ ID NO: 86 - protein, 11 aa, synthetic construct
WGQGTLVTVSS

SEQ ID NO: 87 - protein, 30 aa, synthetic construct
EVQLVESGGGVVQPGDSLRLSCAASGGTFS

SEQ ID NO: 88 - protein, 4 aa, synthetic construct
KQRE

SEQ ID NO: 89 - protein, 32 aa, synthetic construct
RFTISRDNSKNTVYLQLNSLRPEDTALYYCAA

SEQ ID NO: 90 - protein, 10 aa, synthetic construct
GLTFSSYAMG

SEQ ID NO: 91 - protein, 10 aa, synthetic construct
SISRGGGYTY

SEQ ID NO: 92 - protein, 14 aa, synthetic construct
ARYWATGSEYEFDY

SEQ ID NO: 93 - protein, 5 aa, synthetic construct
SYAMG

SEQ ID NO: 94 - protein, 17 aa, synthetic construct
SISRGGGYTYYADSVKG

SEQ ID NO: 95 - protein, 4 aa, synthetic construct
TERE

SEQ ID NO: 96 - protein, 14 aa, synthetic construct
WFRQAPGKERERVV

SEQ ID NO: 97 - protein, 39 aa, synthetic construct
YADSVKGRFTISRDNSKNTVYLQMNSLRPEDTALYYCAA

SEQ ID NO: 98 - protein, 4 aa, synthetic construct
TQRE

SEQ ID NO: 99 - protein, 30 aa, synthetic construct
EVQLVESGGGVVQPGGSLRLSCAASGLTFS

SEQ ID NO: 100 - protein, 4 aa, synthetic construct
KECE

SEQ ID NO: 101 - protein, 32 aa, synthetic construct
RFTISRDNSKNTVYLQMNSLRPEDTALYYCAA

SEQ ID NO: 102 - protein, 124 aa, synthetic construct
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 103 - protein, 64 aa, synthetic construct
VKVKFWPRGEEKVVDTSKIAWVLRADKTVMFKYDDNGKKGYGVVLEKDAPKELLDMLARAEREK

SEQ ID NO: 104 - protein, 45 aa, synthetic construct
LKEAKEKAIEELKKAGITSDYYFDLINKAKTVEGVNALKDEILKA

SEQ ID NO: 105 - protein, 126 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSS

SEQ ID NO: 106 - protein, 118 aa, synthetic construct
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSS

SEQ ID NO: 107 - protein, 120 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSS

SEQ ID NO: 108 - protein, 107 aa, synthetic construct [sequence omitted]

SEQ ID NO: 109 - protein, 609 aa, Homo sapiens [sequence omitted]

SEQ ID NO: 110 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAQTFTFHADICTLSEKERQIKKQMALVELVKHKPKATKEQLKAVMDDFAAFVEKCCKADDKETCFAEEGPKLVAASQAALGL

SEQ ID NO: 111 - protein, 13 aa, synthetic construct
GPLGMWSRAAQPA

SEQ ID NO: 112 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 4 aa, synthetic construct
KQCE

SEQ ID NO: 115 - protein, 216 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 116 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 118 - protein, 39 aa, synthetic construct
YADSVKGRFTISRDNSENTVYLQMNSLRPEDTALYYCAA

SEQ ID NO: 119 - protein, 480 aa, synthetic construct
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGSEVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSA

SEQ ID NO: 120 - protein, 352 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 121 - protein, 469 aa, synthetic construct
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 122 - protein, 352 aa, synthetic construct; identical to SEQ ID NO: 120

SEQ ID NO: 123 - protein, 480 aa, synthetic construct
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGSEVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSA

SEQ ID NO: 124 - protein, 352 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 125 - protein, 8 aa, synthetic construct
GPLGMWSR

SEQ ID NO: 126 - protein, 6 aa, synthetic construct
GPLGVR

SEQ ID NO: 127 - protein, 449 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 449 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 129 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 585 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGSEVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSA

SEQ ID NO: 131 - protein, 15 aa, synthetic construct
CPPCKCPAPNLLGGP

SEQ ID NO: 132 - protein, 13 aa, synthetic construct
CPPCPAPELLGGP

SEQ ID NO: 133 - protein, 574 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 134 - protein, 15 aa, synthetic construct
PRGPTIKPCPPCKCP

SEQ ID NO: 135 - protein, 13 aa, synthetic construct
GPLGMWSRGAQPA

SEQ ID NO: 136 - protein, 574 aa, synthetic construct; identical to SEQ ID NO: 133

SEQ ID NO: 137 - protein, 574 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 138 - protein, 32 aa, synthetic construct
RFTISRDNSENTVYLQMNSLRPEDTALYYCAA

SEQ ID NO: 139 - protein, 514 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSA

SEQ ID NO: 140 - protein, 352 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLG

SEQ ID NO: 141 - protein, 477 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 142 - protein, 4 aa, synthetic construct
RERE

SEQ ID NO: 143 - protein, 503 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSA

SEQ ID NO: 144 - protein, 4 aa, synthetic construct
RQRE

SEQ ID NO: 145 - protein, 503 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 146 - protein, 4 aa, synthetic construct
QERE

SEQ ID NO: 147 - protein, 503 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 148 - protein, 4 aa, synthetic construct
QQRE

SEQ ID NO: 149 - protein, 511 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGGSLRLSCAASGLTFSSYAMGWFRQAPGKERERVVSISRGGGYTYYADSVKGRFTISRDNSKNTVYLQMNSLRPEDTALYYCAAARYWATGSEYEFDYWGQGTLVTVSSA

SEQ ID NO: 150 - protein, 4 aa, synthetic construct
KGRE

SEQ ID NO: 151 - protein, 512 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGDSLRLSCAASGGTFSTYVMGWFRQAPGKEREFVSAISQNSIHTYYANSVKGRFTISRDNSKNTVYLQLNSLRPEDTALYYCAASRFTSWYTADYEYDYWGQGTLVTVSSA

SEQ ID NO: 152 - protein, 4 aa, synthetic construct
KDRE

SEQ ID NO: 153 - protein, 352 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 154 - protein, 511 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLGGGGGSGGGGSGGGGSGGGGSGGGGSGGGGSGGGGSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 155 - protein, 4 aa, synthetic construct
GLEW

SEQ ID NO: 156 - protein, 451 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLGGGGGSGGGGSGGGGSGGGGSGGGGSGGGGSGGGGSVKVKFWPRGEEKVVDTSKIAWVLRADKTVMFKYDDNGKKGYGVVLEKDAPKELLDMLARAEREK

SEQ ID NO: 157 - protein, 5 aa, synthetic construct
NVCEL

SEQ ID NO: 158 - protein, 432 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLGGGGGSGGGGSGGGGSGGGGSGGGGSGGGGSGGGGSLKEAKEKAIEELKKAGITSDYYFDLINKAKTVEGVNALKDEILKA

SEQ ID NO: 159 - skipped

SEQ ID NO: 160 - protein, 972 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLGGGGGSGGGGSGGGGSGGGGSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 161 - skipped

SEQ ID NO: 162 - protein, 972 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLGGGGGSGGGGSGGGGSGGGGSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAQTFTFHADICTLSEKERQIKKQMALVELVKHKPKATKEQLKAVMDDFAAFVEKCCKADDKETCFAEEGPKLVAASQAALGL

SEQ ID NO: 163 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPG

SEQ ID NO: 164 - protein, 217 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 165 - protein, 503 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRDPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 166 - protein, 352 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLMISRDPEVTCVVVDVSQEDPEVQFNWYVDGVEVHNAKTKPREEQFNSTYRVVSVLQVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLG

SEQ ID NO: 167 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 168 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 169 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 170 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 173 - protein, 503 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 174 - protein, 352 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLG

SEQ ID NO: 175 - protein, 514 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSA

SEQ ID NO: 176 - skipped

SEQ ID NO: 177 - protein, 514 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSA

SEQ ID NO: 178 - protein, 503 aa, synthetic construct
DVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSDKTHTCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 179 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPG

SEQ ID NO: 180 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 181 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 182 - protein, 127 aa, synthetic construct
EVQLVESGGGVVQAGGSLSISCAASGGSLSNYVLGWFRQAPGKEREFVAAINWRGDITIGPPNVEGRFTISRDNAKNTGYLQMNSLAPDDTALYYCGAGTPLNPGAYIYDWSYDYWGRGTLVTVSSA

SEQ ID NO: 183 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGK

SEQ ID NO: 184 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPG

SEQ ID NO: 185 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPG

SEQ ID NO: 186 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 187 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLG

SEQ ID NO: 188 - protein, 216 aa, synthetic construct
APEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 189 - protein, 216 aa, synthetic construct
APEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNRFTQKSLSLSLG

SEQ ID NO: 190 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 191 - protein, 446 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 192 - protein, 446 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 193 - protein, 120 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 194 - protein, 213 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 106 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIK

SEQ ID NO: 196 - protein, 117 aa, synthetic construct
DVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSS

SEQ ID NO: 197 - protein, 117 aa, synthetic construct
EVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSS

SEQ ID NO: 198 - protein, 216 aa, synthetic construct
APEAAGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNAYTQKSLSLSLG

SEQ ID NO: 199 - protein, 216 aa, synthetic construct
APEAAGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNAYTQKSLSLSLG

SEQ ID NO: 200 - protein, 10 aa, synthetic construct
DKTHTCPSCP

SEQ ID NO: 201 - protein, 343 aa, synthetic construct
DVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 202 - protein, 343 aa, synthetic construct
DVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 203 - protein, 493 aa, synthetic construct
DVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 204 - protein, 226 aa, synthetic construct
DKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 205 - protein, 495 aa, synthetic construct
DVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSGGGGSGGGGSGGGGSGGGGSEVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSS

SEQ ID NO: 206 - protein, 341 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSDKTHTCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 207 - protein, 343 aa, synthetic construct
DVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSSDKTHTCPPCPAPEAAGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNAYTQKSLSLSLG

SEQ ID NO: 208 - protein, 341 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSDKTHTCPPCPAPEAAGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNAYTQKSLSLSLG

SEQ ID NO: 209 - protein, 493 aa, synthetic construct
DVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSSDKTHTCPPCPAPEAAGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNAYTQKSLSLSLGGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 210 - protein, 226 aa, synthetic construct
DKTHTCPPCPAPEAAGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNAYTQKSLSLSLG

SEQ ID NO: 211 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALKFHYTQKSLSLSPGK

SEQ ID NO: 212 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 214 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 216 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 217 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 218 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 219 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 220 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRAFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 221 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSAGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 222 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFAMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 223 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGASWVRQAPGKGPEWVSSISGSGSDTLYADSVKGRFTISRDNSKNTLYLQMNSLRPEDTAVYYCTIGGSLSRSSQGTLVTVSS

SEQ ID NO: 224 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMAWVRQAPGKGPEWVSSISGSGSDTLYADSVKGRFTISRDNSKNTLYLQMNSLRPEDTAVYYCTIGGSLSRSSQGTLVTVSS

SEQ ID NO: 225 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSAISGSGSDTLYADSVKGRFTISRDNSKNTLYLQMNSLRPEDTAVYYCTIGGSLSRSSQGTLVTVSS

SEQ ID NO: 226 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSASGSGSDTLYADSVKGRFTISRDNSKNTLYLQMNSLRPEDTAVYYCTIGGSLSRSSQGTLVTVSS

SEQ ID NO: 227 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSIAGSGSDTLYADSVKGRFTISRDNSKNTLYLQMNSLRPEDTAVYYCTIGGSLSRSSQGTLVTVSS

SEQ ID NO: 228 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISASGSDTLYADSVKGRFTISRDNSKNTLYLQMNSLRPEDTAVYYCTIGGSLSRSSQGTLVTVSS

SEQ ID NO: 229 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGAGSDTLYADSVKGRFTISRDNSKNTLYLQMNSLRPEDTAVYYCTIGGSLSRSSQGTLVTVSS

SEQ ID NO: 230 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSASDTLYADSVKGRFTISRDNSKNTLYLQMNSLRPEDTAVYYCTIGGSLSRSSQGTLVTVSS

SEQ ID NO: 231 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGADTLYADSVKGRFTISRDNSKNTLYLQMNSLRPEDTAVYYCTIGGSLSRSSQGTLVTVSS

SEQ ID NO: 232 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSATLYADSVKGRFTISRDNSKNTLYLQMNSLRPEDTAVYYCTIGGSLSRSSQGTLVTVSS

SEQ ID NO: 233 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDALYADSVKGRFTISRDNSKNTLYLQMNSLRPEDTAVYYCTIGGSLSRSSQGTLVTVSS

SEQ ID NO: 234 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTAYADSVKGRFTISRDNSKNTLYLQMNSLRPEDTAVYYCTIGGSLSRSSQGTLVTVSS

SEQ ID NO: 235 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLAADSVKGRFTISRDNSKNTLYLQMNSLRPEDTAVYYCTIGGSLSRSSQGTLVTVSS

SEQ ID NO: 236 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYAASVKGRFTISRDNSKNTLYLQMNSLRPEDTAVYYCTIGGSLSRSSQGTLVTVSS

SEQ ID NO: 237 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADAVKGRFTISRDNSKNTLYLQMNSLRPEDTAVYYCTIGGSLSRSSQGTLVTVSS

SEQ ID NO: 238 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSAKGRFTISRDNSKNTLYLQMNSLRPEDTAVYYCTIGGSLSRSSQGTLVTVSS

SEQ ID NO: 239 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVAGRFTISRDNSKNTLYLQMNSLRPEDTAVYYCTIGGSLSRSSQGTLVTVSS

SEQ ID NO: 240 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKARFTISRDNSKNTLYLQMNSLRPEDTAVYYCTIGGSLSRSSQGTLVTVSS

SEQ ID NO: 241 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIAGSLSRSSQGTLVTVSS

SEQ ID NO: 242 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGASLSRSSQGTLVTVSS

SEQ ID NO: 243 - protein, 361 aa, unidentified
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGALSRSSQGTLVTVSS

SEQ ID NO: 244 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSASRSSQGTLVTVSS

SEQ ID NO: 245 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLARSSQGTLVTVSS

SEQ ID NO: 246 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSASSQGTLVTVSS

SEQ ID NO: 247 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGTLSRSSQGTLVTVSS

SEQ ID NO: 248 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFAMSWVRQAPGKGPEWVSSISGSGSDTLYADSVKGRFTISRDNSKNTLYLQMNSLRPEDTAVYYCTIGGALSRSSQGTLVTVSS

SEQ ID NO: 249 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFAMSWVRQAPGKGPEWVSSISASGSDTLYADSVKGRFTISRDNSKNTLYLQMNSLRPEDTAVYYCTIGGSLSRSSQGTLVTVSS

SEQ ID NO: 250 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSIAASGSDTLYADSVKGRFTISRDNSKNTLYLQMNSLRPEDTAVYYCTIGGSLSRSSQGTLVTVSS

SEQ ID NO: 251 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSIAGSGSDTLYADSVKGRFTISRDNSKNTLYLQMNSLRPEDTAVYYCTIGGALSRSSQGTLVTVSS

SEQ ID NO: 252 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSHGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 253 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGHSWVRQAPGKGPEWVSSISGSGSDTLYADSVKGRFTISRDNSKNTLYLQMNSLRPEDTAVYYCTIGGSLSRSSQGTLVTVSS

SEQ ID NO: 254 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSHSDTLYADSVKGRFTISRDNSKNTLYLQMNSLRPEDTAVYYCTIGGSLSRSSQGTLVTVSS

SEQ ID NO: 255 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTHYADSVKGRFTISRDNSKNTLYLQMNSLRPEDTAVYYCTIGGSLSRSSQGTLVTVSS

SEQ ID NO: 256 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIHGSLSRSSQGTLVTVSS

SEQ ID NO: 257 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGHSLSRSSQGTLVTVSS

SEQ ID NO: 258 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSHSRSSQGTLVTVSS

SEQ ID NO: 259 - protein, 361 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSEVQLLESGGGLVQPGGSLRLSCAASGFTFRSFHMSWVRQAPGKGPEWVSSISGSGSDTLYADSVKGRFTISRDNSKNTLYLQMNSLRPEDTAVYYCTIGGHLSRSSQGTLVTVSS

SEQ ID NO: 260 - protein, 372 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALKFHYTQKSLSLSPGGGGGSGGGGSGGGGSGGGGSGGGGSGGGGSEVQLLESGGGLVQPGGSLRLSCAASGFTFRSAGMSWVRQAPGKGPEWVSSISGSGSDTLYADSVKGRFTISRDNSKNTLYLQMNSLRPEDTAVYYCTIGGSLSRSSQGTLVTVSSA